SEQUENCE LISTING (161 sequences)

SEQ ID NO: 1 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methylguanosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluoroguanosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ggctacgaaaatccaacctaa

SEQ ID NO: 2 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
tagtgcatgaatagattctca

SEQ ID NO: 3 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 4 - DNA, 21 bp, synthetic construct
  source 1..21: mol_type = other DNA
  modified_base 1: mod_base = OTHER; note = 2'-O-methylguanosine-3'-phosphorothioate
  modified_base 2: mod_base = OTHER; note = 2'-O-methylguanosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-O-hexadecyl-cytidine-3'-phosphate
  modified_base 7: mod_base = OTHER; note = 2'-O-methylguanosine-3'-phosphate
  modified_base 8: mod_base = OTHER; note = 2'-O-methyladenosine-3'-phosphate
  modified_base 10: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
  misc_feature 1..8: note = RNA
  misc_feature 10: note = RNA
  misc_feature 12..21: note = RNA
  misc_feature 9: note = DNA
  misc_feature 11: note = DNA
ggctacgaaaatccaacctaa

SEQ ID NO: 5 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = 2'-O-hexadecyl-cytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-deoxy-2'-fluoroadenosine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
aaagagcaaaactattcagaa

SEQ ID NO: 6 - DNA, 21 bp, synthetic construct
  source 1..21: mol_type = other DNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = 2'-O-hexadecyl-cytidine-3'-phosphate
  modified_base 8: mod_base = OTHER; note = 2'-O-methyladenosine-3'-phosphate
  modified_base 10: mod_base = OTHER; note = 2'-O-methyladenosine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
  misc_feature 1..8: note = RNA
  misc_feature 10: note = RNA
  misc_feature 12..21: note = RNA
  misc_feature 9: note = DNA
  misc_feature 11: note = DNA
aaagagcaaaactattcagaa

SEQ ID NO: 7 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-hexadecyl-guanosine-3'-phosphate
  modified_base 7: mod_base = OTHER; note = 2'-deoxy-2'-fluoroadenosine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluoroadenosine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
tttatgatttactcattatca

SEQ ID NO: 8 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 9 - RNA, 21 nt, synthetic construct
  source 1..21: mol_type = other RNA
  modified_base 1: mod_base = OTHER; note = 2'-O-hexadecyl-adenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 10 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-docosanyl-cytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 11 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-decyl-cytidine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
aaaatccaacctacaagttca

SEQ ID NO: 12 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluoroguanosine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphate
  modified_base 20: mod_base = OTHER; note = 2'-O-methylcytidine-3'-phosphate
  modified_base 21: mod_base = OTHER; note = 2'-O-methylcytidine-3'-phosphorothioate
  modified_base 22: mod_base = OTHER; note = 2'-O-methylguanosine-3'-phosphorothioate
  modified_base 23: mod_base = OTHER; note = 2'-O-methyluridine
ttaggttggattttcgtagccgt

SEQ ID NO: 13 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methyladenosine-3'-phosphate
  modified_base 7: mod_base = OTHER; note = Thymidine-glycol nucleic acid (GNA), S-Isomer
  modified_base 8: mod_base = OTHER; note = 2'-O-methylcytidine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluoroguanosine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methyluridine
tgagaatctattcatgcactagt

SEQ ID NO: 14 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 15 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphate
  modified_base 20: mod_base = OTHER; note = 2'-O-methylcytidine-3'-phosphate
  modified_base 21: mod_base = OTHER; note = 2'-O-methylcytidine-3'-phosphorothioate
  modified_base 22: mod_base = OTHER; note = 2'-O-methylguanosine-3'-phosphorothioate
  modified_base 23: mod_base = OTHER; note = 2'-O-methyluridine
  misc_feature 1..11: note = RNA
  misc_feature 13..15: note = RNA
  misc_feature 17..23: note = RNA
  misc_feature 12: note = DNA
  misc_feature 16: note = DNA
ttaggttggattttcgtagccgt

SEQ ID NO: 16 - DNA, 23 bp, synthetic construct
  source 1..23: mol_type = other DNA
  modified_base 1: mod_base = OTHER; note = 2'-O-methyluridine-3'-phosphate 5'-(E)-Vinylphosphonate
  modified_base 2: mod_base = OTHER; note = 2'-deoxy-2'-fluorouridine-3'-phosphate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyladenosine-3'-phosphate
  modified_base 8: mod_base = OTHER; note = 2'-O-methyluridine-3'-phosphate
  modified_base 9: mod_base = OTHER; note = 2'-O-methyladenosine-3'-phosphate
  modified_base 10: mod_base = OTHER; note = 2'-O-methylguanosine-3'-phosphate
  modified_base 11: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluorocytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methyluridine
  misc_feature 1..11: note = RNA
  misc_feature 13..23: note = RNA
  misc_feature 12: note = DNA
ttctgaatagttttgctctttct

SEQ ID NO: 17 - DNA, 23 bp, synthetic construct
  source 1..23: mol_type = other DNA
  modified_base 1: mod_base = OTHER; note = 2'-O-methyluridine-3'-phosphate 5'-(E)-Vinylphosphonate
  modified_base 2: mod_base = OTHER; note = 2'-deoxy-2'-fluorouridine-3'-phosphate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 7: mod_base = OTHER; note = 2'-O-methyladenosine-3'-phosphate
  modified_base 8: mod_base = OTHER; note = 2'-O-methyluridine-3'-phosphate
  modified_base 9: mod_base = OTHER; note = 2'-O-methyladenosine-3'-phosphate
  modified_base 10: mod_base = OTHER; note = 2'-O-methylguanosine-3'-phosphate
  modified_base 11: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methyluridine
  misc_feature 1..5: note = RNA
  misc_feature 7..11: note = RNA
  misc_feature 13..15: note = RNA
  misc_feature 17..23: note = RNA
  misc_feature 6: note = DNA
  misc_feature 12: note = DNA
  misc_feature 16: note = DNA
ttctgaatagttttgctctttct

SEQ ID NO: 18 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-O-methylguanosine-3'-phosphate
  modified_base 9: mod_base = OTHER; note = 2'-O-methyladenosine-3'-phosphate
  modified_base 10: mod_base = OTHER; note = 2'-O-methylguanosine-3'-phosphate
  modified_base 11: mod_base = OTHER; note = 2'-O-methyluridine-3'-phosphate
  modified_base 12: mod_base = OTHER; note = 2'-O-methyladenosine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methyluridine-3'-phosphate
  modified_base 16: mod_base = OTHER; note = 2'-deoxy-2'-fluorocytidine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methyluridine-3'-phosphorothioate
  modified_base 23: mod_base = OTHER; note = 2'-O-methylguanosine
tgataatgagtaaatcataaatg

SEQ ID NO: 19 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 20 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 21 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 22 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methyluridine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tgaacttgtaggttggattttcg

SEQ ID NO: 23 - RNA, 23 nt, synthetic construct
  source 1..23: mol_type = other RNA
acggctacgaaaatccaacctaa

SEQ ID NO: 24 - RNA, 23 nt, synthetic construct
  source 1..23: mol_type = other RNA
actagtgcatgaatagattctca

SEQ ID NO: 25 - RNA, 23 nt, synthetic construct
  source 1..23: mol_type = other RNA
cgaaaatccaacctacaagttca

SEQ ID NO: 26 - RNA, 23 nt, synthetic construct
  source 1..23: mol_type = other RNA
acggctacgaaaatccaacctaa

SEQ ID NO: 27 - RNA, 23 nt, synthetic construct
  source 1..23: mol_type = other RNA
agaaagagcaaaactattcagaa

SEQ ID NO: 28 - RNA, 23 nt, synthetic construct
  source 1..23: mol_type = other RNA
agaaagagcaaaactattcagaa

SEQ ID NO: 29 - RNA, 23 nt, synthetic construct
  source 1..23: mol_type = other RNA
catttatgatttactcattatca

SEQ ID NO: 30 - RNA, 23 nt, synthetic construct
  source 1..23: mol_type = other RNA
cgaaaatccaacctacaagttca

SEQ ID NO: 31 - RNA, 23 nt, synthetic construct
  source 1..23: mol_type = other RNA
cgaaaatccaacctacaagttca

SEQ ID NO: 32 - RNA, 23 nt, synthetic construct
  source 1..23: mol_type = other RNA
cgaaaatccaacctacaagttca

SEQ ID NO: 33 - RNA, 23 nt, synthetic construct
  source 1..23: mol_type = other RNA
cgaaaatccaacctacaagttca

SEQ ID NO: 34 - RNA, 21 nt, synthetic construct
  source 1..21: mol_type = other RNA
ggctacgaaaatccaacctaa

SEQ ID NO: 35 - RNA, 21 nt, synthetic construct
  source 1..21: mol_type = other RNA
tagtgcatgaatagattctca

SEQ ID NO: 36 - RNA, 21 nt, synthetic construct
  source 1..21: mol_type = other RNA
aaaatccaacctacaagttca

SEQ ID NO: 37 - RNA, 21 nt, synthetic construct
  source 1..21: mol_type = other RNA
ggctacgaaaatccaacctaa

SEQ ID NO: 38 - RNA, 21 nt, synthetic construct
  source 1..21: mol_type = other RNA
aaagagcaaaactattcagaa

SEQ ID NO: 39 - RNA, 21 nt, synthetic construct
  source 1..21: mol_type = other RNA
aaagagcaaaactattcagaa

SEQ ID NO: 40 - RNA, 21 nt, synthetic construct
  source 1..21: mol_type = other RNA
tttatgatttactcattatca

SEQ ID NO: 41 - RNA, 21 nt, synthetic construct
  source 1..21: mol_type = other RNA
aaaatccaacctacaagttca

SEQ ID NO: 42 - RNA, 21 nt, synthetic construct
  source 1..21: mol_type = other RNA
aaaatccaacctacaagttca

SEQ ID NO: 43 - RNA, 21 nt, synthetic construct
  source 1..21: mol_type = other RNA
aaaatccaacctacaagttca

SEQ ID NO: 44 - RNA, 21 nt, synthetic construct
  source 1..21: mol_type = other RNA
aaaatccaacctacaagttca

SEQ ID NO: 45 - RNA, 23 nt, synthetic construct
  source 1..23: mol_type = other RNA
  modified_base 7: mod_base = OTHER; note = 5'-methyluridine-3'-phosphate
ttaggttggattttcgtagccgt

SEQ ID NO: 46 - RNA, 23 nt, synthetic construct
  source 1..23: mol_type = other RNA
  modified_base 7: mod_base = OTHER; note = 5'-methyluridine-3'-phosphate
tgagaatctattcatgcactagt

SEQ ID NO: 47 - RNA, 23 nt, synthetic construct
  source 1..23: mol_type = other RNA
  modified_base 7: mod_base = OTHER; note = 5'-methyluridine-3'-phosphate
tgaacttgtaggttggattttcg

SEQ ID NO: 48 - RNA, 23 nt, synthetic construct
  source 1..23: mol_type = other RNA
  modified_base 7: mod_base = OTHER; note = 5'-methyluridine-3'-phosphate
  modified_base 12: mod_base = OTHER; note = 5'-methyluridine-3'-phosphate
ttaggttggattttcgtagccgt

SEQ ID NO: 49 - RNA, 23 nt, synthetic construct
  source 1..23: mol_type = other RNA
  modified_base 12: mod_base = OTHER; note = 5'-methyluridine-3'-phosphate
ttctgaatagttttgctctttct

SEQ ID NO: 50 - RNA, 23 nt, synthetic construct
  source 1..23: mol_type = other RNA
  modified_base 12: mod_base = OTHER; note = 5'-methyluridine-3'-phosphate
ttctgaatagttttgctctttct

SEQ ID NO: 51 - RNA, 23 nt, synthetic construct
  source 1..23: mol_type = other RNA
tgataatgagtaaatcataaatg

SEQ ID NO: 52 - RNA, 23 nt, synthetic construct
  source 1..23: mol_type = other RNA
  modified_base 7: mod_base = OTHER; note = 5'-methyluridine-3'-phosphate
tgaacttgtaggttggattttcg

SEQ ID NO: 53 - RNA, 23 nt, synthetic construct
  source 1..23: mol_type = other RNA
  modified_base 7: mod_base = OTHER; note = 5'-methyluridine-3'-phosphate
tgaacttgtaggttggattttcg

SEQ ID NO: 54 - RNA, 23 nt, synthetic construct
  source 1..23: mol_type = other RNA
  modified_base 7: mod_base = OTHER; note = 5'-methyluridine-3'-phosphate
tgaacttgtaggttggattttcg

SEQ ID NO: 55 - RNA, 23 nt, synthetic construct
  source 1..23: mol_type = other RNA
  modified_base 7: mod_base = OTHER; note = 5'-methyluridine-3'-phosphate
tgaacttgtaggttggattttcg

SEQ ID NO: 56 - RNA, 23 nt, synthetic construct
  source 1..23: mol_type = other RNA
acggctacgaaaatccaacctaa

SEQ ID NO: 57 - RNA, 23 nt, synthetic construct
  source 1..23: mol_type = other RNA
actagtgcatgaatagattctca

SEQ ID NO: 58 - RNA, 23 nt, synthetic construct
  source 1..23: mol_type = other RNA
cgaaaatccaacctacaagttca

SEQ ID NO: 59 - RNA, 23 nt, synthetic construct
  source 1..23: mol_type = other RNA
acggctacgaaaatccaacctaa

SEQ ID NO: 60 - RNA, 23 nt, synthetic construct
  source 1..23: mol_type = other RNA
agaaagagcaaaactattcagaa

SEQ ID NO: 61 - RNA, 23 nt, synthetic construct
  source 1..23: mol_type = other RNA
agaaagagcaaaactattcagaa

SEQ ID NO: 62 - RNA, 23 nt, synthetic construct
  source 1..23: mol_type = other RNA
catttatgatttactcattatca

SEQ ID NO: 63 - RNA, 23 nt, synthetic construct
  source 1..23: mol_type = other RNA
cgaaaatccaacctacaagttca

SEQ ID NO: 64 - RNA, 23 nt, synthetic construct
  source 1..23: mol_type = other RNA
cgaaaatccaacctacaagttca

SEQ ID NO: 65 - RNA, 23 nt, synthetic construct
  source 1..23: mol_type = other RNA
cgaaaatccaacctacaagttca

SEQ ID NO: 66 - RNA, 23 nt, synthetic construct
  source 1..23: mol_type = other RNA
cgaaaatccaacctacaagttca

SEQ ID NO: 67 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyluridine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyladenosine-3'-phosphate
  modified_base 6: mod_base = OTHER; note = 2'-O-hexadecyl-uridine-3'-phosphate
  modified_base 7: mod_base = OTHER; note = 2'-O-methyluridine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroadenosine-3'-phosphate
  modified_base 12: mod_base = OTHER; note = 2'-O-methylguanosine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylguanosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methylguanosine-3'-phosphate
  modified_base 19: mod_base = OTHER; note = 2'-O-methylguanosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
atcaatttcgagcagaaggaa

SEQ ID NO: 68 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-cytidine-3'-phosphate
  modified_base 7: mod_base = OTHER; note = 2'-O-methylguanosine-3'-phosphate
  modified_base 8: mod_base = OTHER; note = 2'-O-methyladenosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-O-methyladenosine-3'-phosphate
  modified_base 15: mod_base = OTHER; note = 2'-O-methylguanosine-3'-phosphate
  modified_base 16: mod_base = OTHER; note = 2'-O-methylguanosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
aatttcgagcagaaggaaaga

SEQ ID NO: 69 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methyluridine-3'-phosphorothioate
  modified_base 3: mod_base = OTHER; note = 2'-O-methylcytidine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = 2'-O-hexadecyl-cytidine-3'-phosphate
  modified_base 8: mod_base = OTHER; note = 2'-O-methyladenosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ttcgagcagaaggaaagtaaa

SEQ ID NO: 70 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-hexadecyl-adenosine-3'-phosphate
  modified_base 7: mod_base = OTHER; note = 2'-O-methyladenosine-3'-phosphate
  modified_base 8: mod_base = OTHER; note = 2'-O-methylguanosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methylcytidine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine
aaggaaagtaatggaccagta

SEQ ID NO: 71 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methyladenosine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-O-methyladenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroguanosine-3'-phosphate
  modified_base 12: mod_base = OTHER; note = 2'-O-methyladenosine-3'-phosphate
  modified_base 13: mod_base = OTHER; note = 2'-O-methylcytidine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methylguanosine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
gaaagtaatggaccagtgaaa

SEQ ID NO: 72 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-hexadecyl-cytidine-3'-phosphate
  modified_base 7: mod_base = OTHER; note = 2'-O-methylcytidine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphorothioate
  modified_base 20: mod_base = OTHER; note = 2'-O-methyluridine-3'-phosphorothioate
  modified_base 21: mod_base = OTHER; note = 2'-O-methyladenosine
caggtcctcactttaatccta

SEQ ID NO: 73 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methylcytidine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-O-hexadecyl-cytidine-3'-phosphate
  modified_base 7: mod_base = OTHER; note = 2'-O-methyluridine-3'-phosphate
  modified_base 8: mod_base = OTHER; note = 2'-O-methyluridine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methylcytidine-3'-phosphate
  modified_base 15: mod_base = OTHER; note = 2'-O-methyluridine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
cctcactttaatcctctatca

SEQ ID NO: 74 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyladenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-O-methyladenosine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyluridine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
cactttaatcctctatccaga

SEQ ID NO: 75 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methyladenosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-hexadecyl-uridine-3'-phosphate
  modified_base 7: mod_base = OTHER; note = 2'-O-methylguanosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroadenosine-3'-phosphate
  modified_base 12: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-O-methylguanosine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine
aaggatgaagagaggcatgta

SEQ ID NO: 76 - RNA, 21 nt, synthetic construct
  source 1..21: mol_type = other RNA
  modified_base 1: mod_base = OTHER; note = 2'-O-methyladenosine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-hexadecyl-uridine-3'-phosphate
  modified_base 6: mod_base = OTHER; note = 2'-O-methylguanosine-3'-phosphate
  modified_base 7: mod_base = OTHER; note = 2'-O-methyladenosine-3'-phosphate
  modified_base 8: mod_base = OTHER; note = 2'-O-methyladenosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroguanosine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-O-methylguanosine-3'-phosphate
  modified_base 15: mod_base = OTHER; note = 2'-O-methylcytidine-3'-phosphate
  modified_base 16: mod_base = OTHER; note = 2'-O-methyladenosine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methyluridine-3'-phosphorothioate
  modified_base 20: mod_base = OTHER; note = 2'-O-methyluridine-3'-phosphorothioate
  modified_base 21: mod_base = OTHER; note = 2'-O-methyladenosine
aggatgaagagaggcatgtta

SEQ ID NO: 77 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methylcytidine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-hexadecyl-guanosine-3'-phosphate
  modified_base 7: mod_base = OTHER; note = 2'-deoxy-2'-fluorocytidine-3'-phosphate
  modified_base 8: mod_base = OTHER; note = 2'-O-methylcytidine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyluridine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
ccagagccccattcattgcca

SEQ ID NO: 78 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methylcytidine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methylguanosine-3'-phosphate
  modified_base 6: mod_base = OTHER; note = 2'-O-hexadecyl-guanosine-3'-phosphate
  modified_base 7: mod_base = OTHER; note = 2'-deoxy-2'-fluorocytidine-3'-phosphate
  modified_base 8: mod_base = OTHER; note = 2'-O-methylguanosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methylguanosine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
ccatggcgaccctggaaaaga

SEQ ID NO: 79 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methylguanosine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyladenosine-3'-phosphate
  modified_base 6: mod_base = OTHER; note = 2'-O-hexadecyl-adenosine-3'-phosphate
  modified_base 7: mod_base = OTHER; note = 2'-deoxy-2'-fluoroadenosine-3'-phosphate
  modified_base 8: mod_base = OTHER; note = 2'-O-methylguanosine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluoroguanosine-3'-phosphate
  modified_base 12: mod_base = OTHER; note = 2'-O-methyladenosine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylguanosine-3'-phosphate
  modified_base 18: mod_base = OTHER; note = 2'-O-methylguanosine-3'-phosphate
  modified_base 19: mod_base = OTHER; note = 2'-O-methylcytidine-3'-phosphorothioate
  modified_base 20: mod_base = OTHER; note = 2'-O-methylcytidine-3'-phosphorothioate
  modified_base 21: mod_base = OTHER; note = 2'-O-methyladenosine
tggaaaagctgatgaaggcca

SEQ ID NO: 80 - RNA, 21 nt, synthetic construct
  source 1..21: mol_type = other RNA
  modified_base 1: mod_base = OTHER; note = 2'-O-methyluridine-3'-phosphorothioate
  modified_base 2: mod_base = OTHER; note = 2'-O-methylcytidine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-O-hexadecyl-adenosine-3'-phosphate
  modified_base 7: mod_base = OTHER; note = 2'-deoxy-2'-fluorouridine-3'-phosphate
  modified_base 8: mod_base = OTHER; note = 2'-O-methylcytidine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-O-methylcytidine-3'-phosphate
  modified_base 15: mod_base = OTHER; note = 2'-O-methylguanosine-3'-phosphate
  modified_base 16: mod_base = OTHER; note = 2'-O-methyluridine-3'-phosphate
  modified_base 17: mod_base = OTHER; note = 2'-O-methyluridine-3'-phosphate
  modified_base 18: mod_base = OTHER; note = 2'-O-methyluridine-3'-phosphate
  modified_base 19: mod_base = OTHER; note = 2'-O-methylguanosine-3'-phosphorothioate
  modified_base 20: mod_base = OTHER; note = 2'-O-methyluridine-3'-phosphorothioate
  modified_base 21: mod_base = OTHER; note = 2'-O-methyladenosine
tcataatcacattcgtttgta

SEQ ID NO: 81 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methylcytidine-3'-phosphorothioate
  modified_base 3: mod_base = OTHER; note = 2'-O-methyluridine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-hexadecyl-uridine-3'-phosphate
  modified_base 7: mod_base = OTHER; note = 2'-deoxy-2'-fluoroguanosine-3'-phosphate
  modified_base 8: mod_base = OTHER; note = 2'-O-methyladenosine-3'-phosphate
  modified_base 9: mod_base = OTHER; note = 2'-deoxy-2'-fluoroadenosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorocytidine-3'-phosphate
  modified_base 12: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-O-methyladenosine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methyluridine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
gctggtgaatcggattcctga

SEQ ID NO: 82 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methylcytidine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 5: mod_base = OTHER; note = 2'-O-methyluridine-3'-phosphate
  modified_base 6: mod_base = OTHER; note = 2'-O-hexadecyl-uridine-3'-phosphate
  modified_base 7: mod_base = OTHER; note = 2'-deoxy-2'-fluorocytidine-3'-phosphate
  modified_base 8: mod_base = OTHER; note = 2'-O-methyladenosine-3'-phosphate
  modified_base 9: mod_base = OTHER; note = 2'-deoxy-2'-fluorouridine-3'-phosphate
  modified_base 10: mod_base = OTHER; note = 2'-deoxy-2'-fluoroadenosine-3'-phosphate
  modified_base 11: mod_base = OTHER; note = 2'-deoxy-2'-fluoroadenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methylcytidine-3'-phosphate
  modified_base 16: mod_base = OTHER; note = 2'-O-methyladenosine-3'-phosphate
  modified_base 17: mod_base = OTHER; note = 2'-O-methyluridine-3'-phosphate
  modified_base 18: mod_base = OTHER; note = 2'-O-methyluridine-3'-phosphate
  modified_base 19: mod_base = OTHER; note = 2'-O-methylcytidine-3'-phosphorothioate
  modified_base 20: mod_base = OTHER; note = 2'-O-methylguanosine-3'-phosphorothioate
  modified_base 21: mod_base = OTHER; note = 2'-O-methyladenosine
gctattcataatcacattcga

SEQ ID NO: 83 - RNA, 21 nt, synthetic construct
  source 1..21: mol_type = other RNA
  modified_base 1: mod_base = OTHER; note = 2'-O-methylguanosine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methylcytidine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluorocytidine-3'-phosphate
  modified_base 8: mod_base = OTHER; note = 2'-O-methyluridine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluoroguanosine-3'-phosphate
  modified_base 11: mod_base = OTHER; note = 2'-deoxy-2'-fluoroadenosine-3'-phosphate
  modified_base 12: mod_base = OTHER; note = 2'-O-methyladenosine-3'-phosphate
  modified_base 13: mod_base = OTHER; note = 2'-O-methyladenosine-3'-phosphate
  modified_base 14: mod_base = OTHER; note = 2'-O-methyladenosine-3'-phosphate
  modified_base 15: mod_base = OTHER; note = 2'-O-methylguanosine-3'-phosphate
  modified_base 16: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphorothioate
  modified_base 20: mod_base = OTHER; note = 2'-O-methyluridine-3'-phosphorothioate
  modified_base 21: mod_base = OTHER; note = 2'-O-methyladenosine
gcgaccctggaaaagctgata

SEQ ID NO: 84 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methyluridine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 5: mod_base = OTHER; note = 2'-O-methylguanosine-3'-phosphate
  modified_base 6: mod_base = OTHER; note = 2'-O-hexadecyl-adenosine-3'-phosphate
  modified_base 7: mod_base = OTHER; note = 2'-deoxy-2'-fluoroguanosine-3'-phosphate
  modified_base 8: mod_base = OTHER; note = 2'-O-methyluridine-3'-phosphate
  modified_base 9: mod_base = OTHER; note = 2'-deoxy-2'-fluorocytidine-3'-phosphate
  modified_base 10: mod_base = OTHER; note = 2'-deoxy-2'-fluorocytidine-3'-phosphate
  modified_base 11: mod_base = OTHER; note = 2'-deoxy-2'-fluorocytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methyladenosine-3'-phosphate
  modified_base 15: mod_base = OTHER; note = 2'-O-methyladenosine-3'-phosphate
  modified_base 16: mod_base = OTHER; note = 2'-O-methylguanosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphorothioate
  modified_base 20: mod_base = OTHER; note = 2'-O-methyluridine-3'-phosphorothioate
  modified_base 21: mod_base = OTHER; note = 2'-O-methyladenosine
cttcgagtccctcaagtccta

SEQ ID NO: 85 - RNA, 21 nt, synthetic construct
  source 1..21: mol_type = other RNA
  modified_base 1: mod_base = OTHER; note = 2'-O-methylcytidine-3'-phosphorothioate
  modified_base 2: mod_base = OTHER; note = 2'-O-methylcytidine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyluridine-3'-phosphate
  modified_base 6: mod_base = OTHER; note = 2'-O-hexadecyl-cytidine-3'-phosphate
  modified_base 7: mod_base = OTHER; note = 2'-deoxy-2'-fluoroadenosine-3'-phosphate
  modified_base 8: mod_base = OTHER; note = 2'-O-methylguanosine-3'-phosphate
  modified_base 9: mod_base = OTHER; note = 2'-deoxy-2'-fluoroguanosine-3'-phosphate
  modified_base 10: mod_base = OTHER; note = 2'-deoxy-2'-fluorouridine-3'-phosphate
  modified_base 11: mod_base = OTHER; note = 2'-deoxy-2'-fluorouridine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-O-methylguanosine-3'-phosphate
  modified_base 15: mod_base = OTHER; note = 2'-O-methylcytidine-3'-phosphate
  modified_base 16: mod_base = OTHER; note = 2'-O-methyluridine-3'-phosphate
  modified_base 17: mod_base = OTHER; note = 2'-O-methyluridine-3'-phosphate
  modified_base 18: mod_base = OTHER; note = 2'-O-methyluridine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine
ccggtcaggttctgcttttaa

SEQ ID NO: 86 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methylcytidine-3'-phosphate
  modified_base 4: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = cytidine-2'-phosphate
  modified_base 8: mod_base = OTHER; note = 2'-O-methyluridine-3'-phosphate
  modified_base 9: mod_base = OTHER; note = 2'-O-methylguanosine-3'-phosphate
  modified_base 10: mod_base = OTHER; note = 2'-O-methylcytidine-3'-phosphate
  modified_base 11: mod_base = OTHER; note = 2'-O-methyluridine-3'-phosphate
  modified_base 12: mod_base = OTHER; note = 2'-O-methylcytidine-3'-phosphate
  modified_base 13: mod_base = OTHER; note = 2'-O-methylguanosine-3'-phosphate
  modified_base 14: mod_base = OTHER; note = 2'-deoxy-2'-fluoroadenosine-3'-phosphate
  modified_base 15: mod_base = OTHER; note = 2'-O-methyladenosine-3'-phosphate
  modified_base 16: mod_base = OTHER; note = 2'-deoxy-2'-fluoroadenosine-3'-phosphate
  modified_base 17: mod_base = OTHER; note = 2'-O-methyluridine-3'-phosphate
  modified_base 18: mod_base = OTHER; note = 2'-O-methyluridine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methyluridine-3'-phosphorothioate
  modified_base 22: mod_base = OTHER; note = 2'-O-methylguanosine-3'-phosphorothioate
  modified_base 23: mod_base = OTHER; note = 2'-O-methylguanosine
  misc_feature 1..4: note = RNA
  misc_feature 6..23: note = RNA
  misc_feature 5: note = DNA
ttccttctgctcgaaattgatgg

SEQ ID NO: 87 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorocytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 6: mod_base = OTHER; note = 2'-O-methylcytidine-3'-phosphate
  modified_base 7: mod_base = OTHER; note = cytidine-2'-phosphate
  modified_base 8: mod_base = OTHER; note = 2'-O-methyluridine-3'-phosphate
  modified_base 9: mod_base = OTHER; note = 2'-O-methyluridine-3'-phosphate
  modified_base 10: mod_base = OTHER; note = 2'-O-methylcytidine-3'-phosphate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methylcytidine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluoroguanosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methyladenosine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylguanosine-3'-phosphorothioate
  modified_base 23: mod_base = OTHER; note = 2'-O-methylguanosine
  misc_feature 1..4: note = RNA
  misc_feature 6..23: note = RNA
  misc_feature 5: note = DNA
tctttccttctgctcgaaattgg

SEQ ID NO: 88 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methyladenosine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methyluridine-3'-phosphate
  modified_base 9: mod_base = OTHER; note = 2'-O-methylcytidine-3'-phosphate
  modified_base 10: mod_base = OTHER; note = 2'-O-methylcytidine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluorocytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylguanosine-3'-phosphate
  modified_base 20: mod_base = OTHER; note = 2'-O-methyladenosine-3'-phosphate
  modified_base 21: mod_base = OTHER; note = 2'-O-methyladenosine-3'-phosphorothioate
  modified_base 22: mod_base = OTHER; note = 2'-O-methyladenosine-3'-phosphorothioate
  modified_base 23: mod_base = OTHER; note = 2'-O-methyluridine
  misc_feature 1..4: note = RNA
  misc_feature 6..23: note = RNA
  misc_feature 5: note = DNA
tttactttccttctgctcgaaat

SEQ ID NO: 89 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxyadenosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 6: mod_base = OTHER; note = 2'-O-methylguanosine-3'-phosphate
  modified_base 7: mod_base = OTHER; note = Thymidine-glycol nucleic acid (GNA), S-Isomer
  modified_base 8: mod_base = OTHER; note = 2'-O-methylcytidine-3'-phosphate
  modified_base 9: mod_base = OTHER; note = 2'-O-methylcytidine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-deoxy-2'-fluorocytidine-3'-phosphate
  modified_base 15: mod_base = OTHER; note = 2'-O-methyluridine-3'-phosphate
  modified_base 16: mod_base = OTHER; note = 2'-O-methyluridine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methylcytidine-3'-phosphorothioate
  modified_base 23: mod_base = OTHER; note = 2'-O-methyluridine
  misc_feature 1: note = RNA
  misc_feature 3..4: note = RNA
  misc_feature 6..11: note = RNA
  misc_feature 13..23: note = RNA
  misc_feature 2: note = DNA
  misc_feature 5: note = DNA
  misc_feature 12: note = DNA
tactggtccattactttccttct

SEQ ID NO: 90 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 6: mod_base = OTHER; note = 2'-O-methylcytidine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methylguanosine-3'-phosphate
  modified_base 10: mod_base = OTHER; note = 2'-O-methyluridine-3'-phosphate
  modified_base 11: mod_base = OTHER; note = 2'-O-methylcytidine-3'-phosphate
  modified_base 12: mod_base = OTHER; note = 2'-O-methylcytidine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methyluridine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methylcytidine-3'-phosphorothioate
  modified_base 22: mod_base = OTHER; note = 2'-O-methylcytidine-3'-phosphorothioate
  modified_base 23: mod_base = OTHER; note = 2'-O-methyluridine
  misc_feature 1..4: note = RNA
  misc_feature 6..23: note = RNA
  misc_feature 5: note = DNA
tttcactggtccattactttcct

SEQ ID NO: 91 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxyadenosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 6: mod_base = OTHER; note = 2'-O-methyluridine-3'-phosphate
  modified_base 8: mod_base = OTHER; note = 2'-O-methyladenosine-3'-phosphate
  modified_base 9: mod_base = OTHER; note = 2'-O-methyladenosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-deoxy-2'-fluoroadenosine-3'-phosphate
  modified_base 15: mod_base = OTHER; note = 2'-O-methylguanosine-3'-phosphate
  modified_base 16: mod_base = OTHER; note = 2'-O-methylguanosine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
  misc_feature 1: note = RNA
  misc_feature 3..4: note = RNA
  misc_feature 6: note = RNA
  misc_feature 8..11: note = RNA
  misc_feature 13..23: note = RNA
  misc_feature 2: note = DNA
  misc_feature 5: note = DNA
  misc_feature 7: note = DNA
  misc_feature 12: note = DNA
taggattaaagtgaggacctgcg

SEQ ID NO: 92 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxyguanosine-3'-phosphorothioate
  modified_base 3: mod_base = OTHER; note = 2'-O-methyladenosine-3'-phosphate
  modified_base 4: mod_base = OTHER; note = 2'-O-methyluridine-3'-phosphate
  modified_base 6: mod_base = OTHER; note = 2'-O-methylguanosine-3'-phosphate
  modified_base 7: mod_base = OTHER; note = Adenosine-glycol nucleic acid (GNA), S-isomer
  modified_base 8: mod_base = OTHER; note = 2'-O-methylguanosine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-deoxy-2'-fluoroadenosine-3'-phosphate
  modified_base 15: mod_base = OTHER; note = 2'-O-methyladenosine-3'-phosphate
  modified_base 16: mod_base = OTHER; note = 2'-O-methylguanosine-3'-phosphate
  modified_base 17: mod_base = OTHER; note = 2'-O-methyluridine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylcytidine
  misc_feature 1: note = RNA
  misc_feature 3..4: note = RNA
  misc_feature 6..11: note = RNA
  misc_feature 13..23: note = RNA
  misc_feature 2: note = DNA
  misc_feature 5: note = DNA
  misc_feature 12: note = DNA
tgatagaggattaaagtgaggac

SEQ ID NO: 93 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxycytidine-3'-phosphorothioate
  modified_base 3: mod_base = OTHER; note = 2'-O-methyluridine-3'-phosphate
  modified_base 4: mod_base = OTHER; note = 2'-O-methylguanosine-3'-phosphate
  modified_base 6: mod_base = OTHER; note = 2'-O-methyladenosine-3'-phosphate
  modified_base 8: mod_base = OTHER; note = 2'-O-methyladenosine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-O-methyladenosine-3'-phosphate
  modified_base 17: mod_base = OTHER; note = 2'-O-methyladenosine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphate
  modified_base 20: mod_base = OTHER; note = 2'-O-methyluridine-3'-phosphate
  modified_base 21: mod_base = OTHER; note = 2'-O-methylguanosine-3'-phosphorothioate
  modified_base 22: mod_base = OTHER; note = 2'-O-methyladenosine-3'-phosphorothioate
  modified_base 23: mod_base = OTHER; note = 2'-O-methylguanosine
  misc_feature 1: note = RNA
  misc_feature 3..4: note = RNA
  misc_feature 6: note = RNA
  misc_feature 8..11: note = RNA
  misc_feature 13..23: note = RNA
  misc_feature 2: note = DNA
  misc_feature 5: note = DNA
  misc_feature 7: note = DNA
  misc_feature 12: note = DNA
tctggatagaggattaaagtgag

SEQ ID NO: 94 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroadenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 6: mod_base = OTHER; note = 2'-O-methylguanosine-3'-phosphate
  modified_base 7: mod_base = OTHER; note = cytidine-2'-phosphate
  modified_base 8: mod_base = OTHER; note = 2'-O-methylcytidine-3'-phosphate
  modified_base 9: mod_base = OTHER; note = 2'-O-methyluridine-3'-phosphate
  modified_base 10: mod_base = OTHER; note = 2'-O-methylcytidine-3'-phosphate
  modified_base 11: mod_base = OTHER; note = 2'-O-methyluridine-3'-phosphate
  modified_base 12: mod_base = OTHER; note = 2'-O-methylcytidine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methyluridine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methylguanosine
  misc_feature 1..4: note = RNA
  misc_feature 6..23: note = RNA
  misc_feature 5: note = DNA
tacatgcctctcttcatcctttg

SEQ ID NO: 95 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroadenosine-3'-phosphorothioate
  modified_base 3: mod_base = OTHER; note = 2'-O-methyladenosine-3'-phosphate
  modified_base 4: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = guanosine-2'-phosphate
  modified_base 8: mod_base = OTHER; note = 2'-O-methylcytidine-3'-phosphate
  modified_base 9: mod_base = OTHER; note = 2'-O-methylcytidine-3'-phosphate
  modified_base 10: mod_base = OTHER; note = 2'-O-methyluridine-3'-phosphate
  modified_base 11: mod_base = OTHER; note = 2'-O-methylcytidine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluorouridine-3'-phosphate
  modified_base 15: mod_base = OTHER; note = 2'-O-methyluridine-3'-phosphate
  modified_base 16: mod_base = OTHER; note = 2'-deoxy-2'-fluorocytidine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyluridine-3'-phosphorothioate
  modified_base 22: mod_base = OTHER; note = 2'-O-methyluridine-3'-phosphorothioate
  modified_base 23: mod_base = OTHER; note = 2'-O-methyluridine
  misc_feature 1..4: note = RNA
  misc_feature 6..23: note = RNA
  misc_feature 5: note = DNA
taacatgcctctcttcatccttt

SEQ ID NO: 96 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methyladenosine-3'-phosphate
  modified_base 6: mod_base = OTHER; note = 2'-deoxy-2'-fluoroadenosine-3'-phosphate
  modified_base 7: mod_base = OTHER; note = 2'-O-methyluridine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-deoxy-2'-fluoroguanosine-3'-phosphate
  modified_base 15: mod_base = OTHER; note = 2'-O-methylguanosine-3'-phosphate
  modified_base 16: mod_base = OTHER; note = 2'-deoxy-2'-fluorocytidine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methylguanosine-3'-phosphate
  modified_base 21: mod_base = OTHER; note = 2'-O-methylguanosine-3'-phosphorothioate
  modified_base 22: mod_base = OTHER; note = 2'-O-methylguanosine-3'-phosphorothioate
  modified_base 23: mod_base = OTHER; note = 2'-O-methylcytidine
tggcaatgaatggggctctgggc

SEQ ID NO: 97 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorocytidine-3'-phosphorothioate
  modified_base 3: mod_base = OTHER; note = 2'-O-methyluridine-3'-phosphate
  modified_base 4: mod_base = OTHER; note = 2'-O-methyluridine-3'-phosphate
  modified_base 5: mod_base = OTHER; note = 2'-O-methyluridine-3'-phosphate
  modified_base 6: mod_base = OTHER; note = 2'-deoxy-2'-fluorouridine-3'-phosphate
  modified_base 7: mod_base = OTHER; note = 2'-O-methylcytidine-3'-phosphate
  modified_base 8: mod_base = OTHER; note = 2'-deoxy-2'-fluorocytidine-3'-phosphate
  modified_base 9: mod_base = OTHER; note = 2'-deoxy-2'-fluoroadenosine-3'-phosphate
  modified_base 10: mod_base = OTHER; note = 2'-O-methylguanosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluorocytidine-3'-phosphate
  modified_base 15: mod_base = OTHER; note = 2'-O-methylguanosine-3'-phosphate
  modified_base 16: mod_base = OTHER; note = 2'-deoxy-2'-fluorocytidine-3'-phosphate
  modified_base 17: mod_base = OTHER; note = 2'-O-methylcytidine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methylguanosine-3'-phosphate
  modified_base 21: mod_base = OTHER; note = 2'-O-methylguanosine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
tcttttccagggtcgccatggcg

SEQ ID NO: 98 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroguanosine-3'-phosphorothioate
  modified_base 3: mod_base = OTHER; note = 2'-O-methylguanosine-3'-phosphate
  modified_base 4: mod_base = OTHER; note = 2'-O-methylcytidine-3'-phosphate
  modified_base 5: mod_base = OTHER; note = 2'-O-methylcytidine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylcytidine-3'-phosphate
  modified_base 9: mod_base = OTHER; note = 2'-O-methyladenosine-3'-phosphate
  modified_base 10: mod_base = OTHER; note = 2'-O-methyluridine-3'-phosphate
  modified_base 11: mod_base = OTHER; note = 2'-O-methylcytidine-3'-phosphate
  modified_base 12: mod_base = OTHER; note = 2'-O-methyladenosine-3'-phosphate
  modified_base 13: mod_base = OTHER; note = 2'-O-methylguanosine-3'-phosphate
  modified_base 14: mod_base = OTHER; note = 2'-deoxy-2'-fluorocytidine-3'-phosphate
  modified_base 15: mod_base = OTHER; note = 2'-O-methyluridine-3'-phosphate
  modified_base 16: mod_base = OTHER; note = 2'-deoxy-2'-fluorouridine-3'-phosphate
  modified_base 17: mod_base = OTHER; note = 2'-O-methyluridine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methylcytidine-3'-phosphate
  modified_base 21: mod_base = OTHER; note = 2'-O-methyladenosine-3'-phosphorothioate
  modified_base 22: mod_base = OTHER; note = 2'-O-methylguanosine-3'-phosphorothioate
  modified_base 23: mod_base = OTHER; note = 2'-O-methylguanosine
tggccttcatcagcttttccagg

SEQ ID NO: 99 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroadenosine-3'-phosphorothioate
  modified_base 3: mod_base = OTHER; note = 2'-O-methylcytidine-3'-phosphate
  modified_base 4: mod_base = OTHER; note = 2'-O-methyladenosine-3'-phosphate
  modified_base 5: mod_base = OTHER; note = 2'-O-methyladenosine-3'-phosphate
  modified_base 6: mod_base = OTHER; note = 2'-deoxy-2'-fluoroadenosine-3'-phosphate
  modified_base 7: mod_base = OTHER; note = 2'-O-methylcytidine-3'-phosphate
  modified_base 8: mod_base = OTHER; note = 2'-deoxy-2'-fluoroguanosine-3'-phosphate
  modified_base 9: mod_base = OTHER; note = 2'-deoxy-2'-fluoroadenosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methylguanosine-3'-phosphate
  modified_base 13: mod_base = OTHER; note = 2'-O-methyluridine-3'-phosphate
  modified_base 14: mod_base = OTHER; note = 2'-deoxy-2'-fluoroguanosine-3'-phosphate
  modified_base 15: mod_base = OTHER; note = 2'-O-methyladenosine-3'-phosphate
  modified_base 16: mod_base = OTHER; note = 2'-deoxy-2'-fluorouridine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methyluridine-3'-phosphate
  modified_base 20: mod_base = OTHER; note = 2'-O-methylguanosine-3'-phosphate
  modified_base 21: mod_base = OTHER; note = 2'-O-methyladenosine-3'-phosphorothioate
  modified_base 22: mod_base = OTHER; note = 2'-O-methyladenosine-3'-phosphorothioate
  modified_base 23: mod_base = OTHER; note = 2'-O-methyluridine
tacaaacgaatgtgattatgaat

SEQ ID NO: 100 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorocytidine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-deoxy-2'-fluoroadenosine-3'-phosphate
  modified_base 7: mod_base = OTHER; note = 2'-O-methyladenosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluorocytidine-3'-phosphate
  modified_base 10: mod_base = OTHER; note = 2'-O-methylcytidine-3'-phosphate
  modified_base 11: mod_base = OTHER; note = 2'-O-methylguanosine-3'-phosphate
  modified_base 12: mod_base = OTHER; note = 2'-O-methyladenosine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 16: mod_base = OTHER; note = 2'-deoxy-2'-fluoroadenosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphate
  modified_base 21: mod_base = OTHER; note = 2'-O-methylcytidine-3'-phosphorothioate
  modified_base 22: mod_base = OTHER; note = 2'-O-methyluridine-3'-phosphorothioate
  modified_base 23: mod_base = OTHER; note = 2'-O-methylcytidine
tcaggaatccgattcaccagctc

SEQ ID NO: 101 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorocytidine-3'-phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyladenosine-3'-phosphate
  modified_base 6: mod_base = OTHER; note = 2'-deoxy-2'-fluorouridine-3'-phosphate
  modified_base 7: mod_base = OTHER; note = 2'-O-methylguanosine-3'-phosphate
  modified_base 8: mod_base = OTHER; note = 2'-deoxy-2'-fluorouridine-3'-phosphate
  modified_base 9: mod_base = OTHER; note = 2'-deoxy-2'-fluoroguanosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 12: mod_base = OTHER; note = 2'-O-methyluridine-3'-phosphate
  modified_base 13: mod_base = OTHER; note = 2'-O-methyladenosine-3'-phosphate
  modified_base 14: mod_base = OTHER; note = 2'-deoxy-2'-fluorouridine-3'-phosphate
  modified_base 15: mod_base = OTHER; note = 2'-O-methylguanosine-3'-phosphate
  modified_base 16: mod_base = OTHER; note = 2'-deoxy-2'-fluoroadenosine-3'-phosphate
  modified_base 17: mod_base = OTHER; note = 2'-O-methyladenosine-3'-phosphate
  modified_base 18: mod_base = OTHER; note = 2'-O-methyluridine-3'-phosphate
  modified_base 19: mod_base = OTHER; note = 2'-O-methyladenosine-3'-phosphate
  modified_base 20: mod_base = OTHER; note = 2'-O-methylguanosine-3'-phosphate
  modified_base 21: mod_base = OTHER; note = 2'-O-methylcytidine-3'-phosphorothioate
  modified_base 22: mod_base = OTHER; note = 2'-O-methyladenosine-3'-phosphorothioate
  modified_base 23: mod_base = OTHER; note = 2'-O-methyluridine
tcgaatgtgattatgaatagcat

SEQ ID NO: 102 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroadenosine-3'-phosphorothioate
  modified_base 3: mod_base = OTHER; note = 2'-O-methyluridine-3'-phosphate
  modified_base 4: mod_base = OTHER; note = 2'-O-methylcytidine-3'-phosphate
  modified_base 6: mod_base = OTHER; note = 2'-O-methylguanosine-3'-phosphate
  modified_base 7: mod_base = OTHER; note = cytidine-2'-phosphate
  modified_base 8: mod_base = OTHER; note = 2'-O-methyluridine-3'-phosphate
  modified_base 9: mod_base = OTHER; note = 2'-O-methyluridine-3'-phosphate
  modified_base 10: mod_base = OTHER; note = 2'-O-methyluridine-3'-phosphate
  modified_base 11: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-deoxy-2'-fluoroadenosine-3'-phosphate
  modified_base 15: mod_base = OTHER; note = 2'-O-methylguanosine-3'-phosphate
  modified_base 17: mod_base = OTHER; note = 2'-O-methylguanosine-3'-phosphate
  modified_base 18: mod_base = OTHER; note = 2'-O-methyluridine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methylcytidine-3'-phosphorothioate
  modified_base 22: mod_base = OTHER; note = 2'-O-methylcytidine-3'-phosphorothioate
  modified_base 23: mod_base = OTHER; note = 2'-O-methylguanosine
  misc_feature 1..4: note = RNA
  misc_feature 6..11: note = RNA
  misc_feature 13..15: note = RNA
  misc_feature 17..23: note = RNA
  misc_feature 5: note = DNA
  misc_feature 12: note = DNA
  misc_feature 16: note = DNA
tatcagcttttccagggtcgccg

SEQ ID NO: 103 - RNA, 23 nt, synthetic construct
  source 1..23: mol_type = other RNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluoroadenosine-3'-phosphorothioate
  modified_base 3: mod_base = OTHER; note = 2'-O-methylguanosine-3'-phosphate
  modified_base 4: mod_base = OTHER; note = 2'-O-methylguanosine-3'-phosphate
  modified_base 5: mod_base = OTHER; note = 2'-O-methyladenosine-3'-phosphate
  modified_base 6: mod_base = OTHER; note = 2'-O-methylcytidine-3'-phosphate
  modified_base 7: mod_base = OTHER; note = Thymidine-glycol nucleic acid (GNA), S-Isomer
  modified_base 8: mod_base = OTHER; note = 2'-O-methyluridine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 11: mod_base = OTHER; note = 2'-O-methylguanosine-3'-phosphate
  modified_base 12: mod_base = OTHER; note = 2'-O-methylguanosine-3'-phosphate
  modified_base 13: mod_base = OTHER; note = 2'-O-methylguanosine-3'-phosphate
  modified_base 14: mod_base = OTHER; note = 2'-deoxy-2'-fluoroadenosine-3'-phosphate
  modified_base 15: mod_base = OTHER; note = 2'-O-methylcytidine-3'-phosphate
  modified_base 16: mod_base = OTHER; note = 2'-deoxy-2'-fluorouridine-3'-phosphate
  modified_base 17: mod_base = OTHER; note = 2'-O-methylcytidine-3'-phosphate
  modified_base 18: mod_base = OTHER; note = 2'-O-methylguanosine-3'-phosphate
  modified_base 19: mod_base = OTHER; note = 2'-O-methyladenosine-3'-phosphate
  modified_base 20: mod_base = OTHER; note = 2'-O-methyladenosine-3'-phosphate
  modified_base 21: mod_base = OTHER; note = 2'-O-methylguanosine-3'-phosphorothioate
  modified_base 22: mod_base = OTHER; note = 2'-O-methylguanosine-3'-phosphorothioate
  modified_base 23: mod_base = OTHER; note = 2'-O-methylcytidine
taggacttgagggactcgaaggc

SEQ ID NO: 104 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methyladenosine-3'-phosphate
  modified_base 6: mod_base = OTHER; note = 2'-O-methyladenosine-3'-phosphate
  modified_base 8: mod_base = OTHER; note = 2'-O-methylcytidine-3'-phosphate
  modified_base 9: mod_base = OTHER; note = 2'-O-methyladenosine-3'-phosphate
  modified_base 10: mod_base = OTHER; note = 2'-O-methylguanosine-3'-phosphate
  modified_base 11: mod_base = OTHER; note = 2'-O-methyladenosine-3'-phosphate
  modified_base 12: mod_base = OTHER; note = 2'-O-methyladenosine-3'-phosphate
  modified_base 13: mod_base = OTHER; note = 2'-O-methylcytidine-3'-phosphate
  modified_base 14: mod_base = OTHER; note = 2'-deoxy-2'-fluorocytidine-3'-phosphate
  modified_base 15: mod_base = OTHER; note = 2'-O-methyluridine-3'-phosphate
  modified_base 17: mod_base = OTHER; note = 2'-O-methyladenosine-3'-phosphate
  modified_base 18: mod_base = OTHER; note = 2'-O-methylcytidine-3'-phosphate
  modified_base 19: mod_base = OTHER; note = 2'-O-methylcytidine-3'-phosphate
  modified_base 20: mod_base = OTHER; note = 2'-O-methylguanosine-3'-phosphate
  modified_base 21: mod_base = OTHER; note = 2'-O-methylguanosine-3'-phosphorothioate
  modified_base 22: mod_base = OTHER; note = 2'-O-methylcytidine-3'-phosphorothioate
  modified_base 23: mod_base = OTHER; note = 2'-O-methylcytidine
  misc_feature 1..4: note = RNA
  misc_feature 6: note = RNA
  misc_feature 8..15: note = RNA
  misc_feature 17..23: note = RNA
  misc_feature 5: note = DNA
  misc_feature 7: note = DNA
  misc_feature 16: note = DNA
ttaaaagcagaacctgaccggcc

SEQ ID NO: 105 - RNA, 23 nt, synthetic construct
  source 1..23: mol_type = other RNA
tcatcaatttcgagcagaaggaa

SEQ ID NO: 106 - RNA, 23 nt, synthetic construct
  source 1..23: mol_type = other RNA
tcaatttcgagcagaaggaaagt

SEQ ID NO: 107 - RNA, 23 nt, synthetic construct
  source 1..23: mol_type = other RNA
atttcgagcagaaggaaagtaat

SEQ ID NO: 108 - RNA, 23 nt, synthetic construct
  source 1..23: mol_type = other RNA
agaaggaaagtaatggaccagtg

SEQ ID NO: 109 - RNA, 23 nt, synthetic construct
  source 1..23: mol_type = other RNA
aggaaagtaatggaccagtgaag

SEQ ID NO: 110 - RNA, 23 nt, synthetic construct
  source 1..23: mol_type = other RNA
tgcaggtcctcactttaatcctc

SEQ ID NO: 111 - RNA, 23 nt, synthetic construct
  source 1..23: mol_type = other RNA
gtcctcactttaatcctctatcc

SEQ ID NO: 112 - RNA, 23 nt, synthetic construct
  source 1..23: mol_type = other RNA
ctcactttaatcctctatccaga

SEQ ID NO: 113 - RNA, 23 nt, synthetic construct
  source 1..23: mol_type = other RNA
caaaggatgaagagaggcatgtt

SEQ ID NO: 114 - RNA, 23 nt, synthetic construct
  source 1..23: mol_type = other RNA
aaaggatgaagagaggcatgttg

SEQ ID NO: 115 - RNA, 23 nt, synthetic construct
  source 1..23: mol_type = other RNA
gcccagagccccattcattgccc

SEQ ID NO: 116 - RNA, 23 nt, synthetic construct
  source 1..23: mol_type = other RNA
cgccatggcgaccctggaaaagc

SEQ ID NO: 117 - RNA, 23 nt, synthetic construct
  source 1..23: mol_type = other RNA
cctggaaaagctgatgaaggcct

SEQ ID NO: 118 - RNA, 23 nt, synthetic construct
  source 1..23: mol_type = other RNA
attcataatcacattcgtttgtt

SEQ ID NO: 119 - RNA, 23 nt, synthetic construct
  source 1..23: mol_type = other RNA
gagctggtgaatcggattcctgc

SEQ ID NO: 120 - RNA, 23 nt, synthetic construct
  source 1..23: mol_type = other RNA
atgctattcataatcacattcgt

SEQ ID NO: 121 - RNA, 23 nt, synthetic construct
  source 1..23: mol_type = other RNA
tggcgaccctggaaaagctgatg

SEQ ID NO: 122 - RNA, 23 nt, synthetic construct
  source 1..23: mol_type = other RNA
gccttcgagtccctcaagtcctt

SEQ ID NO: 123 - RNA, 23 nt, synthetic construct
  source 1..23: mol_type = other RNA
ggccgctcaggttctgcttttac

SEQ ID NO: 124 - RNA, 21 nt, synthetic construct
  source 1..21: mol_type = other RNA
atcaatttcgagcagaaggaa

SEQ ID NO: 125 - RNA, 21 nt, synthetic construct
  source 1..21: mol_type = other RNA
aatttcgagcagaaggaaaga

SEQ ID NO: 126 - RNA, 21 nt, synthetic construct
  source 1..21: mol_type = other RNA
ttcgagcagaaggaaagtaaa

SEQ ID NO: 127 - RNA, 21 nt, synthetic construct
  source 1..21: mol_type = other RNA
aaggaaagtaatggaccagta

SEQ ID NO: 128 - RNA, 21 nt, synthetic construct
  source 1..21: mol_type = other RNA
gaaagtaatggaccagtgaaa

SEQ ID NO: 129 - RNA, 21 nt, synthetic construct
  source 1..21: mol_type = other RNA
caggtcctcactttaatccta

SEQ ID NO: 130 - RNA, 21 nt, synthetic construct
  source 1..21: mol_type = other RNA
cctcactttaatcctctatca

SEQ ID NO: 131 - RNA, 21 nt, synthetic construct
  source 1..21: mol_type = other RNA
cactttaatcctctatccaga

SEQ ID NO: 132 - RNA, 21 nt, synthetic construct
  source 1..21: mol_type = other RNA
aaggatgaagagaggcatgta

SEQ ID NO: 133 - RNA, 21 nt, synthetic construct
  source 1..21: mol_type = other RNA
aggatgaagagaggcatgtta

SEQ ID NO: 134 - RNA, 21 nt, synthetic construct
  source 1..21: mol_type = other RNA
ccagagccccattcattgcca

SEQ ID NO: 135 - RNA, 21 nt, synthetic construct
  source 1..21: mol_type = other RNA
ccatggcgaccctggaaaaga

SEQ ID NO: 136 - RNA, 21 nt, synthetic construct
  source 1..21: mol_type = other RNA
tggaaaagctgatgaaggcca

SEQ ID NO: 137 - RNA, 21 nt, synthetic construct
  source 1..21: mol_type = other RNA
tcataatcacattcgtttgta

SEQ ID NO: 138 - RNA, 21 nt, synthetic construct
  source 1..21: mol_type = other RNA
gctggtgaatcggattcctga

SEQ ID NO: 139 - RNA, 21 nt, synthetic construct
  source 1..21: mol_type = other RNA
gctattcataatcacattcga

SEQ ID NO: 140 - RNA, 21 nt, synthetic construct
  source 1..21: mol_type = other RNA
gcgaccctggaaaagctgata

SEQ ID NO: 141 - RNA, 21 nt, synthetic construct
  source 1..21: mol_type = other RNA
cttcgagtccctcaagtccta

SEQ ID NO: 142 - RNA, 21 nt, synthetic construct
  source 1..21: mol_type = other RNA
ccggtcaggttctgcttttaa

SEQ ID NO: 143 - RNA, 23 nt, synthetic construct
  source 1..23: mol_type = other RNA
  modified_base 5: mod_base = OTHER; note = 5'-methyluridine-3'-phosphate
ttccttctgctcgaaattgatgg

SEQ ID NO: 144 - RNA, 23 nt, synthetic construct
  source 1..23: mol_type = other RNA
  modified_base 5: mod_base = OTHER; note = 5'-methyluridine-3'-phosphate
tctttccttctgctcgaaattgg

SEQ ID NO: 145 - RNA, 23 nt, synthetic construct
  source 1..23: mol_type = other RNA
  modified_base 7: mod_base = OTHER; note = 5'-methyluridine-3'-phosphate
tttactttccttctgctcgaaat

SEQ ID NO: 146 - RNA, 23 nt, synthetic construct
  source 1..23: mol_type = other RNA
  modified_base 7: mod_base = OTHER; note = 5'-methyluridine-3'-phosphate
  modified_base 12: mod_base = OTHER; note = 5'-methyluridine-3'-phosphate
tactggtccattactttccttct

SEQ ID NO: 147 - RNA, 23 nt, synthetic construct
  source 1..23: mol_type = other RNA
  modified_base 7: mod_base = OTHER; note = 5'-methyluridine-3'-phosphate
tttcactggtccattactttcct

SEQ ID NO: 148 - RNA, 23 nt, synthetic construct
  source 1..23: mol_type = other RNA
  modified_base 7: mod_base = OTHER; note = 5'-methyluridine-3'-phosphate
  modified_base 12: mod_base = OTHER; note = 5'-methyluridine-3'-phosphate
taggattaaagtgaggacctgcg

SEQ ID NO: 149 - RNA, 23 nt, synthetic construct
  source 1..23: mol_type = other RNA
  modified_base 12: mod_base = OTHER; note = 5'-methyluridine-3'-phosphate
tgatagaggattaaagtgaggac

SEQ ID NO: 150 - RNA, 23 nt, synthetic construct
  source 1..23: mol_type = other RNA
  modified_base 7: mod_base = OTHER; note = 5'-methyluridine-3'-phosphate
tctggatagaggattaaagtgag

SEQ ID NO: 151 - RNA, 23 nt, synthetic construct
  source 1..23: mol_type = other RNA
  modified_base 5: mod_base = OTHER; note = 5'-methyluridine-3'-phosphate
tacatgcctctcttcatcctttg

SEQ ID NO: 152 - RNA, 23 nt, synthetic construct
  source 1..23: mol_type = other RNA
taacatgcctctcttcatccttt

SEQ ID NO: 153 - RNA, 23 nt, synthetic construct
  source 1..23: mol_type = other RNA
tggcaatgaatggggctctgggc

SEQ ID NO: 154 - RNA, 23 nt, synthetic construct
  source 1..23: mol_type = other RNA
tcttttccagggtcgccatggcg

SEQ ID NO: 155 - RNA, 23 nt, synthetic construct
  source 1..23: mol_type = other RNA
  modified_base 7: mod_base = OTHER; note = 5'-methyluridine-3'-phosphate
tggccttcatcagcttttccagg

SEQ ID NO: 156 - RNA, 23 nt, synthetic construct
  source 1..23: mol_type = other RNA
tacaaacgaatgtgattatgaat

SEQ ID NO: 157 - RNA, 23 nt, synthetic construct
  source 1..23: mol_type = other RNA
tcaggaatccgattcaccagctc

SEQ ID NO: 158 - RNA, 23 nt, synthetic construct
  source 1..23: mol_type = other RNA
tcgaatgtgattatgaatagcat

SEQ ID NO: 159 - RNA, 23 nt, synthetic construct
  source 1..23: mol_type = other RNA
tatcagcttttccagggtcgccg

SEQ ID NO: 160 - RNA, 23 nt, synthetic construct
  source 1..23: mol_type = other RNA
  modified_base 7: mod_base = OTHER; note = 5'-methyluridine-3'-phosphate
taggacttgagggactcgaaggc

SEQ ID NO: 161 - RNA, 23 nt, synthetic construct
  source 1..23: mol_type = other RNA
ttaaaagcagaacctgaccggcc

(19) United States
(12) Patent Application Publication
(10) Pub. No.: US 2026/0272974 A1
(43) Pub. Date: Sep. 17, 2026

(54) Formulations for Oligonucleotide Delivery

(71) Applicant: ALNYLAM PHARMACEUTICALS, INC., CAMBRIDGE, MA (US)

(72) Inventors: ANDY PHUNG, BRAINTREE, MA (US); HAN YUEH, WATERTOWN, MA (US); GANG SHEN, ANDOVER, MA (US); MATTHIAS KRETSCHMER, BROOKLINE, MA (US); KAMRAN SIMANI, NEEDHAM, MA (US); LIUWEI JIANG, WALTHAM, MA (US); BO PANG, CAMBRIDGE, MA (US); LUBOMIR NECHEV, ANDOVER, MA (US); LIGANG ZHANG, WINCHESTER, MA (US)

(73) Assignee: ALNYLAM PHARMACEUTICALS, INC., CAMBRIDGE, MA (US)

(21) Appl. No.: 19/474,095

(22) PCT Filed: Apr. 12, 2024
(86) PCT No.: PCT/US2024/024374

§ 371 Date: Oct. 9, 2025

Related U.S. Application Data
(60) Provisional application No. 63/495,652, filed on Apr. 12, 2023.
(60) Provisional application No. 63/631,377, filed on Apr. 8, 2024.

(51) Int. Cl. A61K 31/713; A61K 9/00; A61K 47/02

(52) U.S. Cl. CPC A61K 31/713; A61K 9/0019; A61K 47/02

(57) ABSTRACT

The disclosure relates to compositions and methods for delivery of nucleic acid therapeutics, particularly iRNA agents to target locations and genes residing in the central nervous system (CNS) of a subject.

### CROSS-REFERENCE TO RELATED APPLICATIONS

This application claims the benefit of priority to U.S. Provisional Application No. 63/631,377, filed on Apr. 8, 2024, and to U.S. Provisional Application No. 63/495,652, filed on Apr. 12, 2023. The entire contents of the foregoing applications are hereby incorporated herein by reference.

### FIELD OF THE INVENTION

The instant disclosure relates generally to improved formulations for oligonucleotide, e.g., iRNA agent, delivery. methods of manufacture and methods for their use.

### SEQUENCE LISTING

The instant application contains a Sequence Listing which has been filed electronically in eXtensible Markup Language (XML) format and is hereby incorporated by reference in its entirety. Said XML copy, created on Apr. 5, 2024, is named A108868_1750WO_SL.xml and is 924,746 bytes in size.

### BACKGROUND OF THE INVENTION

Efficient delivery of an inhibitory RNA (iRNA) agent to extrahepatic organs, particularly those of the central nervous system (CNS), has been recently described (see, e.g., WO 2019/217459). While lipophilic modification of iRNA agents was identified to promote efficient CNS delivery of iRNA agents, the formulations ultimately employed for therapeutic delivery of any specific iRNA agent also need to be free of chemistry, manufacturing and control (CMC) issues during drug product development. Issues such as unexpected particulate formation and batch-to-batch variation can arise during the drug product development process for any selected therapeutic agent.

There is therefore a need for viable alternative formulations that can be successfully employed during drug product development if/when a candidate therapeutic agent confronts an issue that might otherwise adversely impact the new drug product's CMC.

### BRIEF SUMMARY OF THE INVENTION

The present disclosure, at least in part, provides compositions for enhancing formulation of iRNA agents during drug product development, as well as resultant drug product formulations, associated methods, kits and other compositions. In particular, the compositions herein may be administered via parenteral (e.g., injectable) administration.

In one aspect, the instant disclosure provides a composition that includes: (a) a double-stranded ribonucleic acid (dsRNA) having a sense strand and an antisense strand, where one of the sense strand or the antisense strand of the dsRNA includes at least one lipophilic modification and the other strand of the dsRNA does not comprise a lipophilic modification, and (b) a divalent ion source, where (i) substantially all of the sense strand or the antisense strand of the dsRNA comprising the at least one lipophilic modification in the composition is duplexed with the strand that does not comprise a lipophilic modification, or (ii) the strand comprising the at least one lipophilic modification is present at less than 1% molar excess relative to the other strand, the strands are present at molar equivalence, or the strand comprising the at least one lipophilic modification is present in a molar excess relative to the other strand. In certain embodiments, the composition is substantially free of inorganic phosphate, as described in further detail below.

In a related aspect, the instant disclosure provides a composition that includes: (a) a dsRNA having a sense strand and an antisense strand, where one of the sense strand or the antisense strand of the dsRNA includes at least one lipophilic modification and the other strand of the dsRNA does not include a lipophilic modification; and (b) a divalent ion source, where: (i) the sense strand or the antisense strand of the dsRNA that comprises at least one lipophilic modification is present at less than 1% molar excess relative to the strand of the dsRNA that does not comprise a lipophilic modification, the strand of the dsRNA that does not comprise a lipophilic modification is present at molar equivalence to the sense strand or the antisense strand of the dsRNA that comprises at least one lipophilic modification, or the strand of the dsRNA that does not comprise a lipophilic modification is present in molar excess relative to the sense strand or the antisense strand of the dsRNA that comprises at least one lipophilic modification. In certain embodiments, the composition is substantially free of inorganic phosphate, as described in further detail below.

In one embodiment, the sense strand of the dsRNA includes at least one lipophilic modification and the antisense strand of the dsRNA does not include a lipophilic modification.

In another embodiment, there is a molar excess of the antisense strand relative to the sense strand. Optionally, there is at least an about 0.1% molar excess of the antisense strand relative to the sense strand. Optionally, there is at least an about 0.2% molar excess of the antisense strand relative to the sense strand. Optionally, there is at least an about 0.3% molar excess of the antisense strand relative to the sense strand. Optionally, there is at least an about 0.4% molar excess of the antisense strand relative to the sense strand. Optionally, there is at least an about 0.5% molar excess of the antisense strand relative to the sense strand. Optionally, there is at least an about 1% molar excess of the antisense strand relative to the sense strand. Optionally, there is at least an about 2% molar excess of the antisense strand relative to the sense strand. Optionally, there is at least an about 3% molar excess of the antisense strand relative to the sense strand. Optionally, there is at least an about 4% molar excess of the antisense strand relative to the sense strand. Optionally, there is about a 5% or greater molar excess of the antisense strand relative to the sense strand.

In another embodiment, the antisense strand of the dsRNA includes the at least one lipophilic modification and the sense strand of the dsRNA does not include a lipophilic modification. In certain embodiments, the antisense strand of the dsRNA includes the at least one lipophilic modification. In a related embodiment, there is a molar excess of the sense strand relative to the antisense strand. In some embodiments, there is at least an about 0.1% molar excess of the sense strand relative to the antisense strand. Optionally, there is at least an about 0.2% molar excess of the sense strand relative to the antisense strand. Optionally, there is at least an about 0.3% molar excess of the sense strand relative to the antisense strand. Optionally, there is at least an about 0.4% molar excess of the sense strand relative to the antisense strand. Optionally, there is at least an about 0.5% molar excess of the sense strand relative to the antisense strand. Optionally, there is at least an about 1% molar excess of the sense strand relative to the antisense strand. Optionally, there is at least an about 2% molar excess of the sense strand relative to the antisense strand. Optionally, there is at least an about 3% molar excess of the sense strand relative to the antisense strand. Optionally, there is at least an about 4% molar excess of the sense strand relative to the antisense strand. Optionally, there is about a 5% or greater molar excess of the sense strand relative to the antisense strand.

In one embodiment, the dsRNA composition is formulated for intravenous, subcutaneous, intramuscular, intradermal, intra-articular and/or intrathecal administration/delivery. In one embodiment, the dsRNA composition is formulated for intrathecal administration/delivery.

In certain embodiments, the lipophilic modification is a saturated or unsaturated C₄-C₃₀ hydrocarbon. Optionally, the lipophilic modification is a C₄-C₃₀ alkyl or alkenyl. Optionally, the lipophilic modification is a linear C₆-C₁₈ alkyl or alkenyl. Optionally, the lipophilic modification is a C₁₆ alkyl. In certain embodiments, the lipophilic modification is attached at the 2′-ribo position of a nucleic acid residue of the dsRNA.

In some embodiments, the dsRNA includes at least one modified nucleotide that is not a 2′-deoxynucleotide.

In certain embodiments, the dsRNA includes at least one modified nucleotide that includes a 5′-phosphate or 5′-phosphate mimic group.

In one embodiment, the divalent ion source is magnesium, calcium, copper, nickel, zinc, or strontium. In one embodiment, the divalent ion source is calcium.

In some embodiments, the molar ratio of the divalent ion-to-the dsRNA is greater than about 2:1.

In certain embodiments, the composition is substantially free of inorganic phosphate and/or includes less than 100 ppm of inorganic phosphate. Optionally, the composition includes less than 50 ppm of inorganic phosphate. Optionally, the composition includes less than 10 ppm of inorganic phosphate. Optionally, the composition includes less than 5 ppm of inorganic phosphate. Optionally, the composition does not include inorganic phosphate.

In some embodiments, the at least one modified nucleotide that is not a 2′-deoxynucleotide is selected from among a 2′-O-methyl modified nucleotide, a 2′-fluoro modified nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a tetrahydropyran modified nucleotide, a 1,5-anhydrohexitol modified nucleotide, a cyclohexenyl modified nucleotide and a nucleotide including 2-hydroxymethyl-tetrahydrofurane-5-phosphate.

In one embodiment, the molar ratio of the divalent ion source-to-the-dsRNA is greater than about 2.5:1. Optionally, the molar ratio of the divalent ion source-to-the-dsRNA is greater than about 3.0:1. Optionally, the molar ratio of the divalent ion source-to-the-dsRNA is greater than about 3.5:1. Optionally, the molar ratio of the divalent ion source-to-the-dsRNA is greater than about 4.0:1.

Herein, the maximum divalent ion (e.g., calcium) content of a composition of the disclosure may be where the amount of divalent ion included in the composition reaches a concentration that causes the composition to reach its gelling point. “Gelling point” herein refers to the point at which the composition cannot be drawn through a 25-gauge needle.

In certain embodiments, the molar ratio of the divalent ion source-to-the-dsRNA is between about 2:1 and about 10:1. Optionally, the molar ratio of the divalent ion source-to-the-dsRNA is between about 3:1 and about 10:1. Optionally, the molar ratio of the divalent ion source-to-the-dsRNA is between about 3:1 and about 9:1. Optionally, the molar ratio of the divalent ion source-to-the-dsRNA is between about 3:1 and about 8:1. Optionally, the molar ratio of the divalent ion source-to-the-dsRNA is between about 3: land about 7:1. Optionally, the molar ratio of the divalent ion source-to-the-dsRNA is between about 3:1 and about 6:1. Optionally, the molar ratio of the divalent ion source-to-the-dsRNA is between about 3:1 and about 5:1.

In one embodiment, the dsRNA includes at least one phosphate (5′-phosphate) or a 5′-phosphate mimic modification.

In one embodiment, the 5′-phosphate mimic modification is a phosphonate modification. In a related embodiment, the phosphonate modification is a 5′-vinyl phosphonate modification. Optionally, the phosphonate modification is a 5′-(E)-vinyl phosphonate modification.

In one embodiment, the 5′-phosphate mimic modification is a 5′-vinyl phosphate modification (i.e., 4′-C(H)═C(H)—OP(O)(OH)₂). Optionally, the phosphonate modification is a 5′-(E)-vinyl phosphate modification.

In a related embodiment, the phosphate mimic modification is a 5′-phosphonate modification of the antisense strand.

In some embodiments, the 5′-phosphate mimic can also include a 5′-phosphate prodrug or 5′-phosphonate prodrug. In some embodiments, the 5′-phosphate prodrug or 5′-phosphonate prodrug has a structure of formulas disclosed in WO2022/147214, which is incorporated herein by reference. In some exemplary embodiments, the 5′-phosphate prodrug or 5′-phosphonate prodrug is

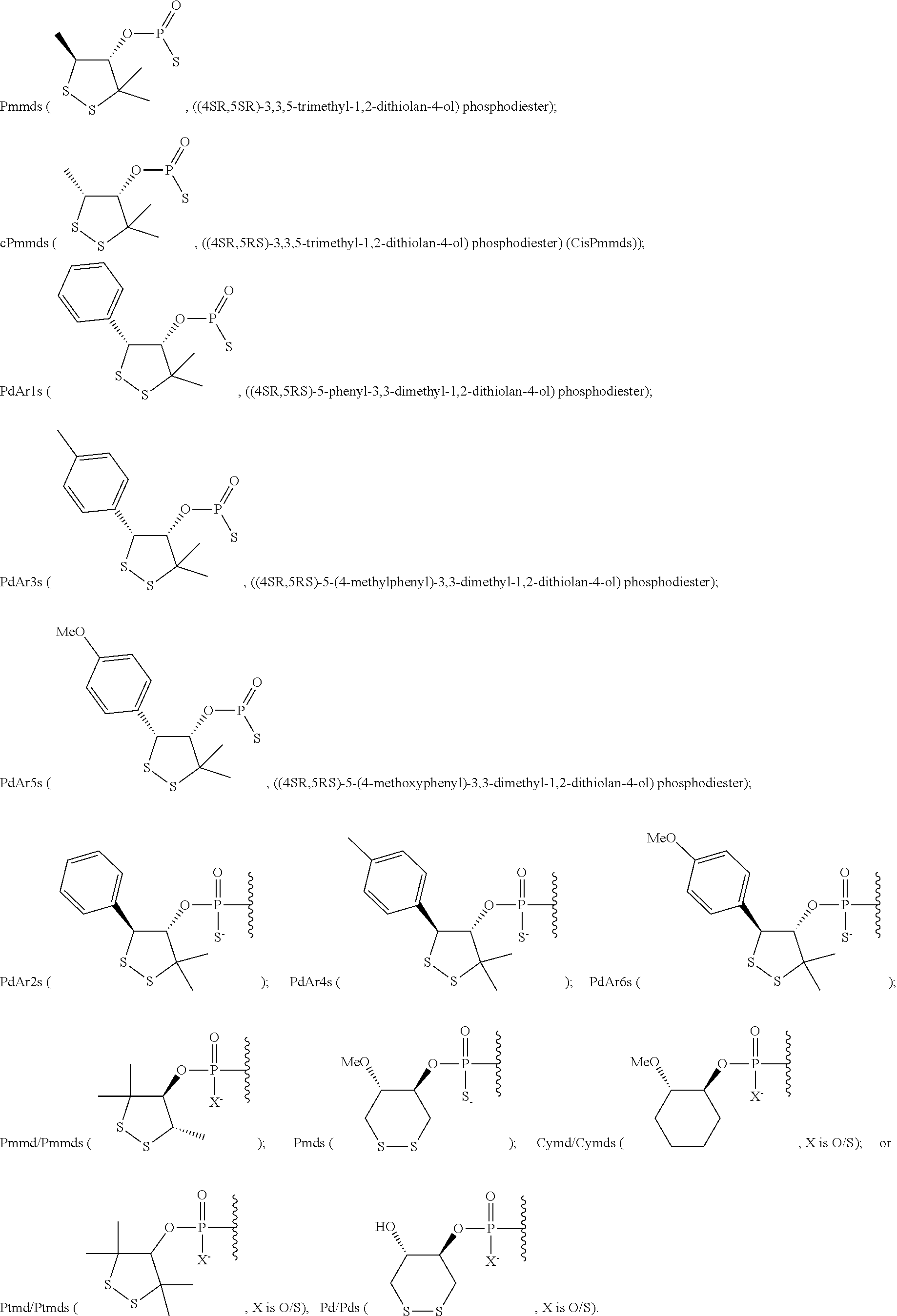

In some exemplary embodiments, the 5′-phosphate prodrug or 5′-phosphonate prodrug is:

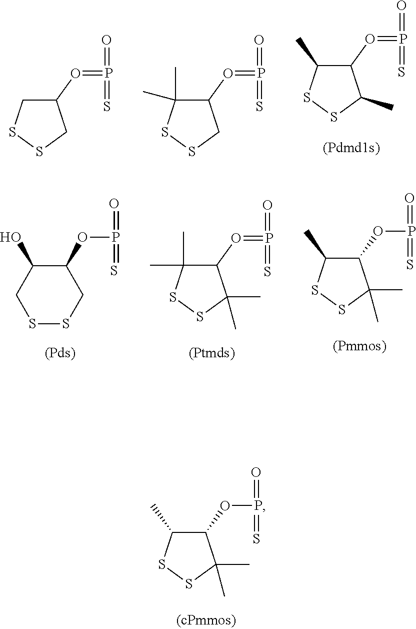

The siRNA containing one of the above list of 5′ modified phosphate prodrugs generally has an activity comparable to that of the siRNA containing 5′-VP. In some exemplary embodiments, the 5′-phosphate prodrug or 5′-phosphonate prodrug is:

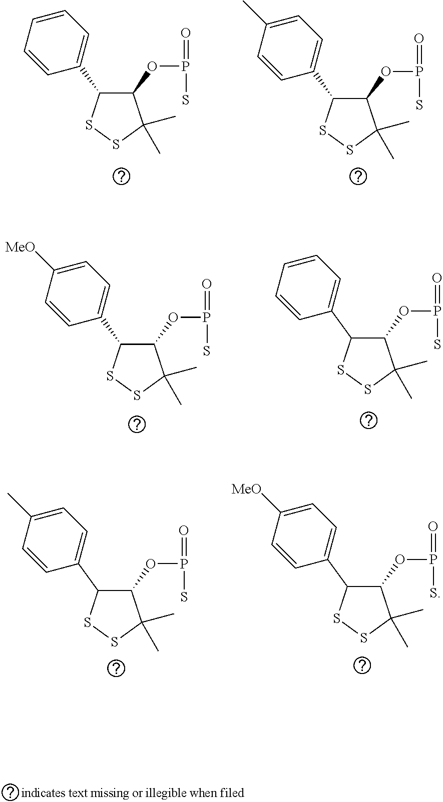

The siRNA containing one of the above list of 5′ modified phosphate prodrugs generally has an improved stability than that of the siRNA containing 5′-VP and has a better or comparable activity than that of the siRNA containing 5′-VP. In some embodiments, the antisense strand is substantially complementary to at least 15 consecutive nucleotides of a target mRNA. Optionally, the sense strand forms a duplex region of at least 15 consecutive base pairs with the antisense strand.

In one embodiment, the sense strand and the antisense strand each independently has 15-40 nucleotides.

In certain embodiments, substantially all of the nucleotides of the sense strand are modified.

In some embodiments, the sense strand includes at least one phosphorothioate internucleotide linkage. In a related embodiment, the sense strand includes at least one block of two consecutive phosphorothioate internucleotide linkages.

In an additional embodiment, the sense strand is 21 nucleotides in length.

In certain embodiments, substantially all of the nucleotides of the antisense strand are modified.

In some embodiments, 7 or fewer of the nucleotides of the antisense strand are 2′-deoxynucleotides. Optionally, 6 or fewer of the nucleotides of the antisense strand are 2′-deoxynucleotides. Optionally, 5 or fewer of the nucleotides of the antisense strand are 2′-deoxynucleotides. Optionally, 4 or fewer of the nucleotides of the antisense strand are 2′-deoxynucleotides. Optionally, 3 or fewer of the nucleotides of the antisense strand are 2′-deoxynucleotides. Optionally, 2 or fewer of the nucleotides of the antisense strand are 2′-deoxynucleotides.

In one embodiment, the antisense strand includes a 5′-terminal or 3′-terminal abasic nucleotide. Optionally, the 5′-terminal or 3′-terminal abasic nucleotide is inverted. Optionally, the 5′-terminal or 3′-terminal abasic nucleotide is 5′-5′ or 3′-3′ linked. Such terminal abasic nucleotide may be attached to the remainder of the oligonucleotide via phosphodiester internucleotide linkage or a phosphorothioate internucleotide linkage. In certain embodiments, one or both terminal abasic nucleotide is attached to the remainder of the oligonucleotide via a phosphorothioate internucleotide linkage.

In another embodiment, the antisense strand includes at least one phosphorothioate internucleotide linkage. In a related embodiment, the antisense strand includes at least one block of two consecutive phosphorothioate internucleotide linkages. Optionally, the antisense strand includes two blocks of two consecutive phosphorothioate internucleotide linkages.

In certain embodiments, the antisense strand includes one thermally destabilizing nucleotide that is not a terminal nucleotide and not a cleavage region nucleotide. Optionally, the thermally destabilizing nucleotide is a glycol nucleic acid (GNA). Optionally, the thermally destabilizing nucleotide is an unlocked nucleic acid (UNA). Optionally, the thermally destabilizing nucleotide is a 2′-5′ linked ribonucleotide (3′-RNA). Optionally, the thermally destabilizing nucleotide is a threose nucleic acid (TNA).

Further examples of thermally destabilizing nucleotide include,

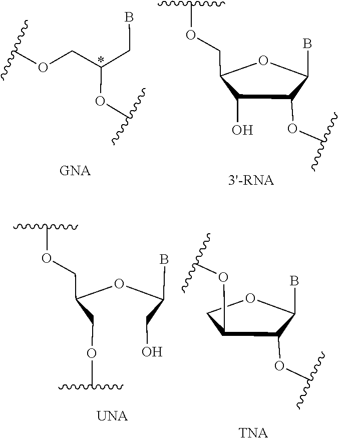

or a stereoisomer thereof, wherein B is a modified or unmodified nucleobase and the asterisk represents either R, S or racemic. In another embodiment, a thermally destabilizing nucleotide includes a nucleobase mismatch between the antisense and the sense strand; for example, the sense strand may have a mismatch to the antisense strand while the latter remains matched at the same position to a target mRNA.

When, the antisense strand includes one thermally destabilizing nucleotide, such nucleotide is not a terminal nucleotide nor at positions 11, 12 and 13, where position 1 is defined as the 5′-terminal nucleotide of the antisense strand. In certain embodiments, the thermally destabilizing nucleotide is at one of positions 2-9, such as position 4; or position 5; or position 6; or position 7; or position 8, counting from the 5′-end of the antisense strand. In one embodiment, the thermally destabilizing nucleotide is at position 6 or position 7, counting from the 5′-end of the antisense strand. In one embodiment, the thermally destabilizing nucleotide is at position 7, counting from the 5′-end of the antisense strand.

In one embodiment, the antisense strand is 23 nucleotides in length. In one embodiment, the antisense strand is 19 nucleotides in length. In one embodiment, the antisense strand is 20 nucleotides in length. In one embodiment, the antisense strand is 21 nucleotides in length. In one embodiment, the antisense strand is 22 nucleotides in length.

In another embodiment, both of the sense and antisense strand is 19 nucleotides in length. In another embodiment, both of the sense and antisense strand is 20 nucleotides in length. In another embodiment, both of the sense and antisense strand is 21 nucleotides in length. In another embodiment, both of the sense and antisense strand is 22 nucleotides in length. In another embodiment, both of the sense and antisense strand is 23 nucleotides in length. In each of these embodiments, the iRNA agent may have blunt ends at both ends of the duplexed region.

In another embodiment, the sense strand is 19 nucleotides in length and the antisense strand is 21 nucleotides in length. In another embodiment, the sense strand is 20 nucleotides in length and the antisense strand is 22 nucleotides in length. In another embodiment, the sense strand is 21 nucleotides in length and the antisense strand is 23 nucleotides in length. In each of these embodiments, the iRNA agent may have a 2 nucleotide overhang at one end of the duplexed region (e.g., the 3′-end of the antisense strand).

In some embodiments, the composition further includes a diluent.

In a related embodiment, the composition is isotonic to cerebrospinal fluid (CSF).

In certain embodiments, the composition further includes a sodium source, a potassium source, a magnesium source, and a calcium source.

In additional embodiments, the composition includes sodium chloride, magnesium chloride, potassium chloride, and calcium chloride.

In some embodiments, the composition has a pH between about 4 and about 10. Optionally, the pH is between about 6 and about 10. Optionally, the pH is between about 6.5 and about 8.0.

In certain embodiments, the composition has an osmolality between about 200 and 400 mOsm/kg.

In one embodiment, the composition does not include hydrogen phosphate or dihydrogen phosphate.

In another embodiment, the composition does not include a buffer.

In an additional embodiment, the composition includes a stabilizing agent, which is optionally sucrose, glucose, mannitol, sorbitol, polyethylene glycol (PEG), histidine, arginine, lysine, phospholipids, or trehalose, or a combination thereof.

In some embodiments, the composition includes greater than 1 mg of the dsRNA per mL of the composition. Optionally, the composition includes greater than 5 mg of the dsRNA per mL of the composition. Optionally, the composition includes greater than 10 mg of the dsRNA per mL of the composition. Optionally, the composition includes greater than 25 mg of the dsRNA per mL of the composition. Optionally, the composition includes greater than 50 mg of the dsRNA per mL of the composition. Optionally, the composition includes about 60 mg of the dsRNA per mL of the composition.

In certain embodiments, the dsRNA is AD-961583, AD-454973, AD-454843, AD-961584, AD-961585, AD-961586, AD-454844, AD-1302922, AD-1302923, or AD-1999409.

An additional aspect of the instant disclosure provides a composition that includes (a) a double-stranded ribonucleic acid (dsRNA) having a sense strand and an antisense strand, where the composition includes about 50 mg to about 70 mg of the dsRNA per mL of the composition; (b) sodium chloride at about 80 mM to about 110 mM; and (c) calcium chloride at about 8.0 mM to about 20.0 mM, where the composition is substantially free of inorganic phosphate.

An additional aspect of the instant disclosure provides a composition that includes (a) a double-stranded ribonucleic acid (dsRNA) having a sense strand and an antisense strand, where the composition includes about 50 mg to about 70 mg of the dsRNA per mL of the composition; (b) sodium chloride at about 95 mM to about 100 mM; and (c) calcium chloride at about 12.0 mM to about 14.0 mM, where the composition is substantially free of inorganic phosphate.

In certain embodiments, the composition further includes (d) potassium chloride at about 1.0 mM to about 2.5 mM; and (e) magnesium chloride at about 0.1 mM to about 1.0 mM.

In certain embodiments, the composition further includes (d) potassium chloride at about 1.8 mM to about 2.0 mM; and (e) magnesium chloride at about 0.4 mM to about 0.6 mM.

In some embodiments, the composition includes about 60 mg of the dsRNA per mL of the composition.

In one embodiment, the composition includes sodium chloride at about 85 mM to about 100 mM.

In one embodiment, the composition includes calcium chloride at about 10 mM to about 15 mM.

In one embodiment, the composition includes potassium chloride at about 1.5 mM to about 2.2 mM.

In one embodiment, the composition includes magnesium chloride at about 0.3 mM to about 0.7 mM.

In one embodiment, the composition includes sodium chloride at about 97.6 mM.

In one embodiment, the composition includes sodium chloride at about 97.0 mM.

In one embodiment, the composition includes sodium chloride at about 89.5 mM.

In another embodiment, the composition includes calcium chloride at about 13.0 mM.

In another embodiment, the composition includes calcium chloride at about 12.9 mM.

In another embodiment, the composition includes calcium chloride at about 13.9 mM.

In certain embodiments, the composition includes potassium chloride at about 1.9 mM.

In certain embodiments, the composition includes potassium chloride at about 1.7 mM.

In some embodiments, the composition includes magnesium chloride at about 0.5 mM.

In one embodiment, the composition is a pharmaceutical composition for intrathecal administration of the dsRNA to a subject. In a related embodiment, the subject is a mammal. Optionally, the subject is human.

In some embodiments, the dsRNA includes at least one modified nucleotide that is not a 2′-deoxynucleotide.

Another aspect of the instant disclosure provides a composition for intrathecal administration that includes (a) a dsRNA that is AD-961583, AD-454973, AD-454843, AD-961584, AD-961585, AD-961586, AD-454844, AD-1302922, AD-1302923, or AD-1999409; (b) a calcium ion source; and (c) a diluent, where (i) the composition is substantially free of inorganic phosphate and (ii) the molar ratio of the dsRNA to calcium ion source to the dsRNA is greater than 3 to 1.

In one embodiment, the dsRNA is AD-454973.

In another embodiment, the dsRNA is AD-454843.

In an alternative embodiment, dsRNA is AD-961583.

In an additional embodiment, the dsRNA is AD-961584.

In certain embodiments, the dsRNA is AD-961585.

In some embodiments, the dsRNA is AD-961586.

In some embodiments, the dsRNA is AD-454844.

In some embodiments, the dsRNA is AD-1302922.

In some embodiments, the dsRNA is AD-1302923.

In some embodiments, the dsRNA is AD-1999409.

An additional aspect of the instant disclosure provides a composition for intrathecal administration that includes (a) a dsRNA that is AD-1395718, AD-1395724, AD-1395731, AD-1395738, AD-1395743, AD-1395756, AD-1395760, AD-1395762, AD-1395764, or AD-1395771; (b) a calcium ion source; and (c) a diluent, where (i) the composition is substantially free of inorganic phosphate and (ii) the molar ratio of the dsRNA to calcium ion source to the dsRNA is greater than 3 to 1.

In one embodiment, the dsRNA is AD-1395718.

In another embodiment, the dsRNA is AD-1395724.

In an alternative embodiment, dsRNA is AD-1395731.

In an additional embodiment, the dsRNA is AD-1395738.

In certain embodiments, the dsRNA is AD-1395743.

In some embodiments, the dsRNA is AD-1395756.

In some embodiments, the dsRNA is AD-1395760.

In some embodiments, the dsRNA is AD-1395762.

In some embodiments, the dsRNA is AD-1395764.

In some embodiments, the dsRNA is AD-1395771.

Another aspect of the instant disclosure provides a composition for intrathecal administration that includes (a) a dsRNA that is AD-1019448, AD-1019465, AD-1271082, AD-1271083, AD-1271084, AD-1271085, AD-1498524, AD-1498526, or AD-1498528; (b) a calcium ion source; and (c) a diluent, where (i) the composition is substantially free of inorganic phosphate and (ii) the molar ratio of the dsRNA to calcium ion source to the dsRNA is greater than 3 to 1.

In one embodiment, the dsRNA is AD-1019448.

In another embodiment, the dsRNA is AD-1019465.

In an alternative embodiment, dsRNA is AD-1271082.

In an additional embodiment, the dsRNA is AD-1271083.

In certain embodiments, the dsRNA is AD-1271084.

In some embodiments, the dsRNA is AD-1271085.

In some embodiments, the dsRNA is AD-1498524.

In some embodiments, the dsRNA is AD-1498526.

In some embodiments, the dsRNA AD-1498528.

Another aspect of the disclosure provides a composition including a double-stranded ribonucleic acid (dsRNA) capable of annealing and reducing expression of an amyloid precursor protein (APP) mRNA, where the dsRNA includes a sense strand and an antisense strand, where one of the sense strand or the antisense strand of the dsRNA includes at least one lipophilic modification and the other strand of the dsRNA does not include a lipophilic modification; and where: (i) substantially all of the sense strand or the antisense strand of the dsRNA including the at least one lipophilic modification in the composition is duplexed with the strand that does not include a lipophilic modification, or (ii) the sense strand or the antisense strand of the dsRNA including the at least one lipophilic modification is present at less than 1% molar excess relative to the strand that does not include a lipophilic modification, the sense strand and the antisense strand are present at molar equivalence, or the strand that does not include a lipophilic modification is present in a molar excess relative to the strand of the dsRNA including the at least one lipophilic modification.

In one embodiment, the composition further includes a divalent ion source.

In some embodiments, the dsRNA is AD-961583, AD-454973, AD-454843, AD-961584, AD-961585, or AD-961586.

An additional aspect of the disclosure provides a composition including a double-stranded ribonucleic acid (dsRNA) capable of annealing and reducing expression of a superoxide dismutase 1 (SOD1) mRNA, where the dsRNA includes a sense strand and an antisense strand, where one of the sense strand or the antisense strand of the dsRNA includes at least one lipophilic modification and the other strand of the dsRNA does not include a lipophilic modification; and where: (i) substantially all of the sense strand or the antisense strand of the dsRNA including the at least one lipophilic modification in the composition is duplexed with the strand that does not include a lipophilic modification, or (ii) the sense strand or the antisense strand of the dsRNA including the at least one lipophilic modification is present at less than 1% molar excess relative to the strand that does not include a lipophilic modification, the sense strand and the antisense strand are present at molar equivalence, or the strand that does not include a lipophilic modification is present in a molar excess relative to the strand of the dsRNA including the at least one lipophilic modification.

In certain embodiments, the dsRNA is AD-1395718, AD-1395724, AD-1395731, AD-1395738, AD-1395743, AD-1395756, AD-1395760, AD-1395762, AD-1395764, or AD-1395771.

Another aspect of the disclosure provides a composition including a double-stranded ribonucleic acid (dsRNA) capable of annealing and reducing expression of a huntingtin (HTT) mRNA, where the dsRNA includes a sense strand and an antisense strand, where one of the sense strand or the antisense strand of the dsRNA includes at least one lipophilic modification and the other strand of the dsRNA does not include a lipophilic modification; and where: (i) substantially all of the sense strand or the antisense strand of the dsRNA including the at least one lipophilic modification in the composition is duplexed with the strand that does not include a lipophilic modification, or (ii) the sense strand or the antisense strand of the dsRNA including the at least one lipophilic modification is present at less than 1% molar excess relative to the strand that does not include a lipophilic modification, the sense strand and the antisense strand are present at molar equivalence, or the strand that does not include a lipophilic modification is present in a molar excess relative to the strand of the dsRNA including the at least one lipophilic modification.

In one embodiment, the dsRNA targets a sequence in exon 1 of the huntingtin gene.

In certain embodiments, the dsRNA is AD-1019448, AD-1019465, AD-1271082, AD-1271083, AD-1271084, AD-1271085, AD-1498524, AD-1498526, or AD-1498528.

An additional aspect of the disclosure provides a composition including a double-stranded ribonucleic acid (dsRNA) including a sense strand and an antisense strand, where one of the sense strand or the antisense strand of the dsRNA includes at least one lipophilic modification at one or more internal residue(s) of the sense or antisense strand and the other strand of the dsRNA does not include a lipophilic modification; and where: (i) substantially all of the sense strand or the antisense strand of the dsRNA including the at least one lipophilic modification in the composition is duplexed with the strand that does not include a lipophilic modification, or (ii) the sense strand or the antisense strand of the dsRNA including the at least one lipophilic modification is present at less than 1% molar excess relative to the strand that does not include a lipophilic modification, the sense strand and the antisense strand are present at molar equivalence, or the strand that does not include a lipophilic modification is present in a molar excess relative to the strand of the dsRNA including the at least one lipophilic modification.

In some embodiments, the dsRNA includes at least one lipophilic modification at one or more internal residue(s) of the sense strand. Optionally, the dsRNA includes at least one lipophilic modification at any one of positions 4-8 or 13-18 counting from the 5′-end of the strand. Optionally, the dsRNA includes at least one lipophilic modification at position 6 counting from the 5′-end of the strand.

In certain embodiments, the dsRNA includes at least one lipophilic modification at one or more internal residue(s) of the antisense strand.

In one embodiment, the at least one lipophilic modification includes a saturated or unsaturated C₄-C₃₀ hydrocarbon. Optionally, the at least one lipophilic modification includes a C₄-C₃₀ alkyl or alkenyl. Optionally, the at least one lipophilic modification includes a linear C₆-C₁₈ alkyl or alkenyl. Optionally, the at least one lipophilic modification includes a C₁₆ alkyl. Optionally, the at least one lipophilic modification is attached at the 2′-ribo position of a nucleic acid residue of the dsRNA.

Another aspect of the disclosure provides a composition including a double-stranded ribonucleic acid (dsRNA) including a sense strand and an antisense strand, where one of the sense strand or the antisense strand of the dsRNA includes at least one lipophilic modification at one or more terminal residue(s) of the sense or antisense strand and the other strand of the dsRNA does not include a lipophilic modification; and where: (i) substantially all of the sense strand or the antisense strand of the dsRNA including the at least one lipophilic modification in the composition is duplexed with the strand that does not include a lipophilic modification, or (ii) the sense strand or the antisense strand of the dsRNA including the at least one lipophilic modification is present at less than 1% molar excess relative to the strand that does not include a lipophilic modification, the sense strand and the antisense strand are present at molar equivalence, or the strand that does not include a lipophilic modification is present in a molar excess relative to the strand of the dsRNA including the at least one lipophilic modification.

In certain embodiments, the dsRNA includes at least one lipophilic modification at the 5′-terminal and/or the 3′-terminal residue(s) of the sense strand. Optionally, the dsRNA includes at least one lipophilic modification at the 3′-terminal residue of the sense strand.

In some embodiments, the dsRNA includes at least one lipophilic modification at the 5′-terminal residue of the sense strand.

In one embodiment, the dsRNA includes at least one lipophilic modification at the 5′-terminal and/or the 3-terminal residue(s) of the antisense strand. Optionally, the dsRNA includes at least one lipophilic modification at the 3′-terminal residue of the antisense strand.

In another embodiment, the dsRNA includes at least one lipophilic modification at the 5′-terminal residue of the antisense strand.

In certain embodiments, the at least one lipophilic modification includes a saturated or unsaturated C₄-C₃₀ hydrocarbon. Optionally, the at least one lipophilic modification includes a C₄-C₃₀ alkyl or alkenyl. Optionally, the at least one lipophilic modification includes a linear C₆-C₁₈ alkyl or alkenyl. Optionally, the at least one lipophilic modification includes a C₁₆ alkyl. Optionally, the at least one lipophilic modification is attached at the 2′-ribo position of a nucleic acid residue of the dsRNA.

Another aspect of the instant disclosure provides a solid prepared by lyophilization of a composition of the disclosure.

A further aspect of the instant disclosure provides a kit that includes (a) a diluent including a divalent cation source and substantially free of inorganic phosphate and (b) a double-stranded ribonucleic acid (dsRNA) having a sense strand and an antisense strand, where the dsRNA includes at least one modified nucleotide that is not a 2′-deoxynucleotide, where the molar ratio of the dsRNA to calcium ion is greater than 1 to 2.

Another aspect of the instant disclosure provides (a) a solid prepared by lyophilization of a composition of the disclosure and (b) a diluent that is substantially free of inorganic phosphate.

A further aspect of the instant disclosure provides a method of treating a subject having a disorder that would benefit from a reduction in expression of a target gene, the method involving administering to the subject a therapeutically effective amount of a composition of the disclosure, thereby treating the subject.

In certain embodiments, the subject is a human.

In some embodiments, the target gene is amyloid precursor protein (APP), superoxide dismutase 1 (SOD1), or the huntingtin gene, optionally exon 1 of the huntingtin gene.

In one embodiment, the subject suffers from an APP-associated disease. In certain embodiments, the APP-associated disease is cerebral amyloid angiopathy (CAA), early onset Alzheimer's disease (EOAD), familial Alzheimer's disease, early onset familial Alzheimer disease (EOFAD), or late onset Alzheimer's disease. In certain embodiments, the APP-associated disease is Alzheimer's disease (AD).

In certain embodiments, APP expression is inhibited by at least about 30%.

In another embodiment, the method further involves administering an additional therapeutic agent to the subject.

In some embodiments, the dsRNA of the composition is administered at a dose of about 0.1 mg/kg to about 50 mg/kg.

In certain embodiments, the composition is administered to the subject intrathecally.

In one embodiment, administration of the composition to the subject causes a decrease in Aβ accumulation. Optionally, the administration of the composition to the subject causes a decrease in Aβ(1-40) and/or Aβ(1-42) accumulation.

In some embodiments administering the composition to the subject causes a decrease in amyloid plaque formation and/or accumulation in the subject.

In certain embodiments, the method reduces the expression of a target gene in a brain or spine tissue. In a related embodiment the brain or spine tissue is cortex, cerebellum, striatum, cervical spine, lumbar spine, and/or thoracic spine.

Another aspect of the instant disclosure provides a method of inhibiting the expression of APP in a subject, the method involving administering to the subject a therapeutically effective amount of a composition of the disclosure, thereby inhibiting the expression of APP in the subject.

An additional aspect of the instant disclosure provides a method for treating or preventing an APP-associated disease or disorder in a subject, the method involving administering to the subject a therapeutically effective amount of a composition of the disclosure, thereby treating or preventing an APP-associated disease or disorder in the subject.

In certain embodiments, the APP-associated disease or disorder is cerebral amyloid angiopathy (CAA) or Alzheimer's disease (AD). In one embodiment, the AD is early onset familial Alzheimer disease (EOFAD). In some embodiments, the APP-associated disease or disorder is early onset Alzheimer's disease (EOAD), familial Alzheimer's disease, or late onset Alzheimer's disease.

In some embodiments, the composition is administered by intrathecal injection. Optionally the intrathecal injection is performed in conjunction with intravenous administration.

Alternatively, the intrathecal administration is used in the absence of intravenous administration.

In some embodiments, administering the composition results in reduced intensity, severity, or frequency, or delayed onset of at least one symptom or feature of the APP-associated disease or disorder.

In certain embodiments, administering the composition results in no significant adverse effects in the subject. Optionally, the subject does not have tremors or twitches upon administering the composition to the subject.

In some embodiments, administering the composition takes place at least once every two weeks, once every month, once every two months, once every three months, one every four months, one every five months, and/or one every six months.

Another aspect of the disclosure provides a method for administering a dsRNA to a subject in need thereof, the method involving administering a composition of the disclosure intrathecally to the subject, thereby administering the dsRNA to the subject.

In one embodiment, the subject is a human.

In another embodiment, the dsRNA targets one or more of the following genes/mRNAs: amyloid precursor protein (APP); superoxide dismutase 1 (SOD1); and/or huntingtin (HTT). In certain embodiments, exon 1 of huntingtin gene mRNA is targeted.

Another aspect of the disclosure provides a method of inhibiting the expression of SOD1 in a cell or tissue of a subject, the method involving administering a composition of the disclosure to the subject in an amount sufficient to reduce SOD1 expression in the cell or tissue of the subject, thereby inhibiting the expression of SOD1 in the cell or tissue of the subject.

In one embodiment, the composition is administered intrathecally to the subject.

In certain embodiments, administering the dsRNA reduces the level of SOD1 mRNA in the cell or tissue of the subject by at least 50%, optionally by at least 80%, as compared to an appropriate control.

In some embodiments, the dsRNA of the composition is administered at a dose of about 0.1 mg/kg to about 50 mg/kg.

Another aspect of the disclosure provides a method of inhibiting the expression of HTT in a cell or tissue of a subject, the method involving administering a composition of the disclosure to the subject in an amount sufficient to reduce HTT expression in the cell or tissue of the subject, thereby inhibiting the expression of HTT in the cell or tissue of the subject.

In one embodiment, the composition includes a dsRNA that targets exon 1 of HTT.

In some embodiments, the dsRNA of the composition is administered at a dose of about 0.1 mg/kg to about 50 mg/kg.

Another aspect of the disclosure provides a kit for performing a method of the disclosure, the kit including a) a composition including dsRNA, and b) instructions for use, and c) optionally, a means for administering the composition to the subject.

An additional aspect of the disclosure provides a method for reducing or preventing particle formation in a solution that includes a divalent ion source and a double-stranded ribonucleic acid (dsRNA) having a sense strand and an antisense strand, where the dsRNA includes at least one lipophilic modification of the sense strand or the antisense strand, and either the sense or the antisense strand (the complementary strand) does not include a lipophilic modification, the method involving maintaining the sense strand or the antisense strand of the dsRNA including the lipophilic modification at less than 1% molar excess relative to the (complementary) strand that does not include a lipophilic modification, thereby reducing or preventing particle formation in the solution that includes the divalent ion source and the double-stranded ribonucleic acid (dsRNA).

In one embodiment, the sense strand and the antisense strand are present at molar equivalence.

In another embodiment, the strand that does not include a lipophilic modification is present in a molar excess relative to the strand of the dsRNA that includes the at least one lipophilic modification.

In an additional embodiment, the sense strand has the at least one lipophilic modification and the antisense strand does not have a lipophilic modification.

In an alternative embodiment, the antisense strand has the lipophilic modification and the sense strand does not have a lipophilic modification.

In certain embodiments, the divalent ion source is calcium, magnesium, copper, nickel, zinc, or strontium. In one embodiment, the divalent ion source is calcium.

In another embodiment, the at least one lipophilic modification is a C16 or longer lipophilic modification.

Another aspect of the disclosure provides a method for preparing a formulation comprising annealing a sense strand and an antisense strand, wherein one of the sense strand and antisense strand contains a lipophilic modification, to form a duplex solution comprising a double stranded RNA (dsRNA); lyophilizing the duplex solution to provide a duplex composition; and dissolving the duplex composition in an injection solution, wherein the injection solution comprises a divalent cation source (e.g., calcium) and does not comprise a phosphate buffer; and the duplex composition comprises 0-5% molar excess (e.g., about 1-2% molar excess) of antisense strand over sense strand.

In certain embodiments, the divalent ion source is calcium, magnesium, copper, nickel, zinc, or strontium. In one embodiment, the divalent ion source is calcium.

In one embodiment, the duplex composition comprises about a 1-2% molar excess of antisense strand over sense strand.

In another embodiment, the sense strand comprises the at least one lipophilic modification and the antisense strand does not comprise a lipophilic modification.

In another embodiment, the at least one lipophilic modification is a C16 or longer lipophilic modification.

In one embodiment, the dsRNA is selected from the group consisting of AD-961583, AD-454973, AD-454843, AD-961584, AD-961585, and AD-961586.

### BRIEF DESCRIPTION OF THE DRAWINGS

FIGS. 1A and 1B show modified duplex structures and the components of exemplified formulations of the instant disclosure, including both diluent and drug product formulations, respectively. FIG. 1A shows graphic representations of four related, modified APP-targeting duplex structures, AD-454844, AD-1302922, AD-1302923, and AD-1999409. The indicated duplexes include the following sequences: AD-454844: sense strand SEQ ID NO: 3, antisense strand SEQ ID NO: 14; AD-1302922: sense strand SEQ ID NO: 9, antisense strand SEQ ID NO: 20; AD1302922: sense strand SEQ ID NO: 10, antisense strand SEQ ID NO: 21; and AD-1999409: sense strand SEQ ID NO: 11, antisense strand SEQ ID NO: 22. Baseline sequences for each of these four duplexes were identical, and all four duplexes included an identical antisense strand (A-882382; SEQ ID NOs: 14 and 20-22 are identical). These four duplexes were distinguished in the following manner: (1) the AD-454844 duplex harbors a 2′-O—C16 modification at the sixth residue from the 5′-terminus of the sense strand (A-882381; SEQ ID NO: 3); (2) the AD-1302922 duplex shifts the 2′-O—C16 modification to the 5′-terminal residue of the sense strand (A-2364988; SEQ ID NO: 9) of the AD-1302922 duplex; (3) the AD-1302923 duplex harbors a 2′-O-docosanyl-cytidine-3′-Phosphate (C22) modification at the sixth residue from the 5′-terminus of the sense strand (A-2365995; SEQ ID NO: 10); and (4) the AD-1999409 duplex harbors a 2′-O-decyl-cytidine-3′-Phosphate (C10) modification at the sixth residue from the 5′-terminus of the sense strand (A-3724055; SEQ ID NO: 11). FIG. 1B shows tabulated components of such formulations.

FIG. 2 shows that the APP-targeting siRNA formulation “APP F3 DP”, which contains a molar excess by 0.1% of the non-lipophile-modified antisense strand relative to the lipophile-modified sense strand, did not form particulates (right panel), nor did particulates form in a new siRNA formulation (“APP F8 DP”, middle panel) possessing a molar excess by 5% of the non-lipophile-modified antisense strand relative to the lipophile-modified sense strand. In contrast, particulates did form in a different new siRNA formulation (“APP F7 DP”, left panel) that featured a molar excess by 5% of the lipophile-modified sense strand relative to the non-lipophile-modified antisense strand. Such pellets were therefore only observed when the C16 lipophilic moiety-modified sense strand was in excess.

FIG. 3 shows that particulate formation in calcium exchange studies that was observed for the “APP F4 DP” siRNA formulation (right panel) that possessed a molar excess by 0.85% of the lipophile-modified sense strand relative to the non-lipophile-modified antisense strand, could effectively be rescued by instead preparing such formulations with an excess of the non-lipophile-modified antisense strand. Neither new formulation “APP F5 DP”, having a molar excess by 1.5% of the non-lipophile-modified antisense strand relative to the lipophile-modified sense strand, nor new formulation “APP F6 DP”, having a molar excess by 7.4% of the non-lipophile-modified antisense strand relative to the lipophile-modified sense strand, were observed to form particulate. As in FIG. 2 above, particulates were only observed when the C16 lipophilic moiety-modified sense strand was in molar excess relative to the non-lipophile-modified antisense strand.

FIGS. 4A to 4E show that increased turbidity was observed in duplex samples prepared in solutions containing divalent cations and having a molar excess of sense strand to antisense strand for such duplexes, and rescue of such turbidity was also assessed under various conditions. FIG. 4A shows the turbidity observed by visual inspection of duplex samples in solution, where each duplex was produced in a sodium solution (“Before”, top row of vials) with a 5% molar excess of sense strand relative to antisense strand. Samples were then subjected to counter ion exchange (calcium exchange was performed upon all duplexes, while magnesium exchange was performed upon one of the C16-containing duplexes, shown at left). Turbid suspensions were seen for all duplex solutions other than a C10-containing duplex, after counter-ion exchange (“After” duplex solutions, of the bottom row, had undergone ultrafiltration (UF) to high salt concentration solution (20 mM CaCl2) or 50 mM MgCl2) and then low salt concentration solution (1 mM CaCl2) or MgCl2)). FIG. 4B shows that position of the lipophilic modification significantly impacted the extent of turbidity observed in duplex solutions. While the terminal C16-containing duplex was highly turbid when formulated with the sense strand in excess, even the terminal C16-containing duplex could be rescued from turbidity when titrated into the antisense strand being in excess. FIG. 4C shows that lipophilic moieties with chain length longer than C10 produced elevated levels of turbidity under conditions of sense strand excess and counter ion presence. While both internal C16-containing and internal C22-containing duplexes showed elevated levels of turbidity relative to an internal C10-containing duplex at all sense strand:antisense strand ratios examined, an internal C22-containing duplex exhibited only slightly increased turbidity relative to a corresponding internal C16-containing duplex. FIG. 4D shows the impact of counter ion upon precipitate formation/turbidity. Both calcium and magnesium-exchanged C16 duplexes exhibited similar turbidity behaviors and were rescued in a similar trend (by eliminating conditions of sense strand excess). FIG. 4E shows that sequence variations between duplexes with parallel nucleotide modification patterns could also significantly impact precipitate formation and associated levels of turbidity. Turbidity of the terminal C16-modified AD-1302922 APP-targeting duplex and of the internal C16-modified AD-454844 APP-targeting duplex, which share common sequences and identical antisense strands, were compared with a distinct, internal C-16 modified APP-targeting duplex, AD-961583, shown in additional detail below and labeled here simply as “APP”. When compared to precipitate rescue data for the AD-961583 duplex, both internal and terminal C16-modified duplexes AD-1302922 and AD-454844, respectively, exhibited far less turbidity than the AD-961583 duplex.

FIGS. 5A and 5B show confirmation of the impact of antisense strand excess in rescuing turbidity in counter ion-containing solutions for an internal C16 lipophile-containing “AD-961583” APP-targeting duplex. FIG. 5A shows a schematic diagram of the APP-targeting AD-961583 duplex (sense strand: SEQ ID NO: 4; antisense strand: SEQ ID NO: 15) and antisense strand titration of a suspension of the AD-961583 duplex, which formed particulates under conditions of sense strand excess and counter ion (divalent cation) presence. In all cases, turbidity (upper row vials) was resolved via antisense strand addition (lower row vials). FIG. 5B shows quantitated and plotted turbidity levels of titrated samples.

FIGS. 6A and 6B show the impact of antisense strand excess upon turbidity in counter ion-containing solutions of an internal C16 lipophile-containing “AD-1395762” superoxide dismutase 1 (SOD1)-targeting duplex. FIG. 6A shows a schematic diagram of the SOD1-targeting AD-1395762 duplex (sense strand: SEQ ID NO: 74; antisense strand: SEQ ID NO: 93) and antisense strand titration of a suspension of the AD-1395762 duplex, which, unlike the APP-targeting duplexes examined herein, did not form significant particulates even under conditions of sense strand excess of 5.8% and counter ion (divalent cation) presence. In all instances, no turbidity was observed by visual inspection. FIG. 6B shows quantitated and plotted turbidity levels of titrated samples.

FIGS. 7A and 7B show confirmation of the impact of antisense strand excess in rescuing turbidity in counter ion-containing solutions for an internal C16 lipophile-containing “AD-1498524” huntingtin gene exon 1 (HTTex1)-targeting duplex. FIG. 7A shows a schematic diagram of the HTT exon 1-targeting AD-1498524 duplex (sense strand: SEQ ID NO: 83; antisense strand: SEQ ID NO: 102) and antisense strand titration of a suspension of the AD-1498524 duplex, which formed particulates under conditions of sense strand excess and counter ion (divalent cation) presence. Any observed turbidity (upper row vials) was resolved via antisense strand addition (lower row vials). FIG. 7B shows quantitated and plotted turbidity levels of titrated samples.

FIG. 8 shows a graphic representation of five related, modified APP-targeting duplex structures, AD-454844, AD-1302922, AD-1302923, AD-1999409 (the preceding also depicted in FIG. 1A above), and AD-960500. The indicated duplexes include the following sequences: AD-454844: sense strand SEQ ID NO: 3, antisense strand SEQ ID NO: 14; AD-1302922: sense strand SEQ ID NO: 9, antisense strand SEQ ID NO: 20; AD-1302922: sense strand SEQ ID NO: 10, antisense strand SEQ ID NO: 21; AD-1999409: sense strand SEQ ID NO: 11, antisense strand SEQ ID NO: 22; and AD-960500: sense strand SEQ ID NO: 162, antisense strand SEQ ID NO: 163. Baseline sequences for each of these five duplexes were identical, and all five duplexes included an identical antisense strand (A-882382; SEQ ID NOs: 14, 20-22, and 163 are identical). These five duplexes were distinguished in the following manner: (1) the AD-454844 duplex harbors a 2′-O—C16 modification at the sixth residue from the 5′-terminus of the sense strand (A-882381, SEQ ID NO: 3); (2) the AD-1302922 duplex shifts the 2′-O—C16 modification to the 5′-terminal residue of the sense strand (A-2364988; SEQ ID NO: 9) of the AD-1302922 duplex; (3) the AD-1302923 duplex harbors a 2′-O-docosanyl-cytidine-3′-Phosphate (C22) modification at the sixth residue from the 5-terminus of the sense strand (A-2365995; SEQ ID NO: 10); (4) the AD-1999409 duplex harbors a 2′-O-decyl-cytidine-3′-Phosphate (C10) modification at the sixth residue from the 5-terminus of the sense strand (A-3724055; SEQ ID NO: 11); and (5) the AD-960500 duplex harbors no lipophilic modification.

FIGS. 9A to 9C show that increased turbidity was observed in duplex samples prepared in solutions containing either Ca²⁺ or Mg²⁺ ions and having a molar excess of sense strand to antisense strand for such duplexes, and that such turbidity could be reversed by adding antisense strand to the samples.

FIG. 9A shows the turbidity observed by visual inspection of duplex samples in solution, where each duplex was produced in a sodium solution with a 5% molar excess of sense strand relative to antisense strand. Samples were then subjected to counter ion exchange (calcium exchange was performed upon duplexes AD-960500 (no lipophile modification), AD-454844 (internal C16 sense strand lipophile modification), AD-1302922 (terminal C16 sense strand lipophile modification), AD-1302923 (internal C22 sense strand lipophile modification), and AD-1999409 (internal C10 sense strand lipophile modification), while magnesium exchange was performed upon parallel solutions for duplex AD-454844 (internal C16 sense strand lipophile modification) and AD-1302922 (terminal C16 sense strand lipophile modification)). Some turbidity was observed for all duplex solutions other than the AD-960500 control duplex (having no lipophile modification) and the C10-containing AD-1999409 duplex, after indicated counter-ion exchange.

FIG. 9B shows that rescue of turbidity was achieved via addition of greater amounts of antisense strand to each calcium exchange duplex solution that produced turbidity under conditions of sense (lipophile-modified) strand excess. For all duplex solutions, effectively no turbidity was observed when a molar excess of antisense strand relative to sense (lipophile-modified) strand was produced and subjected to calcium exchange.

FIG. 9C shows that rescue of turbidity was achieved via addition of greater amounts of antisense strand to each calcium exchange or magnesium exchange duplex solution that produced turbidity under conditions of sense (lipophile-modified) strand excess (noting that calcium exchange terminal C16 solution produced highest levels of turbidity when sense strand was in excess, as contrasted with magnesium exchange C16 solution when sense strand was in excess, which produced significantly lower levels of turbidity). As above, for all duplex solutions examined, effectively no turbidity was observed when a molar excess of antisense strand relative to sense (lipophile-modified) strand was produced and subjected to divalent cation exchange (whether calcium exchange or magnesium exchange).

FIG. 10A depicts a representative ion-paring reverse phase (IPRP) HPLC for annealing of APP duplex, ALN-961583.

FIG. 10B depicts a representative ion-paring reverse phase (IPRP) HPLC for annealing of SOD-duplex, ALN-1395762.

FIG. 10C depicts a representative ion-paring reverse phase (IPRP) HPLC for annealing of HTT duplex, ALN-1498524.

FIG. 11A depicts the sodium salt form of the formulation, including structure and molecular formula, for APP duplex AD-961583.

FIG. 11B depicts the sodium salt form of the formulation, including structure and molecular formula, for HTT duplex AD-1498524.

FIG. 11C depicts the sodium salt form of the formulation, including structure and molecular formula, for SOD1 duplex AD-1395762.

The present disclosure is further illustrated by the following detailed description.

### DETAILED DESCRIPTION OF THE INVENTION

The present disclosure, at least in part, provides compositions for enhanced formulation of iRNA agents for CNS-directed delivery (e.g., via intrathecal injection), as well as resultant drug product formulations, associated methods, kits and other compositions. In order to administer dsRNAs to a subject via parenteral administration, the dsRNA must be formulated into a suitable aqueous solution. In the course of research into preparing such formulations, it has been surprisingly discovered that dsRNAs having at least one lipophilic modification must be formulated in a particular way to avoid long-term stability problems. In particular, formulation of dsRNAs in the presence of a bivalent cation (e.g., calcium, magnesium, copper, nickel, zinc, or strontium) can result in problematic precipitation issues where the stoichiometry of the individual strands of a dsRNA in the formulation should be controlled to prevent precipitation of lipophilic molecules in the aqueous solution.

The oligonucleotides used in the compositions and methods of the disclosure is a double-stranded RNA and is referred to herein as a “double stranded RNAi agent,” “double stranded RNA (dsRNA) molecule,” “dsRNA agent,” “dsRNA”, “RNAi”, “iRNA”, or “iRNA agent”. The term “dsRNA” refers to a complex of one or more (e.g., two) ribonucleic acid molecules, having a duplex structure comprising two anti-parallel and substantially complementary nucleic acid portions (e.g., strands), referred to as having “sense” and “antisense” orientations with respect to a target RNA, i.e., an APP gene. In some embodiments of the disclosure, a double stranded RNA (dsRNA) triggers the degradation of a target RNA, e.g., an mRNA, through a post-transcriptional gene-silencing mechanism referred to herein as RNA interference or RNAi. In one embodiment, the dsRNA contains two separate strands that form the duplex structure. In another embodiment, the dsRNA is a single oligonucleotide having two separate portions that form the duplex structure, the two portions being part of a hairpin or dumbbell type structure. Hairpin and dumbbell type oligomeric compounds may have a duplex region equal to, or at least, 14, 15, 15, 16, 17, 18, 19, 29, 21, 22, 23, 24, or 25 nucleotide pairs. In some embodiments, the duplex region can be equal to or less than 200, 100, or 50, in length. In some embodiments, ranges for the duplex region are 15-30, 17 to 23, 19 to 23, and 19 to 21 nucleotides pairs in length. In some embodiments, the hairpin oligomeric compounds can have a single strand overhang or terminal unpaired region, in some embodiments at the 3, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

In addition to stoichiometry control of the sense and antisense duplexes, certain of the solutions and formulations provided herein may also be substantially free of inorganic phosphate. It was further discovered herein that nucleic acid formulations substantially free of inorganic phosphate but also formulated for intrathecal delivery by the addition of a divalent cation (such as calcium) exhibited significantly reduced particulate formation in solution, as compared to corresponding solutions and formulations having a significant source of inorganic phosphate.

Without wishing to be bound by theory, particulate formation was observed in certain dsRNA formulations prepared for delivery by intrathecal injection that included a calcium ion source. Particulate formation was identified as likely attributable to interaction between calcium ions and excess of a lipophilic moiety-containing strand of a dsRNA (optionally in the presence of an inorganic phosphate source) promoting particulate/crystal formation. Because inclusion of a calcium ion source in intrathecal formulations has been observed to reduce the prevalence and magnitude of certain adverse events (e.g., tremors, twitches, etc.) in subjects administered nucleic acid formulations via intrathecal injection, removal of inorganic phosphate sources from such nucleic acid formulations was attempted herein. Remarkably, particulate-free nucleic acid formulations were discovered, that were capable of robust nucleic acid delivery when administered to subjects via intrathecal injection.

Certain compositions of the disclosure therefore carry an excess of a non-lipophile-modified strand of a dsRNA relative to a lipophile-modified strand of the dsRNA. Some compositions are also substantially free of sources of inorganic phosphate. Compositions that are specifically exemplified herein do not include a source of inorganic phosphate, though it is contemplated in certain embodiments that mitigation against particulate formation in nucleic acid formulations of the disclosure can be achieved by reducing sources of inorganic phosphate in such compositions, e.g., to 100 ppm or less, to 50 ppm or less, or to 10 ppm or less.

In embodiments, the compositions of the disclosure are employed for delivery of nucleic acid agents, e.g., iRNA agents, including dsRNAs as specifically exemplified herein. The level of nucleic acid agent in a composition of the instant disclosure can range, e.g., from about 5 mg/mL to about 300 mg/mL. In a related embodiment, the composition includes a nucleic acid agent at about 10 mg/mL to about 200 mg/mL. In a further embodiment, the composition includes a nucleic acid agent at about 20 mg/mL to about 100 mg/mL. In one embodiment, the composition includes a nucleic acid agent at about 40 mg/mL to about 80 mg/mL. Optionally, the composition includes a nucleic acid agent at about 60 mg/mL.

Compositions of the present disclosure may comprise a number of salts that provide sources of physiologically relevant ionic species, such as Na⁺, K⁺, Mg²⁺, Cl⁻, or Ca²⁺. These may include, without limitation, sodium chloride, potassium chloride, magnesium chloride, and calcium chloride. The compositions may further comprise other trace elements and their salts, including, but not limited to, selenium, copper, chromium, iodine, fluoride, zinc, manganese, molybdenum, and iron.

Sodium ions are included in relatively large concentrations in the formulations of the instant disclosure, at least in part in view of their role in normal physiological functioning. Na⁺ is the major cation of the extracellular fluid. It plays an important role in many physiological processes, including the regulation of blood volume, blood pressure, osmotic equilibrium, and pH, as well as the generation of nerve impulses.

Potassium ions are the major cation of intracellular fluid, and, with the sodium ions of the extracellular fluid, K⁺ is a primary generator of the electrical potential across cellular membranes. Accordingly, it plays a significant role in normal functioning, and is relevant to such body functions as neurotransmission, muscle contraction, and heart function.

Calcium ions are likewise important to many physiological processes. In particular, Ca²⁺ ions are one of the most widespread second messengers used in signal transduction. In endothelial cells, Ca^{2|} ions may regulate several signaling pathways which cause smooth muscles surrounding blood vessels to relax. Dysfunction within Ca²⁺-activated pathways can lead to an increase in tone caused by unregulated smooth muscle contraction. This type of dysfunction can be seen in cardiovascular diseases, hypertension, and diabetes. Inclusion of Ca²⁺ ions in the compositions of the instant disclosure also has been identified to mitigate against certain adverse events (e.g., tremors, twitches, other neurological issues) seen in subjects administered calcium-free or low calcium nucleic acid formulations via intrathecal injection.

Magnesium ions are used in relatively large concentrations in normal metabolism. It is recognized that deficiency of magnesium is rare unless it is accompanied by severe losses in other electrolytes such as in vomiting and diarrhea. It is however frequently recognized as deficient in the modern diet with symptoms such as muscle tremors and weakness. This mineral is important in many enzymatic reactions and will stabilize excitable membranes. Administered intravenously, magnesium may produce an anesthetic action and this is indirect evidence of its action on the vascular wall endothelial component to stabilize and normalize the surface of the vascular wall.

In some embodiments, a composition of the present disclosure includes a sodium ion (Na⁺) source (e.g., provided as sodium chloride) at a concentration between 0.1 mM and 1 M. In a related embodiment, a Na⁺ source is present at a concentration between about 40 mM and about 300 mM. Optionally, a Na⁺ source is present at a concentration between about 70 mM and about 200 mM. In a related embodiment, a Na⁺ source is present at a concentration between about 80 mM and about 120 mM. In a related embodiment, a Na⁺ source is present at a concentration between about 80 mM and about 110 mM. In a related embodiment, a Na⁺ source is present at a concentration between about 85 mM and about 100 mM. In a related embodiment, a Na⁺ source is present at a concentration between about 90 mM and about 100 mM. In an alternative embodiment, a Na⁺ source is present at a concentration between about 90 mM and about 105 mM, optionally between about 95 mM and about 100 mM. In a related embodiment, a Na⁺ source is present at a concentration of about 97 mM to about 98 mM. In one embodiment, a Na⁺ source is present at a concentration of about 97.6 mM. In one embodiment, a Na⁺ source is present at a concentration of about 97.0 mM. In one embodiment, a Na⁺ source is present at a concentration of about 89.5 mM. In one embodiment, a Na⁺ source is present at a concentration of 97.6 mM. In one embodiment, a Na⁺ source is present at a concentration of 97.56 mM.

In certain embodiments, a composition of the present disclosure includes a calcium ion (Ca^{2|}) source (e.g., provided as calcium chloride) at a concentration between 0.1 mM and 1 M. In some embodiments, a Ca²⁺ source is present at a concentration between about 0.1 mM and about 200 mM. In a further embodiment, a Ca²⁺ source is present at a concentration between about 0.5 mM and about 100 mM. Optionally, a Ca²⁺ source is present at a concentration between about 0.8 mM and about 50 mM. In some embodiments, a Ca²⁺ source is present at a concentration between about 1 mM and about 25 mM. In some embodiments, a Ca²⁺ source is present at a concentration between about 2 mM and about 20 mM. In some embodiments, a Ca²⁺ source is present at a concentration between about 8 mM and about 20 mM. In some embodiments, a Ca²⁺ source is present at a concentration between about 5 mM and about 15 mM. Alternatively, a Ca²⁺ source is present at a concentration between about 10 mM and about 15 mM. In some embodiments, a Ca²⁺ source is present at a concentration between about 12 mM and about 15 mM. In some embodiments, a Ca²⁺ source is present at a concentration between about 12 mM and about 14 mM. In some embodiments, a Ca²⁺ source is present at a concentration between about 12.5 mM and about 13.5 mM. Optionally, a Ca²⁺ source is present at a concentration of about 12.5 mM, about 12.75 mM, about 12.9 mM, about 13.0 mM, about 13.25 mM, about 13.5 mM, or about 13.9 mM. In certain embodiments, a Ca²⁺ source is present at a concentration of about 13.0 mM. In certain embodiments, a Ca²⁺ source is present at a concentration of 13.0 mM.

In some embodiments, a composition of the present disclosure comprises a potassium ion (K⁺) source (e.g., provided as potassium chloride) at a concentration between 0.0 mM and 1 M. In some embodiments, a K⁺ source is present at a concentration between about 0.1 mM and about 100 mM. In a further embodiment, a K⁺ source is present at a concentration between about 0.2 mM and about 40 mM. In a further embodiment, a K⁺ source is present at a concentration between about 0.5 mM and about 20 mM. In some embodiments, a K⁺ source is present at a concentration between about 1 mM and about 5 mM. In some embodiments, a K⁺ source is present at a concentration between about 1 mM and about 4 mM. In some embodiments, a K⁺ source is present at a concentration between about 1 mM and about 3 mM. In some embodiments, a K⁺ source is present at a concentration between about 1.0 mM and about 2.5 mM. In some embodiments, a K⁺ source is present at a concentration between about 1.5 mM and about 2.5 mM. In some embodiments, a K⁺ source is present at a concentration between about 1.5 mM and about 2.2 mM. Optionally, a K⁺ source is present at a concentration of about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.1 mM, about 2.2 mM, about 2.3 mM, about 2.4 mM, about 2.5 mM, about 2.6 mM, about 2.7 mM, about 2.8 mM, about 2.9 mM, or about 3.0 mM. In certain embodiments, a K⁺ source is present at a concentration of about 1.9 mM. In certain embodiments, a K⁺ source is present at a concentration of 1.9 mM.

In certain embodiments, a composition of the present disclosure comprises a magnesium ion (Mg²⁺) source (e.g., provided as magnesium chloride) at a concentration between 0.0 mM and 1 M. In some embodiments, a Mg²⁺ source is present at a concentration between about 0.01 mM and about 100 mM. In a further embodiment, a Mg²⁺ source is present at a concentration between about 0.1 mM and about 40 mM. In a further embodiment, a Mg²⁺ source is present at a concentration between about 0.2 mM and about 20 mM. In some embodiments, a Mg²⁺ source is present at a concentration between about 0.3 mM and about 5 mM. In some embodiments, a Mg²⁺ source is present at a concentration between about 0.3 mM and about 4 mM. In some embodiments, a Mg²⁺ source is present at a concentration between about 0.3 mM and about 3 mM. In some embodiments, a Mg²⁺ source is present at a concentration between about 0.3 mM and about 2 mM. In some embodiments, a Mg²⁺ source is present at a concentration between about 0.3 mM and about 1 mM. In some embodiments, a Mg²⁺ source is present at a concentration between about 0.1 mM and about 1 mM. In some embodiments, a Mg²⁺ source is present at a concentration between about 0.3 mM and about 0.7 mM. In some embodiments, a Mg²⁺ source is present at a concentration between about 0.3 mM and about 0.6 mM. In some embodiments, a Mg²⁺ source is present at a concentration between about 0.4 mM and about 0.6 mM. Optionally, a Mg²⁺ source is present at a concentration of about 0.3 mM, about 0.4 mM, about 0.5 mM, about 0.6 mM, about 0.7 mM, about 0.8 mM, or about 0.9 mM. In certain embodiments, a Mg²⁺ source is present at a concentration of about 0.5 mM. In a further embodiment, a Mg²⁺ source is present at a concentration of 0.5 mM. Alternatively, a Mg²⁺ source is present at a concentration of 0.52 mM.

In some embodiments, a composition of the disclosure has a molar ratio of divalent cation source-to-nucleic acid agent of greater than about 2:1. In certain embodiments, a composition of the disclosure has a molar ratio of divalent cation source-to-nucleic acid agent of greater than about 2.5:1. In further embodiments, a composition of the disclosure has a molar ratio of divalent cation source-to-nucleic acid agent of greater than about 3:1. In other embodiments, a composition of the disclosure has a molar ratio of divalent cation source-to-nucleic acid agent of greater than about 3.5:1. In further related embodiments, a composition of the disclosure has a molar ratio of divalent cation source-to-nucleic acid agent of greater than about 4:1. In certain related embodiments, a composition of the disclosure has a molar ratio of divalent cation source-to-nucleic acid agent between about 2:1 and about 10:1. Optionally, a composition of the disclosure has a molar ratio of divalent cation source-to-nucleic acid agent between about 3:1 and about 10:1. Optionally, a composition of the disclosure has a molar ratio of divalent cation source-to-nucleic acid agent between about 3:1 and about 9:1. Optionally, a composition of the disclosure has a molar ratio of divalent cation source-to-nucleic acid agent between about 3:1 and about 8:1. Optionally, a composition of the disclosure has a molar ratio of divalent cation source-to-nucleic acid agent between about 3:1 and about 7:1. Optionally, a composition of the disclosure has a molar ratio of divalent cation source-to-nucleic acid agent between about 3:1 and about 6:1. Optionally, a composition of the disclosure has a molar ratio of divalent cation source-to-nucleic acid agent between about 3:1 and about 5:1.

In some embodiments of the solutions provided herein, the pH of the solution is between 4 and 10, optionally between 6 and 10. In related embodiments, the pH of the solution is between about 6.5 and about 8.0. In a further embodiment, the pH of the solution is 6.5-7.8. In certain embodiments of the solutions provided herein, the pH of the solution is 6.7-7.5. In some embodiments, the pH of the solution is 6.8-7.2. In further embodiments, the pH of the solution is about 6.9.

In some embodiments of the compositions provided herein, an aqueous solution of the disclosure has an osmolality between about 100 and 500 mOsm/kg. In a further embodiment, an aqueous solution of the disclosure has an osmolality between about 200 and 400 mOsm/kg.

In certain embodiments, the compositions (i.e., aqueous compositions) provided herein can be stored for at least 4 hours, at least 6 hours, at least 8 hours, at least 12 hours, at least 18 hours, at least 24 hours, at least 36 hours, at least 48 hours, at least 72 hours, at least 96 hours, at least 120 hours, at least 144 hours, at least one week, at least two weeks, at least three weeks, or at least one month at 25° C. without measurable precipitation of solutes and/or measurable loss of the capability to produce knockdown of a target gene in a subject administered such a solution via intrathecal injection. In some embodiments, the compositions provided herein can be stored for at least 4 hours, at least 6 hours, at least 8 hours, at least 12 hours, at least 18 hours, at least 24 hours, at least 36 hours, at least 48 hours, at least 72 hours, at least 96 hours, at least 120 hours, at least 144 hours, at least one week, at least two weeks, at least three weeks, or at least one month at 2-8° C. without measurable precipitation of solutes and/or measurable loss of the capability of the capability to produce knockdown of a target gene in a subject administered such a solution via intrathecal injection.

### Pharmaceutical Compositions of the Disclosure

The present disclosure provides pharmaceutical compositions and formulations which include the RNAi agents described herein (e.g., AD-454973, AD-454843, AD-961583, AD-961584, AD-961585, AD-961586, AD-454844, AD-1302922, AD-1302923, or AD-1999409, etc.); however, it is expressly contemplated that the formulations of the instant disclosure can be employed for delivery of any RNAi agent. In one embodiment, provided herein are pharmaceutical compositions containing an RNAi agent, as described herein, and a pharmaceutically acceptable carrier. The pharmaceutical compositions containing the RNAi agent are useful for treating a disease or disorder associated with the expression or activity of a gene (e.g., APP, etc.) to treat a disorder such as, for example, Alzheimer's disease.

The pharmaceutical compositions of the disclosure may be administered in dosages sufficient to inhibit expression of an APP gene. In general, a suitable dose of an RNAi agent of the disclosure will be in the range of about 0.001 to about 200.0 milligrams per kilogram body weight of the recipient per day, generally in the range of about 1 to 50 mg per kilogram body weight per day. For example, the dsRNA can be administered at about 0.01 mg/kg, about 0.05 mg/kg, about 0.5 mg/kg, about 1 mg/kg, about 1.5 mg/kg, about 2 mg/kg, about 3 mg/kg, about 10 mg/kg, about 20 mg/kg, about 30 mg/kg, about 40 mg/kg, or about 50 mg/kg per single dose.

For example, a dsRNA as disclosed herein may be administered at a dose of about 0.1, 0.2, 0.3, 0.4, 0.5, 0.6, 0.7. 0.8, 0.9, 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, or about 10 mg/kg. Values and ranges intermediate to the recited values are also intended to be part of this disclosure.

In another embodiment, the dsRNA is administered at a dose of about 0.1 to about 50 mg/kg, about 0.25 to about 50 mg/kg, about 0.5 to about 50 mg/kg, about 0.75 to about 50 mg/kg, about 1 to about 50 mg/mg, about 1.5 to about 50 mg/kb, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.1 to about 45 mg/kg, about 0.25 to about 45 mg/kg, about 0.5 to about 45 mg/kg, about 0.75 to about 45 mg/kg, about 1 to about 45 mg/mg, about 1.5 to about 45 mg/kb, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.1 to about 40 mg/kg, about 0.25 to about 40 mg/kg, about 0.5 to about 40 mg/kg, about 0.75 to about 40 mg/kg, about 1 to about 40 mg/mg, about 1.5 to about 40 mg/kb, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.1 to about 30 mg/kg, about 0.25 to about 30 mg/kg, about 0.5 to about 30 mg/kg, about 0.75 to about 30 mg/kg, about 1 to about 30 mg/mg, about 1.5 to about 30 mg/kb, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.1 to about 20 mg/kg, about 0.25 to about 20 mg/kg, about 0.5 to about 20 mg/kg, about 0.75 to about 20 mg/kg, about 1 to about 20 mg/mg, about 1.5 to about 20 mg/kb, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. Values and ranges intermediate to the recited values are also intended to be part of this disclosure.

For example, the dsRNA may be administered at a dose of about 0.01, 0.02, 0.03, 0.04, 0.05, 0.06, 0.07, 0.08, 0.09, 0.1, 0.2, 0.3, 0.4, 0.5, 0.6, 0.7. 0.8, 0.9, 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, or about 10 mg/kg. Values and ranges intermediate to the recited values are also intended to be part of this disclosure.

In another embodiment, the dsRNA is administered at a dose of about 0.5 to about 50 mg/kg, about 0.75 to about 50 mg/kg, about 1 to about 50 mg/mg, about 1.5 to about 50 mg/kb, about 2 to about 50 mg/kg, about 2.5 to about 50 mg/kg, about 3 to about 50 mg/kg, about 3.5 to about 50 mg/kg, about 4 to about 50 mg/kg, about 4.5 to about 50 mg/kg, about 5 to about 50 mg/kg, about 7.5 to about 50 mg/kg, about 10 to about 50 mg/kg, about 15 to about 50 mg/kg, about 20 to about 50 mg/kg, about 20 to about 50 mg/kg, about 25 to about 50 mg/kg, about 25 to about 50 mg/kg, about 30 to about 50 mg/kg, about 35 to about 50 mg/kg, about 40 to about 50 mg/kg, about 45 to about 50 mg/kg, about 0.5 to about 45 mg/kg, about 0.75 to about 45 mg/kg, about 1 to about 45 mg/mg, about 1.5 to about 45 mg/kb, about 2 to about 45 mg/kg, about 2.5 to about 45 mg/kg, about 3 to about 45 mg/kg, about 3.5 to about 45 mg/kg, about 4 to about 45 mg/kg, about 4.5 to about 45 mg/kg, about 5 to about 45 mg/kg, about 7.5 to about 45 mg/kg, about 10 to about 45 mg/kg, about 15 to about 45 mg/kg, about 20 to about 45 mg/kg, about 20 to about 45 mg/kg, about 25 to about 45 mg/kg, about 25 to about 45 mg/kg, about 30 to about 45 mg/kg, about 35 to about 45 mg/kg, about 40 to about 45 mg/kg, about 0.5 to about 40 mg/kg, about 0.75 to about 40 mg/kg, about 1 to about 40 mg/mg, about 1.5 to about 40 mg/kb, about 2 to about 40 mg/kg, about 2.5 to about 40 mg/kg, about 3 to about 40 mg/kg, about 3.5 to about 40 mg/kg, about 4 to about 40 mg/kg, about 4.5 to about 40 mg/kg, about 5 to about 40 mg/kg, about 7.5 to about 40 mg/kg, about 10 to about 40 mg/kg, about 15 to about 40 mg/kg, about 20 to about 40 mg/kg, about 20 to about 40 mg/kg, about 25 to about 40 mg/kg, about 25 to about 40 mg/kg, about 30 to about 40 mg/kg, about 35 to about 40 mg/kg, about 0.5 to about 30 mg/kg, about 0.75 to about 30 mg/kg, about 1 to about 30 mg/mg, about 1.5 to about 30 mg/kb, about 2 to about 30 mg/kg, about 2.5 to about 30 mg/kg, about 3 to about 30 mg/kg, about 3.5 to about 30 mg/kg, about 4 to about 30 mg/kg, about 4.5 to about 30 mg/kg, about 5 to about 30 mg/kg, about 7.5 to about 30 mg/kg, about 10 to about 30 mg/kg, about 15 to about 30 mg/kg, about 20 to about 30 mg/kg, about 20 to about 30 mg/kg, about 25 to about 30 mg/kg, about 0.5 to about 20 mg/kg, about 0.75 to about 20 mg/kg, about 1 to about 20 mg/mg, about 1.5 to about 20 mg/kb, about 2 to about 20 mg/kg, about 2.5 to about 20 mg/kg, about 3 to about 20 mg/kg, about 3.5 to about 20 mg/kg, about 4 to about 20 mg/kg, about 4.5 to about 20 mg/kg, about 5 to about 20 mg/kg, about 7.5 to about 20 mg/kg, about 10 to about 20 mg/kg, or about 15 to about 20 mg/kg. Values and ranges intermediate to the recited values are also intended to be part of this disclosure.

For example, subjects can be administered a therapeutic amount of an RNAi agent, such as about 0.5, 0.6, 0.7. 0.8, 0.9, 1, 1.1, 1.2, 1.3, 1.4, 1.5, 1.6, 1.7, 1.8. 1.9, 2, 2.1, 2.2, 2.3, 2.4, 2.5, 2.6, 2.7, 2.8. 2.9, 3, 3.1, 3.2, 3.3, 3.4, 3.5, 3.6, 3.7, 3.8. 3.9, 4, 4.1, 4.2, 4.3, 4.4, 4.5, 4.6, 4.7, 4.8. 4.9, 5, 5.1, 5.2, 5.3, 5.4, 5.5, 5.6, 5.7, 5.8. 5.9, 6, 6.1, 6.2, 6.3, 6.4, 6.5, 6.6, 6.7, 6.8. 6.9, 7, 7.1, 7.2, 7.3, 7.4, 7.5, 7.6, 7.7, 7.8. 7.9, 8, 8.1, 8.2, 8.3, 8.4, 8.5, 8.6, 8.7, 8.8. 8.9, 9, 9.1, 9.2, 9.3, 9.4, 9.5, 9.6, 9.7, 9.8. 9.9, 10.5, 11, 11.5, 12, 12.5, 13, 13.5, 14, 14.5, 15, 15.5, 16, 16.5, 17, 17.5, 18, 18.5, 19, 19.5, 20, 21, 22, 23, 24, 25, 26, 27, 28, 29, 30, 31, 32, 33, 34, 34, 35, 36, 37, 38, 39, 40, 41, 42, 43, 44, 45, 46, 47, 48, 49, or about 50 mg/kg. Values and ranges intermediate to the recited values are also intended to be part of this disclosure.

The pharmaceutical composition can be administered once daily, or the RNAi agent can be administered as two, three, or more sub-doses at appropriate intervals throughout the day or even using continuous infusion or delivery through a controlled release formulation. In that case, the RNAi agent contained in each sub-dose must be correspondingly smaller in order to achieve the total daily dosage. The dosage unit can also be compounded for delivery over several days, e.g., using a conventional sustained release formulation which provides sustained release of the iRNA over a several day period. Sustained release formulations are well known in the art and are particularly useful for delivery of agents at a particular site, such as could be used with the agents of the present disclosure. In this embodiment, the dosage unit contains a corresponding multiple of the daily dose.

The effect of a single dose on APP levels can be long-lasting, such that subsequent doses are administered at not more than 3, 4, or 5 day intervals, or at not more than 1, 2, 3, or 4 week intervals, or at not more than 1, 2, 3, 4, 5, or 6 month intervals.

Such pharmaceutical compositions are formulated based on the mode of delivery. The formulations/pharmaceutical compositions disclosed herein are primarily formulated for injection, and in certain applications, for direct delivery into the CNS, e.g., by intrathecal or intravitreal routes of injection, optionally by infusion into the brain (e.g., striatum), such as by continuous pump infusion. However, certain compositions of the instant disclosure can also be formulated for systemic administration via parenteral delivery, e.g., by intravenous (IV), intramuscular (IM), or for subcutaneous (subQ) delivery.

In certain embodiments, the pharmaceutical compositions of the disclosure are substantially free of inorganic phosphate. In some embodiments, the molar ratio of a divalent ion source-to-nucleic acid therapeutic of the disclosure within a formulation is greater than about 2:1.

In certain embodiments, the pharmaceutical compositions of the disclosure are pyrogen free or non-pyrogenic.

The pharmaceutical compositions of the disclosure may be administered in dosages sufficient to inhibit expression of a target gene. In general, a suitable dose of a nucleic acid therapeutic of the disclosure will be in the range of about 0.001 to about 200.0 milligrams per kilogram body weight of the recipient per day, generally in the range of about 1 to 50 mg per kilogram body weight per day.

A repeat-dose regimen may include administration of a therapeutic amount of an RNAi agent on a regular basis, such as monthly to once every six months. In certain embodiments, the RNAi agent is administered about once per quarter (i.e., about once every three months) to about twice per year.

After an initial treatment regimen (e.g., loading dose), the treatments can be administered on a less frequent basis.

In other embodiments, a single dose of the pharmaceutical compositions can be long lasting, such that subsequent doses are administered at not more than 1, 2, 3, 4, 5, or 6 or more month intervals. In some embodiments of the disclosure, a single dose of the pharmaceutical compositions of the disclosure is administered once per month. In other embodiments of the disclosure, a single dose of the pharmaceutical compositions of the disclosure is administered once per quarter to twice per year.

The skilled artisan will appreciate that certain factors can influence the dosage and timing required to effectively treat a subject, including but not limited to the severity of the disease or disorder, previous treatments, the general health or age of the subject, and other diseases present. Moreover, treatment of a subject with a therapeutically effective amount of a composition can include a single treatment or a series of treatments.

The pharmaceutical compositions of the present disclosure are primarily formulated for CNS delivery, administered via an intracranial route, e.g., by intrathecal, intraparenchymal, or intraventricular administration.

The RNAi agent formulations can be delivered in a manner to target a particular tissue of the CNS (e.g., neuronal, glial or vascular tissue of the brain), or both a non-CNS organ (e.g., the liver) and the CNS.

Other formulations amenable to the present disclosure (if made substantially free of sources of inorganic phosphate) are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application number PCT/US2007/080331, filed Oct. 3, 2007, also describes formulations that are amenable to the present disclosure (if made substantially free of sources of inorganic phosphate).

The formulations of the instant disclosure are contemplated to function with inclusion of a wide variety of additional components, provided that such additional components do not detract from efficacy/drug product functionality. Examples of such additional components include, without limitation, fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199), as well as glucose and other sugars/carbon sources.

Compositions and formulations suitable for parenteral administration include, but are not limited to, such suitable for intraparenchymal (into the brain, e.g., intracerebrovascular), intrathecal (e.g., lumbar puncture (LP) or intracisterna magna (ICM) injection), intradiscal, periganglionic, and/or intraventricular administration, and can include sterile aqueous solutions which can also contain buffers, diluents and other suitable additives such as, but not limited to, penetration enhancers, carrier compounds and other pharmaceutically acceptable carriers or excipients. In certain embodiments herein, compositions and formulations suitable for parenteral administration do not include buffering components (e.g., phosphate salts).

Pharmaceutical compositions of the present disclosure include, but are not limited to, solutions and emulsions. These compositions can be generated from a variety of components that include, but are not limited to, preformed liquids.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

### Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Suitable pharmaceutically acceptable excipients include, but are not limited to, gelatin, lactose, amylose, magnesium stearate, talc, silicic acid, viscous paraffin, hydroxymethylcellulose, polyvinylpyrrolidone and the like.

### Other Components

The compositions of the present disclosure can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents. However, such materials, when added, should not unduly interfere with the biological activities of the components of the compositions of the present disclosure, and for the current disclosure should not provide an appreciable source of inorganic phosphate. The formulations can be sterilized and, if desired, mixed with auxiliary agents, e.g., preservatives, stabilizers, emulsifiers, salts for influencing osmotic pressure, colorings, flavorings or aromatic substances and the like which do not deleteriously interact with the nucleic acid(s) of the formulation.

Aqueous suspensions can contain substances which increase the viscosity of the suspension including, for example, sodium carboxymethylcellulose, sorbitol or dextran. The suspension can also contain stabilizers.

In some embodiments, pharmaceutical compositions contemplated by the disclosure include (a) one or more RNAi agents and (b) one or more agents which function by a non-RNAi mechanism and which are useful in treating a disease or disorder, e.g., an APP-associated neurodegenerative disorder. Examples of such agents include, but are not limited to dopamine agonists and promoters, among others, including carbidopa-levodopa, levodopa, entacopone, tolcapone, opicapone, pramipexole, ropinirole, apomorphine, rotigotine, selegiline, rasagiline, safinamide, amantadine, istradefylline, trihexyphenidyl, benztropine, rivastigmine, donepezil, galantamine and memantine.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulating a range of dosage for use in humans. The dosage of compositions featured herein in the disclosure lies generally within a range of circulating concentrations that include the ED₅₀ with little or no toxicity. The dosage can vary within this range depending upon the dosage form employed and the route of administration utilized. For any compound used in the methods featured in the disclosure, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the RNAi agent compositions featured in the disclosure can be administered in combination with other known agents effective in treatment of a disease or disorder. In any event, the administering physician can adjust the amount and timing of RNAi agent administration on the basis of results observed using standard measures of efficacy known in the art or described herein.

### Nucleic Acid Therapeutics of the Disclosure

Certain aspects of the instant disclosure provide formulations suitable for delivery of the nucleic acid therapeutics to the CNS, in certain embodiments via intrathecal injection or via direct injection into the brain. While RNAi agents are specifically exemplified, it is expressly contemplated that the compositions disclosed herein can be employed for delivery of a wide range of nucleic acid therapeutics, optimally to the CNS via injection. Exemplary nucleic acid therapeutics include, but are not limited to, dsRNAs (e.g., siRNAs), antisense oligonucleotides, decoys, miRNAs, shRNAs, guide RNAs (gRNAs) and ribozymes.

### RNAi Agents

The dsRNA includes an antisense strand having a region of complementarity which is complementary to at least a part of an mRNA formed in the expression of a target gene, The region of complementarity is about 30 nucleotides or less in length (e.g., about 30, 29, 28, 27, 26, 25, 24, 23, 22, 21, 20, 19, or 18 nucleotides or less in length). Upon contact with a cell expressing the target gene, the RNAi agent inhibits the expression of the target gene (e.g., a human, a primate, a non-primate, or a bird APP gene) by at least about 10% as assayed by, for example, a PCR or branched DNA (bDNA)-based method, or by a protein-based method, such as by immunofluorescence analysis, using, for example, western Blotting or flowcytometric techniques.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of a target gene (e.g., an APP gene). The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is between 15 and 30 base pairs in length, e.g., between, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain preferred embodiments, the duplex structure is between 18 and 25 base pairs in length, e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length. Ranges and lengths intermediate to the above recited ranges and lengths are also contemplated to be part of the disclosure.

Similarly, the region of complementarity to the target sequence is between 15 and 30 nucleotides in length, e.g., between 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the disclosure.

In some embodiments, the dsRNA is between about 15 and about 23 nucleotides in length, or between about 25 and about 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 9 to 36 base pairs, e.g., about 10-36, 11-36, 12-36, 13-36, 14-36, 15-36, 9-35, 10-35, 11-35, 12-35, 13-35, 14-35, 15-35, 9-34, 10-34, 11-34, 12-34, 13-34, 14-34, 15-34, 9-33, 10-33, 11-33, 12-33, 13-33, 14-33, 15-33, 9-32, 10-32, 11-32, 12-32, 13-32, 14-32, 15-32, 9-31, 10-31, 11-31, 12-31, 13-32, 14-31, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, a RNAi agent useful to target expression of a target gene (e.g., APP) is not generated in the target cell by cleavage of a larger dsRNA.

A dsRNA as described herein can further include one or more single-stranded nucleotide overhangs e.g., 1, 2, 3, or 4 nucleotides. dsRNAs having at least one nucleotide overhang can have unexpectedly superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

A dsRNA can be synthesized by standard methods known in the art as further discussed below, e.g., by use of an automated DNA synthesizer, such as are commercially available from, for example, Biosearch, Applied Biosystems, Inc.

RNAi agents of the disclosure may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the disclosure can be prepared using solution-phase or solid-phase organic synthesis or both.

In one aspect, a dsRNA of the disclosure includes at least two nucleotide sequences, a sense sequence and an antisense sequence. The sense strand sequence may be selected from the group of sequences provided in any one of Tables 16-19, and the corresponding nucleotide sequence of the antisense strand of the sense strand may be selected from the group of sequences of any one of Tables 16-19. In this aspect, one of the two sequences is complementary to the other of the two sequences, with one of the sequences being substantially complementary to a sequence of an mRNA generated in the expression of an APP gene. As such, in this aspect, a dsRNA will include two oligonucleotides, where one oligonucleotide is described as the sense strand (passenger strand) in any one of Tables 16-19, and the second oligonucleotide is described as the corresponding antisense strand (guide strand) of the sense strand in any one of Tables 16-19. Accordingly, by way of example, the following pairwise selections of sense and antisense strand sequences of Table 16 are expressly contemplated as forming duplexes of the instant disclosure: SEQ ID NOs: 1 and 8; SEQ ID NOs: 2 and 9; SEQ ID NOs: 3 and 10; SEQ ID NOs: 4 and 11; SEQ ID NOs: 5 and 12; SEQ ID NOs: 6 and 13; and SEQ ID NOs: 7 and 14. Similarly, pairwise combinations of sense and antisense strands of Table 17 of the instant disclosure are also expressly contemplated, including, e.g., a sense strand selected from Table 16 together with an antisense strand selected from Table 17, or vice versa, etc.

In one embodiment, the substantially complementary sequences of the dsRNA are contained on separate oligonucleotides. In another embodiment, the substantially complementary sequences of the dsRNA are contained on a single oligonucleotide.

It will be understood that, although the sequences in Table 16 and Table 18 are described as modified and/or conjugated sequences, the RNA of the RNAi agent of the disclosure e.g., a dsRNA of the disclosure, may comprise any one of the sequences set forth in Table 16 or Table 18 that is un-modified, un-conjugated, and/or modified and/or conjugated differently than described therein.

The skilled person is well aware that dsRNAs having a duplex structure of between about 20 and 23 base pairs, e.g., 21, base pairs have been hailed as particularly effective in inducing RNA interference (Elbashir et al., (2001) EMBO J., 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of a target gene by not more than about 5, 10, 15, 20, 25, or 30% inhibition from a dsRNA comprising the full sequence, are contemplated to be within the scope of the present disclosure.

In addition, the exemplified RNAs described herein identify a site(s) in an APP transcript that is susceptible to RISC-mediated cleavage. As such, the present disclosure further features RNAi agents that target within this site(s). As used herein, a RNAi agent is said to target within a particular site of an RNA transcript if the RNAi agent promotes cleavage of the transcript anywhere within that particular site. Such a RNAi agent will generally include at least about 15 contiguous nucleotides from one of the sequences provided herein coupled to additional nucleotide sequences taken from the region contiguous to the selected sequence in an APP gene.

A RNAi agent as described herein can contain one or more mismatches to the target sequence. In one embodiment, a RNAi agent as described herein contains no more than 3 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of an APP gene, generally does not contain any mismatch within the central 13 nucleotides. The methods described herein or methods known in the art can be used to determine whether a RNAi agent containing a mismatch to a target sequence is effective in inhibiting the expression of an APP gene. Consideration of the efficacy of RNAi agents with mismatches in inhibiting expression of an APP gene is important, especially if the particular region of complementarity in an APP gene is known to have polymorphic sequence variation within the population.

The present disclosure also provides a method for preparing a formulation comprising annealing a sense strand and an antisense strand, wherein one of the sense strand and antisense strand contains a lipophilic modification, to form a duplex solution comprising a double stranded RNA (dsRNA); lyophilizing the duplex solution to provide a duplex composition; and dissolving the duplex composition in an injection solution, wherein the injection solution comprises a divalent cation source (e.g., calcium) and does not comprise a phosphate buffer; and the duplex composition comprises 0-5% molar excess (e.g., about 1-2% molar excess) of antisense strand over sense strand. A “duplex solution” is meant any solution comprising double stranded RNA (dsRNA). By “duplex composition” is meant any composition comprising a dsRNA. In some embodiments, the duplex composition is prepared by lyophilization. In one embodiment, the duplex composition is a lyophilized powder. An “injection solution” is any solution used for dissolving a duplex composition. In some embodiments, the injection solution comprises a divalent cation source. The divalent cation source is calcium, magnesium, copper, nickel, zinc, or strontium, optionally wherein the divalent ion source is calcium. In one embodiment, the injection solution does not comprise a phosphate buffer. In some embodiments, the duplex composition comprises about a 1-2% molar excess, a 1-3% molar excess, a 1-4% molar excess, a 1-5% molar excess, a 0-5% molar excess, a 0-1% molar excess, a 2-3% molar excess, a 3-4% molar excess, a 3-5% molar excess, a 2-4% molar excess, a 2-5% molar excess of antisense strand over sense strand. In one embodiment, the duplex composition comprises about a 1-2% molar excess of antisense strand over sense strand. In another embodiment, the excess of antisense strand over sense strand is less than 1%.

### Modified RNAi Agents and Other Modified Nucleic Acid Therapeutics of the Disclosure

In one embodiment, the RNA of a nucleic acid therapeutic (e.g., a RNAi agent) of the disclosure e.g., a dsRNA, is un-modified, and does not comprise, e.g., chemical modifications and/or conjugations known in the art and described herein. In another embodiment, the RNA of a nucleic acid therapeutic of the disclosure, e.g., a dsRNA, is chemically modified to enhance stability or other beneficial characteristics. In certain embodiments of the disclosure, substantially all of the nucleotides of a nucleic acid therapeutic of the disclosure are modified. In other embodiments of the disclosure, all of the nucleotides of a nucleic acid therapeutic of the disclosure are modified. nucleic acid therapeutics of the disclosure in which “substantially all of the nucleotides are modified” are largely but not wholly modified and can include not more than 5, 4, 3, 2, or 1 unmodified nucleotides. In still other embodiments of the disclosure, nucleic acid therapeutics of the disclosure can include not more than 5, 4, 3, 2 or 1 modified nucleotides.

The nucleic acids featured in the disclosure can be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. Specific examples of nucleic acid therapeutics useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified nucleic acid therapeutic will have a phosphorus atom in its internucleoside backbone.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

Modified RNAs can also contain one or more substituted sugar moieties. The nucleic acid therapeutics, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)·ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a RNAi agent, or a group for improving the pharmacodynamic properties of a RNAi agent, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-O-NMA (N-methylacetamide, —OCH₂C(O)N(H)Me).

Other modifications include 2′-methoxy (2′-OCH₃), 2′-aminopropoxy (2′-OCH₂CH₂CH₂NH₂), 2′-O-hexadecyl, and 2′-fluoro (2′-F). Similar modifications can also be made at other positions on the RNA of a nucleic acid therapeutic, particularly the 3′ position of the sugar on the 3′ terminal nucleotide or in 2′-5′ linked dsRNAs and the 5′ position of 5′ terminal nucleotide. Nucleic acid therapeutics can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

A nucleic acid therapeutic of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

A nucleic acid therapeutic of the disclosure can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

A nucleic acid therapeutic of the disclosure can also be modified to include one or more bicyclic sugar moities. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the disclosure may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH2-O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193). Examples of bicyclic nucleosides for use in the polynucleotides of the disclosure include without limitation nucleosides comprising a bridge between the 4′ and the 2′ ribosyl ring atoms. In certain embodiments, the antisense polynucleotide agents of the disclosure include one or more bicyclic nucleosides comprising a 4′ to 2′ bridge. Examples of such 4′ to 2′ bridged bicyclic nucleosides, include but are not limited to 4′-(CH2)-O-2′ (LNA); 4′-(CH2)S-2′; 4′-(CH2)2-O-2′ (ENA); 4′-CH(CH3)-O-2′ (also referred to as “constrained ethyl” or “cEt”) and 4′-CH(CH2OCH3)-O-2′ (and analogs thereof, see, e.g., U.S. Pat. No. 7,399,845); 4′-C(CH3)(CH3)-O-2′ (and analogs thereof, see e.g., U.S. Pat. No. 8,278,283); 4′-CH2-N(OCH3)-2′ (and analogs thereof, see e.g., U.S. Pat. No. 8,278,425); 4′-CH2-O—N(CH3)-2′ (see, e.g., U.S. Patent Publication No. 2004/0171570); 4′-CH2-N(R)—O-2′, wherein R is H, C1-C12 alkyl, or a protecting group (see, e.g., U.S. Pat. No. 7,427,672); 4′-CH2-C(H)(CH3)-2′ (see, e.g., Chattopadhyaya et al., J. Org. Chem., 2009, 74, 118-134); and 4′-CH2C(═CH2)-2′ (and analogs thereof, see, e.g., U.S. Pat. No. 8,278,426). The entire contents of each of the foregoing are hereby incorporated herein by reference.

Additional representative U.S. patents and US patenttent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and 3-D-ribofuranose (see WO 99/14226).

A nucleic acid therapeutic of the disclosure can also be modified to include one or more constrained ethyl nucleotides. As used herein, a “constrained ethyl nucleotide” or “cEt” is a locked nucleic acid comprising a bicyclic sugar moiety comprising a 4′-CH(CH3)-O-2′ bridge. In one embodiment, a constrained ethyl nucleotide is in the S conformation referred to herein as “S-cEt.”

A nucleic acid therapeutic of the disclosure may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, a nucleic acid therapeutic of the disclosure comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

### Modified RNAi Agents Comprising Motifs of the Disclosure

In certain aspects of the disclosure, the double-stranded RNAi agents of the disclosure include agents with chemical modifications as disclosed, for example, in WO 2013/075035, filed on Nov. 16, 2012, the entire contents of which are incorporated herein by reference. As shown herein and in PCT Publication No. WO 2013/075035, a superior result may be obtained by introducing one or more motifs of three identical modifications on three consecutive nucleotides into a sense strand and/or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense and/or antisense strand. The RNAi agent may be optionally conjugated with a C16 ligand, for instance on the sense strand. The RNAi agent may be optionally modified with a (S)-glycol nucleic acid (GNA) modification, for instance on one or more residues of the antisense strand. The resulting RNAi agents present superior gene silencing activity.

More specifically, it has been surprisingly discovered that when the sense strand and antisense strand of the double-stranded RNAi agent are completely modified to have one or more motifs of three identical modifications on three consecutive nucleotides at or near the cleavage site of at least one strand of an RNAi agent, the gene silencing activity of the RNAi agent was superiorly enhanced.

Accordingly, the disclosure provides double stranded RNAi agents capable of inhibiting the expression of a target gene (i.e., an APP gene) in vivo. The RNAi agent comprises a sense strand and an antisense strand. Each strand of the RNAi agent may range from 12-30 nucleotides in length. For example, each strand may be between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as an “dsRNA agent” or “RNAi agent.” The terms “dsRNA agent”, “RNAi agent”, “iRNA agent”, and “siRNA agent” are used interchangeably herein. The duplex region of an RNAi agent may be 12-30 nucleotide pairs in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In one embodiment, the RNAi agent may contain one or more overhang regions and/or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2′-F (i.e., 2′-deoxy-2′fluoro), 2′-Omethyl, thymidine (T), and any combinations thereof.

For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double ended bluntmer of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In another embodiment, the RNAi agent is a double ended bluntmer of 20 nucleotides in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In yet another embodiment, the RNAi agent is a double ended bluntmer of 21 nucleotides in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the RNAi agent is blunt, while the other end comprises a 2 nucleotide overhang. Preferably, the 2 nucleotide overhang is at the 3′-end of the antisense strand. When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 3′-fluoro, e.g., in an alternating motif. Optionally, the RNAi agent further comprises a ligand (optionally a C16 ligand).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, there by forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other, then the chemistry of the motifs are distinct from each other; and when the motifs are separated by one or more nucleotide, then the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motifs of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In one embodiment, the RNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and LC is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an A:U base pair. For example, the first base pair within the duplex region from the 5′-end of the antisense strand is an A:U base pair.

In another embodiment, the nucleotide at the 3′-end of the sense strand is deoxy-thymine (dT). In another embodiment, the nucleotide at the 3′-end of the antisense strand is deoxy-thymine (dT). In one embodiment, there is a short sequence of deoxy-thymine nucleotides, for example, two dT nucleotides on the 3′-end of the sense and/or antisense strand.

Various publications describe multimeric RNAi agents that can be used in the methods of the disclosure. Such publications include WO2007/091269, U.S. Pat. No. 7,858,769, WO2010/141511, WO2007/117686, WO2009/014887 WO2011/031520, WO2013/074974, WO2013/165816, WO2016/028649, WO2018/098328, WO2019/126651, WO2019/222479, WO 2019/217459, WO2020/097044, and WO 2022/159158, the entire contents of each of which are hereby incorporated herein by reference.

In certain embodiments, the RNAi agents of the disclosure may include GalNAc ligands, even if such GalNAc ligands are currently projected to be of limited value for the preferred intrathecal/CNS delivery route(s) of the instant disclosure.

Modified RNAi agents Comprising Phosphate Moieties

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Some embodiments featured in the disclosure include RNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Other modifications of a nucleic acid therapeutic of the disclosure include a 5′ phosphate or 5′ phosphate mimic, e.g., a 5′-terminal phosphate or 5′-phosphate mimic on the antisense strand of a RNAi agent. Suitable 5′-phosphate mimics are disclosed in, for example US Patent Publication No. 2012/0157511, the entire contents of which are incorporated herein by reference.

### RNAi Agents Conjugated to Ligands

Another modification of the RNA of a RNAi agent composition of the disclosure involves chemically linking to the RNA one or more ligands, moieties or conjugates that enhance the activity, cellular distribution or cellular uptake of the RNAi. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937). It is expressly contemplated that a wide range of ligands can be attached to a dsRNA of the disclosure, optionally in addition to, e.g., a lipophilic moiety, a GalNAc moiety, or other dsRNA-attached moiety added with the intent to facilitate delivery of such dsRNAs to target cells.

In one embodiment, a ligand alters the distribution, targeting or lifetime of a RNAi agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands will not take part in duplex pairing in a duplexed nucleic acid.

The oligonucleotides used in the conjugates of the present disclosure may be conveniently and routinely made through the well-known technique of solid-phase synthesis. Equipment for such synthesis is sold by several vendors including, for example, Applied Biosystems (Foster City, Calif.), Cytiva Life Sciences etc. Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present disclosure, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present disclosure are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipophilic Moieties

The term “lipophile” or “lipophilic moiety” broadly refers to any compound or chemical moiety having an affinity for lipids. One way to characterize the lipophilicity of the lipophilic moiety is by the octanol-water partition coefficient, logK_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf Comput. Sci. 41: 1407-21 (2001), which is incorporated herein by reference in its entirety). It provides a thermodynamic measure of the tendency of the substance to prefer a non-aqueous or oily milieu rather than water (i.e., its hydrophilic/lipophilic balance). In principle, a chemical substance is lipophilic in character when its logK_{ow} exceeds 0. Typically, the lipophilic moiety possesses a logK_{ow} exceeding 1, exceeding 1.5, exceeding 2, exceeding 3, exceeding 4, exceeding 5, or exceeding 10. For instance, the logK_{ow} of 6-amino hexanol, for instance, is predicted to be approximately 0.7. Using the same method, the logK_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., logK_{ow}) value of the lipophilic moiety.

Alternatively, the hydrophobicity of the double-stranded RNAi agent, conjugated to one or more lipophilic moieties, can be measured by its protein binding characteristics. For instance, in certain embodiments, the unbound fraction in the plasma protein binding assay of the double-stranded RNAi agent could be determined to positively correlate to the relative hydrophobicity of the double-stranded RNAi agent, which could then positively correlate to the silencing activity of the double-stranded RNAi agent.

Exemplary lipophilic moieties and/or lipophilic modifications known in the art include, without limitation, lipids selected from saturated or unsaturated fatty acids, steroids, fat soluble vitamins, phospholipids, sphingolipids, hydrocarbons, mono-, di-, and tri-glycerides, and synthetic derivatives thereof, cholesterol, C₁₀-C₂₆ saturated fatty acid, C₁₀-C₂₆ unsaturated fatty acid, C₁₀-C₂₆ alkyl, triglyceride, tocopherol, or cholic acid. Among exemplary lipophilic moieties are fatty acids and adamantyl groups, including exemplary configurations of lipophilic/hydrophobic moiety modification of oligonucleotides as set forth in PCT/US2021/042469, further including “lipid conjugate” (LC) moieties where each ligand is independently hydrogen, or a hydrophobic moiety selected from adamantyl group and lipid moiety; and/or each LC is independently a lipid conjugate moiety comprising a saturated or unsaturated, straight or branched C₁₋₅₀ hydrocarbon chain, wherein 0-10 methylene units of the hydrocarbon chain are independently replaced by —Cy—, —O—, —NR—, —S—, —C(O)—, —S(O)—, —S(O)₂—, —P(O)OR—, or —P(S)OR—; and/or LC is a lipid conjugate moiety comprising a saturated or partially unsaturated, straight or branched C₁₋₅₀ hydrocarbon chain, wherein 0-10 methylene units of the hydrocarbon chain are independently replaced by —Cy—, —O—, —NR—, —S—, —C(O)—, —S(O)—, —S(O)₂—, —P(O)OR—, or —P(S)OR—; and/or the lipid conjugate moiety is formed from the coupling of a nucleic acid or analogue thereof with a lipophilic compound. In some embodiments, LC is a lipid conjugate moiety comprising an esterified or amidated saturated straight-chain fatty acid. In some embodiments, LC is —OC(O)CH₃ or —NHC(O)CH₃. In some embodiments, LC is —OC(O)C₂H₅ or —NHC(O)C₂H₅. In some embodiments, LC is —OC(O)C₃H₇ or —NHC(O)C₃H₇. In some embodiments, LC is —OC(O)C₄H₉ or —NHC(O)C₄H₉. In some embodiments, LC is —OC(O)C₅Hii or —NHC(O)C₅Hii. In some embodiments, LC is —OC(O)C₆H₁₃ or —NHC(O)C₆H₁₃. In some embodiments, LC is —OC(O)C₇H₁₅ or —NHC(O)C₇H₁₅. In some embodiments, LC is —OC(O)C₈H₁₇ or —NHC(O)C₈H₁₇. In some embodiments, LC is —OC(O)C₉H₁₉ or —NHC(O)C₉H₁₉. In some embodiments, LC is —OC(O)C₁₀H₂₁ or —NHC(O)C₁₀H₂₁. In some embodiments, LC is —OC(O)C₁₁H₂₃ or —NHC(O)C₁₁H₂₃. In some embodiments, LC is —OC(O)C₁₂H₂₅ or —NHC(O)C₁₂H₂₅. In some embodiments, LC is —OC(O)C₁₃H₂₇ or —NHC(O)C₁₃H₂₇. In some embodiments, LC is —OC(O)C₁₄H₂₉ or —NHC(O)C₁₄H₂₉. In some embodiments, LC is —OC(O)C₁₅H₃₁ or —NHC(O)C₁₅H₃₁. In some embodiments, LC is —OC(O)C₁₆H₃₃ or —NHC(O)C₁₆H₃₃. In some embodiments, LC is —OC(O)C₁₇H₃₅ or —NHC(O)C₁₇H₃₅. In some embodiments, LC is —OC(O)C₁₈H₃₇ or —NHC(O)C₁₈H₃₇. In some embodiments, LC is —OC(O)C₁₉H₃₉ or —NHC(O)C₁₉H₃₉. In some embodiments, LC is —OC(O)C₂₀H₄₁ or —NHC(O)C₂₀H₄₁. In some embodiments, LC is —OC(O)C₂₁H₄₃ or —NHC(O)C₂₁H₄₃. In some embodiments, LC is —OC(O)C₂₂H₄₅ or —NHC(O)C₂₂H₄₅. In some embodiments, LC is —OC(O)C₂₃H₄₇ or —NHC(O)C₂₃H₄₇. In some embodiments, LC is —OC(O)C₂₄H₂₉ or —NHC(O)C₂₄H₂₉. In some embodiments, LC is —OC(O)C₂₅H₅₁ or —NHC(O)C₂₅H₅₁. In some embodiments, LC is —OC(O)C₂₆H₅₃ or —NHC(O)C₂₆H₅₃. In some embodiments, LC is —OC(O)C₂₇H₅₅ or —NHC(O)C₂₇H₅₅. In some embodiments, LC is —OC(O)C₂₈H₅₇ or —NHC(O)C₂₈H₅₇. In some embodiments, LC is —OC(O)C₂₉H₅₉ or —NHC(O)C₂₉H₅₉. In some embodiments, LC is —OC(O)C₃₀H₆₁ or —NHC(O)C₃₀H₆₁. In some embodiments, LC is a lipid conjugate moiety comprising an esterified or amidated partially unsaturated straight-chain fatty acid. In some embodiments, LC is esterified or amidated myristoleic acid. In some embodiments, LC is esterified or amidated palmitoleic acid. In some embodiments, LC is esterified or amidated sapienic acid. In some embodiments, LC is esterified or amidated oleic acid, i.e.,

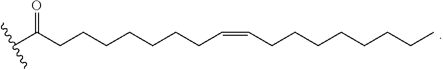 [SMILES: *C(=O)CCCCCCC/C=C\CCCCCCCC]

In some embodiments, LC is esterified or amidated elaidic acid. In some embodiments, LC is esterified or amidated vaccenic acid. In some embodiments, LC is esterified or amidated linoleic acid. In some embodiments, LC is esterified or amidated limoelaidic acid. In some embodiments, LC is esterified or amidated α-linolenic acid, i.e.,

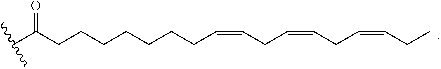 [SMILES: *C(=O)CCCCCCC/C=C\C/C=C\C/C=C\CC]

In some embodiments, LC is esterified or amidated arachidonic acid. In some embodiments, LC is esterified or amidated eicosapentaenoic acid, i.e.,

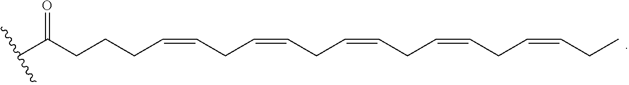 [SMILES: *C(=O)CCC/C=C\C/C=C\C/C=C\C/C=C\C/C=C\CC]

In some embodiments, LC is esterified or amidated erucic acid. In some embodiments, LC is esterified or amidated docosahexaenoic acid, i.e.,

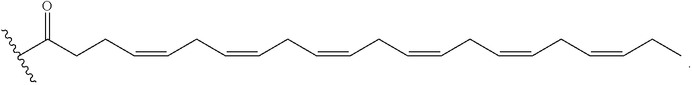 [SMILES: *C(=O)CC/C=C\C/C=C\C/C=C\C/C=C\C/C=C\C/C=C\CC]

In some embodiments, LC is esterified or amidated adamantanecarboxylic acid. In some embodiments, LC is esterified or amidated adamantaneacetic acid. In some embodiments, R⁵ is —C(O)(CH₂)i-ioadamantane.

Other forms of lipophilic moieties known in the art and expressly contemplated for use in the compositions and methods of the instant disclosure include, e.g., compounds of Formula (I) of PCT/US2021/049880:

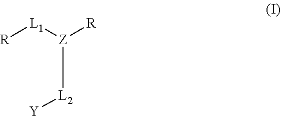

or a pharmaceutically acceptable salt thereof, wherein R is -L_{A}-R_{Z}; L_{A} is a bond or a bivalent moiety connecting R_{Z} to Z; R_{Z} comprises an oligonucleotide-based agent; Z is CH, phenyl or N; L₁ and L₂ are each independently linkers comprising at least about 5 polyethylene glycol (PEG) units; and X and Y are each independently lipids comprising from about 10 to about 50 carbon atoms. In some embodiments, L₁ and L₂ each independently comprise about 15 to about 100 PEG units. In some embodiments, L₁ and L₂ each independently comprise about 20 to about 60 PEG units. In some embodiments, L₁ and L₂ each independently comprise about 20 to about 30 PEG units. In other embodiments, L₁ and L₂ each independently comprise about 40 to about 60 PEG units. And, in some embodiments, one of L₁ and L₂ comprises about 20 to about 30 PEG units and the other comprises about 40 to about 60 PEG units.

Examples of X and Y moieties for above Formula (I) of PCT/US2021/049880 include, e.g.,

| Name | Structure |
|---|---|
| Lipid 1 | 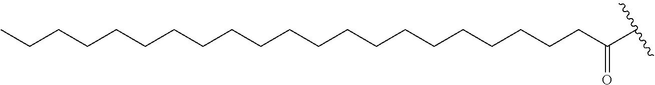 [SMILES: *C(=O)CCCCCCCCCCCCCCCCCCCCC] |
| Lipid 2 | 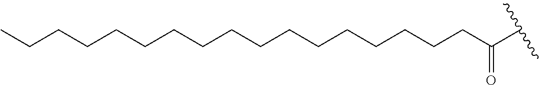 [SMILES: *C(=O)CCCCCCCCCCCCCCCCC] |
| Lipid 3 | 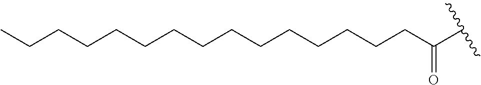 [SMILES: *C(=O)CCCCCCCCCCCCCCC] |
| Lipid 4 (Cholesteryl) | 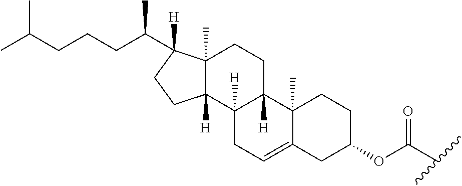 |
| Lipid 5 | 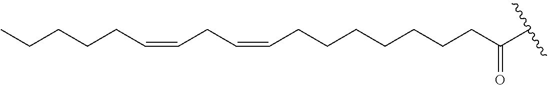 [SMILES: *C(=O)CCCCCCC/C=C\C/C=C\CCCCC] |
| Lipid 6 | 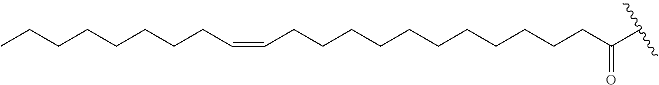 [SMILES: *C(=O)CCCCCCCCCCC/C=C\CCCCCCCC] |
| Lipid 7 | 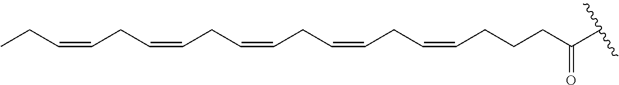 [SMILES: *C(=O)CCC/C=C\C/C=C\C/C=C\C/C=C\C/C=C\CC] |
| Lipid 8 | 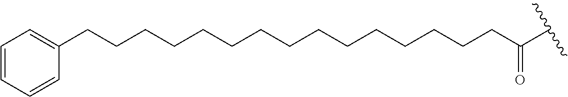 [SMILES: *C(=O)CCCCCCCCCCCCCCCc1ccccc1] |
| Lipid 9 | 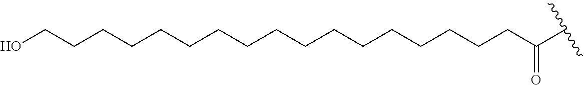 [SMILES: *C(=O)CCCCCCCCCCCCCCCCCO] |
| Lipid 10 | 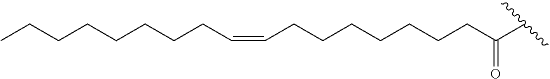 [SMILES: *C(=O)CCCCCCC/C=C\CCCCCCCC] |
| Lipid 11 | 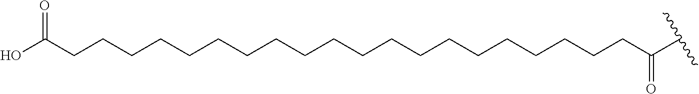 [SMILES: *C(=O)CCCCCCCCCCCCCCCCCCCCC(=O)O] |
| Lipid 12 | 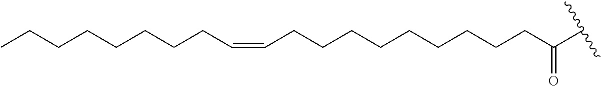 [SMILES: *C(=O)CCCCCCCCC/C=C\CCCCCCCC] |
| Lipid 14 | 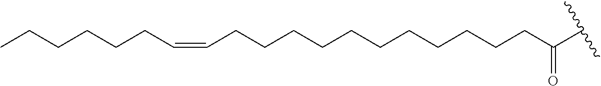 [SMILES: *C(=O)CCCCCCCCCCC/C=C\CCCCCC] |
| Lipid 15 | 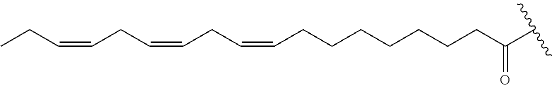 [SMILES: *C(=O)CCCCCCC/C=C\C/C=C\C/C=C\CC] |
| Lipid 16 | 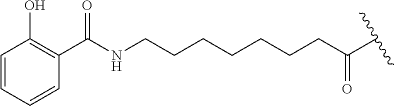 [SMILES: *C(=O)CCCCCCCNC(=O)c1ccccc1O] |
| Lipid 17 | 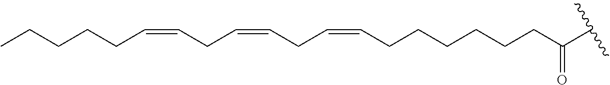 [SMILES: *C(=O)CCCCCC/C=C\C/C=C\C/C=C\CCCCC] |
| Lipid 18 | 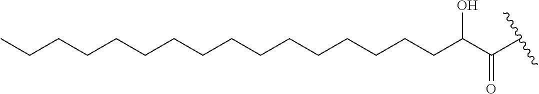 [SMILES: *C(=O)C(O)CCCCCCCCCCCCCCCC] |
| Lipid 19 | 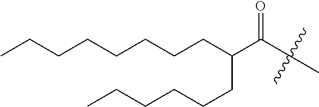 [SMILES: *C(=O)C(CCCCCC)CCCCCCCC] |
| Lipid 20 | 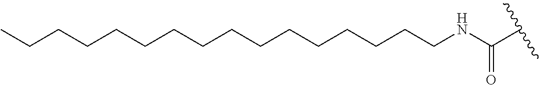 [SMILES: *C(=O)NCCCCCCCCCCCCCCCC] |
| Lipid 21 | 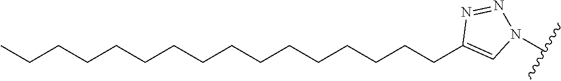 [SMILES: *n1cc(CCCCCCCCCCCCCCCC)nn1] |
| Lipid 22 | 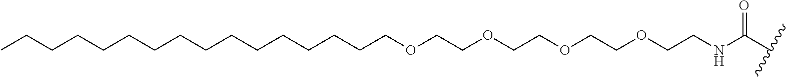 [SMILES: *C(=O)NCCOCCOCCOCCOCCCCCCCCCCCCCCCC] |
| Lipid 23 | 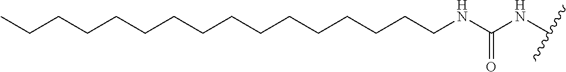 [SMILES: *NC(=O)NCCCCCCCCCCCCCCCC] |
| Lipid 24 | 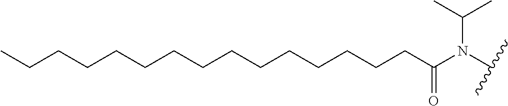 [SMILES: *N(C(=O)CCCCCCCCCCCCCCC)C(C)C] |

Exemplary lipophilic/hydrophobic moieties also contemplated for use in the compositions and methods disclosed herein further include a sterol (e.g., cholesterol), GM1, a lipid, a vitamin, a small molecule, a peptide, or a combination thereof. In some embodiments, the moiety is a lipid. For example, in certain embodiments, the moiety is palmitoyl. In some embodiments, the moiety is a sterol, e.g., cholesterol. Additional hydrophobic moieties include, for example, phospholipids, vitamin D, vitamin E, squalene, and fatty acids. In another exemplary embodiment, the oligonucleotide cargo is conjugated to myristic acid, or a derivative thereof (e.g., myristoylated oligonucleotide cargo—see U.S. Pat. No. 10,513,710).

Additional exemplary structures of lipophilic moieties of PCT/US2016/053836, include:

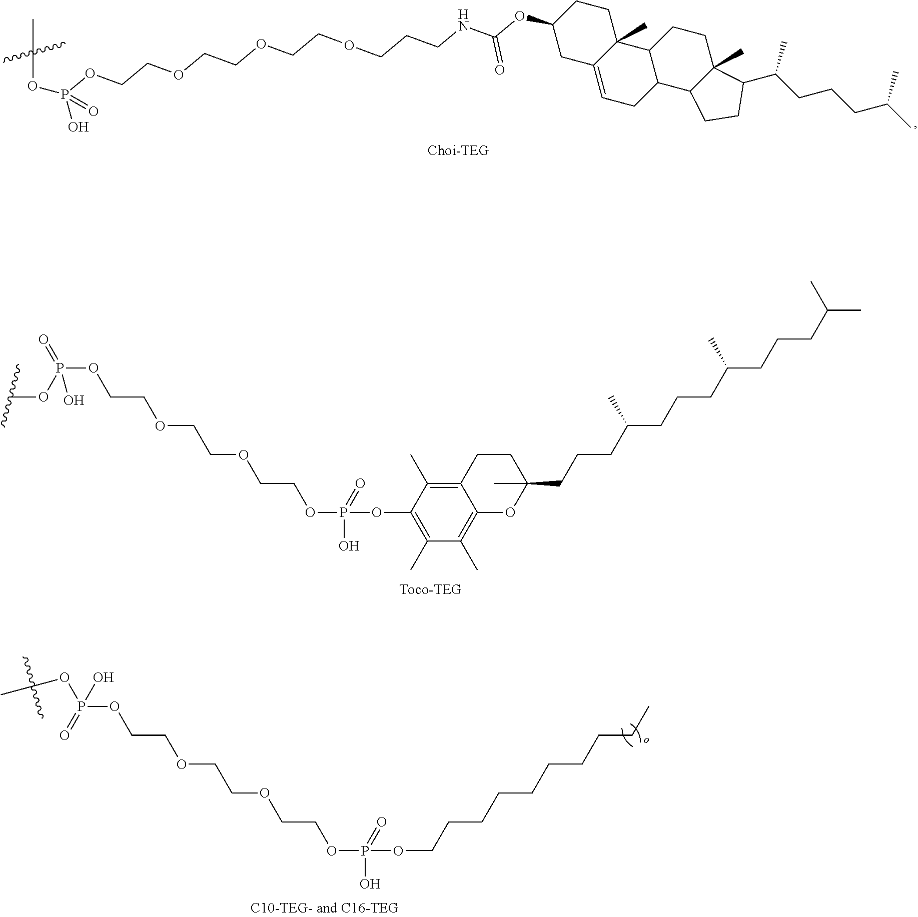

The nucleotide modifications set forth, e.g., in PCT/US2020/046561, are also expressly contemplated for use in the compositions and methods of the instant disclosure.

In one embodiment, the plasma protein binding assay determined is an electrophoretic mobility shift assay (EMSA) using human serum albumin protein. An exemplary protocol of this binding assay is illustrated in detail in, e.g., PCT/US2019/031170. The hydrophobicity of the double-stranded RNAi agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic moieties to the internal position(s) of the double-stranded RNAi agent provides optimal hydrophobicity for the enhanced in vivo delivery of siRNA.

In certain embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprises a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₁₈ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-Cis alkyl or alkenyl). In one embodiment, the lipophilic moiety contains a saturated or unsaturated C₁₆ hydrocarbon chain (e.g., a linear C₁₆ alkyl or alkenyl).

In some embodiments, the lipophilic moiety contains a saturated or unsaturated C₁₀-C₃₀ hydrocarbon chain; or a saturated or unsaturated C₁₂-C₃₀ hydrocarbon chain; or a saturated or unsaturated C₁₄-C₃₀ hydrocarbon chain; or a saturated or unsaturated C₁₆-C₃₀ hydrocarbon chain; or a saturated or unsaturated C₁₈-C₃₀ hydrocarbon chain; or saturated or unsaturated C₂₀-C₃₀ hydrocarbon chain.

In other embodiments, the lipophilic moiety contains a saturated or unsaturated C₁₀-C₂₄ hydrocarbon chain; or a saturated or unsaturated C₁₂-C₂₄ hydrocarbon chain; or a saturated or unsaturated C₁₄-C₂₄ hydrocarbon chain; or a saturated or unsaturated C₁₆-C₂₄ hydrocarbon chain; or a saturated or unsaturated Cis-C₂₄ hydrocarbon chain; or saturated or unsaturated C₂₀-C₂₄ hydrocarbon chain.

In other embodiments, the lipophilic moiety contains a saturated or unsaturated C₁₀ hydrocarbon chain; or a saturated or unsaturated C₁₁ hydrocarbon chain; or a saturated or unsaturated C₁₂ hydrocarbon chain; or a saturated or unsaturated C₁₃ hydrocarbon chain; or a saturated or unsaturated C₁₄ hydrocarbon chain; or a saturated or unsaturated C₁₅ hydrocarbon chain; or a saturated or unsaturated C₁₆ hydrocarbon chain; or a saturated or unsaturated C₁₇ hydrocarbon chain; or a saturated or unsaturated Cis hydrocarbon chain; or a saturated or unsaturated C₁₉ hydrocarbon chain; or a saturated or unsaturated C₂₀ hydrocarbon chain; or a saturated or unsaturated C₂₁ hydrocarbon chain; or a saturated or unsaturated C₂₂ hydrocarbon chain; or a saturated or unsaturated C₂₃ hydrocarbon chain; or a saturated or unsaturated C₂₄ hydrocarbon chain.

In other embodiments, the lipophilic moiety contains a C₁₀ alkyl chain, or a C₁₁ alkyl chain, or a C₁₂ alkyl chain, or a C₁₃ alkyl chain, or a C₁₄ alkyl chain, or a C₁₅ alkyl chain, or a C₁₆ alkyl chain, or a C₁₇ alkyl chain, or a C₁₈ alkyl chain, or a C₁₉ alkyl chain, or a C₂₀ alkyl chain, or a C₂₁ alkyl chain, or a C₂₂ alkyl chain, or a C₂₃ alkyl chain, or a C₂₄ alkyl chain. Each of the preceding, in another embodiment, may be a linear alkyl chain (e.g., n-tetradecyl, or n-pentadecyl, or n-hexadecyl, or n-heptadecyl, or n-octadecyl, or n-nonadecyl; or n-eicosyl, or n-henicosanyl, n-docosanyl, or n-tricosanyl, or n-tetracosanyl.

In exemplary embodiments, a modified nucleotide comprising a lipophilic modification may have the following structure,

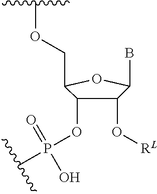

wherein B is an optionally modified nucleobase and R^{L} is any of the lipophilic moieties herein, optionally attached to the 2′-O via a carrier or linking group (e.g., in certain embodiments, the lipophilic moiety is directly bonded to the 2′-0).

In certain embodiments, R^{L} is a CLO-24alkyl chain optionally substituted by one group selected from the group consisting of, halogen, —C(O)OR, —OR, —NR₂, —C(O)R, —C(O)N(R)₂, wherein each R is independently hydrogen or C₁₋₆alkyl.

In certain embodiments, R^{L} is a C₁₀₋₂₄alkyl chain optionally substituted by a carboxy group (e.g., an ω-carboxy group).

In certain embodiments, R^{L} is a C₁₀-24alkyl chain optionally substituted by a hydroxy group (e.g., an ω-hydroxy group).

In certain embodiments, R^{L} is —(CH₂)ₙ—OH, wherein n is 14-24 (e.g., 16 or 22). In certain embodiments, R^{L} is —(CH₂)ₙ—COOH, wherein n is 14-24 (e.g., 16 or 22).

In certain embodiments, R^{L} is

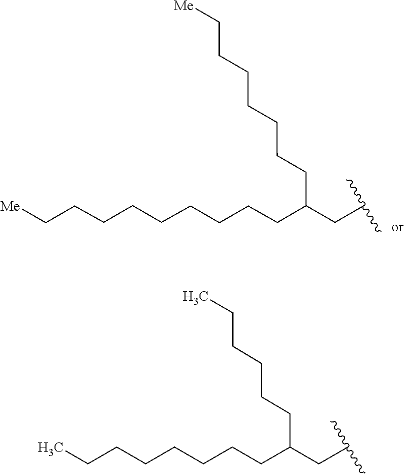 [SMILES: *CC(CCCCCCCC)CCCCCCCCCC ; *CC(CCCCCC)CCCCCCCC]

In certain embodiments, the compositions and methods of the disclosure include a C16 ligand. In exemplary embodiments, the C16 ligand of the disclosure has the following structure (exemplified here below for a uracil base, yet attachment of the C16 ligand is contemplated for a nucleotide presenting any base (C, G, A, etc.) and/or possessing any other modification as presented herein, provided that 2′ ribo attachment is preserved) and is attached at the 2′ position of the ribo within a residue that is so modified:

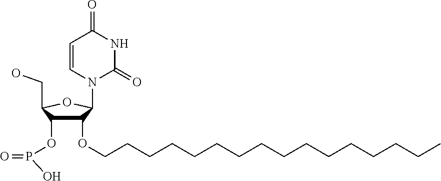

As shown above, a C16 ligand-modified residue presents a straight chain alkyl at the 2′-ribo position of an exemplary residue (here, a Uracil) that is so modified.

In additional exemplary embodiments, a modified nucleotide comprising a lipophilic modification may have any one of the following structures:

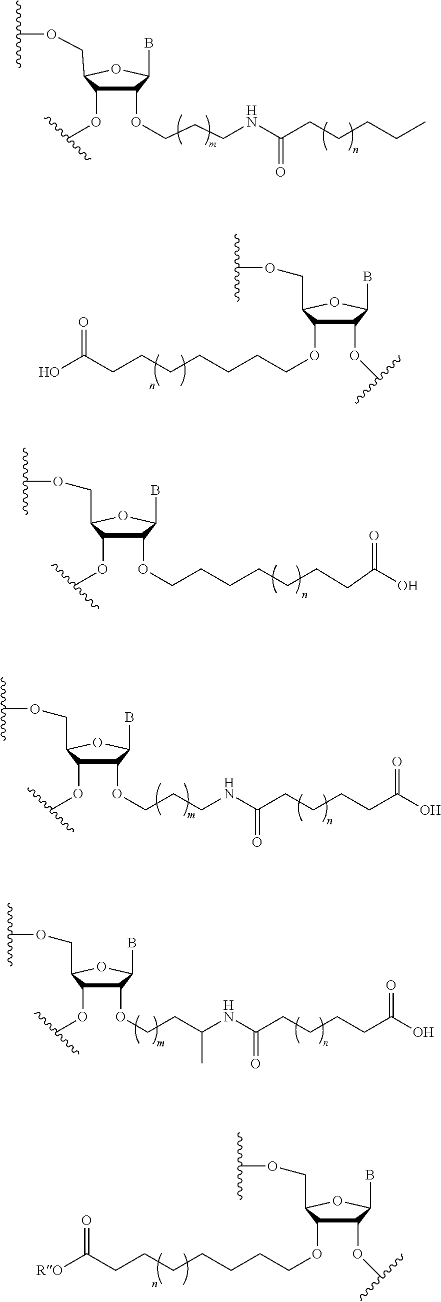

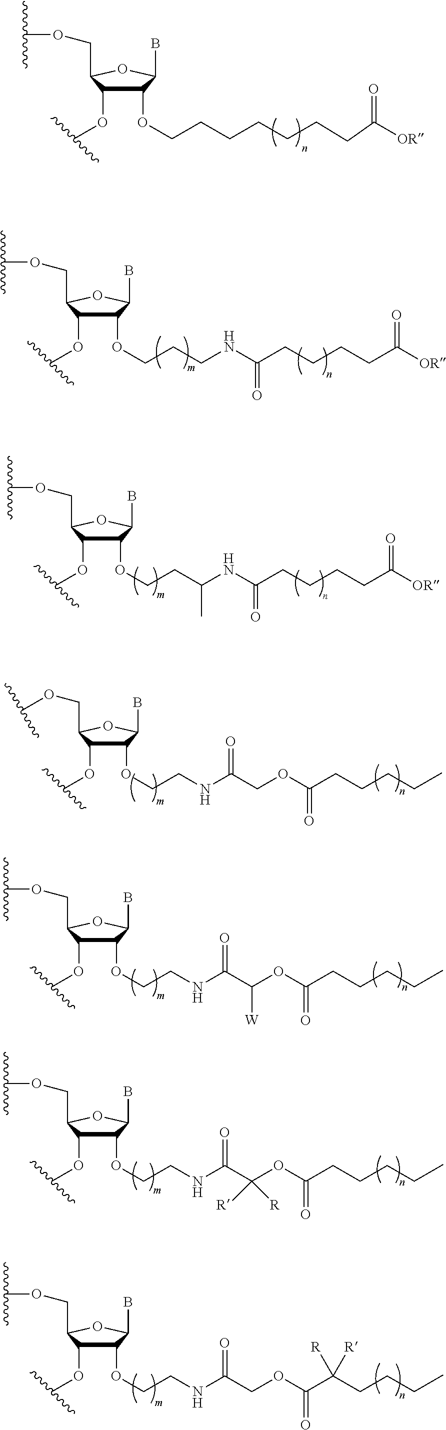

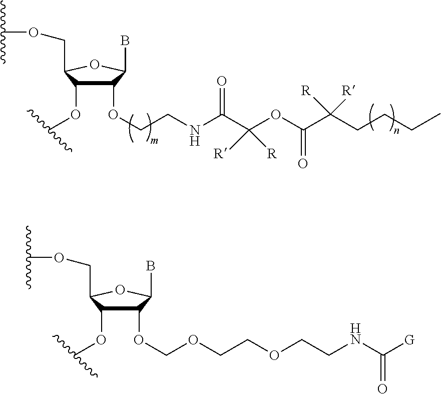

wherein m is 0-8; n is 1-21; W is an alkyl group such as a C₁-C₄ alkyl (e.g., methyl, ethyl, propyl, isopropyl, butyl, isobutyl, t-butyl). R, R′, and R″ are each independently H or an alkyl group such as a C₁-C₄ alkyl (e.g., methyl, ethyl, propyl, isopropyl, t-butyl); and G is a lipophilic moiety, according to any of the embodiment herein (e.g., G is an saturated or unsaturated C₁₀-C₂₄ hydrocarbon chain; or a saturated or unsaturated C₁₂-C₂₄ hydrocarbon chain; or a saturated or unsaturated C₁₄-C₂₄ hydrocarbon chain; or a saturated or unsaturated C₁₆-C₂₄ hydrocarbon chain; or a saturated or unsaturated C₁₈-C₂₄ hydrocarbon chain; or saturated or unsaturated C₂₀-C₂₄ hydrocarbon chain).

The lipophilic moiety may be attached to the RNAi agent by any method known in the art, including via a functional grouping already present in the lipophilic moiety or introduced into the RNAi agent, such as a hydroxy group (e.g., —CO—CH₂—OH). The functional groups already present in the lipophilic moiety or introduced into the RNAi agent include, but are not limited to, hydroxyl, amine, carboxylic acid, sulfonate, phosphate, thiol, azide, and alkyne.

Conjugation of the RNAi agent and the lipophilic moiety may occur, for example, through formation of an ether or a carboxylic or carbamoyl ester linkage between the hydroxy and an alkyl group R—, an alkanoyl group RCO or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosanyl, docosanyl, tricosanyl, or tetracosanyl group, or the like.

In some embodiments, the lipophilic moiety is conjugated to the double-stranded RNAi agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, the lipophilic moiety is conjugated to the double-stranded RNAi agent via a modified phosphodiester linkage. Examples of modified phosphodiester linkages include, but are not limited to,

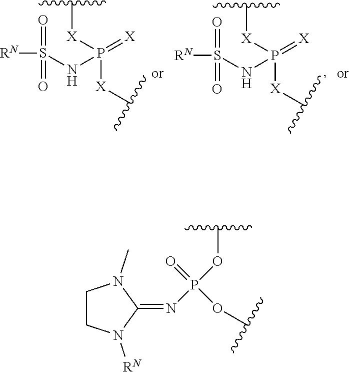

wherein each broken bond is a connection to the preceding and subsequent nucleotides (e.g., 3′-5′), each X is independently O or S and R^{N} is a lipophilic moiety described herein, e.g., a butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, henicosanyl, docosanyl, tricosanyl, or tetracosanyl group. For example, a modified nucleotide comprising a lipophilic modification as a modified phosphodiester may have the following structure,

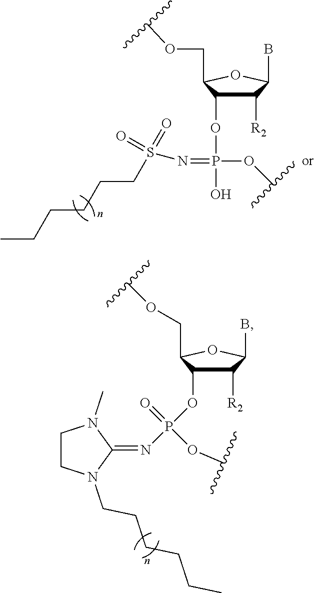

wherein B is an optionally modified nucleobase, n is 1-21 (e.g., 5-19, or 9-19; or 9, 10, 11, 12, 13, 14, 15, 16, 17, 18, or 19), and R² may be any functional group that is an acceptable 2′-modification for a ribose sugar, such as hydrogen, halo, a 2′-O-methoxyalkyl (e.g., 2′-O-methoxymethyl, 2′-O-methoxyethyl, or 2′-O-2-methoxypropanyl) modification, 2′-O-allyl modification, 2′-C-allyl modification, 2′-fluoro modification, 2′-O—N-methylacetamido (2′-O-NMA) modification, 2′-O-dimethylaminoethoxyethyl (2′-O-DMAEOE) modification, 2′-O-aminopropyl (2′-O-AP) modification, or 2′-ara-F modification. For instance, R₂′ may be H, OH, F, OMe, 0-methoxyalkyl, 0-allyl, O—N-methylacetamido, O-dimethylaminoethoxyethyl, or O-aminopropyl. In some embodiments, the lipophilic moiety is conjugated to the double-stranded RNAi agent via a modified nucleobase, such as, cytosine or uracil, either substituted at the 5-position with a lipophilic moiety described herein.

Examples include,

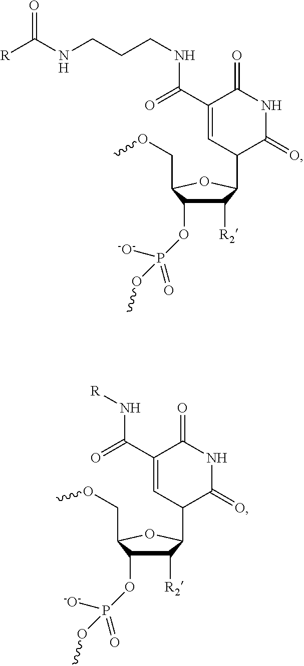

wherein R is a lipophilic moiety as defined herein. R₂′ is H, OH, F, OMe, O-methoxyalkyl, O-allyl, O—N-methylacetamido, O-dimethylaminoethoxyethyl, or O-aminopropyl. B is a modified or unmodified nucleobase.

In some embodiments, the lipophilic moiety is conjugated to the 3′-end of one of the sense and antisense strand with via a direct bond or through a carrier or linker. Examples include, but are not limited to moieties of the formula,

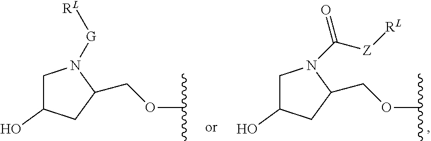

where the broken bond is to the 3′-end (3′-OH) of the oligonucleotide via a phosphodiester or phosphothioate internucleotide linkage; G is a linking group; Z is a bond, O, S, or N(H), and R^{L} is a lipophilic moiety, such as those described herein, including those below:

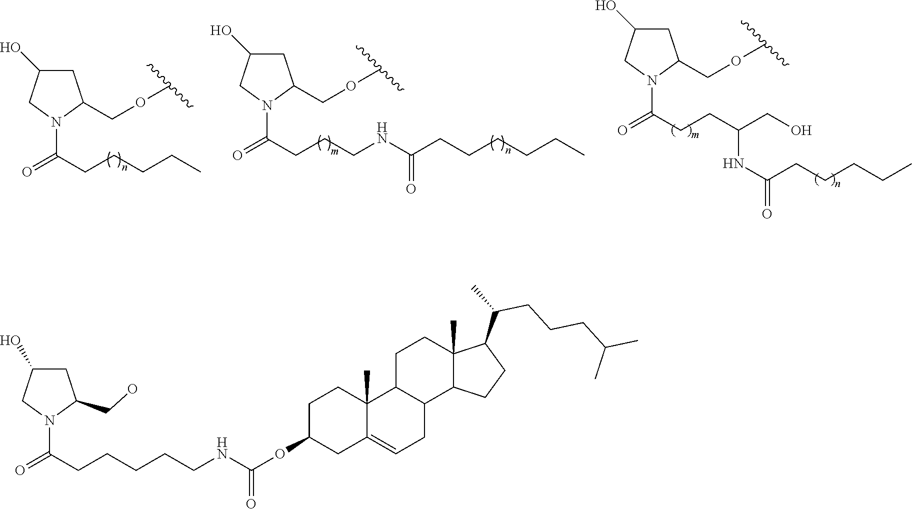

wherein m is 0-8. And n is 1-21.

In some embodiments, the lipophilic moiety is conjugated to the 5′-end of one of the sense and antisense strand with via a direct bond or through a carrier or linker. Examples include, but are not limited to moieties of the formula,

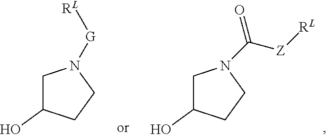

where the broken bond is to the 5′-end (5′-OH) of the oligonucleotide via a phosphodiester or phosphothioate internucleotide linkage; G is a linking group; Z is a bond, O, S, or N(H), and R^{L} is a lipophilic moiety, such as those described herein, including those below:

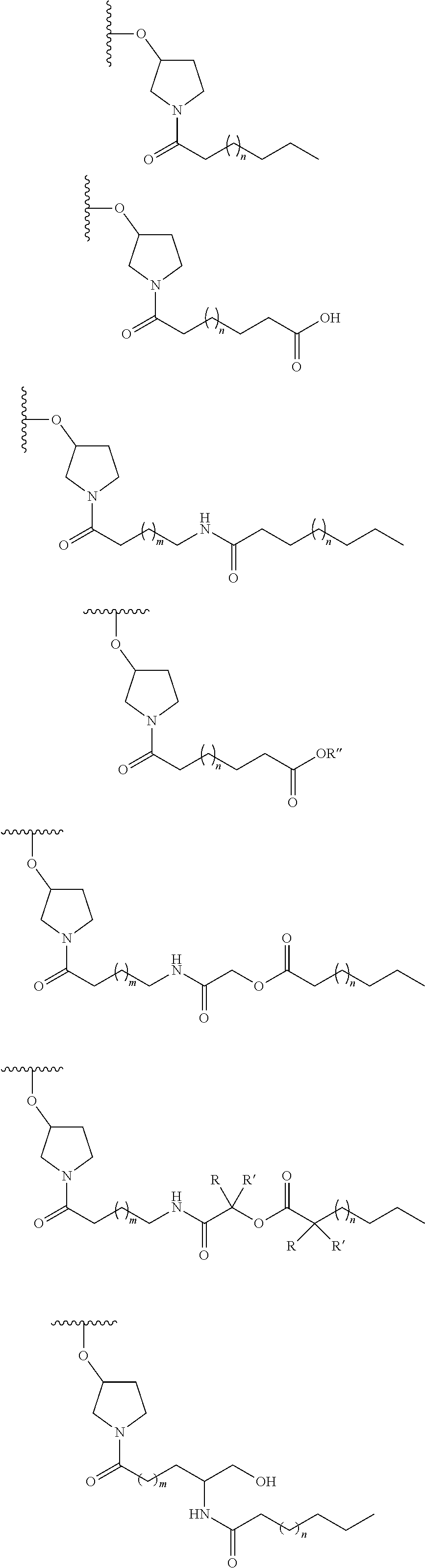

In some embodiments, the lipophilic moiety is conjugated to the 5′-end of one of the sense and anti sense strand with via a direct bond to the 5′-terminal 5′-carbon. For example,

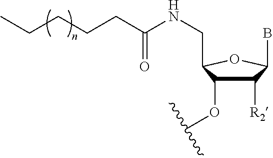

R₂′ is 2′-F, 2′-OMe, B is an optionally modified nucleobase, and n ia 1-21. 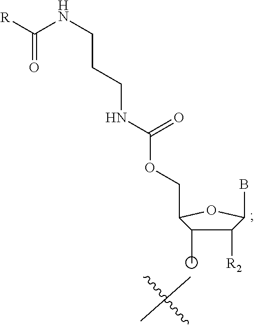
wherein R is the lipophilic moiety as defined herein. R₂′ is H, OH, F, OMe, O-methoxyalkyl, O-allyl, O-N-methylacetamido, O-dimethylaminoethoxyethyl, or O- aminopropyl. B is a optionally modified nucleobase.

In some embodiments, the lipophilic moiety is conjugated to the 3′-end or 5′-end of one of the sense and antisense strand with via a direct bond or through a carrier or linker. In some embodiments, the lipophilic moiety is conjugated to the 3′-end of one of the sense and antisense strand with via a direct bond or through a carrier or linker. In some embodiments, the lipophilic moiety is conjugated to the 5′-end of one of the sense and antisense strand with via a direct bond or through a carrier or linker. In some embodiments, the lipophilic moiety is of the formula,

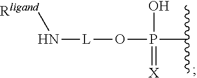

or a salt thereof, wherein X is O or S (e.g., S); L is a divalent linking group; e.g., C₁₋₂₀ alkyl, C₁₋₁₀alkyl-S—S—C₁₋₁₀alkyl. In one particular example, the lipophilic group is of the formula,

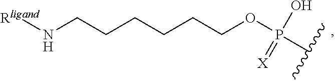

wherein X is O or S (e.g., S), and R^{ligand} is selected from Table 1.

TABLE 1

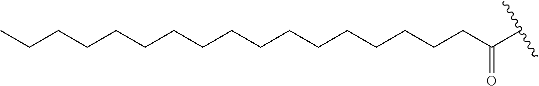 [SMILES: *C(=O)CCCCCCCCCCCCCCCCC]

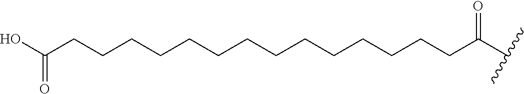 [SMILES: *C(=O)CCCCCCCCCCCCCCC(=O)O]

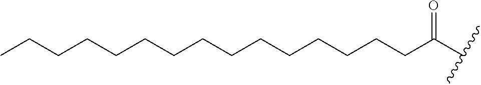 [SMILES: *C(=O)CCCCCCCCCCCCCCC]

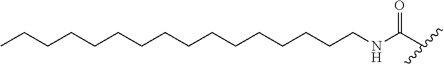 [SMILES: *C(=O)NCCCCCCCCCCCCCCCC]

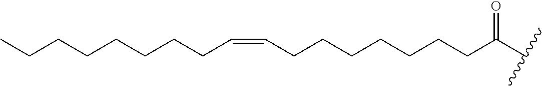 [SMILES: *C(=O)CCCCCCC/C=C\CCCCCCCC]

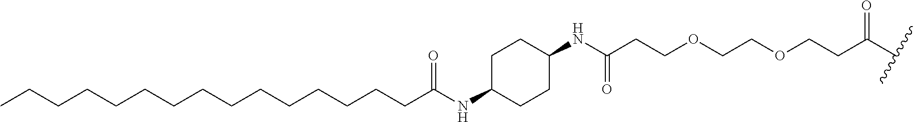 [SMILES: *C(=O)CCOCCOCCC(=O)N[C@H]1CC[C@@H](NC(=O)CCCCCCCCCCCCCCC)CC1]

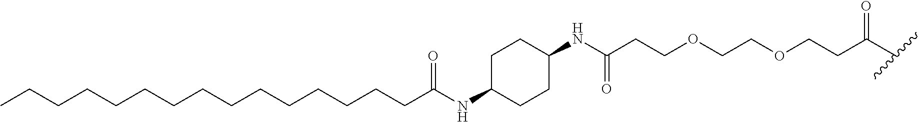 [SMILES: *C(=O)CCOCCOCCC(=O)N[C@H]1CC[C@@H](NC(=O)CCCCCCCCCCCCCCC)CC1]

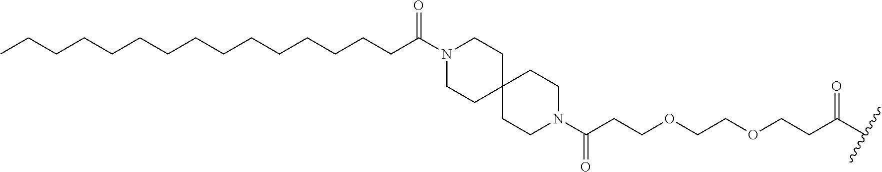 [SMILES: *C(=O)CCOCCOCCC(=O)N1CCC2(CCN(C(=O)CCCCCCCCCCCCCCC)CC2)CC1]

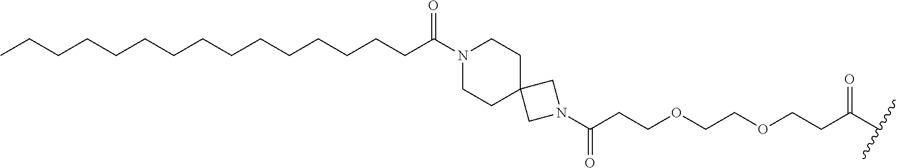 [SMILES: *C(=O)CCOCCOCCC(=O)N1CC2(CCN(C(=O)CCCCCCCCCCCCCCC)CC2)C1]

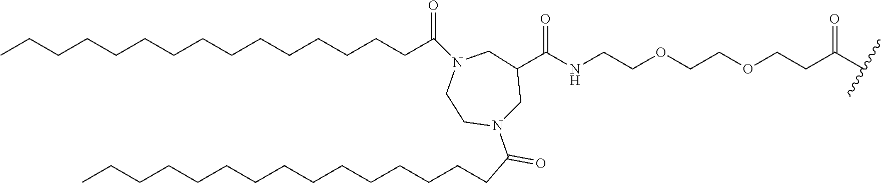 [SMILES: *C(=O)CCOCCOCCNC(=O)C1CN(C(=O)CCCCCCCCCCCCCCC)CCN(C(=O)CCCCCCCCCCCCCCC)C1]

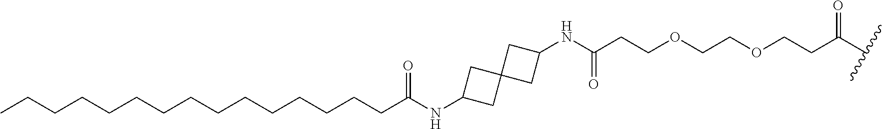 [SMILES: *C(=O)CCOCCOCCC(=O)NC1CC2(CC(NC(=O)CCCCCCCCCCCCCCC)C2)C1]

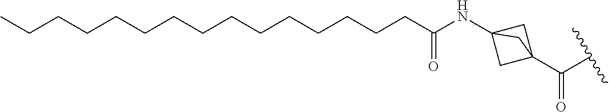 [SMILES: *C(=O)C12CC(NC(=O)CCCCCCCCCCCCCCC)(C1)C2]

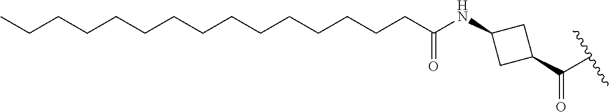 [SMILES: *C(=O)[C@H]1C[C@@H](NC(=O)CCCCCCCCCCCCCCC)C1]

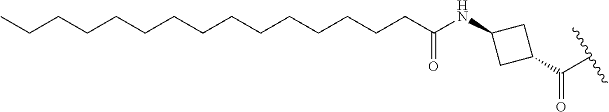 [SMILES: *C(=O)[C@H]1C[C@H](NC(=O)CCCCCCCCCCCCCCC)C1]

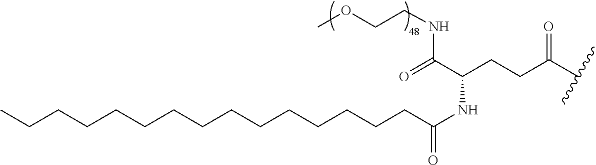

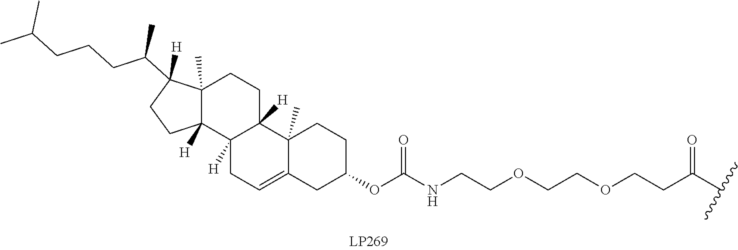 [SMILES: *C(=O)CCOCCOCCNC(=O)O[C@H]1CC[C@@]2(C)C(=CC[C@H]3[C@@H]4CC[C@H]([C@H](C)CCCC(C)C)[C@@]4(C)CC[C@@H]32)C1]

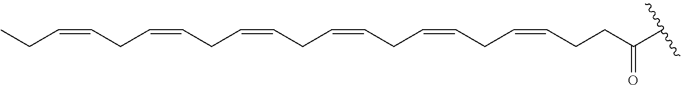 [SMILES: *C(=O)CC/C=C\C/C=C\C/C=C\C/C=C\C/C=C\C/C=C\CC]

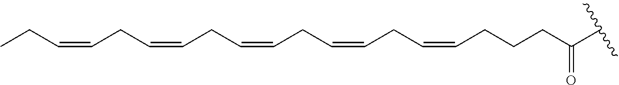 [SMILES: *C(=O)CCC/C=C\C/C=C\C/C=C\C/C=C\C/C=C\CC]

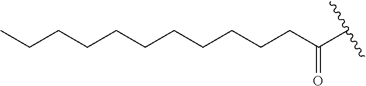 [SMILES: *C(=O)CCCCCCCCCCC]

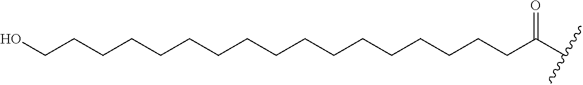 [SMILES: *C(=O)CCCCCCCCCCCCCCCCCO]

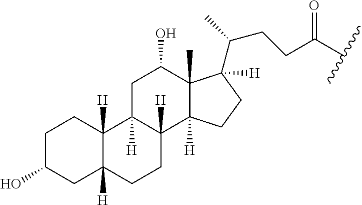

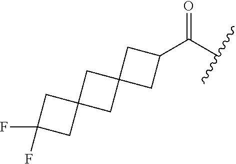 [SMILES: *C(=O)C1CC2(C1)CC1(CC(F)(F)C1)C2]

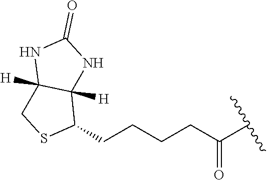 [SMILES: *C(=O)CCCC[C@@H]1SC[C@@H]2NC(=O)N[C@@H]21]

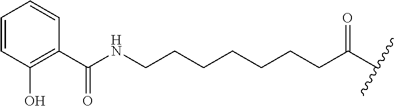 [SMILES: *C(=O)CCCCCCCNC(=O)c1ccccc1O]

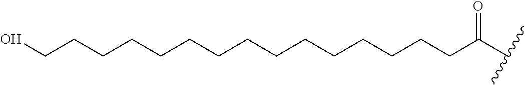 [SMILES: *C(=O)CCCCCCCCCCCCCCCO]

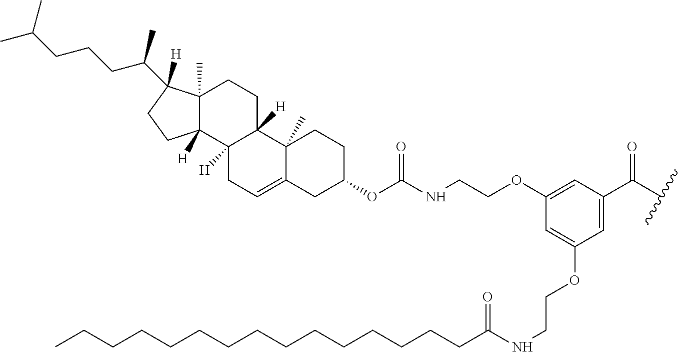 [SMILES: *C(=O)c1cc(OCCNC(=O)CCCCCCCCCCCCCCC)cc(OCCNC(=O)O[C@H]2CC[C@@]3(C)C(=CC[C@H]4[C@@H]5CC[C@H]([C@H](C)CCCC(C)C)[C@@]5(C)CC[C@@H]43)C2)c1]

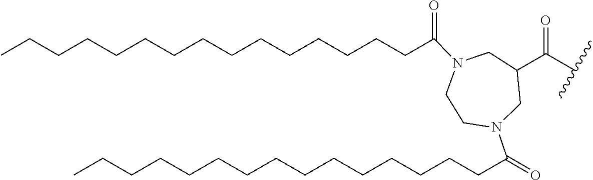 [SMILES: *C(=O)C1CN(C(=O)CCCCCCCCCCCCCCC)CCN(C(=O)CCCCCCCCCCCCCCC)C1]

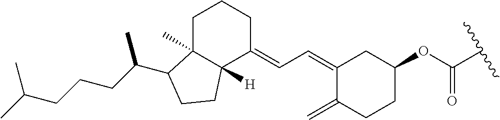 [SMILES: *C(=O)O[C@H]1CCC(=C)/C(=C\C=C2/CCC[C@]3(C)C([C@H](C)CCCC(C)C)CC[C@@H]23)C1]

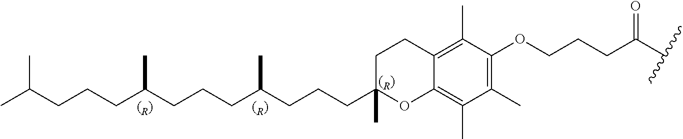

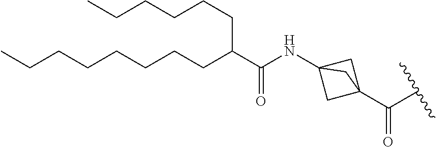 [SMILES: *C(=O)C12CC(NC(=O)C(CCCCCC)CCCCCCCC)(C1)C2]

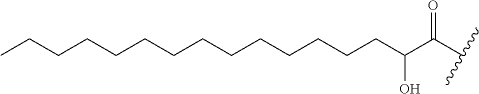 [SMILES: *C(=O)C(O)CCCCCCCCCCCCCC]

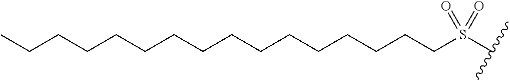 [SMILES: *S(=O)(=O)CCCCCCCCCCCCCCCC]

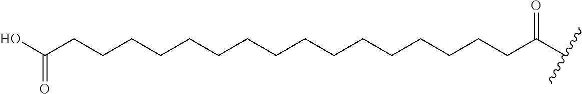 [SMILES: *C(=O)CCCCCCCCCCCCCCCCC(=O)O]

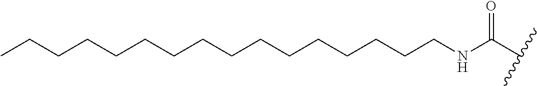 [SMILES: *C(=O)NCCCCCCCCCCCCCCCC]

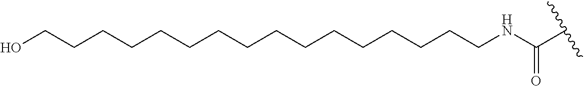 [SMILES: *C(=O)NCCCCCCCCCCCCCCCCO]

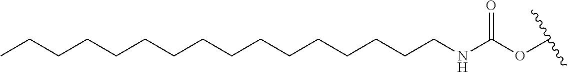 [SMILES: *OC(=O)NCCCCCCCCCCCCCCCC]

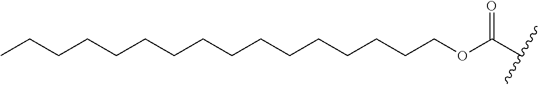 [SMILES: *C(=O)OCCCCCCCCCCCCCCCC]

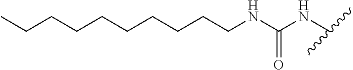 [SMILES: *NC(=O)NCCCCCCCCCC]

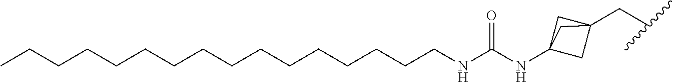 [SMILES: *CC12CC(NC(=O)NCCCCCCCCCCCCCCCC)(C1)C2]

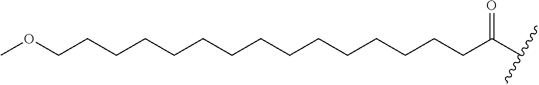 [SMILES: *C(=O)CCCCCCCCCCCCCCCOC]

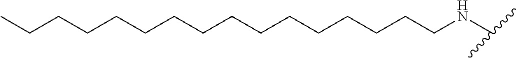 [SMILES: *NCCCCCCCCCCCCCCCC]

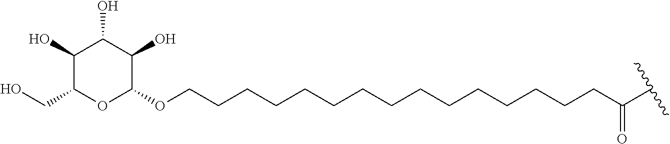 [SMILES: *C(=O)CCCCCCCCCCCCCCCO[C@@H]1O[C@H](CO)[C@@H](O)[C@H](O)[C@H]1O]

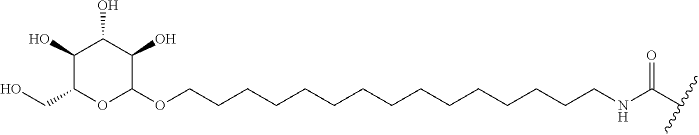 [SMILES: *C(=O)NCCCCCCCCCCCCCCCOC1O[C@H](CO)[C@@H](O)[C@H](O)[C@H]1O]

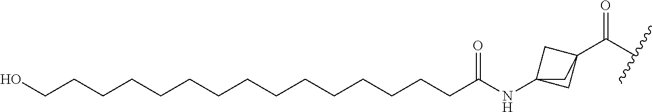 [SMILES: *C(=O)C12CC(NC(=O)CCCCCCCCCCCCCCCO)(C1)C2]

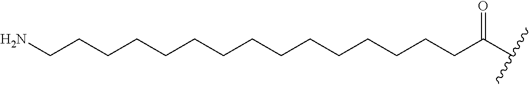 [SMILES: *C(=O)CCCCCCCCCCCCCCCN]

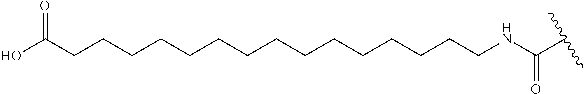 [SMILES: *C(=O)NCCCCCCCCCCCCCCCC(=O)O]

or

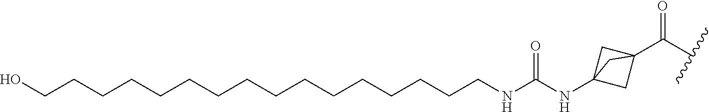 [SMILES: *C(=O)C12CC(NC(=O)NCCCCCCCCCCCCCCCCO)(C1)C2]

In another example, the lipophilic moiety is one of the following that is bonded to the 5′-oxygen of the 5′-terminal nucleotide or the 3′-oxygen of the 3′-terminal nucleotide:

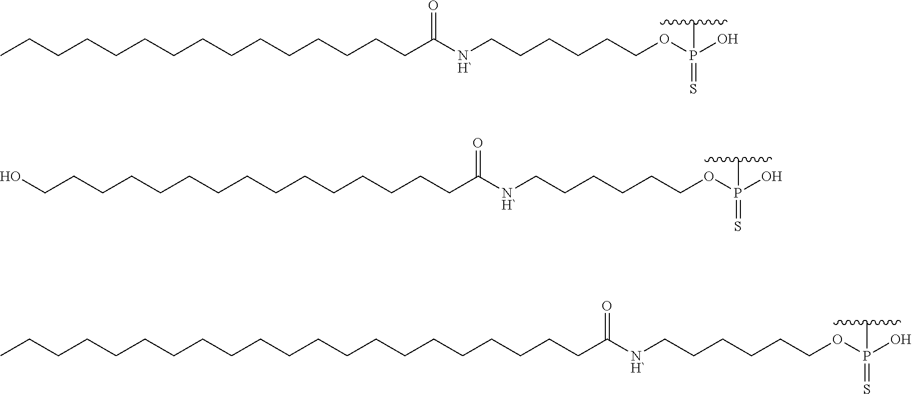

In some embodiments, the carrier or linker is an inverted abasic nucleotide, such as an inverted abasic deoxyribonucleotide or an inverted abasic ribonucleotide, each connected to the remainder of the oligonucleotide via a phosphodiester (PO) or phosphorothioate (PS) linkage. Examples include, but are not limited to,

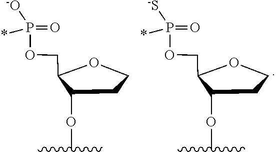

In some embodiments, the lipophilic group is attached to the 5′-oxygen of the 5′-terminal nucleotide or the 3′-oxygen of the 3′-terminal nucleotide of the oligonucleotide and is of the formula,

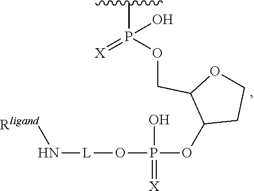

or a salt thereof, each X is independently O or S (e.g., each is S) and R^{ligand} is selected from Table 1, and L is a divalent linking group; e.g., C₁₋₂₀ alkyl, C₁₋₁₀alkyl-S—S—C₁₋₁₀alkyl. The preceding structure can be, for example, 5′-(L1)(inv)- as used herein.

For example, the lipophilic group is attached to the 5′-oxygen of the 5′-terminal nucleotide or the 3′-oxygen of the 3′-terminal nucleotide of the oligonucleotide and is of the formula,

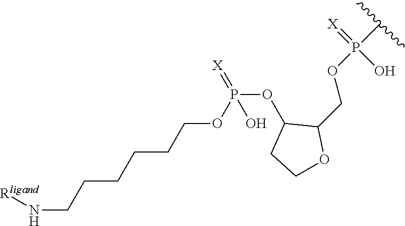

wherein each X is O or S (e.g., each is S) and R^{ligand} is selected from Table 1; examples include wherein R^{ligand} is selected from Table 2:

TABLE 2

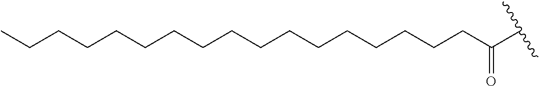 [SMILES: *C(=O)CCCCCCCCCCCCCCCCC]

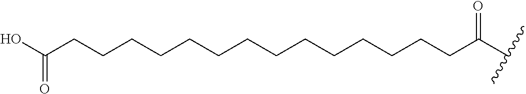 [SMILES: *C(=O)CCCCCCCCCCCCCCC(=O)O]

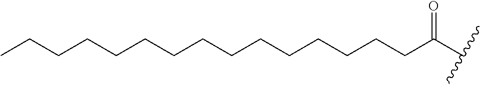 [SMILES: *C(=O)CCCCCCCCCCCCCCC]

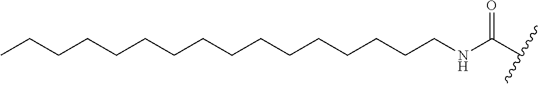 [SMILES: *C(=O)NCCCCCCCCCCCCCCCC]

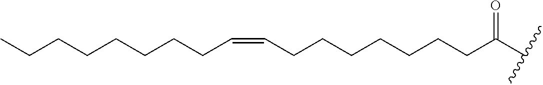 [SMILES: *C(=O)CCCCCCC/C=C\CCCCCCCC]

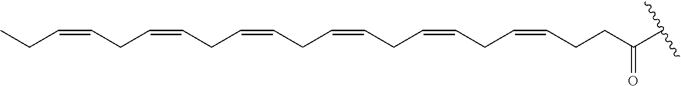 [SMILES: *C(=O)CC/C=C\C/C=C\C/C=C\C/C=C\C/C=C\C/C=C\CC]

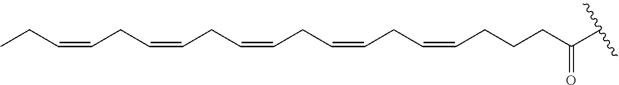 [SMILES: *C(=O)CCC/C=C\C/C=C\C/C=C\C/C=C\C/C=C\CC]

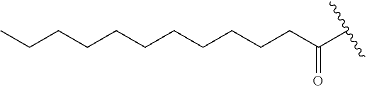 [SMILES: *C(=O)CCCCCCCCCCC]

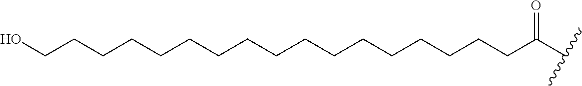 [SMILES: *C(=O)CCCCCCCCCCCCCCCCCO]

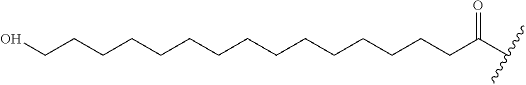 [SMILES: *C(=O)CCCCCCCCCCCCCCCO]

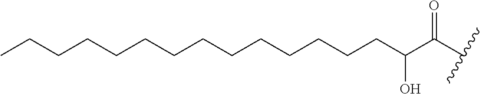 [SMILES: *C(=O)C(O)CCCCCCCCCCCCCC]

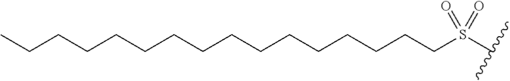 [SMILES: *S(=O)(=O)CCCCCCCCCCCCCCCC]

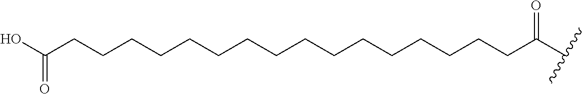 [SMILES: *C(=O)CCCCCCCCCCCCCCCCC(=O)O]

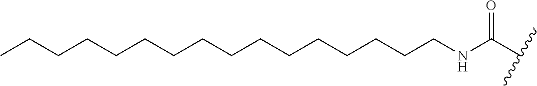 [SMILES: *C(=O)NCCCCCCCCCCCCCCCC]

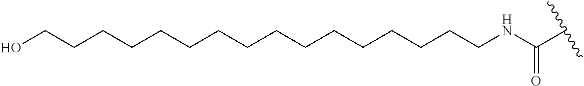 [SMILES: *C(=O)NCCCCCCCCCCCCCCCCO]

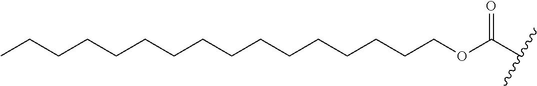 [SMILES: *C(=O)OCCCCCCCCCCCCCCCC]

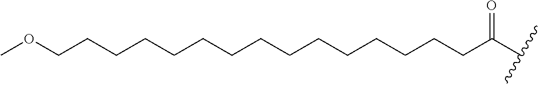 [SMILES: *C(=O)CCCCCCCCCCCCCCCOC]

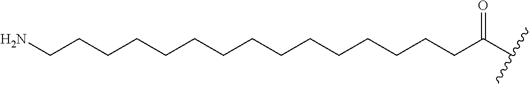 [SMILES: *C(=O)CCCCCCCCCCCCCCCN]

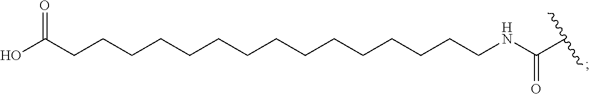 [SMILES: *C(=O)NCCCCCCCCCCCCCCCC(=O)O]

or

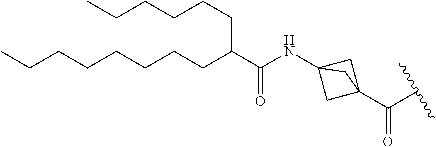 [SMILES: *C(=O)C12CC(NC(=O)C(CCCCCC)CCCCCCCC)(C1)C2]

The preceding structure can be, for example, 5′-(L1)(inv)- as used herein.

In another example, the lipophilic moiety is one of the following that is bonded to the 3′-oxygen of the 3′-terminal nucleotide or the 5′-oxygen of the 5′-terminal nucleotide and is of the formula,

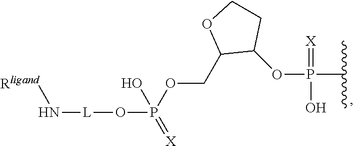

or a salt thereof, wherein each X is independently O or S (e.g., each is S) and R^{ligand} is selected from Table 1 (above), and L is a divalent linking group; e.g., C₁₋₂₀ alkyl, C₁₋₁₀alkyl-S—S—C₁₋₁₀alkyl. The preceding structure can be, for example, -(inv)(L2)-3′ as used herein.

In one particular example, the lipophilic moiety is bonded to the 3′-oxygen of the 3′-terminal nucleotide or the 5′-oxygen of the 5′-terminal nucleotide and is of the formula,

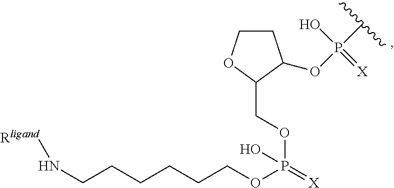

or a salt thereof, wherein each X is O or S (e.g., each is S) and R^{ligand} is selected from Table 1 (above); examples include wherein R^{ligand} is selected from Table 2 (above). The preceding structure can be -(inv)(L2)-3′ as used herein.

In another embodiment, the lipophilic moiety is a steroid, such as sterol. Steroids are polycyclic compounds containing a perhydro-1,2-cyclopentanophenanthrene ring system. Steroids include, without limitation, bile acids (e.g., cholic acid, deoxycholic acid and dehydrocholic acid), cortisone, digoxigenin, testosterone, cholesterol, and cationic steroids, such as cortisone. A “cholesterol derivative” refers to a compound derived from cholesterol, for example by substitution, addition or removal of substituents.

In another embodiment, the lipophilic moiety is an aromatic moiety. In this context, the term “aromatic” refers broadly to mono- and polyaromatic hydrocarbons. Aromatic groups include, without limitation, C₆-C₁₄ aryl moieties comprising one to three aromatic rings, which may be optionally substituted; “aralkyl” or “arylalkyl” groups comprising an aryl group covalently linked to an alkyl group, either of which may independently be optionally substituted or unsubstituted; and “heteroaryl” groups. As used herein, the term “heteroaryl” refers to groups having 5 to 14 ring atoms, preferably 5, 6, 9, or 10 ring atoms; having 6, 10, or 14π electrons shared in a cyclic array, and having, in addition to carbon atoms, between one and about three heteroatoms selected from the group consisting of nitrogen (N), oxygen (O), and sulfur (S).

As employed herein, an “optionally substituted” or a “substituted” alkyl, cycloalkyl, aryl, heteroaryl, or heterocyclic group is one having between one and about four, preferably between one and about three, more preferably one or two, non-hydrogen substituents. Suitable substituents include, without limitation, halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups. The term “acyl” refers to an alkyl group that is connected to another chemical moiety via a carbonyl group (C═O).

In some embodiments, the lipophilic moiety is an aralkyl group, e.g., a 2-arylpropanoyl moiety. The structural features of the aralkyl group are selected so that the lipophilic moiety will bind to at least one protein in vivo. In certain embodiments, the structural features of the aralkyl group are selected so that the lipophilic moiety binds to serum, vascular, or cellular proteins. In certain embodiments, the structural features of the aralkyl group promote binding to albumin, an immunoglobulin, a lipoprotein, α-2-macroglubulin, or α-1-glycoprotein.

In certain embodiments, the ligand is naproxen or a structural derivative of naproxen. Procedures for the synthesis of naproxen can be found in U.S. Pat. Nos. 3,904,682 and 4,009,197, which are hereby incorporated by reference in their entirety. Naproxen has the chemical name (S)-6-Methoxy-α-methyl-2-naphthaleneacetic acid and the structure is

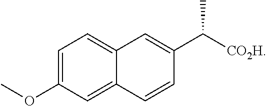 [SMILES: COc1ccc2cc([C@H](C)C(=O)O)ccc2c1]

In certain embodiments, the ligand is ibuprofen or a structural derivative of ibuprofen. Procedures for the synthesis of ibuprofen can be found in U.S. Pat. No. 3,228,831, which are hereby incorporated by reference in their entirety. The structure of ibuprofen is

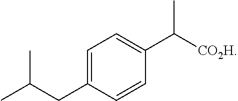

Additional exemplary aralkyl groups are illustrated in U.S. Pat. No. 7,626,014, which is incorporated herein by reference in its entirety.

In another embodiment, suitable lipophilic moieties include lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, 03-(oleoyl)lithocholic acid, 03-(oleoyl)cholenic acid, ibuprofen, naproxen, dimethoxytrityl, or phenoxazine.

In certain embodiments, more than one lipophilic moieties can be incorporated into the double-strand RNAi agent, particularly when the lipophilic moiety has a low lipophilicity or hydrophobicity. In one embodiment, two or more lipophilic moieties are incorporated into the same strand of the double-strand RNAi agent. In one embodiment, each strand of the double-strand RNAi agent has one or more lipophilic moieties incorporated. In one embodiment, two or more lipophilic moieties are incorporated into the same position (i.e., the same nucleobase, same sugar moiety, or same internucleosidic linkage) of the double-strand RNAi agent. This can be achieved by, e.g., conjugating the two or more lipophilic moieties via a carrier, and/or conjugating the two or more lipophilic moieties via a branched linker, and/or conjugating the two or more lipophilic moieties via one or more linkers, with one or more linkers linking the lipophilic moieties consecutively.

The lipophilic moiety may be conjugated to the RNAi agent via a direct attachment to the ribosugar of the RNAi agent. Alternatively, the lipophilic moiety may be conjugated to the double-strand RNAi agent via a linker or a carrier.

In certain embodiments, the lipophilic moiety may be conjugated to the RNAi agent via one or more linkers (tethers).

In one embodiment, the lipophilic moiety is conjugated to the double-stranded RNAi agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

Exemplary linkers, tethers, carriers, nucleic acid modifications, conjugates, ligands and other moieties useful for achieving central nervous system-directed delivery of the APP-targeting RNAi agents of the instant disclosure are described in additional detail, e.g., in International Publication WO2019/217459, the entire contents of which are incorporated herein by this reference.

Additional lipophilic-modified nucleotides include those described in International Publication WO2021/092371 and U.S. Provisional Application Ser. No. 63/357,379, entitled MONOMERS AND METHODS FOR SYNTHESIS OF MODIFIED OLIGONUCLEOTIDES, filed on Jun. 30, 2022, the entire contents of which are incorporated by reference herein.

Further exemplary lipid modified duplex motifs contemplated for use in the compositions and methods disclosed herein may be found in WO2023/245060 and WO2023/245061, which are incorporated herein by reference in their entirety.

### Lipid Conjugates

In one embodiment, the ligand or conjugate is a lipid or lipid-based molecule. Such a lipid or lipid-based molecule preferably binds a serum protein, e.g., human serum albumin (HSA). An HSA binding ligand allows for vascular distribution of the conjugate to a target tissue, e.g., a non-kidney target tissue of the body. In certain embodiments, the target tissue can be the CNS, including glial cells of the brain. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

Optionally, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by target cells such as brain cells. Also included are HSA and low density lipoprotein (LDL).

In some embodiments, a carbohydrate conjugate of a RNAi agent of the instant disclosure further comprises one or more additional ligands as described above, such as, but not limited to, a PK modulator and/or a cell permeation peptide.

Additional carbohydrate conjugates (and linkers) suitable for use in the present disclosure include those described in PCT Publication Nos. WO 2014/179620 and WO 2014/179627, the entire contents of each of which are incorporated herein by reference.

### Exemplary Lipid Modified Duplex Motifs

For example, in certain embodiments, the sense strand of the dsRNA agent has one of the following modification patterns:

| | | |
|---|---|---|
| S1 | 5′-nnnnnnNfnNfNfNfnnnnnnnnnn-3′, | 21 |
| S2 | 5′-nnnnnnnnNfNfNfnnnnnnnnnn-3′, | 21 |
| S3 | 5′-NfnNfnNfnNfnNfNfNfnNfnNfnNfnNfnNf-3′, | 21 |
| S4 | 5′-NfnNfnNfnNfnNfNfNfnNfnnnNfnNfnNf-3′, | 21 |
| S5 | 5′-nnnnnnnn(dN)n(dN)nnnnnnnnnn-3′, | 21 |
| S6 | 5′-nnnnnnNfnNfn(dN)nnnnnnnnnn-3′, | 21 |
| S7 | 5′-nnnnnnnnNfnNfnNfnnnnnnnn-3′ | 21 |
| S8 | 5′-nnnnNfnNfNfNfnnnnnnnnnn-3′, | 19 |
| S9 | 5′-nnnnnnNfNfNfnnnnnnnnnn-3′, | 19 |
| S10 | 5′-NfnNfnNfnNfNfNfnNfnNfnNfnNfnNf-3′, | 19 |
| S11 | 5′-NfnNfnNfnNfNfNfnNfnnnNfnNfnNf-3′, | 19 |
| S12 | 5′-nnnnnn(dN)n(dN)nnnnnnnnnn-3′, | 19 |
| S13 | 5′-nnnnNfnNfn(dN)nnnnnnnnnn-3′, | 19 |
| S14 | 5′-nnnnnnNfnNfnNfnnnnnnnn-3′ | 19 |

wherein
n is a 2′-O-methyl-nucleotide;
Nf is a 2′-fluoro-modified nucleotide,
and sense strand comprises at least one of the following modifications (a)-(c):
(a) a lipophilic-modified nucleotide “(Lipo)” is substituted at any one of positions 4-8 or 13-18 counting from the 5′-end of the strand, examples include:
   (Nhd)—2′-O-hexadecyl-modified nucleotide;
   (Nda)—a 2′-O-docosanyl-modified nucleotide;
   (NhdOH)—2′-O-(omega-hydroxy-hexadecyl)-modified nucleotide); or
   (NdaOH)—a 2′-O-(omega-hydroxy-docosanyl)-modified nucleotide;
(b) 5′-(L1)(inv)- attached to the 5′-terminal nucleotide (5′-5′), optionally via a phosphodiester or phosphorothioate linkage; or
(c) -(inv)(L2)-3′, attached to the 3′-terminal nucleotide (3′-3′), optionally via a phosphodiester or phosphorothioate linkage,
wherein
   each (inv) is an inverted nucleotide (e.g., an inverted abasic nucleotide, such as an inverted abasic ribonucleotide, such as an inverted abasic deoxyribonucleotide);
   (L1) and (L2) are independently absent, a hydrogen, or a ligand comprising a lipophilic group (e.g., comprising an C₁₀-C₃₀ alkyl, or a C₁₀-C₃₀ alkenyl group, e.g., a C₁₆ alkyl, a C₁₆ alkenyl, a Cis alkyl, a C₁₈ alkenyl, a C₂₀alkyl, a C₂₀ alkenyl, a C₂₂ alkyl, a C₂₂ alkenyl, a C₂₄ alkyl, a C₂₄ alkenyl; C₁₅ alkyl, a C₁₅ alkenyl, a C₁₇ alkyl, a C₁₇ alkenyl, a C₁₉alkyl, a C₁₉ alkenyl, a C₂₁ alkyl, a C₂₁ alkenyl, a C₂₃ alkyl, or a C₂₃ alkenyl).

In each of the preceding sense strands, each of the nucleotides are connected in series (i.e., in a 3′->5′ manner) via optionally modified internucleotide linkages. For example, each of the nucleotides are connected by phosphodiester or phosphorothioate internucleotide linkages.

In certain embodiments, the nucleotides at position 1 and 2 are connected by a phosphorothioate internucleotide linkage; the nucleotides at position 2 and 3 are connected by a phosphorothioate internucleotide linkage; and the remaining nucleotides are connected in via phosphodiester bonds, counting from the 5′-end of the oligonucleotide.

In certain embodiments, the nucleotides at position 1 and 2 are connected by a phosphorothioate internucleotide linkage; the nucleotides at position 2 and 3 are connected by a phosphorothioate internucleotide linkage; the nucleotides at position 3 and 4 are connected by a phosphorothioate internucleotide linkage; and the remaining nucleotides are connected in via phosphodiester bonds, counting from the 5′-end of the oligonucleotide.

In certain embodiments, where the nucleotide in m nucleotides in length, the nucleotides at positions m-1 and m are connected by a phosphorothioate internucleotide linkage, counting from the 5′-end of the oligonucleotide. That is, for a nucleotide in 23 nucleotides in length, the nucleotides at positions 22 and 23 are connected by a phosphorothioate internucleotide linkage, counting from the 5′-end of the oligonucleotide; and for a nucleotide in 21 nucleotides in length, the nucleotides at positions 20 and 21 are connected by a phosphorothioate internucleotide linkage, counting from the 5′-end of the oligonucleotide.

In certain embodiments, where the nucleotide in m nucleotides in length, the nucleotides at positions m-2 and m-1 are connected by a phosphorothioate internucleotide linkage, and the nucleotides at positions m-1 and m are connected by a phosphorothioate internucleotide linkage, counting from the 5′-end of the oligonucleotide. That is, for a nucleotide in 23 nucleotides in length, the nucleotides at positions 21 and 22 are connected by a phosphorothioate internucleotide linkage; and positions 22 and 23 are connected by a phosphorothioate internucleotide linkage, counting from the 5′-end of the oligonucleotide; and for a nucleotide in 21 nucleotides in length, the nucleotides at positions 20 and 21 are connected by a phosphorothioate internucleotide linkage, counting from the 5′-end of the oligonucleotide.

In other embodiments, each (inv) attached to a 5′-terminal nucleotide is connected via a phosphorothioate linkage (5′-5′).

In other embodiments, each (inv) attached to a 3′-terminal nucleotide is connected via a phosphorothioate linkage (3′-3′).

In certain other embodiments, each (inv) attached to a 5′-terminal nucleotide is connected via a phosphorothioate linkage (5′-5′), and each (inv) attached to a 3′-terminal nucleotide is connected via a phosphorothioate linkage (3′-3′).

For example, in certain embodiments, the sense strand of the dsRNA agent has one of the following modification patterns (with variables as defined above):

| | |
|---|---|
| S15 | 5′-(Lipo)snsnnnnNfnNfNfNfnnnnnnnnsnsn-3′ |
| S16 | 5′-ns(Lipo)snnnnNfnNfNfNfnnnnnnnnsnsn-3′ |
| S17 | 5′-nsns(Lipo)nnnNfnNfNfNfnnnnnnnnsnsn-3′ |
| S18 | 5′-nsnsn(Lipo)nnNfnNfNfNfnnnnnnnnsnsn-3′ |
| S19 | 5′-nsnsnn(Lipo)nNfnNfNfNfnnnnnnnnsnsn-3′ |
| S20 | 5′-nsnsnnn(Lipo)NfnNfNfNfnnnnnnnnsnsn-3′ |
| S21 | 5′-nsnsn(Lipo)nnnnNfNfNfnnnnnnnnsnsn-3′ |
| S23 | 5′-nsnsnn(Lipo)nnnNfNfNfnnnnnnnnsnsn-3′ |
| S24 | 5′-nsnsnnn(Lipo)nnNfNfNfnnnnnnnnsnsn-3′ |
| S25 | 5′-nsnsnnnn(Lipo)nNfNfNfnnnnnnnnsnsn-3′ |
| S26 | 5′-nsnsnnnnNfnNfNfNfn(Lipo)nnnnnnsnsn-3′ |
| S27 | 5′-nsnsnnnnNfnNfNfNfnn(Lipo)nnnnnsnsn-3′ |
| S28 | 5′-nsnsnnnnNfnNfNfNfnnn(Lipo)nnnnsnsn-3′ |
| S29 | 5′-nsnsnnnnNfnNfNfNfnnnn(Lipo)nnnsnsn-3′ |
| S30 | 5′-nsnsnnnnNfnNfNfNfnnnnn(Lipo)nnsnsn-3′ |
| S31 | 5′-nsnsnnnnNfnNfNfNfnnnnn(Lipo)nnsns(Lipo)-3′ |
| S32 | 5′-nsnsnnnnnnNfNfNfn(Lipo)nnnnnnsnsn-3′ |
| S33 | 5′-nsnsnnnnnnNfNfNfnn(Lipo)nnnnnsnsn-3′ |
| S34 | 5′-nsnsnnnnnnNfNfNfnnn(Lipo)nnnnsnsn-3′ |
| S35 | 5′-nsnsnnnnnnNfNfNfnnnn(Lipo)nnnsnsn-3′ |
| S36 | 5′-nsnsnnnnnnNfNfNfnnnnn(Lipo)nnsnsn-3′ |
| S37 | 5′-nsnsnnnnnnNfNfNfnnnnn(Lipo)nnsns(Lipo)-3′ |
| S38 | 5′-(L1)(inv)snnnnnnnnNfNfNfnnnnnnnnns(inv)(L2)-3′, |
| S39 | 5′-(L1)(inv)snnnnnnnnNfNfNfnnnnnnnnns(inv)-3′, |
| S40 | 5′-(inv)snnnnnnnnNfNfNfnnnnnnnnns(inv)(L2)-3′, |
| S41 | 5′NfsnsNfnNfnNfnNfNfNfnNfnNfnNfnNfnsNf-3′, where (Lipo) is |
| | substituted at any one of positions 4-8 or 13-18 counting from the 5′-end of |
| | the strand |
| S42 | 5′NfsnsNfnNfnNfnNfNfNfnNfnnnNfnNfsnsNf-3′, where (Lipo) is |
| | substituted at any one of positions 4-8 or 13-18 counting from the 5′-end of |
| | the strand |
| S43 | 5′-nsnsnnnnnn(dN)n(dN)nnnnnnnnsnsn-3′, where (Lipo) is substituted at |
| | any one of positions 4-8 or 13-18 counting from the 5′-end of the strand |
| S44 | 5′-nsnsnnnnNfnNfn(dN)nnnnnnnnsnsn-3′, where (Lipo) is substituted at |
| | any one of positions 4-8 or 13-18 counting from the 5′-end of the strand |
| S45 | 5′-(L1)(inv)snnnnnnnnNfnNfnNfnnnnnnnns(inv)-3′, |
| S46 | 5′-(inv)snnnnnnnnNfnNfnNfnnnnnnnns(inv)-3′, where (Lipo) is substituted |
| | at any one of positions 4-8 or 13-18 counting from the 5′-end of the strand |
| S47 | 5′-(L1)(inv)snnnnnnNfnNfNfNfnnnnnnnnnns(inv)-3′ |
| S48 | 5′-(inv)snnnnnnNfnNfNfNfnnnnnnnnnns(inv)-3′, where (Lipo) is |
| | substituted at any one of positions 4-8 or 13-18 counting from the 5′-end of |
| | the strand |
| S49 | 5′-(L1)(inv)snnnnnnnnNfNfNfnnnnnnnnnns(inv)-3′ |
| S50 | 5′-(inv)snnnnnnnnNfNfNfnnnnnnnnnns(inv)-3′, where (Lipo) is substituted |
| | at any one of positions 4-8 or 13-18 counting from the 5′-end of the strand |
| S51 | 5′-(L1)(inv)snnnnnnnn(dN)n(dN)nnnnnnnnnns(inv)-3′, |
| S52 | 5′-(inv)snnnnnnnn(dN)n(dN)nnnnnnnnnns(inv)-3′, where (Lipo) is |
| | substituted at any one of positions 4-8 or 13-18 counting from the 5′-end of |
| | the strand |
| S53 | 5′-(L1)(inv)snnnnnnNfnNfn(dN)nnnnnnnnnns(inv)-3′, |
| S54 | 5′-(inv)snnnnnnNfnNfn(dN)nnnnnnnnnns(inv)-3′, where (Lipo) is |
| | substituted at any one of positions 4-8 or 13-18 counting from the 5′-end of |
| | the strand |

wherein s is a phosphorothioate internucleotide linkage.

In some embodiments, the antisense strand of the dsRNA agent has one of the following modification patterns:

| | |
|---|---|
| AS1 | 5′-Zn(dN)nn(dN)n(dN)nnnn(dN)nNfnnnnnnnnn-3′ |
| AS2 | 5′-Zn(dN)nn(dN)n(G)nnnn(dN)nNfnnnnnnnnn-3′ |
| AS3 | 5′-ZnNfnn(dN)n(G)nnnnnnNfnNfnnnnnnn-3′ |
| AS4 | 5′-ZnNfnnnNfnnnnnnnNfnNfnnnnnnn-3′ |
| AS5 | 5′-ZnNfnnnNfnnNfnnnnNfnNfnnnnnnn-3′ |
| AS6 | 5′-ZnNfnnnNfnNfNfnnnnNfnNfnnnnnnn-3′ |
| AS7 | 5′-ZnNfnnnNf(G)nnnnnnNfnNfnnnnnnn-3′ |
| AS8 | 5′-ZnNfnnnNf(G)nNfnnnnNfnNfnnnnnnn-3′ |
| AS9 | 5′-ZnNfnnnNf(G)NfNfnnnnNfnNfnnnnnnn-3′ |
| AS10 | 5′-ZnNfnNfnNfnNfnNfnNfnNfnNfnNfnNfn-3′ |
| AS11 | 5′-ZnNfnNfnNfnNfnNfnNfnNfnNfnNfnnn-3′ |
| AS12 | 5′-ZnNfnNfnNfnnnnnNfnNfnNfnNfnNfn-3′ |
| AS13 | 5′-ZnNfnNfnNfnNfnNfnNfnNfnNfnNfnNfn-3′ 2 |
| AS14 | 5′-ZnNfnNfnNfnnnnnNfnNfnNfnNfnNfn-3′, |
| AS15 | 5′-ZnNfnNfnNfnNfnNfnNfnNfnNfnNfnnn-3′ |
| AS16 | 5′-ZnNfnNfnNfNfNfnNfnnnNfnNfnNfnNfnNfn-3′ |
| AS17 | 5′-ZnNfnNfnNfnNfnNfnnnNfnNfnNfnNfnnn-3′ |
| AS18 | 5′-Zn(dN)nn(dN)n(dN)nnnn(dN)n(dN)n(dN) |
| | nnnnnnn-3′ |
| AS19 | 5′-ZnNfnNfnnnnnnnNfnNfnNfnNfnNfn-3′ |
| AS20 | 5′-ZnNfnNfnnnnnnnNfnNfnNfnnnnn-3′ |

n is a 2′-O-methyl-modified nucleotide; s is a phosphorothioate internucleotide linkage (3′-5′); (dN) is a 2′-deoxy-nucleotide; Nf is a 2′-fluoro-modified nucleotide;
(G) is a thermally destabilizing modification, such as
   (Ngn)—a glycol nucleic acid, S-isomer;
   (N2p)—a 2′-phosphate nucleotide (i.e., a 3′-RNA connected by 3′-5′ and 2′-5′ internucleotide linkages on the 5′ and 3′ directions, respectively);
   (Tna)—a threose nucleotide (connected by 3′-3′ and 2′-5′ internucleotide linkages on the 5′ and 3′ directions, respectively);
   (MM) a nucleobase mismatch to the sense strand;
   (Nul) an unlocked nucleic acid; and
Z is a 5′-phosphate or 5′-phosphate mimic, such as VP is Vinyl-phosphonate (e.g., 5′-(E)-vinylphosphonate,

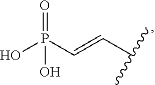 [SMILES: */C=C/P(=O)(O)O]

or a salt thereof, where the preceding structure replaces the 4′-CH₂OH group within the ribose ring of a 5′-terminal nucleotide), or 5′-cyclopropylphosphonate′(5′-CP) or 4′-O-methylphosphonate (4′-OMP) or 4′-O-methylphosphonate methyl ester (4′-OMPMe).

The structure of 5′-CP, as used herein, is

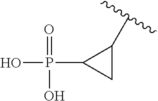 [SMILES: *C1CC1P(=O)(O)O]

or a salt thereof, where the broken bond is connected to the 4′-C of the ribose (i.e., the preceding structure replaces the 4′-CH₂OH group within the ribose ring of a 5′-terminal nucleotide). The structure of 4′-OMP, as used herein, is

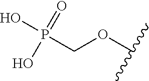 [SMILES: *OCP(=O)(O)O]

or a salt thereof, where the broken bond is connected to the 4′-C of the ribose (i.e., the preceding structure replaces the 4′-CH₂OH group within the ribose ring of a 5′-terminal nucleotide). The structure of 4′-OMPMe, as used herein, is

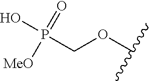 [SMILES: *OCP(=O)(O)OC]

or a salt thereof, where the broken bond is connected to the 4′-C of the ribose (i.e., the preceding structure replaces the 4′-CH₂OH group within the ribose ring of a 5′-terminal nucleotide).

In each of the preceding antisense strands, each of the nucleotides are connected in series (i.e., in a 3′->5′ manner) via phosphodiester or phosphorothioate internucleotide linkages.

In certain embodiments, the nucleotides at position 1 and 2 are connected by a phosphorothioate internucleotide linkage; the nucleotides at position 2 and 3 are connected by a phosphorothioate internucleotide linkage; and the remaining nucleotides are connected in via phosphodiester bonds, counting from the 5′-end of the oligonucleotide. In certain embodiments, the nucleotides at position 1 and 2 are connected by a phosphorothioate internucleotide linkage; the nucleotides at position 2 and 3 are connected by a phosphorothioate internucleotide linkage; the nucleotides at position 3 and 4 are connected by a phosphorothioate internucleotide linkage; and the remaining nucleotides are connected in via phosphodiester bonds, counting from the 5′-end of the oligonucleotide.

In certain embodiments, where the nucleotide in m nucleotides in length, the nucleotides at positions m-1 and m are connected by a phosphorothioate internucleotide linkage, counting from the 5′-end of the oligonucleotide. That is, for a nucleotide in 23 nucleotides in length (e.g., an antisense strand), the nucleotides at positions 22 and 23 are connected by a phosphorothioate internucleotide linkage, counting from the 5′-end of the oligonucleotide; and for a nucleotide in 21 nucleotides in length (e.g., a sense strand), the nucleotides at positions 20 and 21 are connected by a phosphorothioate internucleotide linkage, counting from the 5′-end of the oligonucleotide.

In certain embodiments, where the nucleotide in m nucleotides in length, the nucleotides at positions m-2 and m-1 are connected by a phosphorothioate internucleotide linkage, and the nucleotides at positions m-1 and m are connected by a phosphorothioate internucleotide linkage, counting from the 5′-end of the oligonucleotide. That is, for a nucleotide in 23 nucleotides in length (e.g., an antisense strand), the nucleotides at positions 21 and 22 are connected by a phosphorothioate internucleotide linkage; and positions 22 and 23 are connected by a phosphorothioate internucleotide linkage, counting from the 5′-end of the oligonucleotide; and for a nucleotide in 21 nucleotides in length (e.g., a sense strand), the nucleotides at positions 20 and 21 are connected by a phosphorothioate internucleotide linkage, counting from the 5′-end of the oligonucleotide.

In certain embodiments, where the nucleotide in m nucleotides in length:
(a) the nucleotides at position 1 and 2 are connected by a phosphorothioate internucleotide linkage;
(b) the nucleotides at position 2 and 3 are connected by a phosphorothioate internucleotide linkage;
(c) the nucleotides at positions m-2 and m-1 are connected by a phosphorothioate internucleotide linkage; and
(d) the nucleotides at positions m-1 and m are connected by a phosphorothioate internucleotide linkage, and the remaining nucleotides are connected in via phosphodiester bonds, counting from the 5′-end of the oligonucleotide.

That is, for a nucleotide in 23 nucleotides in length (e.g., an antisense strand), the nucleotides at positions 1 and 2; 2 and 3; 21 and 22; and 22 and 23 are connected by a phosphorothioate internucleotide linkage, and the remaining nucleotides are connected in via phosphodiester bonds, counting from the 5′-end of the oligonucleotide. And, for a nucleotide in 21 nucleotides in length (e.g., a sense strand), the nucleotides at positions 1 and 2; 2 and 3; 19 and 20; and 20 and 21 are connected by a phosphorothioate internucleotide linkage, and the remaining nucleotides are connected in via phosphodiester bonds, counting from the 5′-end of the oligonucleotide.

In certain embodiments, where the nucleotide in m nucleotides in length:
(a) the nucleotides at position 1 and 2 are connected by a phosphorothioate internucleotide linkage;
(b) the nucleotides at position 2 and 3 are connected by a phosphorothioate internucleotide linkage;
(c) the nucleotides at position 3 and 4 are connected by a phosphorothioate internucleotide linkage; and
(d) the nucleotides at positions m-1 and m are connected by a phosphorothioate internucleotide linkage,
   and the remaining nucleotides are connected in via phosphodiester bonds, counting from the 5′-end of the oligonucleotide.

That is, for a nucleotide in 23 nucleotides in length (e.g., an antisense strand), the nucleotides at positions 1 and 2; 2 and 3; 3 and 4; and 22 and 23 are connected by a phosphorothioate internucleotide linkage, and the remaining nucleotides are connected in via phosphodiester bonds, counting from the 5′-end of the oligonucleotide. And, for a nucleotide in 21 nucleotides in length (e.g., a sense strand), the nucleotides at positions 1 and 2; 2 and 3; 3 and 4; and 20 and 21 are connected by a phosphorothioate internucleotide linkage, and the remaining nucleotides are connected in via phosphodiester bonds, counting from the 5′-end of the oligonucleotide.

For example, in some embodiments, the antisense strand of the dsRNA agent has one of the following modification patterns (with variables as defined above):

| | |
|---|---|
| AS23 | 5′-Zns(dN)snn(dN)n(dN)nnnn(dN)nNfnnnnnnnsnsn-3′ |
| AS24 | 5′-Zns(dN)snn(dN)n(G)nnnn(dN)nNfnnnnnnnsnsn-3′ |
| AS25 | 5′-ZnsNfsnn(dN)n(G)nnnnnnNfnNfnnnnnsnsn-3′ |
| AS26 | 5′-ZnsNfsnnnNfnnnnnnnNfnNfnnnnnsnsn-3′ |
| AS27 | 5′-ZnsNfsnnnNfnnNfnnnnNfnNfnnnnnsnsn-3′ |
| AS28 | 5′-ZnsNfsnnnNfnNfNfnnnnNfnNfnnnnnsnsn-3′ |
| AS29 | 5′-ZnsNfsnnnNf(G)nnnnnnNfnNfnnnnnsnsn-3′ |
| AS30 | 5′-ZnsNfsnnnNf(G)nNfnnnnNfnNfnnnnnsnsn-3′ |
| AS31 | 5′-ZnsNfsnnnNf(G)NfNfnnnnNfnNfnnnnnsnsn-3′ |
| AS32 | 5′-ZnsNfsnNfnNfnNfnNfnNfnNfnNfnNfnsNfsn-3′ |
| AS33 | 5′-ZnsNfsnNfnNfnNfnNfnNfnNfnNfnNfnsnsn-3′ |
| AS34 | 5′-ZnsNfsnNfnNfnnnnnNfnNfnNfnNfnsNfsn-3′ |
| AS35 | 5′-ZnsNfsnsNfnNfnNfnNfnNfnNfnNfnNfnNfsn-3′ |
| AS36 | 5′-ZnsNfsnsNfnNfnnnnnNfnNfnNfnNfnNfsn-3′, |
| AS37 | 5′-ZnsNfsnsNfnNfnNfnNfnNfnNfnNfnNfnnsn-3′ |
| AS38 | 5′-ZnNfnNfnNfNfNfnNfnnnNfnNfnNfnNfnNfsn-3′ |
| AS39 | 5′-ZnsNfsnNfnNfnNfnNfnnnNfnNfnNfnNfnsnsn-3′ |
| AS40 | 5′-Zns(dN)snn(dN)n(dN)nnnn(dN)n(dN)n(dN) |
| | nnnnnsnsn-3′ |
| AS41 | 5′-ZnsNfsnsNfnnnnnnnNfnNfnNfnNfnNfsn-3′ |
| AS42 | 5′-ZnsNfsnNfnnnnnnnNfnNfnNfnNfnsNfsn-3′ |
| AS43 | 5′-ZnsNfsnsNfnnnnnnnNfnNfnNfnnnnsn-3′ |
| AS44 | 5′-ZnsNfsnNfnnnnnnnNfnNfnNfnnnsnsn-3′ |

In a further embodiment of each of the preceding exemplary sense and antisense strands, each of sense strand Si through S54 may be duplexes with any one of antisense strands AST through AS44.

### Delivery

The delivery of an RNAi agent (or other nucleic acid therapeutic agent) composition of the disclosure to a cell e.g., a cell within a subject, such as a human subject (e.g., a subject in need thereof, such as a subject having a target gene-associated disorder, e.g., AD, CAA, EOFAD, etc., can be achieved in a number of different ways. For example, delivery may be performed by contacting a cell with an RNAi agent composition of the disclosure either in vitro or in vivo. In vivo delivery may also be performed directly by administering a composition comprising an RNAi agent, e.g., a dsRNA, to a subject. Alternatively, in vivo delivery may be performed indirectly by administering one or more vectors that encode and direct the expression of the RNAi agent.

In general, methods of delivering a nucleic acid molecule (in vitro or in vivo) can be adapted for use with an RNAi agent composition of the disclosure (see e.g., Akhtar S. and Julian R L., (1992) Trends Cell. Biol. 2(5): 139-144 and WO94/02595, which are incorporated herein by reference in their entireties). For in vivo delivery, factors to consider for delivering an RNAi agent composition include, for example, biological stability of the delivered agent, prevention of non-specific effects, and accumulation of the delivered agent in the target tissue. The non-specific effects of an RNAi agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. Several studies have shown successful knockdown of gene products when an RNAi agent is administered locally. For example, intraocular delivery of a VEGF dsRNA by intravitreal injection in cynomolgus monkeys (Tolentino, M J. et al., (2004) Retina 24: 132-138) and subretinal injections in mice (Reich, S J. et al. (2003) Mol. Vis. 9: 210-216) were both shown to prevent neovascularization in an experimental model of age-related macular degeneration. In addition, direct intratumoral injection of a dsRNA in mice reduces tumor volume (Pille, J. et al. (2005) Mol. Ther. 11: 267-274) and can prolong survival of tumor-bearing mice (Kim, W J. et al., (2006) Mol. Ther. 14: 343-350; Li, S. et al., (2007) Mol. Ther. 15: 515-523). RNA interference has also shown success with local delivery to the CNS by direct injection (Dorn, G. et al., (2004) Nucleic Acids 32: e49; Tan, P H. et al. (2005) Gene Ther. 12: 59-66; Makimura, H. et a.l (2002) BMC Neurosci. 3: 18; Shishkina, G T., et al. (2004) Neuroscience 129: 521-528; Thakker, E R., et al. (2004) Proc. Natl. Acad Sci. U.S.A. 101: 17270-17275; Akaneya, Y., et al. (2005) J Neurophysiol. 93: 594-602) and to the lungs by intranasal administration (Howard, K A. et al., (2006) Mol. Ther. 14: 476-484; Zhang, X. et al., (2004) J. Biol. Chem. 279: 10677-10684; Bitko, V. et al., (2005) Nat. Med. 11: 50-55).

Certain aspects of the instant disclosure relate to a method of reducing the expression of a target gene in a cell or subject, involving contacting said cell or subject with the double-stranded RNAi agent composition of the disclosure. In one embodiment, the cell is an extrahepatic cell, optionally a CNS cell.

Another aspect of the disclosure relates to a method of reducing the expression of a target gene in a subject, involving administering to the subject the double-stranded RNAi agent composition of the disclosure.

Another aspect of the disclosure relates to a method of treating a subject having a target gene-associated disorder, involving administering to the subject a therapeutically effective amount of the double-stranded RNAi agent-containing composition of the disclosure, thereby treating the subject.

In one embodiment, the double-stranded RNAi agent is administered intrathecally. By intrathecal administration of the double-stranded RNAi agent, the method can reduce the expression of a target gene in a brain (e.g., striatum) or spine tissue, for instance, cortex, cerebellum, cervical spine, lumbar spine, and thoracic spine.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the disclosure.

The RNAi agent compositions of the disclosure can be further incorporated into pharmaceutical compositions suitable for parenteral administration. Such compositions typically include one or more species of RNAi agent and a pharmaceutically acceptable carrier. As used herein the language “pharmaceutically acceptable carrier” is intended to include any and all solvents, antibacterial and antifungal agents, isotonic agents, and the like, compatible with pharmaceutical administration, but in certain aspects, excluding such agents comprising inorganic phosphate (e.g., excluding phosphate-buffered saline (PBS) as an isotonic solution in certain embodiments). The use of such media and agents for pharmaceutically active substances is well known in the art. Except insofar as any conventional media or agent is incompatible with the active compound, use thereof in the compositions is contemplated. Supplementary active compounds can also be incorporated into the compositions.

While the primary route of administration for the pharmaceutical compositions of the present disclosure is via parenteral administration, such as intrathecal injection, the pharmaceutical compositions of the present disclosure may be administered in a number of ways depending upon whether local or systemic treatment is desired and upon the area to be treated. Parenteral administration includes intravenous drip, subcutaneous, intraperitoneal or intramuscular injection, or intrathecal or intraventricular administration.

Compositions for intrathecal or intraventricular administration may include sterile aqueous solutions (though excluding agents comprising inorganic phosphate in certain aspects) which may also contain diluents and other suitable additives.

Formulations for parenteral administration may include sterile aqueous solutions which may also contain diluents and other suitable additives. Intraventricular injection may be facilitated by an intraventricular catheter, for example, attached to a reservoir. For intravenous use, the total concentration of solutes may be controlled to render the preparation isotonic.

In one embodiment, the administration of the dsRNA compound, e.g., a double-stranded siRNA compound, or ssiRNA compound, composition is parenteral, e.g., intravenous (e.g., as a bolus or as a diffusible infusion), intradermal, intraperitoneal, intramuscular, intrathecal, intraventricular, intracranial, subcutaneous or ocular injection. Administration can be provided by the subject or by another person, e.g., a health care provider. Selected modes of delivery are discussed in more detail below.

### Intrathecal Administration

In certain embodiments, a nucleic acid agent formulation (e.g., a double-stranded RNAi agent composition) is delivered by intrathecal injection (i.e., injection into the spinal fluid which bathes the brain and spinal cord tissue). Intrathecal injection of nucleic acid (e.g., RNAi) agents into the spinal fluid can be performed as a bolus injection or via minipumps which can be implanted beneath the skin, providing a regular and constant delivery of nucleic acid into the spinal fluid. The circulation of the spinal fluid occurs from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, allows molecules delivered intrathecally potentially to hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in WO 2015/116658, which is incorporated by reference in its entirety.

The amount of intrathecally injected nucleic acid agents (e.g., RNAi agents) may vary from one target gene to another target gene and the appropriate amount that has to be applied may also be determined individually for each target gene. In embodiments, this amount ranges from 10 g to 100 mg/mL of injectate, optionally 50 μg to 150 mg/mL of injectate, more optionally 20 mg to 100 mg/mL of injectate.

mRNA Knockdown to Treat Associated Diseases

As used herein, a “subject” is an animal, such as a mammal, including a primate (such as a human, a non-human primate, e.g., a monkey, and a chimpanzee), or a non-primate (such as a rat, or a mouse). In a preferred embodiment, the subject is a human, such as a human being treated or assessed for a disease, disorder, or condition that would benefit from reduction in target gene expression; a human at risk for a disease, disorder, or condition that would benefit from reduction in target gene expression; a human having a disease, disorder, or condition that would benefit from reduction in target gene expression; or human being treated for a disease, disorder, or condition that would benefit from reduction in target gene expression as described herein.

As used herein, the terms “treating” or “treatment” refer to a beneficial or desired result including, but not limited to, alleviation or amelioration of one or more symptoms associated with a disease or disorder. In certain embodiments, “treating” or “treatment” refers to a beneficial or desired result including, but not limited to, alleviation or amelioration of one or more symptoms associated with modulation of APP gene expression and/or APP protein production, e.g., APP-associated diseases or disorders such as Alzheimer's disease (AD), cerebral amyloid angiopathy (CAA; e.g., hereditary CAA), early onset familial Alzheimer disease (EOFAD or eFAD), early onset Alzheimer's disease (EOAD), familial Alzheimer's disease, or late onset Alzheimer's disease, among others. “Treatment” can also mean prolonging survival as compared to expected survival in the absence of treatment.

The term “lower” in the context of the level of target gene in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of target gene in a subject is optionally down to a level accepted as within the range of normal for an individual without such disorder. In certain embodiments, “lower” is the decrease in the difference between the level of a marker or symptom for a subject suffering from a disease and a level accepted within the range of normal for an individual, e.g., the level of decrease in memory or cognition in an individual having Alzheimer's and an individual not having Alzheimer's or having symptoms that are within the range of normal.

As used herein, “prevention” or “preventing,” when used in reference to a disease or disorder, that would benefit from a reduction in expression of a target gene or production of target protein, e.g., in a subject susceptible to a target gene-associated disorder due to, e.g., genetic factors or age, wherein the subject does not yet meet the diagnostic criteria for the target gene-associated disorder. As used herein, prevention can be understood as administration of an agent to a subject who does not yet meet the diagnostic criteria for the target gene-associated disorder to delay or reduce the likelihood that the subject will develop the target gene-associated disorder. As the agent is a pharmaceutical agent, it is understood that administration typically would be under the direction of a health care professional capable of identifying a subject who does not yet meet the diagnostic criteria for a target gene-associated disorder as being susceptible to developing a target gene-associated disorder.

| | mRNA Accession | | PCT Publication |
|---|---|---|---|
| Gene Target | Number | Diseases | No(s). |
| APP | NM_201414; | cerebral amyloid angiopathy (CAA); | WO/2020/132227 |
| | NM_000484.3; | early onset familial Alzheimer disease | WO/2022/165172 |
| | NM_201413.2; | (EOFAD); Alzheimer's disease (AD; | WO/2023/010134 |
| | | refer to PCT/US19/67449 for | WO/2023/039503 |
| | NM_001136016.3; | additional details). | |
| | NM_001136129.2; | | |
| | NM_001136130.2; | | |
| | NM_001136131.2; | | |
| | NM_001204301.1; | | |
| | NM_001204302.1; | | |
| | NM_001204303.1 | | |
| SOD1 | NM_000454.4 | Familial amyotrophic lateral sclerosis; | WO/2022/174000 |
| | | | WO/2023/010134 |
| SCN9a | NM_002977.3 | Primary erythermalgia; Channelopathy- | WO/2021/207189 |
| | | associated insensitivity to pain; and | WO/2023/010134 |
| | | Paroxysmal extreme pain disorder | |
| HTT | NM_001388492.1 | Huntington Disease; | WO/2021/087036 |
| | | Lopes-Maciel-Rodan Syndrome | WO/2022/212231 |
| | | | WO/2023/010134 |
| | | | WO/2023/076450 |
| APOE | NM_001302688.2 | Familial dysbetalipoproteinemia (aka type | WO/2011/029016 |
| | NM_000041.4 | III Hyperlipoproteinemia (HLP III) | WO/2021/222065 |
| | | | WO/2023/010134 |
| LRRK2 | NM_198578.4 | Parkinson's disease | WO/2021/150969 |
| | | | WO/2023/010134 |
| | | | WO/2023/278607 |
| PRNP | NM_000311.5 | Creutzfeldt-Jakob disease; | WO/2022/182864 |
| | | Fatal familial insomnia; | WO/2023/010134 |
| | | Gerstmann-Straussler disease; | |
| | | Huntington disease-like 1; and kuru | |
| SCD5 | NM_001037582.3 | Obesity; | WO/2022/197975 |
| | | Dislipidemia; | WO/2023/010134 |
| | | Deafness; | |
| | | Autosomal Dominant 79; and | |
| | | Chronic Maxillary Sinusitis | |
| GPR75 | NM_006794.4 | Obesity | WO/2022/076291 |
| | | | WO/2023/010134 |
| MAPT | NM_016835.5 | Alzheimer's disease; | WO/2021/202511 |
| | | Pick's disease; | WO/2023/049871 |
| | | Frontotemporal dementia; | WO/2023/010134 |
| | | Cortico-basal degeneration; and | WO/2023/154900 |
| | | Progressive supranuclear palsy | |
| SNCA | NM_000345.4 | Alzheimer's disease; | WO/2005/004794 |
| | NM_007308.3 | Parkinson's disease | WO/2009/079399 |
| | | | WO/2012/027713 |
| | | | WO/2012/177906 |
| | | | WO/2022/072447 |
| | | | WO/2023/010134 |
| | | | WO/2023/192977 |
| ABLIM3 | NM_001301015.3 | PTSD; age-related memory loss | WO/2023/215805 |
| ADRA2A | NM_000681.4 | Alzheimer's disease; | WO/2021/202902 |
| | | Frontotemporal dementia; | WO/2023/010134 |
| | | Frontotemporal lobar degeneration; | |
| | | Progressive supranuclear palsy; | |
| | | Corticobasal degeneration; | |
| | | Chronic traumatic encephalopathy; Pick's | |
| | | disease; | |
| | | Argyrophilic grain disease; Primary age- | |
| | | related tauopathy | |
| ATXN1 | NM_000332 | | WO/2011/097388 |
| ATXN2 | NM_002973.3 | Spinocerebellar ataxia (SCA), | WO/2011/097388 |
| | | Spinocerebellar ataxia 2 (SCA2), | WO/2022/026531 |
| | | Amyotrophic Lateral Sclerosis (ALS) | WO/2023/010134 |
| ATXN3 | NM_001127697.2 | Spinocerebellar ataxia type 3 (SCA3) | WO/2011/097388 |
| | NM_001164782.2 | (Machado-Joseph Disease) | WO/2021/102373 |
| | NM_004993 | | WO/2023/010134 |
| ELOVL1 | NM_022821.4 | X-Linked Adrenoleukodystrophy (X-ALD) | WO/2022/204440 |
| FLNA | NM_001110556.2 | Alzheimer's disease | WO/2022/271972 |
| | | | WO/2023/010134 |
| NOGO-L OR | NM_020532 | Treatment of regeneration for neurons | WO/2006/081192 |
| NOGO-R | NM_023004 | damaged by trauma, infarction and | |
| | | degenerative disorders of the CNS; | |
| | | Glioblastoma | |
| HIF- 1α | None listed | Age- related macular degeneration | WO/2007/002718 |
| RHO-A | NM_001664 | Nerve injury or damage | WO/2007/014077 |
| HUNTINGTIN | NM_002111 | Huntington's disease | WO/2007/051045 |
| NAV1.8 | NM_006514 | Multiple sclerosis, Myelitis or syphilis, | WO/2007/056326 |
| | | Ischemia, Syringomyelia | |
| CD45 | NM_002838.2 | Graves' disease; Multiple sclerosis | WO/2009/137128 |
| GSK-3 | NM_019884.2 | Bipolar disorder; Alzheimer's disease, | WO/2010/006342 |
| | NM_002093 | Parkinson's disease, Huntington's Disease | |
| GSK3α | NM_019884.3 | Fragile X Syndrome | WO/2022/192519 |
| | | | WO/2023/010134 |
| MIG-12 | NM_021242.4 | Opitz syndrome; Diabetes or | WO/2010/099341 |
| | | Atherosclerosis | WO/2011/038031 |
| Mgat1 | NM_001114619.1 | Gaucher's disease and other lysosomal | WO/2011/109600 |
| | | storage diseases such as e.g., Pompe | |
| | | disease and Fabry's disease | |
| Mgat4 | NM_012214.2 | Gaucher's disease and other lysosomal | WO/2011/109600 |
| | NM_014275.4 | storage diseases such as e.g., Pompe | |
| | | disease and Fabry's disease | |
| SLC35A1 | NM_006416.4 | Gaucher's disease and other lysosomal | WO/2011/109600 |
| | | storage diseases such as e.g., Pompe | |
| | | disease and Fabry's disease | |
| SLC35A2 | NM_001032289.1 | Gaucher's disease and other lysosomal | WO/2011/109600 |
| | | storage diseases such as e.g., Pompe | |
| | | disease and Fabry's disease | |
| GNE | NM_005476.4 | Gaucher's disease and other lysosomal | WO/2011/109600 |
| | | storage diseases such as e.g., Pompe | |
| | | disease and Fabry's disease | |
| TMPRSS6 | NM_153609.2 | β-thalassemia intermedia; | WO/2012/135246 |
| | NM_153609.4 | Parkinson's Disease, Alzheimer's Disease, | WO/2014/190157 |
| | | or Friedreich's Ataxia | WO/2016/085852 |
| | | | WO/2022/231999 |
| Complement | NM_000064.3 | Paroxysmal nocturnal hemoglobinuria; | WO/2015/089368 |
| Component | | Atypical hemolytic uremic syndrome; | WO/2019/089922 |
| C3 | | Systemic lupus erythematosus; Cold | WO/2021/081026 |
| | | agglutinin disease; Alzheimer's disease, | WO/2021/178607 |
| | | Amyotrophic Lateral Aclerosis, | WO/2023/044370 |
| | | Schizophrenia, Parkinson's disease, and | |
| | | Creutzfeldt-Jakob disease | |
| APCS | NM_001639.3 | Amyloidosis; Alzheimer's disease, | WO/2019/100039 |
| | | Cardiovascular disease | |
| C9orf72 | NM_018325.4 | Frontotemporal dementia; Huntington's | WO/2021/119226 |
| | NM_001256054.2 | disease; Amyotrophic lateral sclerosis | WO/2022/256290 |
| | NM_145005.6 | | WO/2023/010134 |
| CHI3L1/ | NM_001276.4 | Alzheimer's disease | WO/2022/226267 |
| YKL-40 | | | WO/2023/010134 |
| EXT 1 | NM_000127.3 | Mucopolysaccaridosis type III | WO/2023/141314 |
| EXT2 | NM_000401.3 | Mucopolysaccaridosis type III | WO/2023/141314 |
| | NM_207122.2 | | |
| | NM_001178083.3 | | |
| | NM_001389628.1 | | |
| | NM_001389630.1 | | |
| NDST2 | NM_003635.4 | Mucopolysaccaridosis type III | WO/2023/141314 |
| | NM_001301063.2 | | |
| RPS25 | NM_001028.3 | C9orf72 ALS/FTD, Huntington-Like | WO/2021/030522 |
| | | Syndrome Due To C9orf72 Expansions, | |
| | | Fragile X syndrome (FXS), Myotonic | |
| | | dystrophy (i.e., DM1, and DM2), | |
| | | CAG/polyglutamine disease (e.g., | |
| | | Huntington's disease, Spinal and bulbar | |
| | | muscular atrophy (SBMA), | |
| | | Dentatorubral-pallidoluysian atrophy, | |
| | | Spinocerebellar ataxia type I, type 2, type | |
| | | 3, type 6, type 7, type 8, type 12, and type | |
| | | 17, Friedreich ataxia, Unverricht- | |
| | | Lundborg myoclonic epilepsy (EPM1), | |
| | | Oculopharyngeal muscular dystrophy | |
| | | (OPMD), and Fuchs endothelial corneal | |
| | | dystrophy (FECD) | |
| ALK | NM_004304.4 | obesity, type 2 diabetes | WO/2021/257568 |

Certain aspects of the instant disclosure are directed to RNAi agent-mediated knockdown of APP-associated diseases or disorders, which include CAA and AD, including hereditary CAA and EOFAD, as well as sporadic and/or late onset AD. Hereditary CAA (hCAA) is a vascular proteinopathy, for which the amyloid therapeutic hypothesis is relatively straightforward and clinically testable. It is a devastating and rare disease, with no existing therapy. Both biochemical and imaging biomarkers exist for clinical validation of anti-APP siRNA-mediated treatment of hCAA. Various types of hCAA have been described, including sporadic CAA, HCHWA-Dutch and Italian type EOFAD, LOAD, and Trisomy 21 as AP-linked forms of hAPP. hCAA can be associated with AD, EOFAD and/or Down syndrome, among other conditions.

Soluble forms of APP, particularly including APPu and APPO can serve as cerebrospinal fluid (CSF) biomarkers for assessing APP knockdown efficiency. Additional consideration of hCAA and APP can be found in PCT/US19/67449, among other references.

Available assays can also be used to detect soluble APP levels in human CSF samples. In particular, sAPP□ and sAPPβ are soluble forms of APP and have been identified as serving as PD (pharmacodynamic) biomarkers. Analytes have also been detected in non-human primate (NHP) CSF samples, and such assays can enable efficacy studies in NHPs. Detection of Aβ40/42/38 peptides and Total tau/P181 Tau has also been described.

Animal models of CAA have been identified, which allow for determination of the effect of APP knockdown on CAA pathology and identification of translatable biomarkers. In particular, multiple rodent models that express mutant human APP and show CAA pathology have been developed, including Tg-SwDI/NOS2−/−(Hall and Roberson. Brain Res Bull. 2012; 88(1): 3-12; Attems et al., Nephrology and Applied Neurobiology, 2011, 37, 75-93).

Certain aspects of the instant disclosure are directed to RNAi agent-mediated knockdown of SOD1-associated diseases or disorders. The protein encoded by the SOD1 gene is Superoxide Dismutase 1, which binds to both copper and zinc ions. Rare transcript variants have been reported for this gene. The SOD1 protein is one of two isozymes that facilitates removal of free superoxide radicals in the body by converting naturally-occurring superoxide radicals to molecular oxygen and hydrogen peroxide. Mutations in SOD1 have been implicated as causes of familial amyotrophic lateral sclerosis.

Certain aspects of the instant disclosure are directed to RNAi agent-mediated knockdown of SCN9a-associated diseases or disorders. The SCN9a gene encodes a voltage-gated sodium channel that plays a significant role in nociception signaling. Mutations in this gene have been associated with primary erythermalgia, channelopathy-associated insensitivity to pain, and paroxysmal extreme pain disorder.

Certain aspects of the instant disclosure are directed to RNAi agent-mediated knockdown of HTT-associated diseases or disorders. HTT (Huntingtin) is a disease gene linked to Huntington's disease, a neurodegenerative disorder characterized by the loss of striatal neurons, which is believed to be caused by an expanded, unstable trinucleotide repeat in the HTT gene that translates as a polyglutamine repeat in the protein product. The HTT locus is large, spanning approximately 180 kb and including of 67 exons. Without being bound by theory, the genetic defect leading to Huntington's disease may not necessarily eliminate transcription, but may confer a new property on the mRNA or alter the function of the protein.

Certain aspects of the instant disclosure are directed to RNAi agent-mediated knockdown of APOE-associated diseases or disorders. The protein encoded by the APOE gene is a major apoprotein of the chylomicron. It binds to a specific liver and peripheral cell receptor, and is essential for the normal catabolism of triglyceride-rich lipoprotein constituents. Mutations in APOE result in familial dysbetalipoproteinemia, or type III hyperlipoproteinemia (HLP III), in which increased plasma cholesterol and triglycerides are the consequence of impaired clearance of chylomicron and VLDL remnants.

Certain aspects of the instant disclosure are directed to RNAi agent-mediated knockdown of LRRK2-associated diseases or disorders. The LRRK2 gene is a member of the leucine-rich repeat kinase family and encodes a protein with an ankryin repeat region, a leucine-rich repeat (LRR) domain, a kinase domain, a DFG-like motif, a RAS domain, a GTPase domain, a MLK-like domain, and a WD40 domain. The LRRK2 protein is present largely in the cytoplasm, but also associates with the mitochondrial outer membrane. Mutations in this gene have been associated with Parkinson disease.

Certain aspects of the instant disclosure are directed to RNAi agent-mediated knockdown of PRNP-associated diseases or disorders. The PRNP protein encodes a membrane glycosylphosphatidylinositol-anchored glycoprotein that tends to aggregate into rod-like structures. The PRNP protein contains a highly unstable region of five tandem octapeptide repeats. This gene is found on chromosome 20, approximately 20 kbp upstream of a gene which encodes a biochemically and structurally similar protein to the one encoded by this gene. Mutations in the repeat region as well as elsewhere in this gene have been associated with Creutzfeldt-Jakob disease, fatal familial insomnia, Gerstmann-Straussler disease, Huntington disease-like 1, and kuru.

Certain aspects of the instant disclosure are directed to RNAi agent-mediated knockdown of SCD5-associated diseases or disorders. Stearoyl-CoA desaturase is an integral membrane protein of the endoplasmic reticulum that catalyzes the formation of monounsaturated fatty acids from saturated fatty acids. Four SCD isoforms, SCD1 through SCD4, have been identified in mouse, while only 2 SCD isoforms—SCD1 and SCD5—have been identified in humans. SCD1 shares about 85% amino acid identity with all 4 mouse SCD isoforms, as well as with rat Scd1 and Scd2. However, SCD5 shares limited homology with the rodent SCDs and appears to be unique to primates. Diseases associated with SCD5 include Deafness, Autosomal Dominant 79 and Chronic Maxillary Sinusitis.

Certain aspects of the instant disclosure are directed to RNAi agent-mediated knockdown of GPR75-associated diseases or disorders. GPR75 encodes a member of the G protein-coupled receptor family. GPR proteins are cell surface receptors that activate guanine-nucleotide binding proteins upon the binding of a ligand. GPR75 is activated by the chemokine CCL5/RANTES. Without being bound by theory, it is believed that GPR75 is coupled to heterotrimeric Gq proteins, where it stimulates inositol trisphosphate production and calcium mobilization upon activation. Together with CCL5/RANTES, GPR75 may play a role in neuron survival through activation of a downstream signaling pathway involving the PI3, Akt and MAP kinases. CCL5/RANTES may also regulate insulin secretion by pancreatic islet cells through activation of this receptor. GPR75 is believed to play a role in obesity.

Certain aspects of the instant disclosure are directed to RNAi agent-mediated knockdown of MAPT-associated diseases or disorders. The MAPT gene encodes the microtubule-associated protein tau (MAPT) whose transcript undergoes complex, regulated alternative splicing, which gives rise to several mRNA species. MAPT transcripts are differentially expressed in the nervous system, depending on stage of neuronal maturation and neuron type. MAPT gene mutations have been associated with several neurodegenerative disorders such as Alzheimer's disease, Pick's disease, frontotemporal dementia, cortico-basal degeneration and progressive supranuclear palsy.

Certain aspects of the instant disclosure are directed to RNAi agent-mediated knockdown of SNCA-associated diseases or disorders. SNCA (Synuclein Alpha) is a member of the synuclein family, which also includes beta- and gamma-synuclein. Synucleins are abundantly expressed in the brain and alpha- and beta-synuclein inhibit phospholipase D2 selectively. SNCA may serve to integrate presynaptic signaling and membrane trafficking. Defects in SNCA have been implicated in the pathogenesis of Parkinson disease. Additionally, SNCA peptides are a major component of amyloid plaques in the brains of patients with Alzheimer's disease.

Thus, APP has been identified as a target for hereditary cerebral amyloid angiopathy (CAA). Mutations in APP that have been reported to cause severe forms of CAA include A692G (Flemish), E693Q (Dutch), E693K (Italian), and D694N (Iowa). Meanwhile, mutations in APP that have been described to cause early onset AD include E665D, K670N, M671L (Swedish), T714A (Iranian), T714I (Austrian), V715M (French), V715A (German), I716V (Florida), I716T, V717I (London), V717F, V717G and V717L. In particular, the APP E693Q (Dutch) mutation causes severe CAA with few parenchymal neurofibrillary tangles; E693Q increases amyloid beta aggregation and toxicity; E693K (Italian) is similar but E693G (Arctic), E693A and E693delta mutations cause EOFAD with little or no CAA; and APP D694N (Iowa) causes severe CAA with typical AD pathology. In addition to the preceding point mutations, APP duplications that result in APP overexpression have also been identified to cause Aβ deposition. Meanwhile, no known APP mutations have been described that prevent or delay APP-hCAA. In addition to APP mutants, Aβ CAA has also been observed for PSEN1 (L282V) and PSEN2 (N141I) mutations. Meanwhile, ApoE ε2 (independent of AD) and ApoE ε4 (dependent on AD) have also been reported as risk factors for CAA (Rensink A et al., Brain Research Reviews, 43 (2) 2003).

Certain aspects of the instant disclosure are directed towards targeting of APP for knockdown in individuals having APP-hCAA. A need exists for such agents because there are currently no disease-modifying therapies for CAA. In certain embodiments, the RNAi agents of the instant disclosure should provide approximately 60-80% knockdown of both mutant and WT APP levels throughout the CNS.

The term “APP” amyloid precursor protein (APP), also known as amyloid beta precursor protein, Alzheimer disease amyloid protein and cerebral vascular amyloid peptide, among other names, refers to a polypeptide having an amino acid sequence from any vertebrate or mammalian source, including, but not limited to, human, bovine, chicken, rodent, mouse, rat, porcine, ovine, primate, monkey, and guinea pig, unless specified otherwise. The term also refers to fragments and variants of native APP that maintain at least one in vivo or in vitro activity of a native APP (including, e.g., the beta-amyloid peptide(1-40), beta-amyloid peptide(1-38) and beta-amyloid peptide(1-42) forms of Aβ peptide, among others), including variants of APP fragments that maintain one or more activities of an APP fragment that are neurotoxic in character (e.g., variant forms of Aβ42 peptide that maintain neurotoxic character are expressly contemplated). The term encompasses full-length unprocessed precursor forms of APP as well as mature forms resulting from post-translational cleavage of the signal peptide. The term also encompasses peptides that derive from APP via further cleavage, including, e.g., Aβ peptides. The nucleotide and amino acid sequence of a human APP can be found at, for example, GenBank Accession No. GI: 228008405 (NM 201414). The nucleotide and amino acid sequence of a human APP may also be found at, for example, GenBank Accession No. GI: 228008403 (NM_000484.4); GenBank Accession No. GI: 228008404 (NM_201413.3); GenBank Accession No. GI: 324021746 (NM_001136016.3); GenBank Accession No. GI: 228008402 (NM_001136129.3); GenBank Accession No. GI: 228008401 (NM_001136130.3); GenBank Accession No. GI: 324021747 (NM_001136131.3); GenBank Accession No. GI: 324021737 (NM_001204301.2); GenBank Accession No. GI: 324021735 (NM_001204302.2); and GenBank Accession No. GI: 324021739 (NM 001204303.2); and GenBank Accession No. GI: 1370481385 (XM_024452075.1). Additional examples of APP sequences are readily available using publicly available databases, e.g., GenBank, UniProt, and OMIM.

The term “APP” as used herein also refers to a particular polypeptide expressed in a cell by naturally occurring DNA sequence variations of the APP gene, such as a single nucleotide polymorphism in the APP gene. Numerous SNPs within the APP gene have been identified and may be found at, for example, NCBI dbSNP (see, e.g., www.ncbi.nlm.nih.gov/snp). Non-limiting examples of SNPs within the APP gene may be found at, e.g., NCBI dbSNP Accession Nos. rs193922916, rs145564988, rs193922916, rs214484, rs281865161, rs364048, rs466433, rs466448, rs532876832, rs63749810, rs63749964, rs63750064, rs63750066, rs63750151, rs63750264, rs63750363, rs63750399, rs63750445, rs63750579, rs63750643, rs63750671, rs63750734, rs63750847, rs63750851, rs63750868, rs63750921, rs63750973, rs63751039, rs63751122 and rs63751263. Certain exemplary rare APP variants that have been previously described to play a role in development of EOFAD were identified in Hooli et al. (Neurology 78: 1250-57). In addition, various “non-classical” APP variants that harbor an intraexonic junction within sequenced cDNA have recently been identified as associated with the occurrence of somatic gene recombination in the brains of AD patients (PCT/US2018/030520, which is incorporated herein by reference in its entirety). Examples of such “non-classical” APP variants include cAPP-R3/16, cAPP-R3/16-2, cAPP-R2/18, cAPP-R6/18, cAPP-R3/14, cAPP-R3/17, cAPP-R1/11, cAPP-R1/13, cAPP-R1/11-2, cAPP-R1/14, cAPP-R2/17, cAPP-R2/16, cAPP-R6/17, cAPP-R2/14, cAPP-R14/17-d8 and cAPP-D2/18-3. It is expressly contemplated that RNAi agents of the instant disclosure can be used to target “non-classical” APP variants and/or that RNAi agents optionally specific for such “non-classical” APP variants can be designed and used, optionally in combination with other RNAi agents of the instant disclosure, including those that target native forms of APP. Such “non-classical” APP variants were described as notably absent from an assayed HIV patient population, with prevalence of AD in the HIV patient population significantly diminished as compared to expected levels, which indicated that reverse transcriptase inhibitors and/or other anti-retroviral therapies commonly used to treat HIV patients likely also exerted a therapeutic/preventative role against AD. It is therefore expressly contemplated that the RNAi agents of the instant disclosure can optionally be employed in combination with reverse transcriptase inhibitors and/or other anti-retroviral therapies, for therapeutic and/or preventative purposes.

Humans with heterozygous APP mutations exist in the general population with pLI score of 0.3; however, no Human APP knockout has been identified thus far.
Pharmacological attempts to treat human CAA include the following: Ponezumab, an amyloid beta 40 antibody was studied by Pfizer in 36 individuals with late-onset CAA Three infusions of ponezumab or placebo over the course of 60 days were evaluated for changes in cerebrovascular reactivity as measured by BOLD fmRI, as well as for cerebral edema, infarcts, Ap, cognitive change and other secondary outcomes. Ponezumab showed drug-placebo differences, but did not meet the primary endpoint.
BAN2401. Amyloid beta therapeutic antibodies delivered systemically were identified to be safe but also could cause local cerebral edema. In a recent phase II 18-mo trial of BAN2401 in LOAD, the incidence of SAEs was 17.6% for placebo and 15.5% for the highest dose (10 mg/kg biweekly). Amyloid Related Imaging Abnormalities-Edema (ARIA-E) was 14.6% at the highest dose in APOE4 carriers.

Against animal CAA models, ponezumab was noted as effective in a mouse model of CAA with respect to lowering amyloid beta burden and vascular reactivity (Bales, 2018). Meanwhile, global APP knockout mice have further been noted as viable.

The following exemplary biomarker and pathological data have also provided further validation for the primary role for amyloid beta protein in pathogenesis of CAA:
Hereditary forms of “pure” CAA (i.e., lacking parenchymal plaque amyloid) have been observed as characterized by predominant Aβ40 deposition in amyloid, as opposed to Aβ42 in parenchymal AD;
CAA has been observed as not a “tauopathy”, with normal levels of T-tau and P-tau in the CSF, in contrast to elevated levels observed in AD;
The inverse correlation of increasing brain amyloid burden, measured by PiB PET, with decreasing CSF Aβ40 levels has been identified as unique to CAA; and
In vitro and in vivo experimental data have provided increasing support to a prion hypothesis in CAA, wherein Aβ40 containing hereditary CAA mutations has a propensity to misfold and induce misfolding in WT protein, so that both are present in amyloid fibrils (akin to transthyretin (TTR)).

RNAi agent-mediated treatment of EOFAD is also expressly contemplated. Like hCAA, EOFAD is a devastating and rare disease and—as for hCAA—a causal role of APP is well-established and phenotyping of the disease can be performed with greater accuracy and over a shorter duration of time than, e.g., sporadic and/or late onset AD (optionally late onset AD with severe CAA as a subclass of late onset AD). EOFAD is a progressive, dementing neurodegenerative disease in young adults, possessing an age of onset before age 60 to 65 years and often before 55 years of age.

The prevalence of EOFAD has been estimated to be 41.2 per 100,000 for the population at risk (i.e., persons aged 40-59 years), with 61% of those affected by EOFAD having a positive family history of EOFAD (among these, 13% had affected individuals in three generations). EOFAD comprises less than 3% of all AD (Bird, Genetics in Medicine, 10: 231-239; Brien and Wang. Annu Rev Neu Sci, 2011, 34: 185-204; NCBI Gene Reviews).

Providing human genetic validation of the APP target (OMIM 104300), certain APP mutations have been identified that cause EOFAD, including E665D, K670N, M671L (Swedish), T714A (Iranian), T714I (Austrian), V715M (French), V715A (German), I716V (Florida), I716T, V717I (London), V717F, V717G and V717L, as described above. In addition, dominant amyloid beta precursor protein mutations have also been identified that cause EOFAD and CAA.

Without wishing to be bound by theory, the pathogenesis of AD is believed to begin in the hippocampus, a ridge of grey matter immediately superior to both lateral ventricles. Degeneration of this tissue is believed to cause the memory loss characteristic of early disease. While the mechanism of neurodegeneration at the protein level has been a matter of great debate, duplications of APP associated with EOFAD have indicated that overexpression of APP may be sufficient to cause AD. (Haass and Selkoe. Nature Reviews Molecular Cell Biology, 8: 101-112).

In contrast to EOFAD and CAA, the pathogenic mechanisms of sporadic AD are not yet understood and the population of clinically defined sporadic AD is probably mechanistically heterogeneous.

Certain aspects of the instant disclosure are directed towards targeting of APP for knockdown in individuals having EOFAD. A need exists for such agents because only symptom-directed treatments (of limited efficacy) exist for AD more generally and EOFAD in particular. In certain embodiments, the RNAi agents of the instant disclosure should provide approximately 60-80% knockdown of both mutant and WT APP levels throughout the CNS. One further observation from human genetics that speaks to the likely therapeutic efficacy of an APP-targeted therapy capable of knocking down APP levels in CNS cells is that an A673T mutation was identified that protected carriers from AD and dementia in the general population (Jonsson et al. Nature Letter, 488. doi:doi:10.1038/naturel 1283). The A673T substitution is adjacent to a β-secretase cleavage site, and has been described as resulting in a 40% reduction in amyloid beta in cell assays. Thus, a dominant negative APP point mutant appeared to protect families from AD, further reinforcing that RNAi agent-mediated knockdown of APP could exert a similar protective and/or therapeutic effect in at least certain forms of AD, including EOFAD.

Aiding initial stages of APP-targeting RNAi agent development, it has been noted that APP knockout mice are viable (OMIM 104300), which is expected to allow for viable use of mouse as a model system during lead compound development. In contrast to mice, while humans possessing heterozygous APP mutations exist in the general population with EXAC score of 0.3, no human APP knockout has been identified to date. Biomarkers available for development of APP-targeting RNAi agents include APP and MAPT peptides in CSF, which should allow for rapid assessment and useful efficacy even in a genetically homogeneous population (Mo et al. (2017) Alzheimers & Dementia: Diagnosis, Assessment & Disease Monitoring, 6: 201-209).

As noted above, attempts to treat sporadic forms of AD and EOFAD have to date proven unsuccessful—for example, all trials of BACE1 (β-secretase) inhibitors (BACE1i) for treatment of sporadic AD have thus far failed (Egan et al. The New England Journal of Medicine, 378: 1691-1703; Hung and Fu. Journal of Biomedical Science, 24: 47). In such BACEi testing, there have been no completed studies in genetically-defined populations (only studies initiated). Notably, the most recent BACE1i study showed that verubecestat lowered amyloid beta levels by 60% in a population selected based on age and clinical criteria that suggested a probable diagnosis of AD (Egan et al. The New England Journal of Medicine, 378: 1691-1703; Hung and Fu. Journal of Biomedical Science, 24: 47). Meanwhile, among Aβ-directed immunotherapies, one such immunotherapy demonstrated proof-of-concept in a recent trial in sporadic AD, supporting initiation of an ongoing Phase III trial (Selkoe and Hardy. EMBO Molecular Medicine, 8: 595-608). Given its role in APP cleavage, γ-secretase has also been targeted in certain AD-directed trials. However, to date no γ-secretase inhibitor trials have been completed in a genetically-defined population; and several programs have been discontinued for toxicity (Selkoe and Hardy).

A need therefore exists for pharmaceutically acceptable formulated agents that can treat or prevent APP-associated diseases or disorders in an affected individual.

It is expressly contemplated that all APP-associated diseases or disorders can ultimately be targeted using the RNAi agent compositions of the instant disclosure—specifically, targeting of sporadic CAA and sporadic and/or late onset AD is also contemplated for the RNAi agent compositions of the instant disclosure, even in view of the diagnostic/phenotyping issues presently confronted for these particular APP-associated diseases (it is further contemplated that diagnostics for these diseases will also continue to improve).

### Kits

In certain aspects, the instant disclosure provides kits that include a suitable container containing a pharmaceutical formulation of a nucleic acid agent (e.g., a siRNA compound, e.g., a double-stranded siRNA compound, or a precursor to a siRNA compound (e.g., a precursor, e.g., a larger siRNA compound which can be processed into a siRNA compound, or a DNA which encodes an siRNA compound, e.g., a double-stranded siRNA compound, or siRNA compound, or precursor thereof)), and instructions for its use. In certain embodiments the individual components of the pharmaceutical formulation may be provided in one container. Alternatively, it may be desirable to provide the components of the pharmaceutical formulation separately in two or more containers, e.g., one container for a nucleic acid agent (e.g., a siRNA compound) preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

In some embodiments, the disclosure further provides methods for the use of an RNAi agent or a pharmaceutical composition thereof, e.g., for treating a subject that would benefit from reduction or inhibition of APP expression, e.g., a subject having an APP-associated neurodegenerative disorder, in combination with other pharmaceuticals or other therapeutic methods, e.g., with known pharmaceuticals or known therapeutic methods, such as, for example, those which are currently employed for treating these disorders. For example, in certain embodiments, an RNAi agent targeting APP is administered in combination with, e.g., an agent useful in treating an APP-associated neurodegenerative disorder as described elsewhere herein or as otherwise known in the art. For example, additional agents and treatments suitable for treating a subject that would benefit from reduction in APP expression, e.g., a subject having an APP-associated neurodegenerative disorder, may include agents currently used to treat symptoms of APP. The RNAi agent and additional therapeutic agents may be administered at the same time or in the same combination, e.g., intrathecally, or the additional therapeutic agent can be administered as part of a separate composition or at separate times or by another method known in the art or described herein.

Exemplary additional therapeutics and treatments include dopamine-modulating agents, among others, for example, carbidopa-levodopa, levodopa, entacopone, tolcapone, opicapone, pramipexole, ropinirole, apomorphine, rotigotine, selegiline, rasagiline, safinamide, amantadine, istradefylline, trihexyphenidyl, benztropine, rivastigmine, donepezil, galantamine and memantine, as well as physical, occupational and speech therapy, an exercise program including cardiorespiratory, resistance, flexibility, and gait and balance exercises, and deep brain stimulation (DBS) involving the implantation of an electrode into a targeted area of the brain.

In one embodiment, the method includes administering a composition featured herein such that expression of the target gene is decreased, for at least one month. In certain embodiments, expression is decreased for at least 2 months, 3 months, or 6 months.

Optionally, the RNAi agents useful for the methods and compositions featured herein specifically target RNAs (primary or processed) of the target gene (e.g., APP). Compositions and methods for inhibiting the expression of these genes using RNAi agents can be prepared and performed as described herein.

Administration of the dsRNA according to the methods of the disclosure may result in a reduction of the severity, signs, symptoms, or markers of such diseases or disorders in a patient with a target gene-associated disorder. By “reduction” in this context is meant a statistically significant or clinically significant decrease in such level. The reduction can be, for example, at least 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of an APP-associated neurodegenerative disorder may be assessed, for example, by periodic monitoring of a subject's cognition, learning, or memory. Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an RNAi agent targeting APP or pharmaceutical composition thereof, “effective against” an APP-associated neurodegenerative disorder indicates that administration in a clinically appropriate manner results in a beneficial effect for at least a statistically significant fraction of patients, such as an improvement of symptoms, a cure, a reduction in disease, extension of life, improvement in quality of life, or other effect generally recognized as positive by medical doctors familiar with treating APP-associated neurodegenerative disorders and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and optionally at least 20%, 30%, 40%, 50% or more can be indicative of effective treatment. Efficacy for a given RNAi agent drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using an RNAi agent or RNAi agent formulation as described herein.

Subjects can be administered a therapeutic amount of dsRNA, such as about 0.01 mg/kg to about 200 mg/kg.

The RNAi agent can be administered intrathecally, via intravitreal injection, or by intravenous infusion over a period of time, on a regular basis. In certain embodiments, after an initial treatment regimen, the treatments can be administered on a less frequent basis. Administration of the RNAi agent can reduce target gene levels, e.g., in a cell, tissue, blood, CSF sample or other compartment of the patient by at least 20%, 30%, 40%, 50%, 55%, 60%, 65%, 70%, 75%, 80%, 85%, 90%, 95%, 96%, 97%, 98%, or at least about 99% or more. In a preferred embodiment, administration of the RNAi agent can reduce target transcript and/or protein levels, e.g., in a cell, tissue, blood, CSF sample or other compartment of the patient by at least 50%.

Before administration of a full dose of the RNAi agent, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Alternatively, the RNAi agent can be administered subcutaneously, i.e., by subcutaneous injection. One or more injections may be used to deliver the desired, e.g., monthly dose of RNAi agent to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of RNAi agent on a regular basis, such as monthly or extending to once a quarter, twice per year, once per year. In certain embodiments, the RNAi agent is administered about once per month to about once per quarter (i.e., about once every three months).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the RNAi agents and methods featured in the disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Definitions

That the present disclosure may be more readily understood, certain terms are first defined. In addition, it should be noted that whenever a value or range of values of a parameter are recited, it is intended that values and ranges intermediate to the recited values are also intended to be part of this disclosure.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

In the event of a conflict between an indicated target site and the nucleotide sequence for a sense or antisense strand, the indicated sequence takes precedence.

In the event of a conflict between a chemical structure and a chemical name, the chemical structure takes precedence.

As used herein, “excess” is any amount of excess of one component over a second component. “excess” can be measured/estimated by non-denaturing Ion-pair reverse phase HPLC (nd-IPRP), which if not equivalent to, approximates molar excess. As such, herein the terms “molar excess” and “excess” are used interchangeably. For example, an excess of antisense strand over sense strand or an excess of sense strand over antisense strand. The molar excess can be any amount. The excess can be about a 1-2% molar excess, a 1-3% molar excess, a 14% molar excess, a 1-5% molar excess, a 0-5% molar excess, a 0-1% molar excess, a 2-3% molar excess, a 3-4% molar excess, a 3-5% molar excess, a 2-4% molar excess, a 2-5% molar excess, a 0-10% molar excess, a 5-10% molar excess, a 2-6% molar excess, a 3-7% molar excess, or a 4-8% molar excess of one component over a second component (e.g., of antisense strand over sense strand or sense strand over antisense strand).

The term “inorganic phosphate”, as used herein, refers to the total amount of free phosphate (PO₄³⁻) in a solution and/or composition of the instant disclosure, as determined by art-recognized means, including, e.g., taking a measured amount of an aqueous sample and adding ammonium heptamolybdate reagent in a mixing tube. The tube is then stoppered and vigorously shaken. Dilute stannous chloride reagent, which has been freshly prepared from concentrated stannous chloride reagent and distilled water, to the mixture in the tube. This will produce a blue color (due to the formation of molybdenum blue) and the depth of the blue color indicates the amount of phosphate in the boiler water. The absorbance of the blue solution can be measured with a colorimeter and the concentration of phosphate in the original solution can be calculated. Alternatively, a direct (but approximate) reading of phosphate concentration can be obtained by using a Lovibond comparator. In certain embodiments, the total amount of inorganic phosphate is less than 100 parts per million (ppm), or is less than 0.64 μg/L.

As used herein, “target sequence” refers to a contiguous portion of the nucleotide sequence of an mRNA molecule formed during the transcription of a target gene, including mRNA that is a product of RNA processing of a primary transcription product. In one embodiment, the target portion of the sequence will be at least long enough to serve as a substrate for RNAi-directed cleavage at or near that portion of the nucleotide sequence of an mRNA molecule formed during the transcription of a target gene. In one embodiment, the target sequence is within the protein coding region of the target gene. In another embodiment, the target sequence is within the 3′ UTR of the target gene.

The target sequence may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. In some embodiments, the target sequence is about 19 to about 30 nucleotides in length. In other embodiments, the target sequence is about 19 to about 25 nucleotides in length. In still other embodiments, the target sequence is about 19 to about 23 nucleotides in length. In some embodiments, the target sequence is about 21 to about 23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G,” “C,” “A,” “T”, and “U” each generally stand for a ribonucleotide that contains guanine, cytosine, adenine, thymidine, and uracil as a base, respectively in the context of a modified or unmodified nucleotide. However, it will be understood that the term “ribonucleotide” or “nucleotide” can also refer to a modified nucleotide, as further detailed below, or a surrogate replacement moiety (see, e.g., Table 15).

As used herein, the term “modified nucleotide” refers to a nucleotide having, independently, a modified sugar moiety, a modified internucleotide linkage, or a modified nucleobase, or a combination thereof. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a siRNA type molecule, are encompassed by “RNAi agent” for the purposes of this specification and claims.

As used herein, the term “2′-deoxynucleotide” refers to a 2′-deoxyribonucleotide, or to a 2′-deoxy nucleotide including a ribose analog. In certain embodiments, a “2′-deoxynucleotide” refers to a 2′-deoxyribonucleotide. It is understood that, when present within an RNAi agent, a 2′-deoxy modification is considered a modified nucleotide.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double stranded over its entire length.

The term “antisense strand” or “guide strand” refers to the strand of an RNAi agent, e.g., a dsRNA, which includes a region that is substantially complementary to a target sequence, e.g., an APP mRNA.

As used herein, the term “region of complementarity” refers to the region on the antisense strand that is substantially complementary to a sequence, for example a target sequence, e.g., an APP nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- or 3′-terminus of the RNAi agent.

The term “sense strand” or “passenger strand” as used herein, refers to the strand of an RNAi agent that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNAi agent, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an RNAi agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a target protein). For example, a polynucleotide is complementary to at least a part of a target gene mRNA if the sequence is substantially complementary to a non-interrupted portion of an mRNA encoding the target protein.

The phrase “contacting a cell with an RNAi agent,” such as a dsRNA, as used herein, includes contacting a cell by any possible means. Contacting a cell with an RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., the central nervous system (CNS), optionally via intrathecal, intravitreal or other injection, or to the bloodstream or the subcutaneous space, such that the agent will subsequently reach the tissue where the cell to be contacted is located. For example, the RNAi agent may contain or be coupled to a ligand, e.g., a lipophilic moiety or moieties as described below and further detailed, e.g., in PCT/US2019/031170, which is incorporated herein by reference, that directs or otherwise stabilizes the RNAi agent at a site of interest, e.g., the CNS. In some embodiments, the RNAi agent may contain or be coupled to a ligand, e.g., one or more GalNAc derivatives as described below, that directs or otherwise stabilizes the RNAi agent at a site of interest, e.g., the liver. In other embodiments, the RNAi agent may contain or be coupled to a lipophilic moiety or moieties and one or more GalNAc derivatives. Combinations of in vitro and in vivo methods of contacting are also possible. For example, a cell may also be contacted in vitro with an RNAi agent and subsequently transplanted into a subject.

In one embodiment, contacting a cell with an RNAi agent includes “introducing” or “delivering the RNAi agent into the cell” by facilitating or effecting uptake or absorption into the cell. Absorption or uptake of an RNAi agent can occur through unaided diffusive or active cellular processes, or by auxiliary agents or devices. Introducing an RNAi agent into a cell may be in vitro or in vivo. For example, for in vivo introduction, an RNAi agent can be injected into a tissue site or administered systemically. In vitro introduction into a cell includes methods known in the art such as electroporation and lipofection. Further approaches are described herein below or are known in the art.

The term “artificial cerebrospinal fluid (aCSF)” refers to a solution prepared with a composition representative of human cerebrospinal fluid (hCSF) that closely matches the electrolyte concentrations of hCSF, and may include glucose. Exemplary ion concentrations, as prepared in high purity water, include (in mM): Na⁺ 150; K⁺ 3.0; Ca²⁺ 1.4; Mg²⁺ 0.8; P 1.0; Cl⁻ 155 (for example, Tocris Bioscience™ ACSF, Cat. No. 3525, available from Thermo Fisher Scientific, Hampton, NH, USA). An exemplary aCSF formulation includes 127 mM NaCl; 1.0 mM KCl; 1.2 mM KH₂PO₄; 26 mM NaHCO₃; 10 mM D-glucose; 2.4 mM CaCl₂); and 1.3 mM MgCl₂, with the pH and oxygen level stabilized by bubbling with carbogen (95% 02 and 5% CO₂).

A solution is considered “isotonic to aCSF” when its effective osmole concentration is the same as that of aCSF. For example, the solutions on either side of a cell membrane are isotonic if the concentration of solutes outside the cell is equal to the concentration of solutes inside the cell.

Where the two substantially complementary strands of a dsRNA are comprised by separate RNA molecules, those molecules need not, but can, be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

As used herein, the expression “optionally substituted” means that at least one hydrogen present on a group (e.g., a carbon of an alkyl, alkenyl, or alkynyl) is replaced with a permissible substituent, e.g., a substituent which upon substitution results in a stable compound, e.g., a compound which does not spontaneously undergo transformation such as by rearrangement, cyclization, elimination, or other reaction. Heteroatoms, such as nitrogen, may have substituents, such as any suitable substituent described herein which satisfies the valencies of the heteroatoms and results in the formation of a stable moiety. For example, each instance of an alkyl group may be independently optionally substituted, i.e., unsubstituted (an “unsubstituted alkyl”) or substituted (a “substituted alkyl”) with one or more substituents. Suitable substituent groups may include, but are not limited to, hydroxyl, nitro, amino (e.g., —NH₂ or dialkyl amino), imino, cyano, halo (e.g., F, Cl, Br, I, and the like), haloalkyl (e.g., —CCl₃, —CF₃, and the like), thio, sulfonyl, thioamido, amidino, imidino, oxo, oxamidino, methoxamidino, imidino, guanidino, sulfonamido, carboxyl, formyl, alkyl, alkoxy, alkoxy-alkyl, alkylcarbonyl, alkylcarbonyloxy (e.g., —OCOR), aminocarbonyl, arylcarbonyl, aralkylcarbonyl, carbonylamino, heteroarylcarbonyl, heteroaralkyl-carbonyl, alkylthio, aminoalkyl, cyanoalkyl, carbamoyl (e.g., —NHCOOR— or —OCONHR—), urea (e.g., —NHCONHR—), cycloalkyl, aryl, heteroaryl, heteroalicyclic, hydroxy, alkoxy, aryloxy, mercapto, alkylthio, arylthio, cyano, halo, carbonyl, (═O), thiocarbonyl, O-carbamyl, N-carbamyl, O-thiocarbamyl, N-thiocarbamyl, C-amido, N-amido, C-carboxy, O-carboxy, nitro, amino, heterocycle, —CN, and the like. An “alkyl” as used herein may be combined with other groups, such as those provided above, to form a functionalized alkyl.

In one embodiment, a target gene-associated disease or disorder is one of Alzheimer's disease (AD), cerebral amyloid angiopathy (CAA; e.g., hereditary CAA), early onset familial Alzheimer disease (EOFAD or eFAD), early onset Alzheimer's disease (EOAD), familial Alzheimer's disease, or late onset Alzheimer's disease.

“Therapeutically effective amount,” as used herein, is intended to include the amount of an RNAi agent that, when administered to a subject having a target gene-associated disease, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an RNAi agent that, when administered to a subject having a target gene-associated disorder, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired local or systemic effect at a reasonable benefit/risk ratio applicable to any treatment. An RNAi agent employed in the methods of the present disclosure may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

As used herein, the phrase “pharmaceutically acceptable salt” refers to both pharmaceutically acceptable acid and base addition salts and solvates. Such pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as lithium, sodium, potassium, calcium, magnesium, ammonium, and the like. Those skilled in the art will recognize a wide variety of non-toxic pharmaceutically acceptable addition salts.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the brain (e.g., whole brain or certain segments of brain, e.g., striatum, or certain types of cells in the brain, such as, e.g., neurons and glial cells (astrocytes, oligodendrocytes, microglial cells)). In other embodiments, a “sample derived from a subject” refers to liver tissue (or subcomponents thereof) derived from the subject. In some embodiments, a “sample derived from a subject” refers to blood drawn from the subject or plasma or serum derived therefrom. In further embodiments, a “sample derived from a subject” refers to brain tissue (or subcomponents thereof) or retinal tissue (or subcomponents thereof) derived from the subject.

It will be understood that, although the sequences in Table 16 and Table 18 are described as modified or conjugated sequences, the RNA of the RNAi agent of the disclosure e.g., a dsRNA of the disclosure, may comprise any one of the sequences set forth in Tables 16-19 that is un-modified, un-conjugated, or modified or conjugated differently than described therein. That is, the modified sequences provided in Table 16 and Table 18 do not require the indicated hexadecyl lipophilic moiety, or any ligand. A lipophilic ligand can be included in any of the positions provided in the instant application.

### EXAMPLES

### Example 1: Materials and Methods

This Example describes methods for the design, synthesis and selection of exemplified APP-targeting RNAi agents, as well as the improved formulation process disclosed herein for preparation of drug products harboring RNAi agents.

### Source of Reagents

Where the source of a reagent is not specifically given herein, such reagent can be obtained from any supplier of reagents for molecular biology at a quality/purity standard for application in molecular biology.

siRNA Agents

siRNA agents targeting the human amyloid beta precursor protein gene (APP), are described in International Publication No. WO2020/132227 (International application no. PCT/US2019/067449); International Publication No. WO2022/165172 (International application no. PCT/US2022/014309); and International Publication No. WO2023/039503 (International application no. PCT Application No: PCT/US2022/076159); each of which is hereby incorporated by reference in its entirety.

siRNA agents targeting the human superoxide dismutase 1 gene (SOD1), are described in International Publication No. WO2022/174000 (International application no. PCT/US2022/016046), which is hereby incorporated by reference in its entirety.

siRNA agents targeting the human huntingtin gene (HTT), are described in International Publication No. WO2021/087036 (International application no. PCT/US2020/057849), International Publication No. WO2022/212231 (International application no. PCT/US2022/022093), and International Publication No. WO2023/076450 (International application no. PCT/US2022/047986, entitled “HUNTINGTIN (HTT) iRNA AGENT COMPOSITIONS AND METHODS OF USE THEREOF” and filed Oct. 27, 2022), each of which is hereby incorporated by reference in its entirety.

Synthesis of siRNA Agents

All oligonucleotides were prepared on an automatic solid phase synthesizer using universal or custom supports (see, e.g., F. Eckstein (ed.), Oligonucleotides and Analogues, a Practical Approach (Oxford University Press, New York 1991)). 3′-RNA containing agents can be prepared according to the process disclosed in International Publication no. WO 2021/108291 which is hereby incorporated by reference in its entirety.

Annealing of APP single strands was performed on a Tecan liquid handling robot. Sense and antisense single strands were combined in an equimolar ratio in 96 well plates and buffered with 10×PBS to provide a final duplex concentration of 10 M in 1×PBS. After combining the complementary single strands, the 96 well plate was sealed tightly and heated in an oven at 100° C. for 40 minutes and allowed to come slowly to room temperature over a period of 2-3 hours and subsequently used directly for in vitro screening assays at the appropriate concentrations.

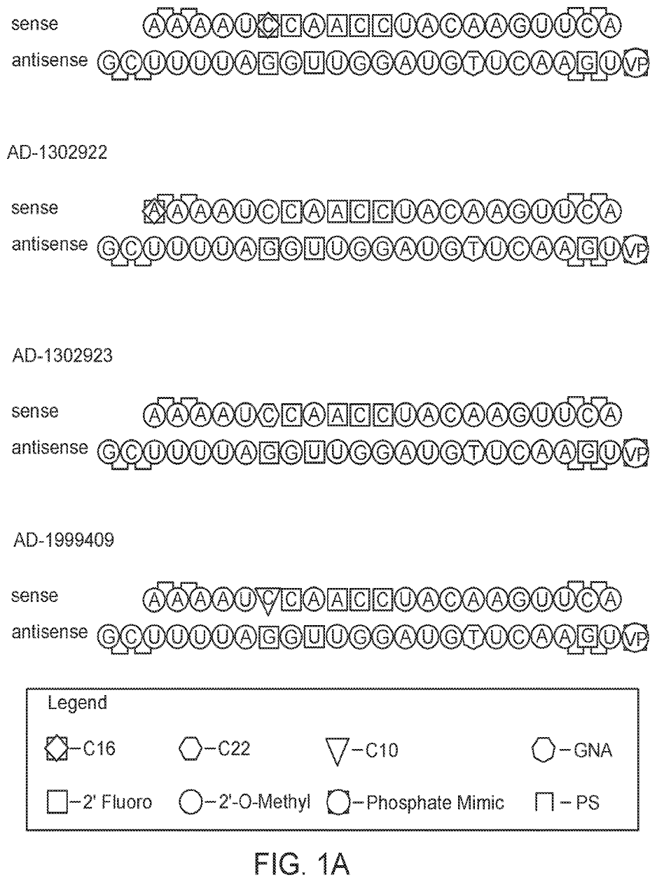

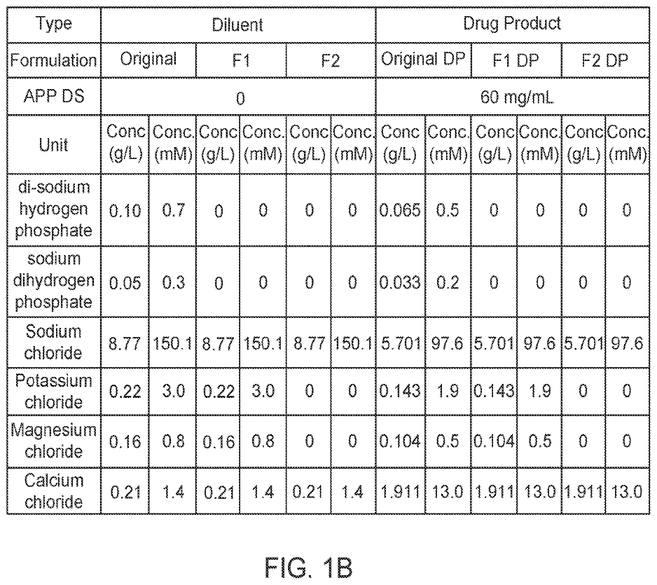

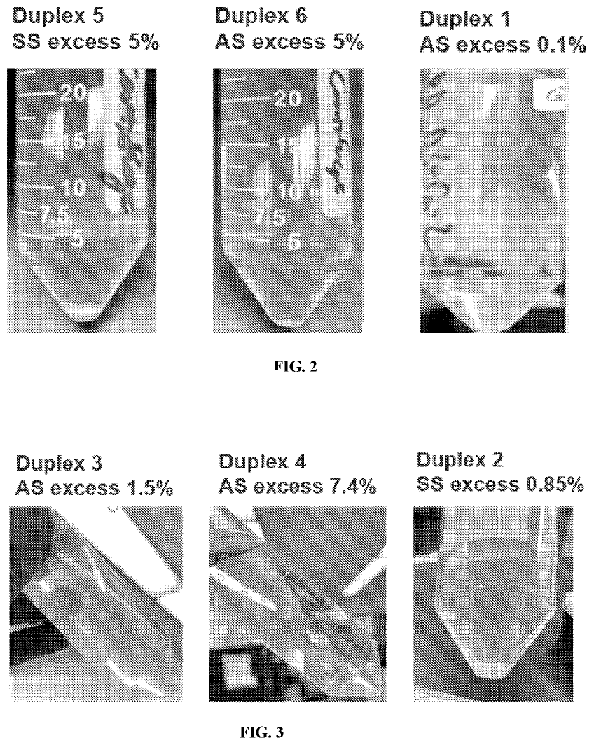

A detailed list of the modified APP sense and antisense strand sequences is shown in FIG. 1A and Table 16, and a detailed list of the unmodified APP sense and antisense strand sequences is shown in Table 17.

APP drug product (DP) was prepared from an APP drug substance (DS) lyophilized powder. APP DP formulation began by dissolving the powdered APP DS (such as, e.g., AD-961583, AD-454973, AD-454843, AD-961584, AD-961585, AD-961586, AD-454844, AD-1302922, AD-1302923, or AD-1999409) in buffer and thoroughly mixing by hand for a concentration of ~80 mg/mL ALN DS at ambient temperature. The pH was adjusted to 6.8+/−0.2 with a 0.25 M solution of sodium hydroxide or hydrochloric acid, as needed. The concentration of APP DS in solution was measured and adjusted to the final target concentration of approximately 60 mg/mL AD-961583 (Free Acid Form) with the buffer. The concentration was determined by a UV spectrophotometry-based assay. The solution was passed through one polyethersulfone (PES) 0.22 m membrane filter for bioburden reduction and filtered again for sterility. The sterile filtered solution was aseptically filled into a Type I glass vial, aseptically finished with fluoropolymer coated stopper, and an aluminum over seal. After the aseptic fill, vials were spot checked for any damage to the container closure or for obvious particulates.

### Calcium Exchange Experiment

For each respective duplex, a titration was performed to target a 5% respective sense strand excess. This excess was confirmed via nd-IPRP (non-denaturing-Ion-Pair Reversed-Phase liquid chromatography) integration prior to annealing. Once the ratio was confirmed to be close to the target, annealing was performed.

Following the duplex production, approximately 5 g of each respective duplex was aliquoted for use in the calcium exchange. The material amount was determined based on calculations, using the single strand solution concentrations, titration amounts, and single strand conversion factors to calculate the volume necessary for approx. 5 g of duplex. Two calcium solutions (1 mM and 20 mM) were prepared by dissolving calcium chloride dihydrate salt in RODI via mixing and filtered prior to use. The pH and conductivity of each calcium solution was measured for reference.

All four duplexes underwent the same procedure for the calcium exchange. The calcium exchange was performed using a standard UF set up, with 5 kD MWCO Sartorius Hydrosart membrane cassettes and applying standard crossflow filtration (CFF) and transmembrane pressure (TMP) parameters. The retentate material was targeting 30 mg/mL concentration in the tank to maintain the diafiltration volume (DV). Based on the determined DV, the UF exchanges were performed by doing 15 DV exchanges using 20 mM CaCl₂) followed by 10 DV exchanges using 1 mM CaCl₂). The permeate was monitored for conductivity to assess completion of the exchange, ensuring that the permeate conductivity was close to that of the respective feed solution at the end of the respective exchange volumes. The retentate tank was also monitored for observations of precipitation or turbidity, but the process was not adjusted regardless of the observations. After completion of the 1 mM CaCl₂) exchanges, the material was harvested and set aside. An additional rinse of 1 mM CaCl₂) was performed but not combined with the initial harvest materials.

For the calcium exchange assessment, a ThermoScientific Orion AQ4500 Turbidity Meter was used in conjunction with nd-IPRP analysis. For each respective calcium-form duplex, a series of samples was made by aliquoting four 10 mL samples of the calcium-form duplex solution into falcon tubes. Each aliquot was then titrated with increasing amounts of the sodium-form antisense strand, in intervals of 100 μL, so the 100-400 μL range. The samples were capped, vortexed, and heated on a pre-heated hotplate set for 85° C. for 5 minutes (uncapping the samples in that time to prevent pressure build-up). Following the 5 minutes, the samples were removed from the hot plate and set on the bench to cool to room temperature. Once at room temperature, a nd-IPRP sample was prepared for each sample and analyzed to determine the single strand excess. The remainder of the sample was transferred to a cleaned vial for turbidity measurement (the vial was specific to the ThermoScientific Orion AQ4500 instruments) and allowed to settle at least overnight without moving. One hour prior to turbidity measurement, the samples were gently inverted 10× to mix the samples while not introducing air bubbles, and then analyzed all together. Initial data analysis included graphing the nd-IPRP single strand excess (x-axis, designating the sense excess as negative and antisense excess as positive) versus the turbidity (y-axis).

### Particulate Mitigation Protocol

Duplexes were annealed with sense strand excess. The extent of sense strand excess was confirmed by non-denaturing ion-pair reversed-phase high performance liquid chromatography (nd-IPRP) analysis. Calcium exchange was then performed upon the duplexes by ultrafiltration following an established protocol. Precipitates suspensions/turbid solutions were then harvested, and rescue was initiated. For rescue studies, Precipitates suspensions/turbid solutions were aliquoted into a set of vials, and various amounts of antisense were added into vials serially. After incubation, turbidity was measured to determine the effectiveness of the particulate mitigation strategy.

### Formulations Discussed in the Examples

As shown in FIG. 1B, the “original” formulations (both placebo and drug product) included di-sodium hydrogen phosphate and sodium dihydrogen phosphate as respective sources of inorganic phosphate. Formulation 1 placebo (“F1”) and drug product (“F1 DP”) formulations removed the preceding sources of inorganic phosphate, while retaining all other components. Formulation 2 placebo (“F2”) and drug product (“F2 DP”) formulations not only removed the preceding sources of inorganic phosphate but also removed potassium chloride and magnesium chloride components, thereby retaining only sodium chloride and calcium chloride in the placebo/diluent formulation (F2) and only sodium chloride, calcium chloride and the APP drug substance (RNAi agent) in the drug product formulation (F2 DP). Compositions of F3 DP through F8 FP were the same as F1 DP, only changing the relative amounts of sense and antisense strand as summarized in the following:

| Formulation | Composition |
|---|---|
| Original DP | potassium chloride, magnesium chloride, sodium |
| | chloride, calcium chloride, di-sodium hydrogen |
| | phosphate and sodium dihydrogen phosphate |
| F1 DP | potassium chloride, magnesium chlorid, sodium |
| | chloride, calcium chloride |
| F2 DP | sodium chloride, calcium chloride, F1 DP |
| F3 DP | formulation having 0.1% molar excess of the |
| | non-lipophile-modified antisense strand |
| F4 DP | F1 DP formulation having 0.85% molar excess of the |
| | lipophile-modified sense strand |
| F5 DP | F1 DP formulation having 1.5% molar excess of the |
| | non-lipophile-modified antisense strand |
| F6 DP | F1 DP formulation having 7.4% molar excess of the |
| | non-lipophile-modified antisense strand |
| F7 DP | F1 DP formulation having 5% molar excess by the |
| | lipophile-modified sense strand |
| F8 DP | F1 DP formulation having 5% molar excess by of |
| | the non-lipophile-modified antisense strand |

### Example 2: Phosphate-Free Solutions Improved iRNA Agent Formulation Properties

Particulates formed in a phosphate-buffered formulation of an APP-targeting iRNA agent, prepared for central nervous system (CNS) delivery via an intrathecal route of administration. The particulates occurred in an artificial cerebrospinal fluid (aCSF)-based isotonic aqueous buffer (containing sodium, potassium, magnesium, calcium and phosphate) harboring 60 mg/mL of an APP-targeting drug substance (the AD-961583 duplex of Table 16 below, as a free acid) prepared for intrathecal (IT) injection. Such observed particulates appeared to result from accumulation of calcium phosphate as a precipitate and dramatically reduced the utility of the APP-targeting formulations for clinical applications. A search for an improved iRNA drug product formulation—specifically an improved formulation for iRNA drug substances intended for intrathecal and/or central nervous system (CNS) delivery—was therefore initiated.

While calcium phosphate particles were believed to be the primary source of the observed particulates in the aCSF-based formulations, inclusion of calcium as a supplement had also been identified as helpful for mitigation of certain clinical signs of intrathecal formulation-induced effects (e.g., tremors, twitches, etc.) that were observed upon injection of such iRNA drug product formulations to the CNS of animals.

Phosphate-free solutions were investigated for formulation of iRNA drug substances. Specifically, sodium phosphate excipients were removed from the original, particulate-forming aCSF-based iRNA agent test formulation solutions. While an original drug product formulation (harboring 60 mg/mL of iRNA agent AD-961583) included di-sodium hydrogen phosphate at 0.5 mM, sodium dihydrogen phosphate at 0.2 mM, sodium chloride at 97.6 mM, potassium chloride at 1.9 mM, magnesium chloride at 0.5 mM and calcium chloride at 13.0 mM, a “F1” drug product (“F1 DP”) formulation (harboring 60 mg/mL of iRNA agent AD-961583) included sodium chloride at 97.6 mM, potassium chloride at 1.9 mM, magnesium chloride at 0.5 mM and calcium chloride at 13.0 mM (FIG. 1B). Meanwhile, a “F2” drug product (“F2 DP”) formulation (again harboring 60 mg/mL of iRNA agent AD-961583) included only sodium chloride at 97.6 mM and calcium chloride at 13.0 mM. In such phosphate-free drug product formulations, it was observed that the APP-targeting drug substance (AD-961583 at 60 mg/mL) inherently possessed buffer capacity comparable to the di-sodium hydrogen phosphate and sodium dihydrogen phosphate buffering moieties removed from the original aCSF-based formulation in forming the “F1 DP” and “F2 DP” formulations, thereby identifying the free phosphate-containing components of the original formulation(s) as removable without significant impact upon buffering.

Removal of the phosphate salts (di-sodium hydrogen phosphate and sodium dihydrogen phosphate) from the original DP formulation, as well as further removal of not only all phosphate salts but also potassium chloride and magnesium chloride from the F1 DP formulation, caused minimal impact upon tonicity of the respective F1 DP and F2 DP formulations, relative to the starting aCSF-based DP formulation. The F1 DP formulation was noted as closest among the new formulations tested to the original aCSF-based formulation, while the F2 DP formulation was essentially a saline formulation with a necessary calcium concentration.

Diluent formulations corresponding to the above-described new DP formulations (F1 DP and F2 DP) were also developed and tested in parallel. As noted above, sodium phosphate excipients found in the original aCSF-based formulations were removed to minimize formation of calcium phosphate precipitation during the in-use dilution process. The phosphate-free placebo formulations, F1 and F2, respectively, maintained the same formulation components as corresponding drug products, F1 DP and F2 DP, respectively (FIG. 1B). Specifically, the F1 placebo/diluent formulation included sodium chloride at 150.1 mM, potassium chloride at 3.0 mM, magnesium chloride at 0.8 mM and calcium chloride at 1.4 mM, while the F2 placebo/diluent formulation included only sodium chloride at 150.1 mM and calcium chloride at 1.4 mM (FIG. 1B). The pH ranges of each of these formulations were similar to the pH of saline solution.

Stress and stability studies performed upon each of the above-described formulations revealed the new, phosphate-free formulations of the instant disclosure to possess improved properties (most notably, absence of particulate formation under relevant tested conditions) capable of enhancing their respective use as drug product formulations.

Stability studies of tested modified formulations included the following: (1) performance of full visual inspection of formulations in vials prior to study initiation; (2) initiation of testing of long-term stability at 5° C., 25° C. and 30° C. for up to five years (at 1, 3, 6, 9, 12, 18, 24, 36, 48 and 60 months); (3) initiation of an accelerated stability study at 40° C. for six months (0, 1, 3 and 6 months); (4) testing of samples at the same time intervals and using the same methods employed for studies that identified deleterious particulate formation in aCSF-based formulations, while adding high accuracy (HIAC) testing to all time points and storage conditions; and (5) performance of an additional visual inspection of 40° C. stored samples at 2, 4, 6, 8 hour and 1, 3 and 7 days post-study initiation time points.

The following stress studies were also performed upon the formulations of the instant disclosure: (1) initiate stress studies after performing full visual inspection of formulations in vials; (2) initially stress formulations via incubation at −20° C. and if no particulates were observed, stress studies were performed with the −20° C. condition included—if particulates were observed at −20° C., assayed stress test temperatures were limited to only those above 0° C.

Stage 1 of stress testing was therefore a thermal stress test, which included freezing of formulations at −20° C. for 24 hours and checking for particulates (n=10). If no particulates were identified, 5 out of the 10 vials were transferred to 37° C. for one week, with further particulate checks performed at 2, 4, 6, 8 hours, and 1, 3 and 7 days. Meanwhile, single vials from the preceding “stage 1” steps were tested for pH, assay, osmolality, non-denaturing ion pairing reversed-phase (IPRP) chromatography, non-denaturing anion exchange (AX) high performance liquid chromatography (HPLC) and HIAC liquid particle counting (assessment of light obscuration).

Stage 2 of stress testing involved temperature cycling. Specifically, in stage 2, samples were cycled between −20° C. and 60° C., unless exposure to −20° C. was determined not to be viable, in which case cycling was performed between 5° C. and 60° C. Samples were initially cycled 3 times (minimum 3 days at each of 60° C. and 5° C. conditions and/or 1 day at −20° C. per cycle). The appearance of all vials was checked at the start of the stage 2 study and at the end of each temperature cycle. One vial from cycle 3 was tested for pH, assay, osmolality, non-denaturing ion pairing reversed-phase (IPRP) chromatography, non-denaturing anion exchange (AX) liquid chromatography (LC), while three vials were tested by HIAC liquid particle counting (assessment of light obscuration) for particulates (particle count values obtained by HIAC of less than the following United States Pharmacopeial Convention (USP) threshold values: >10 μm=6000 particles per container; >25 μm=600 particles per container).

After 7 days of stage 2 incubation at 40° C., the new, phosphate-free formulations (both F1 DP (“Formulation 1”) and F2 DP (“Formulation 2”)) were observed to be clear, colorless solutions, free of any observable particulates on visual inspection. Both tested formulations were also measured by HIAC to exhibit particulate count values well within USP acceptable limits, with no apparent trends observed across time points/conditions (data not shown). Accordingly, HIAC and appearance assessment identified both new, phosphate-free formulations to be stable at 40° C., extending out to a 7 day time point.

In addition to testing phosphate-free DP formulations, diluent formulations were also tested for stability, absence of particulate formation, and for other properties. In such diluent studies, a full visual inspection was performed upon vials prior to study initiation. Long-term stability studies were then initiated at 5° C., 25° C. and 30° C., for assessment over a duration of five years (with 1, 3, 6, 9, 12, 18, 24, 36, 48 and 60 month timepoints). An accelerated placebo (diluent) stability study at 40° C. was also initiated, for assessment over a duration of six months (assessed at 0, 1, 3 and 6 month timepoints). Visual inspection of vials was performed at 7, 14 and 21 days, and samples were checked at other storage conditions if particles were detected. Samples were tested using the same methods and at the same time intervals utilized for the above-described formulation studies. Tests performed therefore included visual inspection for appearance, pH, osmolality and HIAC. For visual appearance, clear, colorless to pale yellow solutions essentially free of particulates were identified as passing the specifications of the test. All samples were assessed as visually clear, colorless solutions free of particulates, across initial and early timepoints (e.g., to the 21 day timepoint in the 40° C. accelerated placebo (diluent) stability study).

Thus, tested phosphate-free APP DP formulations (F1 DP and F2 DP formulations) were subjected to both stress studies (at −20° C., −20° C. to 37° C. & 3 cycles between −20° C. and 60° C.) and initial long term stability studies (after 7 days at 40° C.), with no particles yet observed in any such type of study and sub-visible particulate counts remaining well below USP specification limits. In addition, no apparent trends were observed. For stress studies of phosphate-free APP DP formulations, no reportable change in other quality attributes (purity, impurities, pH, osmolality, assay) was detected. Finally, placebo (diluent) long term stability studies identified no change in product appearance after 21 days at 40° C.

### Example 3: Long-Term Stability and in-Use Testing of Formulation APP F1 DP Showed Evidence of Particulate Formation

Stability testing of APP F1 DP was continued to the 3-month time point under following conditions
2-8° C.
25° C.±2° C./60%±5% relative humidity (RH)
30° C.±2° C./75%±5% relative humidity
40° C.±2° C./75%±5% relative humidity
   At the 3-month time point, fine particulates were observed in samples stored under the 40° C. condition. No particulate was observed at the other storage temperatures (25° C., 30° C., and 2-8° C.). All other quality attributes continued to meet specifications with no apparent trends.

F1 DP clinical in-use stability was also assessed for a 60 m/mL DP vial of APP DP, and F1 DP diluted with diluent to a concentration of 1.25 mg/mL with and without filtering the F1 DP through a 0.2 micron filter prior to dilution Specifically, for unfiltered samples, 0.4 mL of 60 mg/mL DP was combined with 19.6 mL of the placebo/diluent in two 30-mL polypropylene syringes with a 21G needle (one syringe was stored at 2-8° C. for 48 hours+1 hour at 37° C.; another syringe was stored at 25° C. for 8 hours+1 hour at 37° C.). For filtered samples, 1.0 mL of 60 mg/mL DP was drawn into a syringe from DP vial using 21-gauge needle, 0.2 micron filter, connected to a syringe; 19.6 mL of placebo/diluent was drawn up using 21-gauge needle with a syringe connected. The two syringes were then connected, and the DP was slowly depressed into the diluent syringe (one syringe was stored at 2-8° C. for 48 hours+1 hour at 37° C.; another syringe was stored at 25° C. for 8 hours+1 hour at 37° C.).

While no particulates were observed under storage at 5° C. for 48 hours followed by 1 hour at 37° C., fine particulates were observed for the syringe stored at 25° C. for 8 hours followed by 1 hour at 37° C. Filtration did not have an effect on the results, as particles formed in samples prepared with and without initial filtration. Clarity remained clear and color remained colorless for all samples under both storage conditions.

The effect of the initial storage time at 25° C. was further examined for the 1.25 mg/mL unfiltered solution, prepared by the preceding method. Such samples, following preparation, were stored and observed for particulate according to the schedule:
Sample 1: 25° C. for 30 min-->37° C. for 1 hour-->testing
Sample 2: 25° C. for 1 hour-->37° C. for 1 hour-->testing
Sample 3: 25° C. for 2 hours-->37° C. for 1 hour-->testing
Sample 4: 25° C. for 4 hours-->37° C. for 1 hour-->testing
   One unopened clinical vial was kept at 2-8° C. for the duration of study as control.

Samples 1 and 2 remained essentially free of particulate for the course of the study. Sample 3 exhibited light formation of fine particles, while both sample 4 and the control, the latter quite unexpectedly, displayed fine particles formation. Clarity remained clear and color remained colorless for all samples and control vial under both storage conditions.

### Example 4: No Particulates Formed in siRNA Formulations Having Molar Excess of Non-Lipophilic Moiety-Modified Strands Relative to Lipophilic Moiety-Modified Strands

Calcium exchange studies were then performed as a further modeling of particulate formation in APP-targeting formulations. Calcium exchange through ultrafiltration has the advantage of offering an accelerated process for monitoring of potential particulate formation in the process of converting an oligonucleotide counterion from monovalent cation Na⁺ to the divalent cation Ca²⁺. For the APP-targeting siRNA formulation “APP F4 DP” that had previously shown particulate formation, pellets were also observed to form during the calcium exchange process. Evaluation of the pellets showed compositions that mainly exhibited n-1 impurities (e.g., having a single nucleoside missing from the sequence of the oligonucleotide) and other impurities of the C16-modified strand (SS). Meanwhile, the siRNA formulation “APP F3 DP” exhibited no pellets formed in calcium exchange, and the APP-targeting antisense strand oligonucleotide formulation “APP AS” also exhibited no pellet formation in calcium exchange studies. In contrast, the APP sense strand oligonucleotide formulation “APP SS” exhibited a very similar purity/impurity profile to the “APP F4 DP” siRNA formulation, as the full-length C16-modified sense strand itself, and also formed particles/pellets. The C16-modified full length product in calcium form therefore shared the low solubility observed forthe “APP F4 DP” formulation, which produced particles containing C16-modified “shortmers” (C16-modified oligonucleotides having “n-1” impurities).

Having identified that particulate formation during calcium exchange studies appeared to track with having the C16-modified sense strand of the APP-targeting dsRNA present in abundance, particle-forming and non-particle-forming formulations were evaluated for batch differences that might provide further insight into forces driving particulate formation. Such further evaluation revealed an approximate three-fold increase in n-1 impurities in the C16-modified sense strand oligonucleotides in the particle-forming “APP F4 DP” siRNA formulation, as compared to the non-particle-forming “APP F3 DP” siRNA formulation. Intriguingly, the particle-forming “APP F4 DP” siRNA formulation was identified to have been formulated with the C16-modified sense strand present at a molar excess of 0.85%, relative to the non-lipophile-modified antisense strand of the APP-targeting duplex. In contrast, the non-particle-forming “APP F3 DP” siRNA formulation was identified to have the non-lipophile-modified antisense strand in a molar excess of 0.1%, as compared to the C16-modified sense strand of the APP-targeting duplex. Pellet formation during calcium exchange therefore appeared more likely to have been driven by excess of the C16-modified sense strand, than being simply the result of elevated n-1 impurities present in the C16-modified sense strand.

To evaluate whether having a molar excess of the C16-modified sense strand relative to the non-lipophile-modified antisense strand could indeed cause particle formation, additional calcium exchange studies were initiated. First, two new formulations were evaluated, one of which possessed a 5% molar excess of sense strand relative to antisense strand (termed “APP F7 DP”) and a second which alternatively possessed a 5% molar excess of antisense strand relative to sense strand (termed “APP F8 DP”). Results of these studies are shown in Table 3.

TABLE 3
Calcium Exchange Study Results for Two New Formulations

| | APP F7 DP | APP F8 DP |
|---|---|---|
| Single Strand Excess | Sense Strand by 5% | Antisense Strand by 5% |
| Pellets forming in | Yes | No |
| Ca²⁺ Exchange | | |

As observed previously for the “APP F3 DP” siRNA formulation, which possesses an antisense strand molar excess of 0.1%, no pellets formed. However, the “APP F7 DP” formulation, which possessed a 5% molar excess of the C16-modified sense strand relative to the antisense strand was observed to form particles/a pellet in performing calcium exchange studies. In contrast, no particulates were observed in the “APP F8 DP” formulation possessing a 5% molar excess of the antisense strand relative to the C16-modified sense strand.

To confirm the effect of carrying a molar excess of the non-lipophile-modified antisense strand relative to the C16-modified sense strand in preventing a siRNA formulation from forming particulates, two new test formulations were then designed and evaluated in calcium exchange studies using material corresponding to the particle-forming “APP F4 DP” siRNA formulation. While the original “APP F4 DP” formulation carried an excess of sense strand by 0.85%, the two new formulations, named “APP F5 DP” and “APP F6 DP” were prepared with the antisense strand in molar excess relative to the C16-modified sense strand, by 1.5% and by 7.4%, respectively. Results of calcium exchange characterization studies of these two new duplexes are shown in

TABLE 4
Calcium Exchange Study Results for New Duplexes
of the “APP F4 DP” Formulation

| | APP F5 DP | APP F6 DP | APP F4 DP |
|---|---|---|---|
| Re-titration | Yes | Yes | No |
| with GMP AS | | | |
| % Ca²⁻ after Ca²⁺ | 5.6 | 5.9 | 5.6 |
| Exchange | | | |
| Single Strand | Antisense | Antisense | Sense Strand |
| Excess | Strand by 1.5% | Strand by 7.4% | by 0.85% |
| Pellets forming | No | No | Yes |
| in Ca²⁺ Exchange | | | |

While the “APP F4 DP” formulation having a molar excess of sense strand was observed to form particulates, no particulates were observed in either “APP F5 DP” or “APP F6 DP”, which confirmed that even a small excess of non-lipophile-modified antisense strand relative to the C16-modified sense strand could effectively prevent particulate formation. Indeed, calcium exchange data from multiple APP-targeting siRNA duplexes demonstrated that particulate (pellet) only formed when the C16 lipophile-modified sense strand was in relative excess in the duplex (when sense strand levels exceeded antisense strand levels). There is therefore a robust benefit to having a slight excess of the antisense strand (highly hydrophilic) in duplex samples, especially in those subjected to calcium exchange, such as siRNA formulations prepared for CNS dosing. Preparation of such formulations with a molar excess of antisense strand relative to sense strand can therefore help mitigate against risk of particulate formation in such lipophilic moiety-modified siRNA formulations.

### Example 5: Hydrophobicity and Divalent Ions Both Impacted Particulate Formation of Lipid-Containing Molecules

The impact of hydrophobicity and divalent ion content upon the particulate formation observed herein was investigated by assessing whether any of the following altered particulate formation: (1) changes in the position of the C16 modification (whether at an internal residue (i.e., C16 attached at the internal sixth position of sense strand) or upon a terminal residue); (2) changes in lipophilic chain length (comparisons between C10, C16 and C22 chain lengths); and (3) changes in the divalent ion (i.e., calcium vs. magnesium were compared herein).

The robustness of the mitigation strategies identified above (e.g., having antisense strand in excess relative to sense strand during annealing) in molecules with various lipophilic modification(s) and in the presence of divalent ions was also examined.

Such further studies involved production of the four distinct double-stranded agents depicted in FIG. 1A. While baseline duplex sequences were identical and shared an identical antisense strand (A-882382) across all four duplexes, the four duplexes were notably distinguished in the following manner: (1) the AD-454844 duplex harbors a 2′-O—C16 modification at the sixth residue from the 5′-terminus of the sense strand (A-882381); (2) the AD-1302922 shifts the 2′-O—C16 modification to the 5′-terminal residue of the sense strand (A-2364988) of the AD-1302922 duplex; (3) the AD-1302923 duplex harbors a 2′-O-docosanyl-cytidine-3′-phosphate (C22) modification at the sixth residue from the 5′-terminus of the sense strand (A-2365995); and (4) the AD-1999409 duplex harbors a 2′-O-decyl-cytidine-3′-phosphate (C10) modification at the sixth residue from the 5′-terminus of the sense strand (A-3724055). Material production therefore involved synthesis of five distinct single strands to produce the four duplexes, with sense (lipid-containing) strands produced in excess.

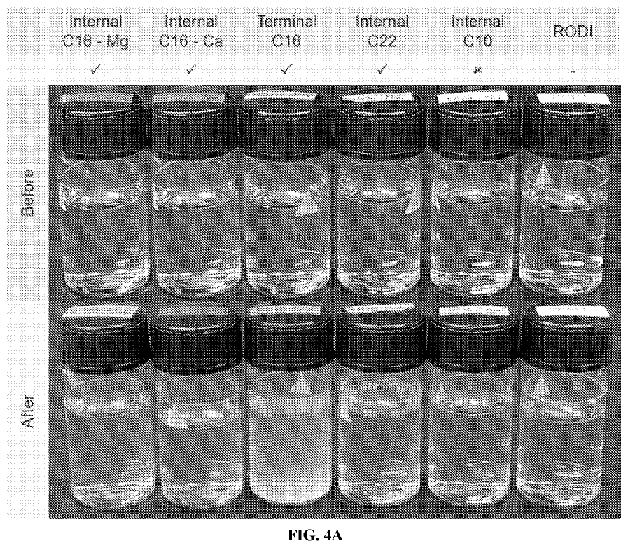

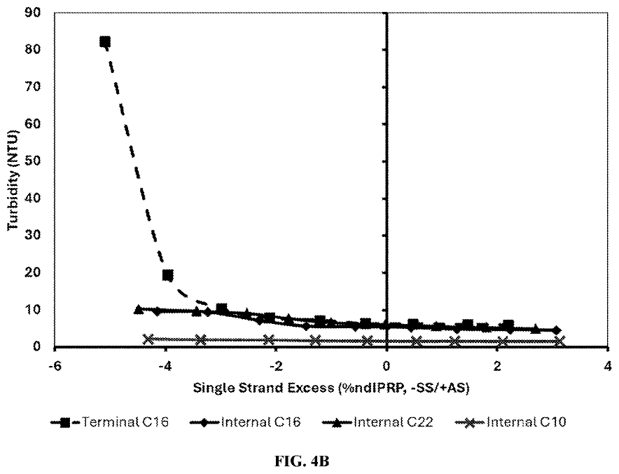

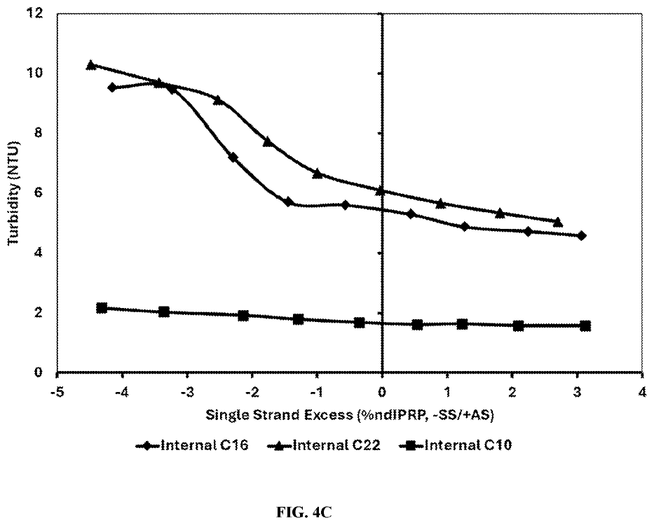

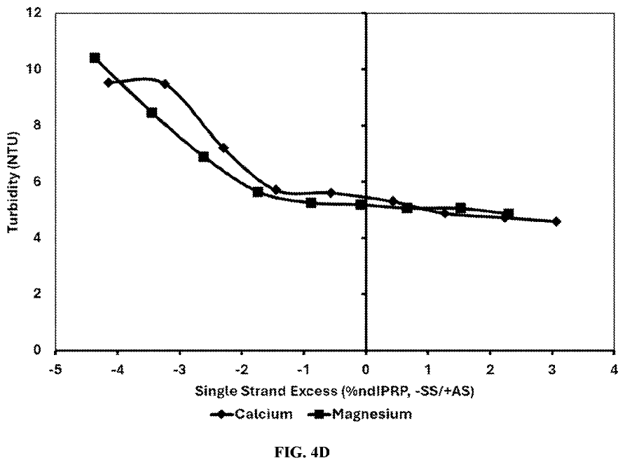

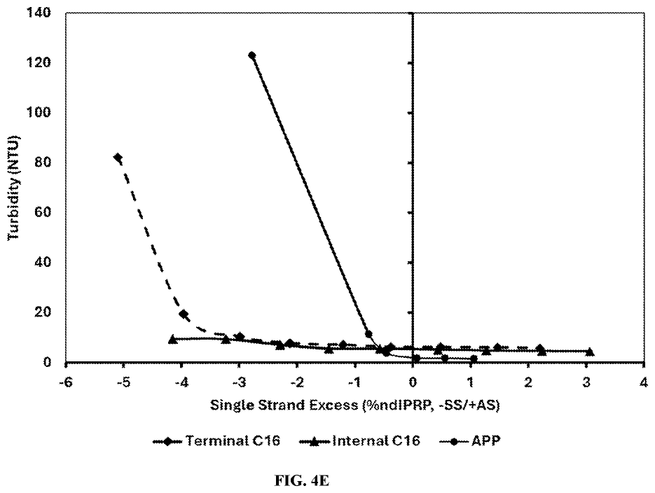

Once the single strands of each of the four duplexes were produced, all four duplexes were respectively annealed and subjected to calcium precipitation assessment, which involved calcium exchange, assessment of precipitate formation, and subsequent precipitate rescue. One C16-modified duplex was also subjected to magnesium precipitation assessment, which involved magnesium concentration screening, magnesium exchange, assessment of precipitate formation, and subsequent precipitate rescue, in parallel with calcium exchange-treated duplex solutions. To perform the assessments, each duplex was produced with a 5% sense strand excess and underwent the ultrafiltration procedure to exchange counter ion with high divalent salt concentration solution (20 mM CaCl₂) or 50 mM MgCl₂) and then low salt concentration solution (1 mM CaCl₂) or MgCl₂)), which produced a turbid suspension of different extent by visual inspection, for each duplex assayed (FIG. 4A). By visual assessment, sodium solution forms of each duplex were clear, with no visible differences between duplex-containing samples and controls observed (FIG. 4A, top row). After divalent counter ion exchange, turbidity was observed in all conditions, except for the internal-C10 duplex, which did not have obvious visual differences before and after exchange (FIG. 4A, bottom row). Turbidity in the terminal-C16 duplex solution was noted as significantly more than in solutions of other duplexes, and the terminal-C16 duplex precipitate tended to settle into a layer over time (FIG. 4A).

In an initial rescue experiment, varying volumes of the complementary antisense strand were added to the duplex suspension, and the samples were annealed, which achieved precipitate rescue of all duplexes examined (FIG. 4B). Turbidity measurements were used as a semi-quantitative measurement of precipitate rescue effectiveness, in combination with non-denaturing ion-pair reversed-phase high performance liquid chromatography (nd-IPRP) analysis. Notably, the terminal C16-containing duplex with sense strand excess exhibited a significantly higher turbidity than any internal lipid-containing duplex at sense strand excess, regardless of the lipid length (as an internal C22-modified duplex displayed less turbidity than the terminal C16-containing duplex). Location of the lipid modification therefore had a significant impact on the turbidity measurements. Despite the large difference observed, the observed turbidity of the terminal C16-containing duplex solution could still be rescued when titrated into antisense strand excess (FIG. 4B). To examine further whether lipophilic moiety chain length could impact turbidity levels under conditions of sense strand excess and counter ion presence, solutions having an internal C10-containing duplex, an internal C16-containing duplex, or an internal C22-containing duplex were prepared and compared for relative turbidity. While both internal C16-containing and internal C22-containing duplexes showed elevated levels of turbidity relative to an internal C10-containing duplex (which showed little to no precipitation) at all sense strand:antisense strand ratios examined, the internal C22-containing duplex exhibited only slightly increased turbidity relative to a corresponding internal C16-containing duplex (FIG. 4C, where C16- and C22-containing duplexes did not show significant trend differences). All such observed turbidity effects could be rescued by elevating antisense strand levels relative to sense strand levels. Such results indicated that short chains (C10) have a low turbidity even in sense strand excess, and that longer chains (C22) did not have significant impact on the severity of the turbidity.

The impact of counter ion upon precipitate formation/turbidity was then assessed. Both calcium- and magnesium-exchanged C16 duplexes exhibited similar turbidity behaviors and were rescued in a similar trend (by eliminating sense strand excess; FIG. 4D). There did not appear to be any significant difference between the two counter ions (calcium vs. magnesium as divalent cations), so the turbidity behavior was likely more impacted by sequence and/or modifications (FIG. 4D).

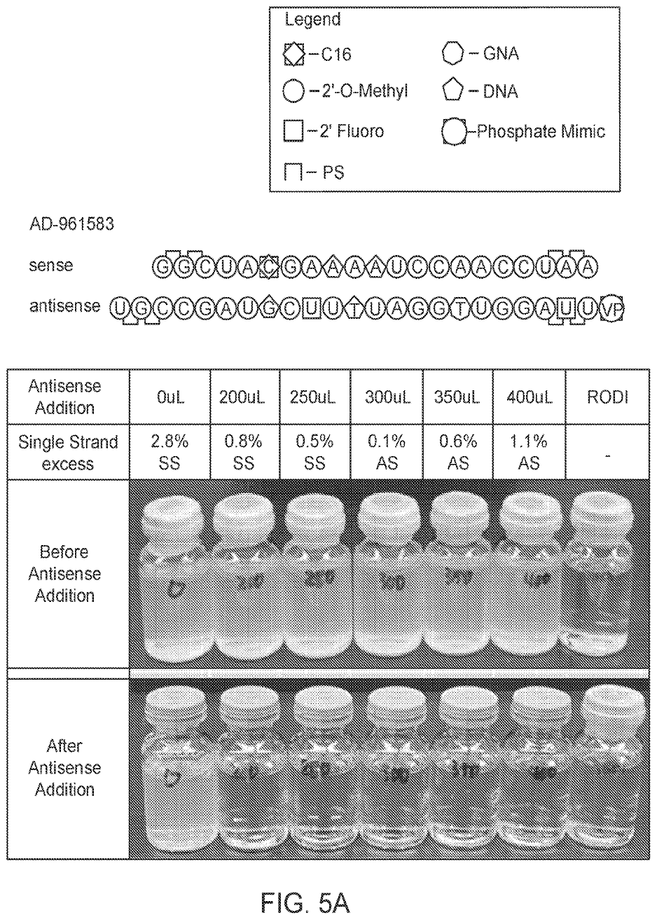

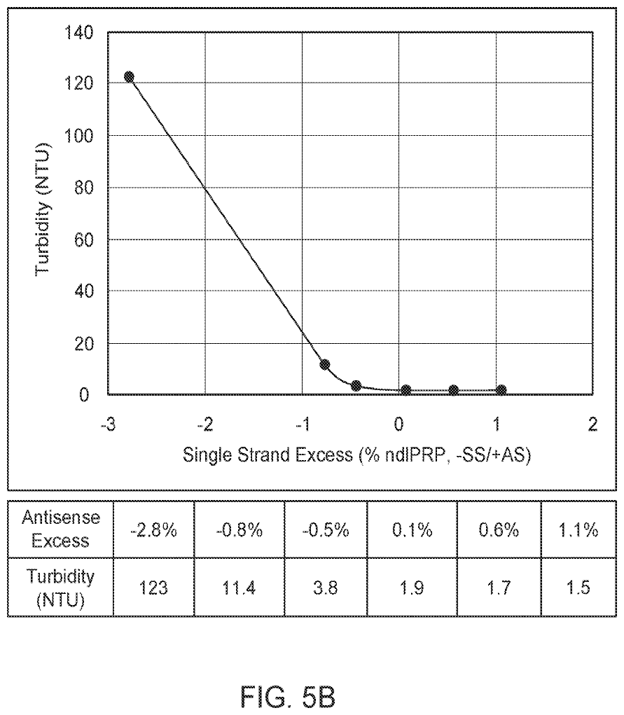

Sequence variations between duplexes harboring parallel nucleotide modification patterns were examined and identified also to impact precipitate formation and associated levels of turbidity. Turbidity of the terminal C16-modified AD-1302922 APP-targeting duplex and of the internal C16-modified AD-454844 APP-targeting duplex, which share common sequences and identical antisense strands, were compared in solution with a distinct, internal C-16 modified APP-targeting duplex, AD-961583 (shown in FIG. 5A and described in additional detail below). When compared to precipitate rescue data for the AD-961583 duplex, both internal and terminal C16-modified duplexes AD-1302922 and AD-454844, respectively, exhibited far less turbidity than the AD-961583 duplex (FIG. 4E). Since the internal C16-modified AD-454844 APP-targeting duplex and the internal C-16 modified APP-targeting duplex AD-961583 shared similar chemistries and lipid positioning, these results indicated that sequence could exert a significant impact on duplex precipitate formation.

As demonstrated above, duplexes with almost all studied chemistries/modifications exhibited turbidity increases when sense strand was in excess and after counter ion exchange, yet all such turbidity was successfully rescued by addition/titration of antisense strand. An example of an exception was an internal-C10-containing duplex, which did not exhibit turbidity/precipitate formation. Impacts on precipitate formation included, for example: (1) lipid position, for example, a terminal C16 lipid-containing duplex was significantly more turbid than internal C16 lipid-containing or internal C22 lipid-containing duplexes; (2) hydrophobicity—for example, longer lipid lengths than C16 (i.e., C22) did not exhibit significant differences in turbidity as compared to C16 lipid-containing duplexes, shorter lengths (C10 lipid-modified duplexes) were observed to have effectively no observable precipitation; and (3) counter ion, for example, no difference in turbidity behavior and rescue was observed between calcium and magnesium. For solutions that include a counter ion (e.g., divalent cation such as calcium or magnesium), preparing and maintaining a 1-2% antisense strand excess during duplex preparation/titration should ensure that no such particulates are formed.

### Example 6: Confirmation of Antisense Strand Excess Ranges in Additional APP-Targeting Duplexes

The impact of antisense strand excess upon rescuing turbidity in counter ion-containing duplex solutions was examined and confirmed for the internal C16 lipophile-containing AD-961583 APP-targeting duplex. For the APP-targeting AD-961583 duplex, precipitate formation and associated turbidity was observed for a series of solutions prepared such that there was a 2.8% sense strand excess relative to antisense strand (FIG. 5A, top row of vials, all turbid, alongside a control duplex-free vial). When sufficient antisense strand was added to such vials to reduce relative sense strand levels below a 1% excess of sense strand relative to antisense strand (addition of 200 μL of antisense strand resulted in a reduction of sense strand excess to a 0.8% sense strand excess; addition of 250 μL of antisense strand resulted in a reduction of sense strand excess to a 0.5% sense strand excess; addition of 300 μL of antisense strand converted the initial sense strand excess to a 0.1% antisense strand excess; addition of 350 μL of antisense strand converted the initial sense strand excess to a 0.6% antisense strand excess; addition of 400 μL of antisense strand converted the initial sense strand excess to a 1.1% antisense strand excess), rescue of precipitate and associated turbidity was uniformly achieved (FIG. 5A, bottom row of vials, in which all solutions that were administered antisense strand were clarified). In addition to visual inspection, turbidity levels of titrated samples were quantitated and plotted (FIG. 5B). Based on calcium exchange, antisense titration, and the current turbidity study, a 1-2% antisense strand excess in duplex annealing should likely be employed during manufacture of APP-targeting duplexes, to mitigate any potential particulate formation in duplex solutions.

### Example 7: Examination of Antisense Strand Excess Ranges in Superoxide Dismutase (SOD)-Targeting Duplexes

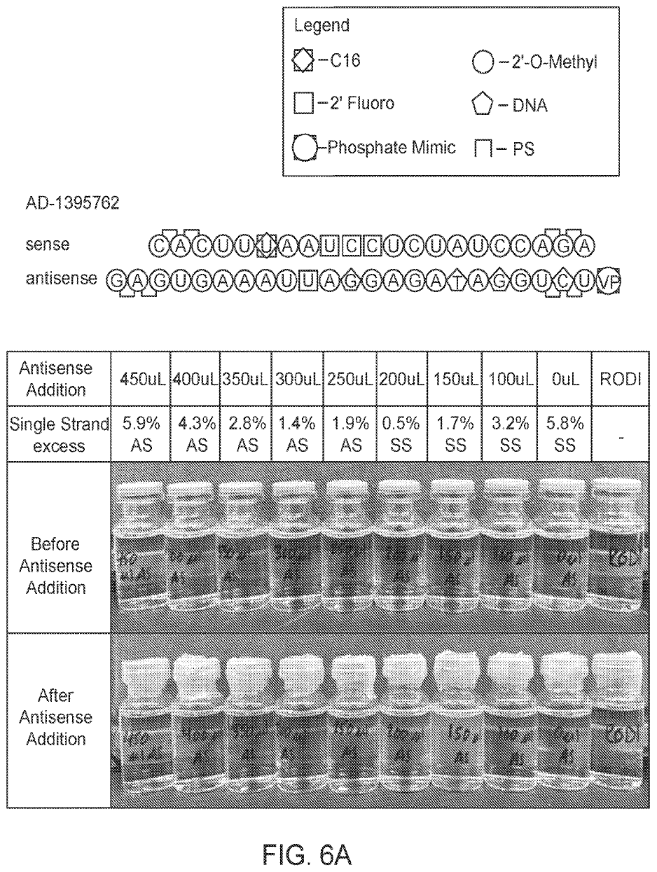

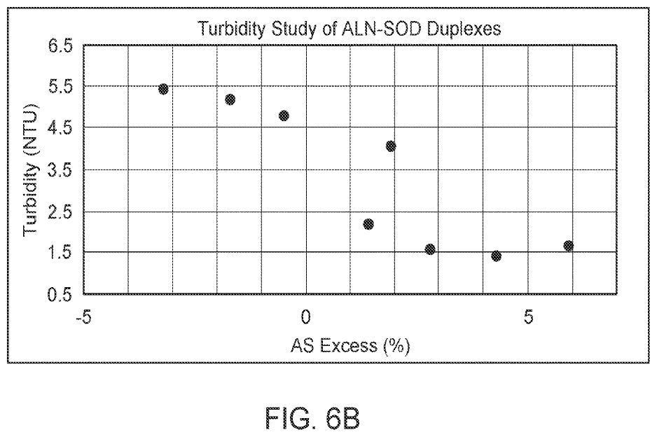

The impact of antisense strand excess upon turbidity in counter ion-containing duplex solutions of an internal C16 lipophile-containing AD-1395762 superoxide dismutase 1 (SOD1)-targeting duplex was examined. Unlike the APP-targeting duplexes examined herein, the AD-1395762 duplex did not form significant particulates even under conditions of sense strand excess of 5.8% and counter ion (divalent cation) presence (FIG. 6A, which showed clear vials at 5.8% antisense strand excess, as well as across the following titration: addition of 100 μL of antisense strand resulted in a reduction of sense strand excess to a 3.2% sense strand excess; addition of 150 μL of antisense strand resulted in a reduction of sense strand excess to a 1.7% sense strand excess; addition of 200 μL of antisense strand resulted in a reduction of sense strand excess to a 0.5% sense strand excess; addition of 250 μL of antisense strand converted the initial sense strand excess to a 1.9% antisense strand excess; addition of 300 μL of antisense strand converted the initial sense strand excess to a 1.4% antisense strand excess (one of the two preceding data points appeared to be an outlier; however, both points have been included for data integrity); addition of 350 μL of antisense strand converted the initial sense strand excess to a 2.8% antisense strand excess; addition of 400 μL of antisense strand converted the initial sense strand excess to a 4.3% antisense strand excess; addition of 450 μL of antisense strand converted the initial sense strand excess to a 5.9% antisense strand excess). In all instances, no turbidity was observed by visual inspection (FIG. 6A). Turbidity levels for solutions containing the AD-1395762 duplex were also quantitated and plotted (FIG. 6B), but the AD-1395762 duplex showed low turbidity values across various single strand excess conditions. Based on calcium exchange, antisense titration, and the current turbidity study, a 1-2% antisense strand excess in duplex annealing will still likely be employed during manufacture of SOD1-targeting duplexes, to mitigate any potential particulate formation in duplex solutions.

### Example 8: Confirmation of Antisense Strand Excess Ranges in Huntingtin-Targeting Duplexes

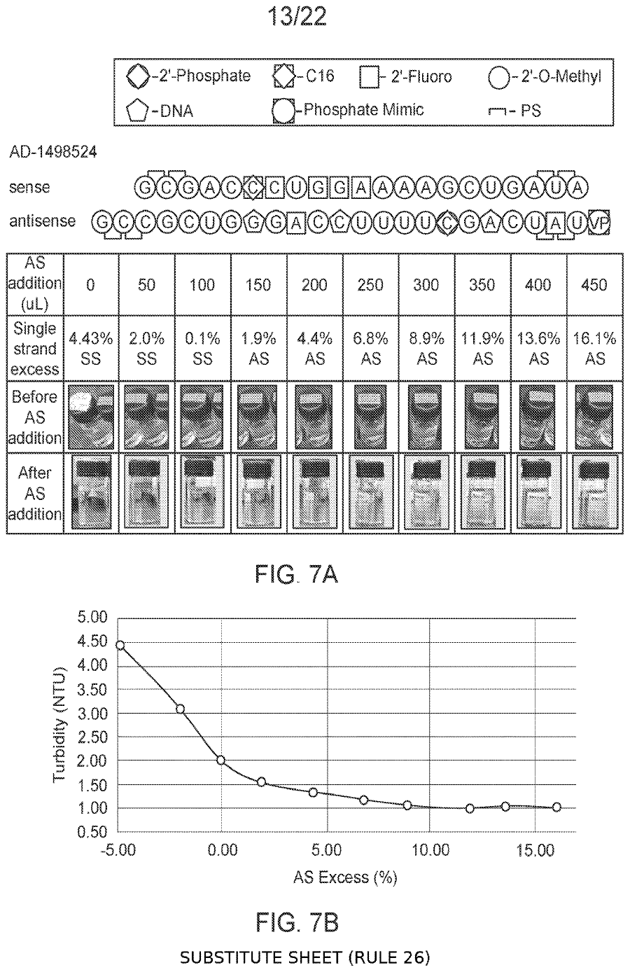

The impact of antisense strand excess upon rescuing turbidity in counter ion-containing duplex solutions was examined for an internal C16 lipophile-containing AD-1498524 huntingtin gene-targeting duplex. For AD-1498524 duplex, while sodium solutions of the duplex at 4.9% sense strand excess exhibited a 0.5 NTU measured turbidity value, the duplex suspension after calcium exchange rose to 78.50 NTU. While AD-1498524 with sense strand excess by 4.9% showed precipitates during calcium exchange process, the precipitates were greatly reduced by heating (data not shown). Precipitate formation and associated turbidity was observed for a series of solutions prepared such that there was a 4.43% sense strand excess relative to antisense strand of the AD-1498524 duplex (FIG. 7A, top row of vials). When sufficient antisense strand was added to such vials to reduce relative sense strand levels below a 1% excess of sense strand relative to antisense strand (addition of 50 μL of antisense strand resulted in a reduction of sense strand excess to a 2.0% sense strand excess; addition of 100 μL of antisense strand resulted in a reduction of sense strand excess to a 0.1% sense strand excess; addition of 150 μL of antisense strand converted the initial sense strand excess to a 1.9% antisense strand excess; addition of 200 μL of antisense strand converted the initial sense strand excess to a 4.4% antisense strand excess; addition of 250 μL of antisense strand converted the initial sense strand excess to a 6.8% antisense strand excess; addition of 300 μL of antisense strand converted the initial sense strand excess to a 8.9% antisense strand excess; addition of 350 μL of antisense strand converted the initial sense strand excess to a 11.9% antisense strand excess; addition of 400 μL of antisense strand converted the initial sense strand excess to a 13.6% antisense strand excess; addition of 450 μL of antisense strand converted the initial sense strand excess to a 16.1% antisense strand excess), rescue of precipitate and associated turbidity was achieved (FIG. 7A, bottom row of vials). In addition to visual inspection, turbidity levels of titrated samples were quantitated and plotted (FIG. 7B). Based on calcium exchange, antisense titration, and the current turbidity study, a 1-2% antisense strand excess in duplex annealing should likely be employed during manufacture of HTT exon 1-targeting duplexes, to mitigate any potential particulate formation in duplex solutions.

### Example 9: Further Assessment of Antisense Strand Excess During Annealing as Preventative of Particulate Formation in the Divalent Cation Solutions

In the above examples, a calcium exchange process was developed and used as an accelerated model for assessment of particulate formation in duplex-containing samples. As identified above, precipitation/particulate formation was observed when the calcium exchange process was performed upon duplex solutions containing an excess of sense strand (relative to antisense strand). A C16 modification of the sense strand (as well as related impurities) was identified as a major component of the precipitate, and precipitation was shown to be rescued by addition of complementary antisense strand to such solutions. For the respective APP- and SOD-targeting duplexes tested above, it was observed that maintenance of a 1-2% antisense strand excess in duplex solutions during annealing prevented particulate formation from occurring.

The impact of hydrophobicity and divalent ion upon particulate formation was examined further in the current example. Specifically, additional investigation of changes in lipophilic modification chain length (C10 versus C16 versus C22), changes in C16 modification position, and changes in divalent cation employed (calcium versus magnesium) was performed. Such assessments were performed to confirm the robustness of the above-identified mitigation strategies (i.e., maintaining an excess of antisense strand during annealing, where the sense strand of a duplex carries a lipophilic modification) in duplexes having various lipophilic modification patterns, and in the presence of divalent ions (specifically, calcium and magnesium).

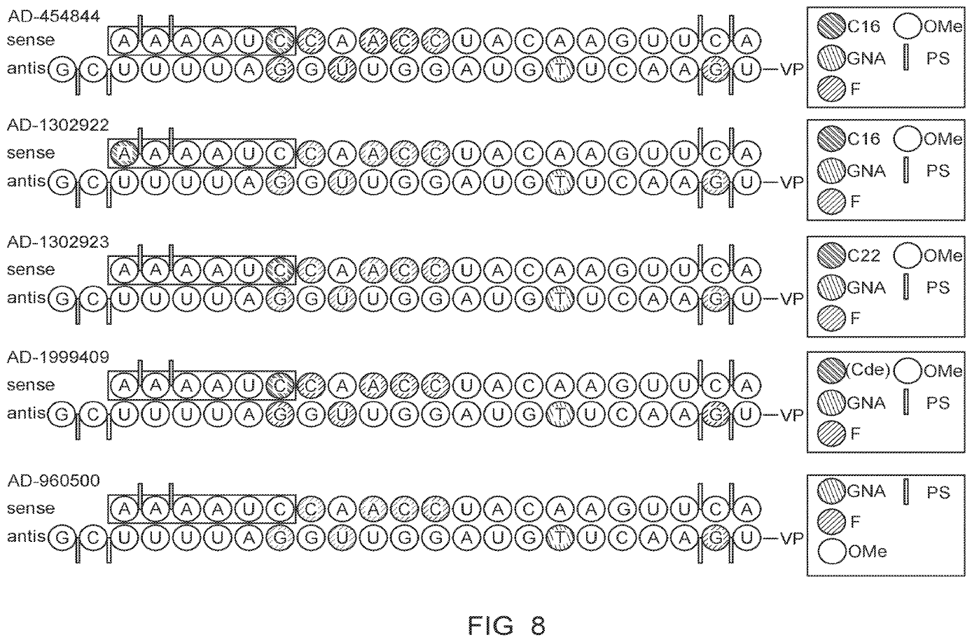

A total of five distinct duplexes were produced for the current experiments (FIG. 8, which shows the same four duplexes shown in FIG. 1A, together with a corresponding non-lipophile-modified duplex, AD-960500, added for the current comparisons). Each duplex shared the same antisense strand sequence (A-882382), while sense strand sequences varied only at the 5′ n6 or n1 positions, depending upon the different lipophilic modification used:
(1) the first duplex, AD-454844, features a sense strand (A-882381) having an internal (n6 position) C16 lipophile;
(2) the second duplex, AD-1302922, features a sense strand (A-2364988) having a terminal (n1 position) C16 lipophile;
(3) the third duplex, AD-1302923, features a sense strand (A-2365995) having an internal (n6 position) C22 lipophile;
(4) the fourth duplex, AD-1999409, features a sense strand (A-3724055) having an internal (n6 position) C10 lipophile; and
(5) the fifth duplex, AD-960500, features a sense strand (A-1690014) having no lipophilic modification.

Each of the above-described five duplex preparations were subjected to calcium precipitation assessment (including calcium ion exchange and precipitate rescue assessments), while two parallel duplex preparations (AD-454844 and AD-1302922) were separately subjected to magnesium precipitation assessment (including magnesium ion exchange and precipitate rescue assessments).

The precipitation investigation process involved three steps:

(A) Duplex making and divalent cation exchange, in which: each duplex was produced with a 5% sense strand excess; all five duplexes were subjected to calcium exchange; and two duplexes containing internal or terminal C16 lipophilic modifications (AD-454844 and AD-1302922 duplexes, respectively) also were subjected to magnesium exchange. Cation exchange processes involved exchange of sodium cations with respective divalent cation, using an ultrafiltration process in which sodium cations were exchanged out with the respective divalent cation using an ultrafiltration process with high salt concentration solution (20 mM CaCl₂) or 50 mM MgCl₂) followed by a low salt concentration solution (1 mM CaCl₂) or MgCl₂).
(B) Performance of rescue experiments, in which varying volumes of the complementary antisense strand was added to each duplex suspension, and the samples were annealed to perform precipitate rescue.
(C) Turbidity measurement, which was used as a semi-quantitative measurement of precipitate rescue effectiveness, in combination with non-denaturing ion pairing-reversed phase (nd-IPRP) chromatography analysis.

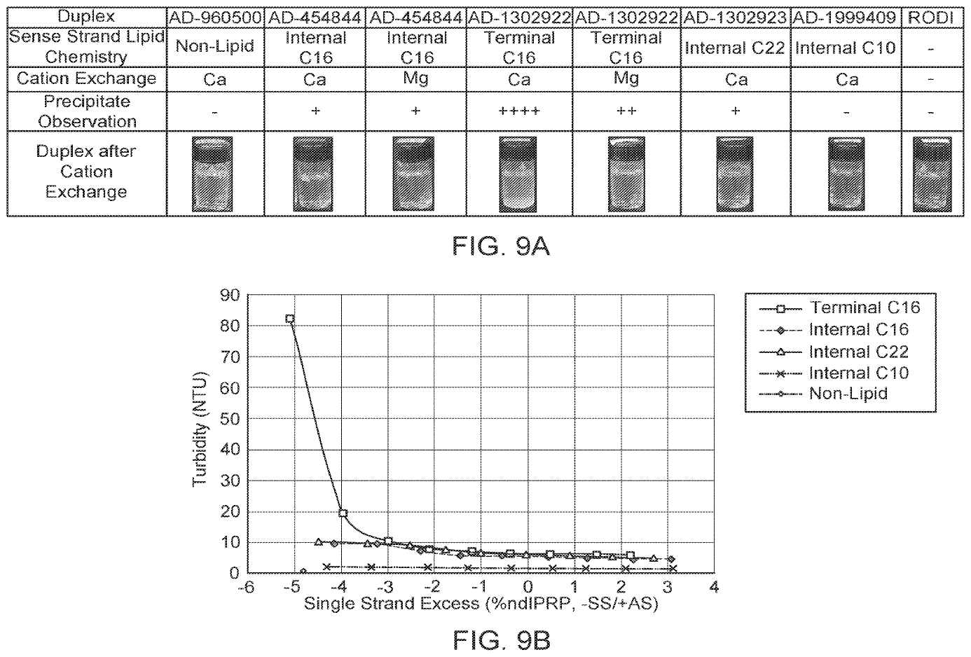

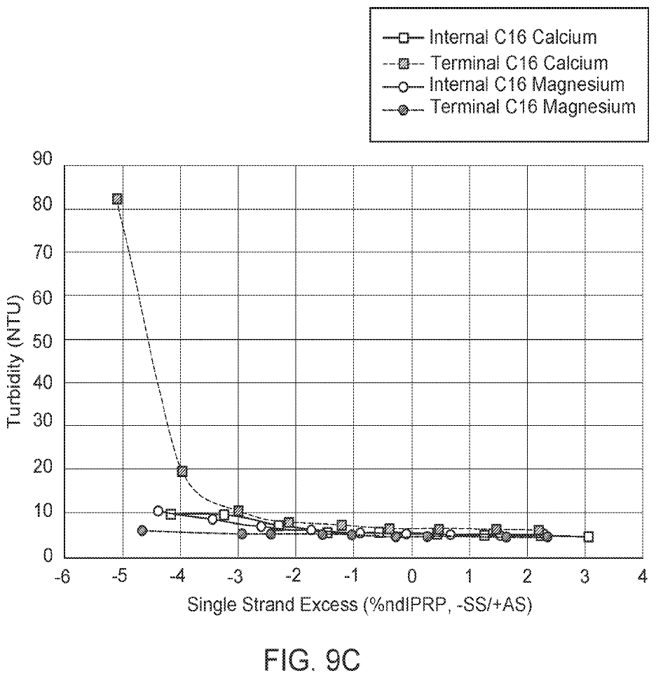

After performance of the above-described cation exchanges, visual assessment of the various solutions of duplexes was performed. Sodium solution forms of each tested duplex were clear, with no visible differences between sodium solution preparations detected. However, at least some turbidity was observed after cation exchange in most solutions, with the exception of the internal-C10 (AD-1999409) and non-lipophile-modified (AD-960500) duplexes (FIG. 9A). Notably, observed turbidity in the terminal-C16 duplex (AD-1302922) calcium exchange solution was significantly more than in other tested duplexes, and the terminal-C16 duplex (AD-1302922) calcium exchange solution was also observed to settle into a layer of precipitation after time (FIG. 9A).

As identified above, even highly turbid duplex solutions (e.g., calcium-exchanged AD-1302922, possessing a terminal C16 lipophile and which was made at sense strand excess, thereby establishing a significantly higher turbidity than other examined duplexes) were capable of being rescued via introduction of an excess of complementary antisense strand to such turbid duplex solutions (FIG. 9B). In addition to the calcium-exchanged AD-1302922 duplex, it was also notable that calcium-exchanged internal C22 and internal C16-containing duplexes showed similar turbidity levels to one another (FIG. 9B). Meanwhile, a calcium-exchanged internal C10-modified duplex exhibited few if any precipitates (FIG. 9B). Hydrophobicity requirements of lipophilic modifications (C16 or longer carbon chain) in the duplex therefore impacted precipitation during Ca²⁺ exchange (FIG. 9B).

All observed examples of turbidity could be rescued when duplexes were titrated into antisense excess, including rescue of calcium-exchanged terminal C₁₆-containing duplexes, despite this duplex's much higher levels of turbidity (FIG. 9B).

Duplex solutions were further assessed for whether the form of divalent cation employed (Ca²⁺ or Mg²⁻) in cation exchange reactions impacted turbidity development/particulate formation. In particular, sense strand excess solutions of the AD-454844 duplex, having an internal (n6 position) C16 lipophile, and the AD-1302922 duplex, having a terminal (n1 position) C16 lipophile, were each subjected to magnesium exchange and assessed for turbidity/particulate formation. Unlike the above-noted calcium exchange experiments, these magnesium exchange experiments showed little turbidity difference between the two duplexes harboring a C16 lipid modification at different locations (FIG. 9C; n6 position or n1 position). For the AD-454844 duplex having the internal C16 lipophile, sense strand excess under conditions of magnesium exchange showed similar impact on turbidity level as calcium exchange, and turbidity was rescued in a similar trend as the same duplex under conditions of calcium exchange (FIG. 9C). In contrast, for the AD-1302922 duplex having the terminal C16 lipophile, sense strand excess under conditions of magnesium exchange was observed to exert a significantly lower impact on turbidity, as compared to the same duplex under conditions of calcium exchange (FIG. 9C).

In sum, almost all duplexes prepared at sense (lipid-containing) strand excess exhibited turbidity increases upon divalent cation exchange (Ca²⁺ or Mg²⁺). All such observed turbidity was successfully rescued via antisense strand titration of such initially sense strand excess solutions. The only exception to such observations occurred with an internal-C10 lipophile-containing duplex, which showed no turbidity increase after calcium exchange.

Accordingly, the following features were identified to impact the extent of precipitate formation in tested duplexes including, for example: (i) lipid position—for example, the terminal C16 lipophile-possessing duplex was identified as significantly more turbid than a corresponding internal C16 lipophile-possessing duplex following calcium exchange; (ii) lipid length—for example, shorter length lipophile groups (i.e., C10) were observed to have little-to-no observed turbidity upon calcium exchange, whereas longer length lipophile groups (C16 and C22) exhibited increased turbidity after calcium exchange; (iii) divalent cation—for example, magnesium exchange exhibited lower impact on turbidity of sense (C16 lipophile-presenting) strand excess duplex solutions, as compared to calcium exchange performed upon the same duplexes. Magnesium exchange also appeared to be less sensitive to the position of the C16 lipophile within the sense strand, as compared to calcium exchange.

A particulate mitigation strategy of maintaining 1-2% antisense (non-lipid) strand excess during duplex titration can therefore be generalized to other lipid-containing duplexes, to mitigate particulate risk in formulations containing divalent cations.

### Example 10: Duplex Annealing

### A. APP-Duplex

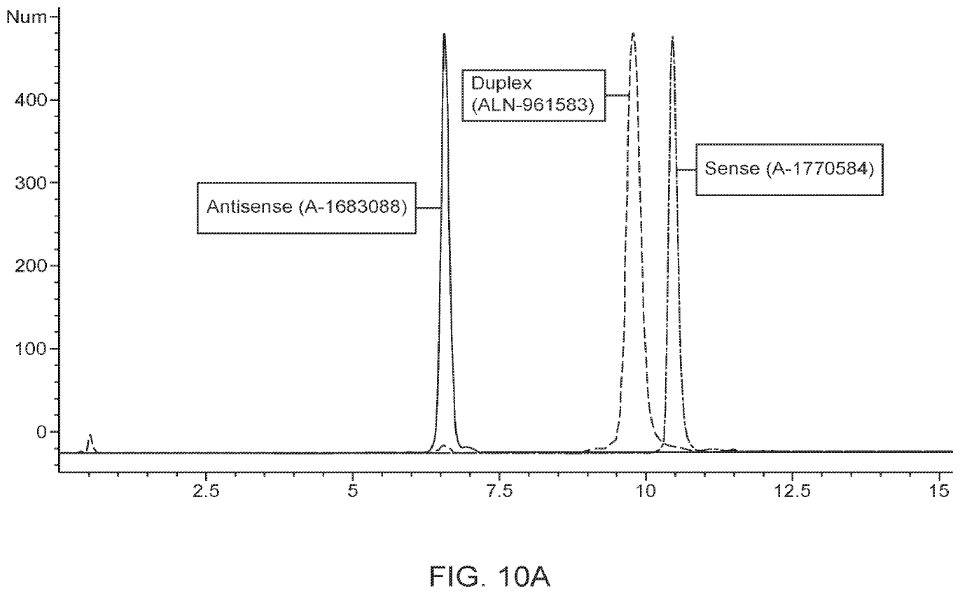

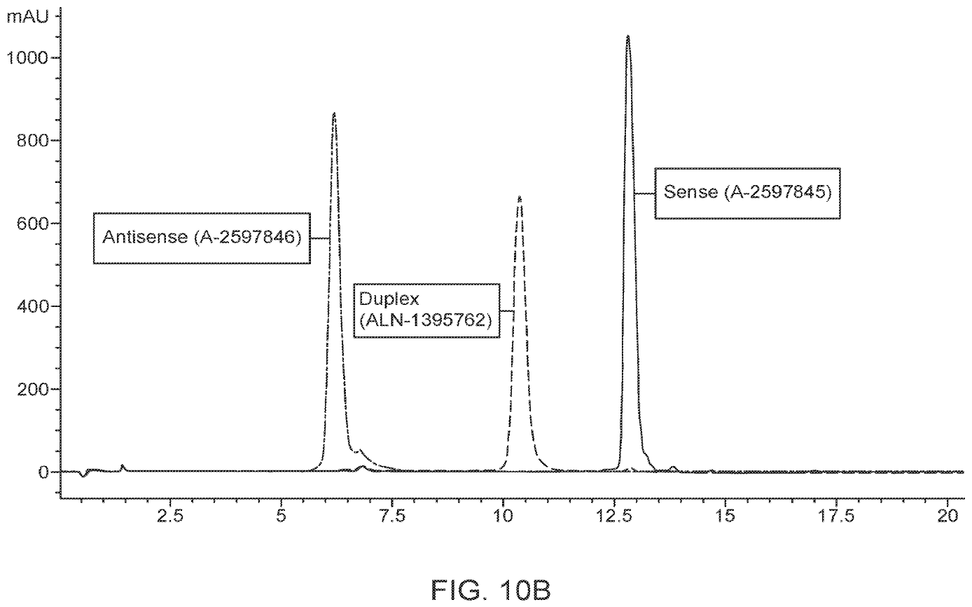

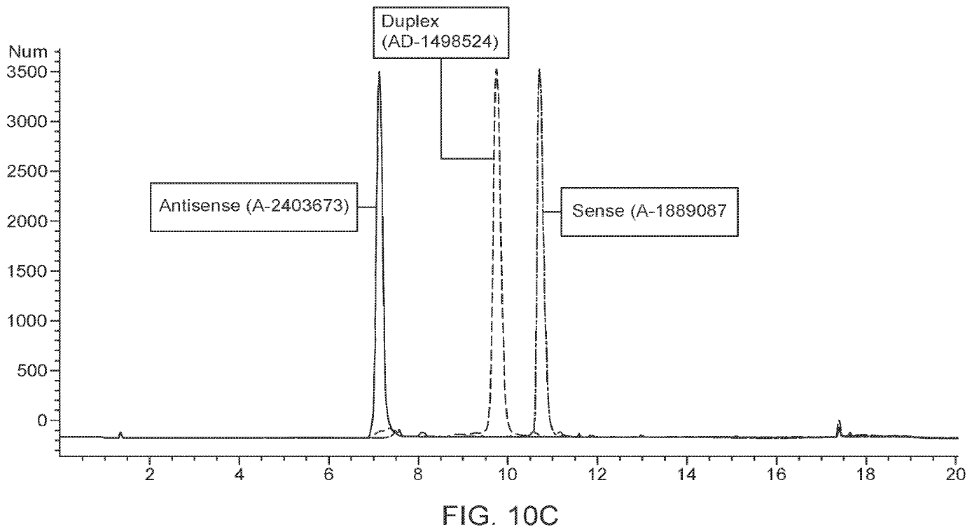

To ensure a controlled ratio between the two single strands, the solution of one strand was titrated with the other until 1-2% single strand excess (antisense strand excess). The titration process was monitored through non-denaturing ion-paring reverse phase (IPRP) HPLC. After annealing, the duplex solution was filtered through a 0.2 μm filter. The solution may be lyophilized for storage and compounding. An exemplary non-denaturing IPRP chromatogram is presented in FIG. 10A. The HPLC results were used to calculate the percentage of single strand excess.

### B. SOD-Duplex

To ensure a controlled ratio between the two single strands, the solution of one strand was titrated with the other strand until 1-2% single strand excess (antisense strand excess). The titration process was monitored through non-denaturing ion-paring reverse phase (IPRP) HPLC. Sense strand excess in the duplex to minimize solubility risk in drug product. After annealing, the duplex solution was filtered through a 0.2 μm filter. The solution may be lyophilized for storage and compounding. An exemplary nondenaturing IPRP chromatogram is presented in FIG. 10B. The HPLC results were used to calculate the percentage of single strand excess.

### C. HTT-Duplex

To ensure a controlled ratio between the two single strands, the solution of one strand was titrated with the other strand until 1-2% single strand excess (antisense strand excess). The titration process was monitored through non-denaturing ion-paring reverse phase (IPRP) HPLC. After annealing, the duplex solution was filtered through a 0.2 μm filter into a HDPE sterile container of appropriate volume. The solution may be lyophilized for storage and compounding. An exemplary chromatogram from the duplex annealing method is presented in FIG. 10C. The HPLC results were used to calculate the percentage of single strand excess.

### Example 11: Formulation (DP) Details

The formulation details are as follows and are provided in Table 5. An excipient solution was prepared by adding sodium chloride, potassium chloride, magnesium chloride hexahydrate, and calcium chloride dihydrate to a compounding container followed by addition of water for injection (WFI) with stirring. The excipient solution was filtered through a 0.2-micron filter and stored at 2-8° C. until day of compounding. Drug Substance (DS), for example, prepared as in Example 10 and lyophilized, was equilibrated at room temperature (15-25° C.) for 2-24 h and compounded using the excipient solution. The pH was measured and, if necessary, adjusted with NaOH (0.25 N) or HCl (0.1N) to meet the acceptable range (6.6-7.0, for target 6.8). The excipient solution was used to dissolve the DS, and used to arrive at final concentration of 60 mg/mL (free acid basis). The solution is filtered through a 0.2-micron filter and stored at 2-8° C. until day of fill. Release osmolality of the formulations was 210-390 mOsm/kg, and the release particulate was >=10 um, NMT 6000 per container; >=25 um, NMT 600 per container where “container” is 5 mL in a 10R vial.

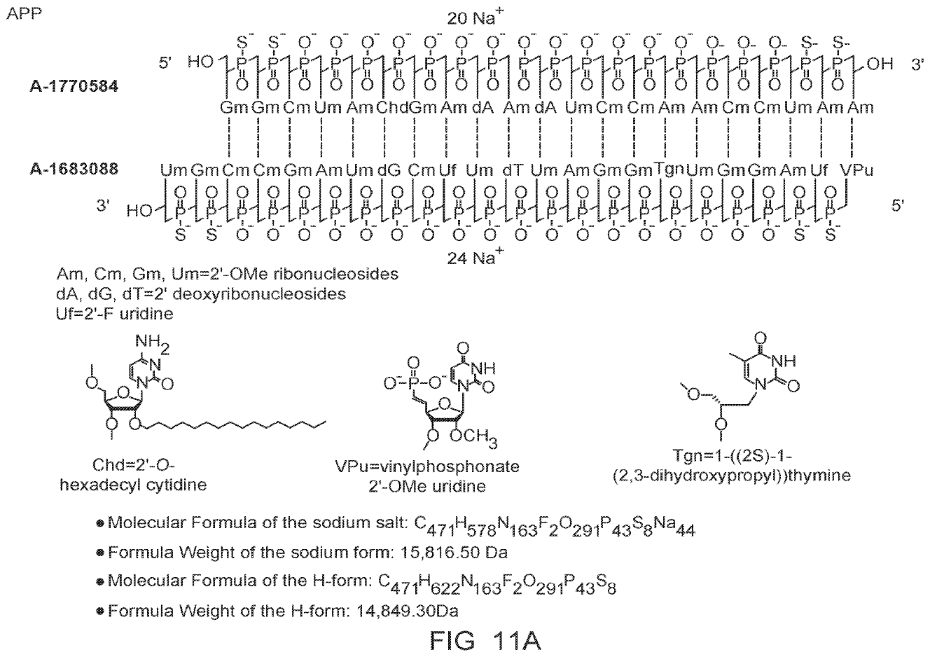

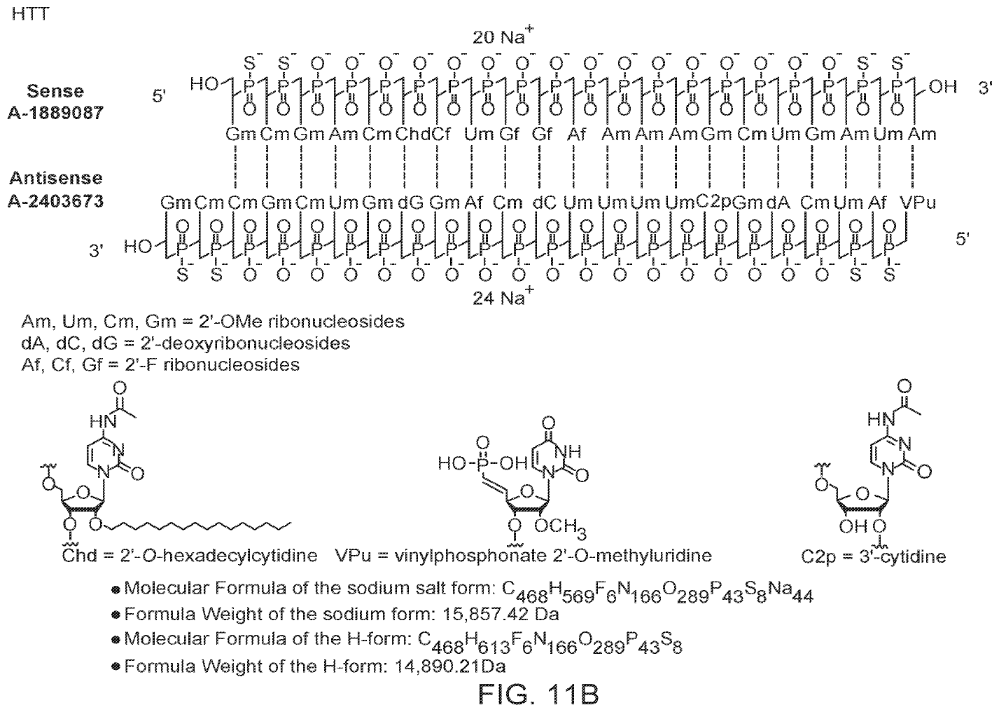

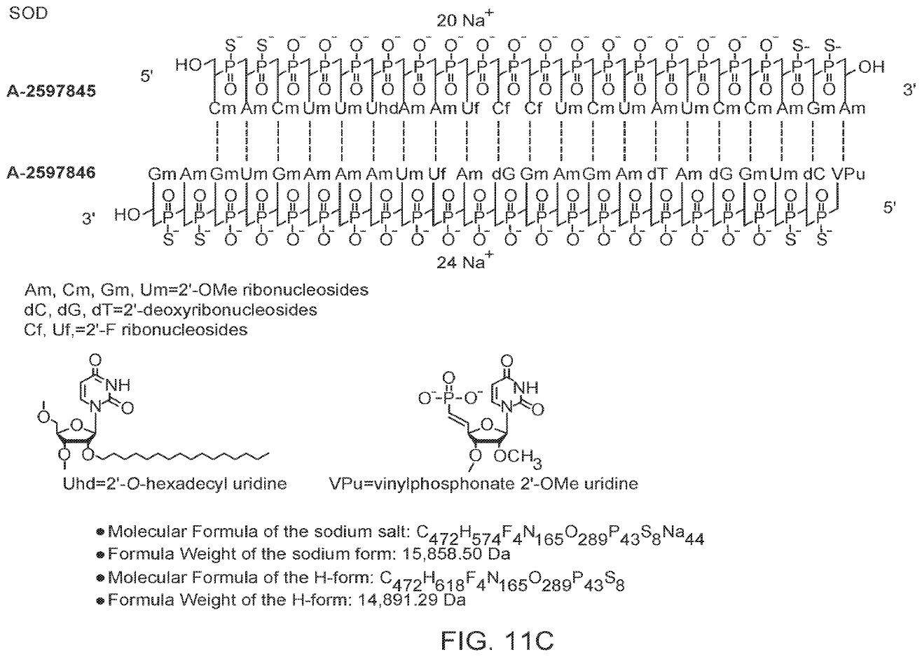

Information on the sodium salt forms of the formulations, including structure and molecular formula, are shown in FIG. 11A for APP duplex AD-961583, FIG. 11B for HTT duplex AD-1498524, and FIG. 11C for SOD1 duplex AD-1395762.

TABLE 5
Formulation Summary

| Component | AD-961583 | AD-1395762 | AD-1498524 |
|---|---|---|---|
| API (mg/mL)* | 60 | 60 | 60 |
| NaCl (g/L) | 5.70 | 5.26 | 5.23 |
| KCl (g/L) | 0.14 | 0.13 | 0.13 |
| MgCl₂*6H₂O (g/L) | 0.10 | 0.10 | 0.10 |
| CaCl₂*2H₂O (g/L) | 1.91 | 1.91 | 2.04 |

*excess AS strand controlled during annealing of DS

### Example 12. Comparative APP Formulation Stability

Stability testing of APP DP (see formulation in Table 5) without phosphate buffer was conducted out to 24-month time point under the following conditions:
2-8° C.
25° C.±2° C./60%±5% relative humidity (RH)
30° C.±2° C./75%±5% relative humidity
40° C.±2° C./75%±5% relative humidity
   The abbreviations used in the stability data tables are defined in Table 6. The DP was prepared with DS annealed with a 1-2% antisense strand excess. The results for a representative lot are shown in Tables 7-10.

TABLE 6
Abbreviations Used in Stability Data

| Abbreviation | Definition |
|---|---|
| AX | Anion-Exchange |
| CCS | Clear, colorless solution |
| CCYS | Clear, colorless to yellow solution |
| CPYS | Clear, Pale Yellow Solution |
| CSY or CSYS | Clear, slightly yellow solution |
| CYS | Clear yellow solution |
| DP | Drug Product |
| EFFP | “Essentially free of foreign particles” or |
| | “Essentially free of fine particles” |
| FFP | Free of Foreign Particles |
| HIAC | (High Accuracy) particle counter |
| IPRP | Ion-Pair Reverse Phase |
| LC | Liquid Chromatography |
| LSNPO | Long, straight, needle-like particulates observed |
| DS | Drug substance |

TABLE 7
Stability Data: ALN-APP DP Representative Lot Stored at 2-8° C.

| | | | | Storage Time (months) | | | | |
|---|---|---|---|---|---|---|---|---|
| Attribute | Method | Acceptance Criterion | | 0 | 1 | 3 | 6 | 9 |
| Appearance | Visual Inspection | Clear, colorless to yellow | | CCS | CCS | CCS | CCS | CCS |
| | | solution that may contain trace | | EFFP | EFFP | TPS | EFFP | EFFP |
| | | amounts of fine particles | | | | | | |
| Purity | IPRP LC UV | | | | | | | |
| Duplex Purity | (non-denaturing) | NLT 90.0 area % | | 97.8 | 97.7 | 97.9 | 98.0 | 97.8 |
| Impurities | | NMT 10.0 area % | | 2.2 | 2.2 | 2.1 | 2.0 | 2.1 |
| (Sum of all other peaks ≥0.050 area %) | | | | | | | | |
| Assay (free acid) | UV | 54-66 | mg/mL | 60 | 60 | 60 | 60 | 60 |
| | spectrophotometry | | | | | | | |
| Osmolality | Ph. Eur. 2.2.35; | 210-390 | mOsm/kg | 314 | 313 | 313 | 311 | 315 |
| | USP <785> | | | | | | | |
| pH | Ph. Eur. 2.2.3; | 6.0-7.5 | | 6.6 | 6.6 | 6.5 | 6.6 | 6.6 |
| | USP <791> | | | | | | | |
| Particulate matter | Ph. Eur. 2.9.19; | | | | | | | |

| | | | | | | | |
|---|---|---|---|---|---|---|---|
| ≥10 μm | USP <788> | NMT 6,000 per container | 61 | 24 | 36 | 53 | 67 |
| ≥25 μm | | NMT 600 per container | 0 | 1 | 1 | 1 | 1 |

TABLE 8
Stability Data: ALN-APP DP Representative Lot Stored at 25° C. ± 2° C./60% ± 5% RH

| | | | | Storage Time (months) | | | | |
|---|---|---|---|---|---|---|---|---|
| Attribute | Method | Acceptance Criterion | | 0 | 1 | 3 | 6 | 9 |
| Appearance | Visual Inspection | Clear, colorless to yellow | | CCS | CCS | CCS | CCS | CCS |
| | | solution that may contain trace | | EFFP | EFFP | TPS | TPS | EFFP |
| | | amounts of fine particles | | | | | | |
| Purity | IPRP LC UV | | | | | | | |
| Duplex Purity | (non-denaturing) | NLT 90.0 area % | | 97.8 | 97.7 | 98.0 | 97.9 | 97.9 |
| Impurities | | NMT 10.0 area % | | 2.2 | 2.2 | 2.0 | 2.0 | 2.1 |
| (Sum of all other peaks ≥0.050 area %) | | | | | | | | |
| Assay (free acid) | UV | 54-66 | mg/mL | 60 | 61 | 60 | 60 | 60 |
| | spectrophotometry | | | | | | | |
| Osmolality | Ph. Eur. 2.2.35; | 210-390 | mOsm/kg | 314 | 313 | 315 | 311 | 314 |
| | USP <785> | | | | | | | |
| pH | Ph. Eur. 2.2.3; | 6.0-7.5 | | 6.6 | 6.7 | 6.6 | 6.6 | 6.6 |
| | USP <791> | | | | | | | |
| Particulate matter | Ph. Eur. 2.9.19; | | | | | | | |

| | | | | | | | |
|---|---|---|---|---|---|---|---|
| ≥10 μm | USP <788> | NMT 6,000 per container | 61 | 64 | 225 | 133 | 99 |
| ≥25 μm | | NMT 600 per container | 0 | 0 | 2 | 2 | 6 |

TABLE 9
Stability Data: ALN-APP DP Representative Lot Stored at 30° C. ± 2° C./75% ± 5% RH

| | | | | Storage Time (months) | | | | |
|---|---|---|---|---|---|---|---|---|
| Attribute | Method | Acceptance Criterion | | 0 | 1 | 3 | 6 | 9 |
| Appearance | Visual Inspection | Clear, colorless to yellow | | CCS | CCS | CCS | CCS | CCS |
| | | solution that may contain | | EFFP | EFFP | TPS | EFFP | EFFP |
| | | trace amounts of fine | | | | | | |
| | | particles | | | | | | |
| Purity | IPRP LC UV | | | | | | | |
| Duplex Purity Impurities | (non-denaturing) | NLT 90.0 area % | | 97.8 | 97.7 | 97.9 | 98.0 | 97.9 |
| (Sum of all other peaks ≥0.050 area %) | | NMT 10.0 area % | | 2.2 | 2.2 | 2.0 | 2.0 | 2.0 |
| Assay (free acid) | UV | 54-66 | mg/mL | 60 | 61 | 60 | 59 | 60 |
| | spectrophotometry | | | | | | | |
| Osmolality | Ph. Eur. 2.2.35; | 210-390 | mOsm/kg | 314 | 314 | 313 | 311 | 316 |
| | USP <785> | | | | | | | |
| pH | Ph. Eur. 2.2.3; | 6.0-7.5 | | 6.6 | 6.7 | 6.6 | 6.6 | 6.6 |
| | USP <791> | | | | | | | |
| Particulate matter | Ph. Eur. 2.9.19; | | | | | | | |

| | | | | | | | |
|---|---|---|---|---|---|---|---|
| ≥10 μm | USP <788> | NMT 6,000 per container | 61 | 117 | 312 | 175 | 161 |
| ≥25 μm | | NMT 600 per container | 0 | 2 | 2 | 3 | 6 |

TABLE 10
Stability Data: ALN-APP DP Representative Lot Stored at 40° C. ± 2° C./75% ± 5% RH

| | | | | Storage Time (months) | | | |
|---|---|---|---|---|---|---|---|
| Attribute | Method | Acceptance Criterion | | 0 | 1 | 3 | 6 |
| Appearance | Visual Inspection | Clear, colorless to yellow | | CCS | CCS | CCS | CCS |
| | | solution that may contain trace | | EFFP | EFFP | TPS | TPS |
| | | amounts of fine particles | | | | | |
| Purity | IPRP LC UV | | | | | | |
| Duplex Purity Impurities | (non-denaturing) | NLT 90.0 area % | | 97.8 | 97.8 | 97.9 | 97.9 |
| (Sum of all other peaks ≥0.050 area %) | | NMT 10.0 area % | | 2.2 | 2.2 | 2.0 | 1.9 |
| Assay (free acid) | UV | 54-66 | mg/mL | 60 | 61 | 60 | 60 |
| | spectrophotometry | | | | | | |
| Osmolality | Ph. Eur. 2.2.35; | 210-390 | mOsm/kg | 314 | 314 | 315 | 313 |
| | USP <785> | | | | | | |
| pH | Ph. Eur. 2.2.3; | 6.0-7.5 | | 6.6 | 6.7 | 6.6 | 6.6 |
| | USP <791> | | | | | | |
| Particulate matter | Ph. Eur. 2.9.19; | | | | | | |

| | | | | | | |
|---|---|---|---|---|---|---|
| ≥10 μm | USP <788> | NMT 6,000 per container | 61 | 239 | 674 | 564 |
| ≥25 μm | | NMT 600 per container | 0 | 9 | 5 | 7 |

### Example 13. Comparative SOD1 Formulation Stability

Stability testing of ALN-SOD DP (see formulation in Table 5) was conducted out to 18-month time point under the following conditions:
5±3° C.
25° C.±2° C./60%±5% relative humidity (RH)
30° C.±2° C./75%±5% relative humidity
40° C.±2° C./75% 5% relative humidity

The DP was prepared with DS annealed with a 1-2% antisense strand excess. The results for a representative lot are shown in Tables 11-14.

TABLE 11
Stability Data: ALN-SOD DP Representative Lot Stored at 5 ± 3° C.

| | | | | Storage Time (months) | | | |
|---|---|---|---|---|---|---|---|
| Attribute | Method | Acceptance Criterion | | 0 | 3 | 6 | 9 |
| Appearance | Visual Inspection | Clear, colorless to yellow | | CSYS | CSYS | CSYS | CSYS |
| | | solution, essentially free of | | EFFP | EFFP | EFFP | EFFP |
| | | foreign particles | | | | | |
| Purity | IPRP LC UV | | | | | | |
| Duplex Purity Total | (non-denaturing) | NLT 90.0 area % | | 97.9 | 97.0 | 97.2 | 96.8 |
| Impurities ^{a} | | NMT 10.0 area % | | 2.1 | 3.0 | 2.8 | 3.2 |
| Assay (Free acid) | UV | 54-66 | mg/mL | 61 | 61 | 58 | 60 |
| | Spectrophotometry | | | | | | |
| Osmolality | USP <785>, Ph. | 210-390 | mOsm/kg | 292 | 295 | 292 | 294 |
| | Eur. 2.2.35 | | | | | | |
| pH | USP <791>, Ph. | 6.0-7.5 | | 6.8 | 6.7 | 6.7 | 6.7 |
| | Eur. 2.2.3 | | | | | | |
| Particulate Matter | Ph. Eur 2.9.19 | | | | | | |

| | | | | | | |
|---|---|---|---|---|---|---|
| ≥10 μm | USP <788> | NMT 6000 per container | 64 | 7 | 42 | 21 |
| ≥25 μm | | NMT 600 per container | 1 | 0 | 2 | 1 |

^{a} Sum of all other peaks ≥0.050 area %

TABLE 12
Stability Data: ALN-SOD DP Representative Lot Stored at 25° C. ± 2° C./60% ± 5% RH

| | | | | Storage Time (months) | | | |
|---|---|---|---|---|---|---|---|
| Attribute | Method | Acceptance Criterion | | 0 | 3 | 6 | 9 |
| Appearance | Visual Inspection | Clear, colorless to yellow | | CSYS | CSYS | CSYS | CSYS |
| | | solution, essentially free of | | EFFP | EFFP | EFFP | EFFP |
| | | foreign particles | | | | | |
| Purity | IPRP LC UV | | | | | | |
| Duplex Purity | (non-denaturing) | NLT 90.0 area % | | 97.9 | 97.1 | 97.3 | 96.9 |
| Total Impurities ^{a} | | NMT 10.0 area % | | 2.1 | 2.9 | 2.6 | 3.0 |
| Assay (Free acid) | UV | 54-66 | mg/mL | 61 | 61 | 60 | 60 |
| | Spectrophotometry | | | | | | |
| Osmolality | USP <785>, Ph. | 210-390 | mOsm/kg | 292 | 294 | 293 | 294 |
| | Eur. 2.2.35 | | | | | | |
| pH | USP <791>, Ph. | 6.0-7.5 | | 6.8 | 6.7 | 6.7 | 6.7 |
| | Eur. 2.2.3 | | | | | | |
| Particulate Matter | Ph. Eur 2.9.19 | | | | | | |

| | | | | | | |
|---|---|---|---|---|---|---|
| ≥10 μm | USP <788> | NMT 6000 per container | 64 | 61 | 145 | 80 |
| ≥25 μm | | NMT 600 per container | 1 | 0 | 4 | 1 |

^{a} Sum of all other peaks ≥0.050 area %

TABLE 13
Stability Data: ALN-SOD DP Representative Lot Stored at 30° C. ± 2° C./75% ± 5%

| | | | | Storage Time (months) | | | |
|---|---|---|---|---|---|---|---|
| Attribute | Method | Acceptance Criterion | | 0 | 3 | 6 | 9 |
| Appearance | Visual Inspection | Clear, colorless to yellow solution, | | CSYS | CSYS | CSYS | CSYS |
| | | essentially free of foreign particles | | EFFP | EFFP | EFFP | EFFP |
| Purity | IPRP LC UV | | | | | | |
| Duplex Purity | (non-denaturing) | NLT 90.0 area % | | 97.9 | 97.1 | 97.4 | 96.9 |
| Total Impurities ^{a} | | NMT 10.0 area % | | 2.1 | 2.9 | 2.6 | 3.0 |
| Assay (Free acid) | UV | 54-66 | mg/mL | 61 | 61 | 60 | 60 |
| | Spectrophotometry | | | | | | |
| Osmolality | USP <785>, Ph. | 210-390 | mOsm/kg | 292 | 295 | 294 | 294 |
| | Eur. 2.2.35 | | | | | | |
| pH | USP <791>, Ph. | 6.0-7.5 | | 6.8 | 6.7 | 6.7 | 6.7 |
| | Eur. 2.2.3 | | | | | | |
| Particulate Matter | Ph. Eur 2.9.19 | | | | | | |

| | | | | | | |
|---|---|---|---|---|---|---|
| ≥10 μm | USP <788> | NMT 6000 per container | 64 | 227 | 221 | 162 |
| ≥25 μm | | NMT 600 per container | 1 | 0 | 9 | 1 |

^{a} Sum of all other peaks ≥0.050 area %

TABLE 14
Stability Data: ALN-SOD DP Representative Lot Stored at 40° C. ± 2° C./75% ± 5% RH

| | | | | Storage Time (months) | | | |
|---|---|---|---|---|---|---|---|
| Attribute | Method | Acceptance Criterion | | 0 | 1 | 3 | 6 |
| Appearance | Visual Inspection | Clear, colorless to yellow solution, | | CSYS | CSYS | CSYS | CSYS |
| | | essentially free of foreign particles | | EFFP | EFFP | EFFP | EFFP |
| Purity | IPRP LC UV | | | | | | |
| Duplex Purity | (non-denaturing) | NLT 90.0 area % | | 97.9 | 96.8 | 97.1 | 97.4 |
| Total Impurities ^{a} | | NMT 10.0 area % | | 2.1 | 3.1 | 2.9 | 2.6 |
| Assay (Free acid) | UV | 54-66 | mg/mL | 61 | 57 | 61 | 59 |
| | Spectrophotometry | | | | | | |
| Osmolality | USP <785>, Ph. | 210-390 | mOsm/kg | 292 | 294 | 297 | 293 |
| | Eur. 2.2.35 | | | | | | |
| pH | USP <791>, Ph. | 6.0-7.5 | | 6.8 | 6.7 | 6.7 | 6.7 |
| | Eur. 2.2.3 | | | | | | |
| Particulate Matter | Ph. Eur 2.9.19 | | | | | | |

| | | | | | | |
|---|---|---|---|---|---|---|
| ≥10 μm | USP <788> | NMT 6000 per container | 64 | 347 | 570 | 975 |
| ≥25 μm | | NMT 600 per container | 1 | 3 | 0 | 18 |

^{a} Sum of all other peaks ≥0.050 area %

### Example 14. Comparative HTT Formulation Stability

Stability testing of ALN-HTT DP (see formulation in Table 5) is conducted at various time points under the following conditions:
−20±5° C.
5±3° C.
25° C.±2° C./60%±5% relative humidity (RH)
30° C.±2° C./75%±5% relative humidity
40° C.±2° C./75%±5% relative humidity.

The DP is prepared with DS annealed with a 1-2% antisense strand excess.

The “mRNA” sequences of the Informal Sequence Listing and certain of the “mRNA target” sequences listed herein may be noted as reciting thymine (T) residues rather than uracil (U) residues. As is apparent to one of ordinary skill in the art, such sequences reciting “T” residues rather than “U” residues can be derived from NCBI accession records that list, as “mRNA” sequences, the DNA sequences (not RNA sequences) that directly correspond to mRNA sequences. Such DNA sequences that directly correspond to mRNA sequences technically constitute the DNA sequence that is the complement of the cDNA (complementary DNA) sequence for an indicated mRNA. Thus, while the mRNA target sequence does, in fact, actually include uracil (U) rather than thymine (T), the NCBI record-derived “mRNA” sequence includes thymine (T) residues rather than uracil (U) residues.

TABLE 15
Abbreviations of nucleotide monomers used in nucleic acid sequence
representation. It will be understood that these monomers, when present in an oligonucleotide,
are mutually linked by 3′->5′-phosphodiester bonds, unless otherwise noted; it is further
understood that the follow abbreviations represent the nucleotide omitting the 3′-phosphate when
placed at the 3′-terminal position of an oligonucleotide (i.e., the nucleotide is 3′-OH).

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-deoxy-2′-fluoroadenosine-3′-phosphate |
| Afs | 2′-deoxy-2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-deoxy-2′-fluorocytidine-3′-phosphate |
| Cfs | 2′-deoxy-2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-deoxy-2′-fluoroguanosine-3′-phosphate |
| Gfs | 2′-deoxy-2′-fluoroguanosine-3′-phosphorothioate |
| Gs | guanosine-3′-phosphorothioate |
| T | 5′-methyluridine-3′-phosphate |
| Tf | 2′-deoxy-2′-fluoro-5-methyluridine-3′-phosphate |
| Tfs | 2′-deoxy-2′-fluoro-5-methyluridine-3′-phosphorothioate |
| Ts | 5-methyluridine-3′-phosphorothioate |
| U | Uridine-3′-phosphate |
| Uf | 2′-deoxy-2′-fluorouridine-3′-phosphate |
| Ufs | 2′-deoxy-2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| CS | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| S | phosphorothioate linkage |
| Y34 | 2-hydroxymethyl-tetrahydrofuran-4-methoxy-3-phosphate (abasic 2′-OMe |
| | furanose) |
| Y44 | inverted abasic DNA (2-hydroxymethyl-tetrahydrofurane-5-phosphate) |
| (Agn) | Adenosine-glycol nucleic acid (GNA) S-Isomer |
| (Cgn) | Cytidine-glycol nucleic acid (GNA) S-Isomer |
| (Ggn) | Guanosine-glycol nucleic acid (GNA) S-Isomer |
| (Tgn) | Thymidine-glycol nucleic acid (GNA) S-Isomer |
| P | Phosphate |
| VP | Vinyl-phosphonate (e.g., 5-(E)-vinyl phosphonate) |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| (Ahd) | 2′-O-hexadecyl-adenosine-3′-phosphate |
| (Ahds) | 2′-O-hexadecyl-adenosine-3′-phosphorothioate |
| (Ghd) | 2′-O-hexadecyl-guanosine-3′-phosphate |
| (Ghds) | 2′-O-hexadecyl-guanosine-3′-phosphorothioate |
| (Chd) | 2′-O-hexadecyl-cytidine-3′-phosphate |
| (Chds) | 2′-O-hexadecyl-cytidine-3′-phosphorothioate |
| (Uhd) | 2′-O-hexadecyl-uridine-3′-phosphate |
| (Uhds) | 2′-O-hexadecyl-uridine-3′-phosphorothioate |
| (pshe) | Hydroxyethylphosphorothioate |
| (Cda) | 2′-O-docosanyl-cytidine-3′-phosphate 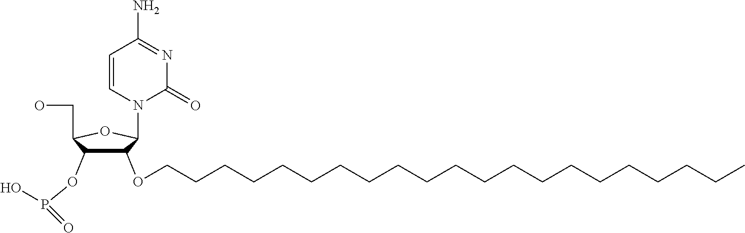 |
| (Ada) | 2′-O-docosanyl-adenosine-3′-phosphate |
| (Gda) | 2′-O-docosanyl-guanosine-3′-phosphate |
| (Uda) | 2′-O-docosanyl-uridine-3′-phosphate |
| (Cde) | 2′-O-decyl-cytidine-3′-phosphate 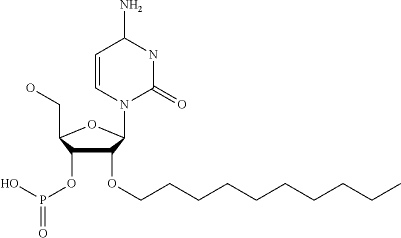 |
| (Ade) | 2′-O-decyl-adenosine-3′-phosphate |
| (Gde) | 2′-O-decyl-guanosine-3′-phosphate |
| (Ude) | 2′-O-decyl-uridine-3′-phosphate |
| (Ah) | 2′-O-hexyl-adenosine-3′-phosphate |
| (Ch) | 2′-O-hexyl-cytidine-3′-phosphate |
| (Gh) | 2′-O-hexyl-guanosine-3′-phosphate |
| (Uh) | 2′-O-hexyl-uridine-3′-phosphate |
| (A2p) | adenosine-2′-phosphate |
| (C2p) | cytidine-2′-phosphate |
| (G2p) | guanosine-2′-phosphate |
| (U2p) | uridine-2′-phosphate |

| | | SEQ | | SEQ | | SEQ |
|---|---|---|---|---|---|---|
| | Sense Strand Oligo | ID | Antisense Strand Oligo | ID | | ID |
| Agent | Sequence | NO: | Sequence | NO: | mRNA target sequence | NO: |
| AD- | csgscua(Chd)GfaAf | 1 | VPusUfsaggu(Tgn)ggauuu | 12 | ACGGCUACGAAAAUCCAACCUAA | 23 |
| 454973 | AfAfuccaaccusasa | | UfcGfuagccsgsu | | | |
| AD- | usasgup(Chd) AfuGf | 2 | VPusGfsagaa(Tgn)cuauuc | 13 | ACUAGUGCAUGAAUAGAUUCUCA | 24 |
| 454843 | AfAfuagauucuscsa | | AfuGfcacuascsu | | | |
| AD- | asassau(Chd)CfaAfC | 3 | VPusGfsaacu(Tgn)guaggu | 14 | CGAAAAUCCAACCUACAAGUUCA | 25 |
| 454844 | fCfuacaaguuscsa | | UfpGfauuuascsg | | | |
| AD- | gsgscua(Chd)gadAad | 4 | VPusUfsaggu(Tgn)ggaudT | 15 | ACGGCUACGAAAAUCCAACCUAA | 26 |
| 961583 | Auccaaccusasa | | uUfcdGuagccsgsu | | | |
| AD- | asasagag(Chd)gAfaA | 5 | VPuUfcugAfauagudTuUfg | 16 | AGAAAGAGCAAAACUAUUCAGAA | 27 |
| 961584 | fcuauucagsasa | | Cfucuuuscsu | | | |
| AD- | asasagag(Chd)adAad | 6 | VPuUfcugdAauagudTuUfg | 17 | AGAAAGAGCAAAACUAUUCAGAA | 28 |
| 961585 | Acuauucagsasa | | dCucuuuscsu | | | |
| AD- | ususuau(Ghd)AfuUfU | 7 | VPusGfsauaAfugagusaAf | 18 | CAUUUAUGAUUUACUCAUUAUCA | 29 |
| 961586 | fAfcucauuauscsa | | uCfauuaasusg | | | |
| AD- | asasaau(Chd)CfaAfC | 8 | VPusGfsaacu(Tgn)guagg | 19 | CGAAAAUCCAACCUACAAGULUCA | 30 |
| 454344 | fCfuacasguuscsa | | uUfgGfauuuuscsg | | | |
| AD- | (Ahds)asaaucCfaAfC | 0 | VPusGfssacu(Tgn)guagg | 20 | CGAAAAUCCAACCUACAAGUUCA | 31 |
| 1302922 | fCfuacaaguuscsa | | uUfgGfauuuuscsg | | | |
| AD- | asasaau(Cda)CfaAfC | 10 | VPusGfsaacu(Tgn)guagg | 21 | CGAAAAUCCAACCUACAAGUUCA | 32 |
| 1302923 | fCfuacaaguuscsa | | uUfgGfauuuuscsg | | | |
| AD- | asasaau(Cdc)CfaAfC | 11 | VPusGfsaacu(Tgn)guagg | 22 | CGAAAAUCCAACCUACAAGLUCA | 33 |
| 1999409 | fCfuacaaguuscsa | | uUfgGfauuuascsg | | | |

| | | SEQ | | SEQ | | SEQ |
|---|---|---|---|---|---|---|
| | Sense Strand Oligo | ID | Antisense Strand Oligo | ID | | ID |
| Agent | Sequence | NO: | Sequence | NO: | mRNA target sequence | NO: |
| AD- | GGCUACGAAAAUCCAACCUAA | 34 | UUAGGUTGGAUUUUCGUAGCCGU | 45 | ACGGCUACGAAAAUCCAACCUAA | 56 |
| 454973 | | | | | | |
| AD- | UAGUGCAUGAAUAGAUUCUCA | 35 | UGAGAATCUAUUCAUGCACUAGU | 46 | ACUAGUGCAUGAAUAGAUUCUCA | 57 |
| 454843 | | | | | | |
| AD- | AAAAUCCAACCUACAAGUUCA | 36 | UGAACUTGUAGGUUGGAUUUUCG | 47 | CGAAAAUCCAACCUACAAGUUCA | 58 |
| 454844 | | | | | | |
| AD- | GGCUACGAAAAUCCAACCUAA | 37 | UUAGGUTGGAUTUUCGUAGCCGU | 48 | ACGGCUACGAAAAUCCAACCUAA | 59 |
| 961583 | | | | | | |
| AD- | AAAGAGCAAAACUAUUCAGAA | 38 | UUCUGAAUAGUTUUGCUCUUUCU | 49 | AGAAAGAGCAAAACUAUUCAGAA | 60 |
| 961584 | | | | | | |
| AD- | AAAGAGCAAAACUAUUCAGAA | 39 | UUCUGAAUAGUTUUGCUCUUUCU | 50 | AGAAAGAGCAAAACUAUUCAGAA | 61 |
| 961585 | | | | | | |
| AD- | UUUAUGAUUUACUCAUUAUCA | 40 | UGAUAAUGAGUAAAUCAUAAAUG | 51 | CAUUUAUGAUUUACUCAUUAUCA | 62 |
| 961586 | | | | | | |
| AD- | AAAAUCCAACCUACAAGUUCA | 41 | UGAACUTGUAGGUUGGAUUUUCG | 52 | CGAAAAUCCAACCUACAAGUUCA | 63 |
| 454844 | | | | | | |
| AD- | AAAAUCCAACCUACAAGUUCA | 42 | UGAACUTGUAGGUUGGAUUUUCG | 53 | CGAAAAUCCAACCUACAAGUUCA | 64 |
| 1302922 | | | | | | |
| AD- | AAAAUCCAACCUACAAGUUCA | 43 | UGAACUTGUAGGUUGGAUUUUCG | 54 | CGAAAAUCCAACCUACAAGUUCA | 65 |
| 1302923 | | | | | | |
| AD- | AAAAUCCAACCUACAAGUUCA | 44 | UGAACUTGUAGGUUGGAUUUUCG | 55 | CGAAAAUCCAACCUACAAGUUCA | 66 |
| 1999409 | | | | | | |

TABLE 18
hsSOD1-Targeting and hsHTT Exon 1-Targeting iRNA Agents, Modified

| | | | SEQ | | SEQ | | SEQ |
|---|---|---|---|---|---|---|---|
| Target | | Modified | ID | Modified | ID | mRNA | ID |
| Gene | Duplex # | Sense Strand | NO: | Antisense Strand | NO: | target sequence | NO: |
| SOD1 | AD-1395718 | asuscaa(Uhd)uuC | 67 | VPusUfsccdTu(Ca | 86 | UCAUCAAUUUCGAGC | 105 |
| | | fGfAfccagaaggsa | | p)ugcucgAfaAfuu | | AGAAGGAA | |
| | | sa | | gausgsg | | | |
| SOD1 | AD-1395724 | asasuuu(Chd)gaG | 68 | VPusCfsuudTc(C2 | 87 | UCAAUUUCGAGCAGA | 106 |
| | | fCfAfgaaggaaasg | | p)uucugcUfcGfaa | | AGGAAAGU | |
| | | sa | | auusgsg | | | |
| SOD1 | AD-1395731 | ususcgag(Chd)aG | 69 | VPusUfsuadCu(Tg | 88 | AUUUCGAGCAGAAGG | 107 |
| | | fAfAfggnaagunsa | | n)uccuucUfgCfuc | | AAAGUAAU | |
| | | sa | | gaasasu | | | |
| SOD1 | AD-1395738 | asasgga(Ahd)agU | 70 | VPusdAscudGg(Tg | 89 | AGAAGGAAAGUAAUG | 108 |
| | | fAfAfugcaccagsu | | n)ccaudTaCfuuuc | | GACCAGUG | |
| | | sa | | cuuscsu | | | |
| SOD1 | AD-1395713 | gsasaag(Uhd)aaU | 71 | VPusUfsucdAc(Tg | 90 | AGGAAAGUAAUGGAC | 109 |
| | | fGfGfaccagugasa | | n)gguccaUfuAfcu | | CAGUGAAG | |
| | | sa | | uucscsu | | | |
| SOD1 | AD-1395756 | csasggu(Chd)uuC | 73 | VPusdAsggdAudTa | 91 | UGCAGGUCCUCACUU | 110 |
| | | fAfCfuuuaauccsu | | aagdTgAfggaccug | | UAAUCCUC | |
| | | sa | | scsg | | | |
| SOD1 | AD-1395760 | cscsuca(Chd)uuU | 73 | VPusdGsaudAg(Ag | 92 | GUCCUCACUUUAAUC | 111 |
| | | fAfAfuccncuausc | | n)ggaudTaAfagug | | CUCUAUCC | |
| | | sa | | aggsasc | | | |
| SOD1 | AD-1395762 | csascuu(Und)aaU | 74 | VPusdCsugdGadTa | 93 | CUCACTUUAAUCCUC | 112 |
| | | fCfCfucuauccasg | | gagdGaUfuuuaguc | | UAUCCAGA | |
| | | sa | | sasg | | | |
| SOD1 | AD-1395764 | asasgga(Uhd)gaA | 75 | VPusAfscadTc(C2 | 94 | CAAAGGAUGAAGAGA | 113 |
| | | fGfAfgaggcaugsu | | p)cucucuUfcAfcc | | GGCAUGUU | |
| | | sa | | uususg | | | |
| SOD1 | AD-1395771 | asgsga(Uhd)gaaG | 76 | VPusAfsacdAu(G2 | 95 | AAAGGAUGAAGAGAG | 114 |
| | | fAfGfaggcaugusu | | p)ccucucUfuCfau | | GCAUGUUG | |
| | | sa | | ccususu | | | |
| HTT | AD-1019448 | cscsaga(Ghd)Cfc | 77 | VPusGfsccaAfuGf | 96 | GCCCAGAGCCCCAUU | 115 |
| | | CfCfAfuucauugcs | | AfauggGfgCfucug | | CAUUGCCC | |
| | | csa | | csgsc | | | |
| HTT | AD-1019465 | cscsaug(Ghd)Cfg | 78 | VPusgfsuuwUfcCf | 97 | CGCCAUGGCGACCCU | 116 |
| | | AfCfCfcuggaaaas | | AfggguCfpCfcaug | | GGAAAAGC | |
| | | gsa | | gscsg | | | |
| HTT | AD-1271082 | Usgsgua(Ahd)Afu | 79 | VPasGhsgccu(Tgn | 98 | CCUGGAAAAGCUGAU | 117 |
| | | CHUfGfaugaaggcs | | )caucagCfuUfuuc | | GAAGGCCU | |
| | | csa | | casgsg | | | |
| HTT | AD-1271083 | uscsaua(Ahd)Ufc | 80 | VPasAfscaaAfcGf | 99 | AUUCAUAAUCACAUU | 118 |
| | | AfCfAfuucguuugs | | AfauguGfaUfuaug | | CGUUUGUU | |
| | | usa | | asasu | | | |
| HTT | AD-1271084 | gscsugc(Uhd)Gfa | 81 | VPusCfsaggAfaUf | 100 | GAGCUGGUGAAUCGG | 119 |
| | | AfUfCfggaunccus | | CfcgauUfcAfccag | | AUUCCUGC | |
| | | csa | | csusc | | | |
| HTT | AD-1272085 | gscsuau(Uhd)Cfa | 82 | VPusCfsgaaUfaUf | 101 | AUGCUAUUCAVAAUC | 120 |
| | | UfAfAfucacauucs | | GfauuaUfgAfauag | | ACAUUCGU | |
| | | gsa | | csasu | | | |
| HIT | AD-1498524 | gscsgac(Chd)Cfu | 83 | VPusAfsucdAg(C2 | 102 | UGGCGACCCUGGAAA | 121 |
| | | GfGfAfaaagcugas | | p)uuuudCcAfgdGg | | AGCUGAUG | |
| | | usa | | ucgcscsg | | | |
| HTT | AD-1498526 | csusucc(Ahd)Gru | 84 | VPusAfsggac(Tgn | 103 | GCCUUCGAGUCCCUC | 122 |
| | | CfCfCfucaaguccs | | )ugagggAfcUfcga | | AAGUCCUU | |
| | | usa | | agsgsc | | | |
| HIT | AD-1498528 | cscsggu(Chd)Afg | 85 | VPasUfsaadAadGc | 104 | GGCCGCUCAGGUUCU | 123 |
| | | GfUfUfcugcuuuus | | agaacCfudGaccgg | | GCUUUUAC | |
| | | asa | | scsc | | | |

| | | | SEQ | | SEQ ID |
|---|---|---|---|---|---|
| target | Duplex Name | transSeq Sense | ID NO: | transSeq Antisense | NO: |
| SOD1 | AD-1395718 | AUCAAUUUCGAGCAGAAGGAA | 124 | UUCCTUCUGCUCGAAAUUGAUGG | 143 |
| SOD1 | AD-1395724 | AAUUUCGAGCAGAAGGAAAGA | 125 | UCUUTCCUUCUGCUCGAAAUUGG | 144 |
| SOD1 | AD-1395731 | UUCGAGCAGAAGGAAAGUAAA | 126 | UUUACUTUCCUUCUGCUCGAAAU | 145 |
| SOD1 | AD-1395738 | AAGGAAAGUAAUGGACCAGUA | 127 | UACUGGTCCAUTACUUUCCUUCU | 146 |
| SOD1 | AD-1395743 | GAAAGUAAUGGACCAGUGAAA | 128 | UUUCACTGGUCCAUUACUUUCCU | 147 |
| SOD1 | AD-1395756 | CAGGUCCUCACUUUAAUCCUA | 129 | UAGGAUTAAAGTGAGGACCUGCG | 148 |
| SOD1 | AD-1395760 | CCUCACUUUAAUCCUCUAUCA | 130 | UGAUAGAGGAUTAAAGUGAGGAC | 149 |
| SOD1 | AD-1395762 | CACUUUAAUCCUCUAUCCAGA | 131 | UCUGGATAGAGGAUUAAAGUGAG | 150 |
| SOD1 | AD-1395764 | AAGGAUGAAGAGAGGCAUGUA | 132 | UACATGCCUCUCUUCAUCCUUUG | 151 |
| SOD1 | AD-1395771 | AGGAUGAAGAGAGGCAUGUUA | 133 | UAACAUGCCUCUCUUCAUCCUUU | 152 |
| HTT | AD-1019448 | CCAGAGCCCCAUUCAUUGCCA | 134 | UGGCAAUGAAUGGGGCUCUGGGG | 153 |
| HTT | AD-1019465 | CCAUGGCGACCCUGGAAAAGA | 135 | UCUUUUCCAGGGUCGCCAUGGCG | 154 |
| HTT | AD-1271082 | UGGAAAAGCUGAUGAAGGCCA | 136 | UGGCCUTCAUCAGCUUUUCCAGG | 155 |
| HTT | AD-1271083 | UCAUAAUCACAUUCGUUUGUA | 137 | UACAAACGAAUGUGAUUAUGAAU | 156 |
| IITT | AD-1271084 | GCUGGUGAAUCGGAUUCCUGA | 138 | UCAGGAAUCCGAUUCACCAGCUC | 157 |
| HTT | AD-1271085 | GCUAUUCAUAAUCACAUUCGA | 139 | UCGAAUGUGAUUAUGAAUAGCAU | 158 |
| HTT | AD-1498524 | GCGACCCUGGAAAAGCUGAUA | 140 | UAUCAGCUUUUCCAGGGUCGCCG | 159 |
| HTT | AD-1498526 | CUUCGAGUCCCUCAAGUCCUA | 141 | UAGGACTUGAGGGACUCGAAGGC | 160 |
| HTT | AD-1498528 | CCGGUCAGGUUCUGCUUUUAA | 142 | UUAAAAGCAGAACCUGACCGGCC | 161 |

### EQUIVALENTS

Those skilled in the art will recognize or be able to ascertain using no more than routine experimentation, many equivalents to the specific embodiments and methods described herein. Such equivalents are intended to be encompassed by the scope of the following claims.

1. A composition comprising
(a) a double-stranded ribonucleic acid (dsRNA) comprising a sense strand and an antisense strand, wherein one of the sense strand or the antisense strand of the dsRNA comprises at least one lipophilic modification and the other strand of the dsRNA does not comprise a lipophilic modification; and
a divalent ion source,
wherein:
(i) substantially all of the sense strand or the antisense strand of the dsRNA comprising the at least one lipophilic modification in the composition is duplexed with the strand that does not comprise a lipophilic modification, or
(ii) the sense strand or the antisense strand of the dsRNA comprising the at least one lipophilic modification is present at less than 1% molar excess relative to the strand that does not comprise a lipophilic modification, the sense strand and the antisense strand are present at molar equivalence, or the strand that does not comprise a lipophilic modification is present in a molar excess relative to the strand of the dsRNA comprising the at least one lipophilic modification; or
(b) a double-stranded ribonucleic acid (dsRNA) comprising a sense strand and an antisense strand, wherein one of the sense strand or the antisense strand of the dsRNA comprises at least one lipophilic modification and the other strand of the dsRNA does not comprise a lipophilic modification; and a divalent ion source,
wherein:
the sense strand or the antisense strand of the dsRNA that comprises at least one lipophilic modification is present at less than 1% molar excess relative to the strand of the dsRNA that does not comprise a lipophilic modification, the strand of the dsRNA that does not comprise a lipophilic modification is present at molar equivalence to the sense strand or the antisense strand of the dsRNA that comprises at least one lipophilic modification, or the strand of the dsRNA that does not comprise a lipophilic modification is present in molar excess relative to the sense strand or the antisense strand of the dsRNA that comprises at least one lipophilic modification.

2. (canceled)

3. The composition of claim 1, wherein the composition is substantially free of inorganic phosphate.

4. The composition of claim 1, wherein the sense strand of the dsRNA comprises at least one lipophilic modification and the antisense strand of the dsRNA does not comprise a lipophilic modification.

5. The composition of claim 1, wherein
(a) the composition comprises a molar excess of the antisense strand relative to the sense strand; and/or
(b) the composition comprises a molar excess of the antisense strand relative to the sense strand, wherein at least a 0.1% molar excess of the antisense strand relative to the sense strand, optionally wherein there is at least a 0.2% molar excess of the antisense strand relative to the sense strand, optionally wherein there is at least a 0.3% molar excess of the antisense strand relative to the sense strand, optionally wherein there is at least a 0.4% molar excess of the antisense strand relative to the sense strand, optionally wherein there is at least a 0.5% molar excess of the antisense strand relative to the sense strand, optionally wherein there is at least a 1% molar excess of the antisense strand relative to the sense strand, optionally wherein there is at least a 2% molar excess of the antisense strand relative to the sense strand, optionally wherein there is at least a 3% molar excess of the antisense strand relative to the sense strand, optionally wherein there is at least a 4% molar excess of the antisense strand relative to the sense strand, optionally wherein there is about a 5% or greater molar excess of the antisense strand relative to the sense strand.

6. (canceled)

7. The composition of claim 1, wherein
(a) the antisense strand of the dsRNA comprises the at least one lipophilic modification and the sense strand of the dsRNA does not comprise a lipophilic modification, optionally wherein the antisense strand of the dsRNA comprises the at least one lipophilic modification, optionally wherein the composition comprises a molar excess of the sense strand relative to the antisense strand; and/or
(b) the composition comprises a molar excess of the sense strand relative to the antisense strand, wherein there is at least a 0.1% molar excess of the sense strand relative to the antisense strand, optionally wherein there is at least a 0.2% molar excess of the sense strand relative to the antisense strand, optionally wherein there is at least a 0.3% molar excess of the sense strand relative to the antisense strand, optionally wherein there is at least a 0.4% molar excess of the sense strand relative to the antisense strand, optionally wherein there is at least a 0.5% molar excess of the sense strand relative to the antisense strand, optionally wherein there is at least a 1% molar excess of the sense strand relative to the antisense strand, optionally wherein there is at least a 2% molar excess of the sense strand relative to the antisense strand, optionally wherein there is at least a 3% molar excess of the sense strand relative to the antisense strand, optionally wherein there is at least a 4% molar excess of the sense strand relative to the antisense strand, optionally wherein there is about a 5% or greater molar excess of the sense strand relative to the antisense strand.

8. (canceled)

9. The composition of claim 1, wherein
(a) the composition is formulated for a route of administration selected from the group consisting of intravenous, subcutaneous, intramuscular, intradermal, intra-articular and intrathecal,
(b) the lipophilic modification is a saturated or unsaturated C₄-C₃₀ hydrocarbon, optionally a C₄-C₃₀ alkyl or alkenyl, optionally a linear C₆-C₁₈ alkyl or alkenyl, optionally a C₁₆ alkyl, optionally wherein the lipophilic modification is attached at the 2′-ribo position of a nucleic acid residue of the dsRNA;
(c) the divalent ion source is selected from the group consisting of magnesium, calcium, copper, nickel, zinc, and strontium;
(d) the molar ratio of the divalent ion-to-the dsRNA is greater than about 2:1;
(e) the composition is substantially free of inorganic phosphate and/or comprises less than 100 ppm of inorganic phosphate, optionally less than 50 ppm of inorganic phosphate, optionally less than 10 ppm of inorganic phosphate, optionally less than 5 ppm of inorganic phosphate, optionally wherein the composition does not comprise inorganic phosphate;
(f) the molar ratio of the divalent ion source-to-the-dsRNA is greater than about 2.5:1, optionally greater than about 3.0:1, optionally greater than about 3.5:1, optionally greater than about 4.0:1; and/or
(g) the molar ratio of the divalent ion source-to-the-dsRNA is between about 2:1 and about 10:1, optionally between about 3:1 and about 10:1, optionally between about 3:1 and about 9:1, optionally between about 3:1 and about 8:1, optionally between about 3:1 and about 7:1, optionally between about 3:1 and about 6:1, optionally between about 3:1 and about 5:1.

10.-15. (canceled)

16. The composition of claim 1, wherein the dsRNA comprises a 5′-phosphate or 5′-phosphate mimic modification.

17. The composition of claim 16, wherein
(a) the 5′-phosphate mimic modification is 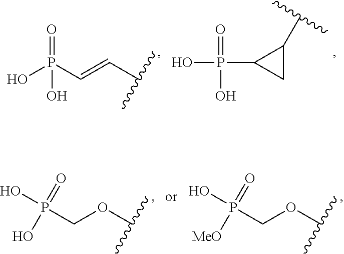 [SMILES: */C=C/P(=O)(O)O ; *C1CC1P(=O)(O)O ; *OCP(=O)(O)O ; *OCP(=O)(O)OC]
where the preceding structure replaces the 4′-CH₂OH group within the ribose ring of a 5′-terminal nucleotide;
(b) the dsRNA comprises a 5′-phosphate modification, wherein the phosphonate modification is a 5′-phosphonate modification:
(c) the dsRNA comprises a 5′-phosphate modification, wherein the phosphonate modification is a 5′-vinyl phosphonate modification, optionally a 5′-(E)-vinyl phosphonate modification; and/or
d) the dsRNA comprises a 5′-phosphate mimic modification, wherein the phosphate mimic modification is a 5′-vinyl phosphate modification.

18.-20. (canceled)

21. The composition of claim 1, further comprising a diluent.

22. The composition of claim 21, wherein the composition is isotonic to cerebrospinal fluid (CSF).

23. The composition of claim 22, further comprising a sodium source, a potassium source, a magnesium source, and a calcium source.

24. The composition of claim 23, comprising sodium chloride, magnesium chloride, potassium chloride, and calcium chloride.

25. The composition of claim 1, wherein
(a) the composition has a pH between about 4 and about 10, optionally wherein the pH is between about 6 and about 10, optionally wherein the pH is between about 6.5 and about 8.0;
(b) the composition has an osmolality between about 200 and 400 mOsm/kg;
(c) the composition does not comprise hydrogen phosphate or dihydrogen phosphate;
(d) the composition does not comprise a buffer;
(e) the composition further comprises a stabilizing agent selected from the group consisting of sucrose, glucose, mannitol, sorbitol, polyethylene glycol (PEG), histidine, arginine, lysine, phospholipids, trehalose and a combination thereof; and/or
(f) the composition comprises greater than 1 mg of the dsRNA per mL of the composition, optionally wherein the composition comprises greater than 5 mg of the dsRNA per mL of the composition, optionally wherein the composition comprises greater than 10 mg of the dsRNA per mL of the composition, optionally wherein the composition comprises greater than 25 mg of the dsRNA per mL of the composition, optionally wherein the composition comprises greater than 50 mg of the dsRNA per mL of the composition, optionally wherein the composition comprises about 60 mg of the dsRNA per mL of the composition.

26.-31. (canceled)

32. A composition comprising:
(a) a double-stranded ribonucleic acid (dsRNA) comprising a sense strand and an antisense strand, wherein one of the sense strand and antisense strand comprises at least one lipophilic modification,
and the composition comprises about 50 mg to about 70 mg of the dsRNA per mL of the composition;
(b) sodium chloride at about 80 mM to about 110 mM; and
(c) calcium chloride at about 8.0 mM to about 20.0 mM.

33. The composition of claim 32, wherein
(a) the composition is substantially free of inorganic phosphate and/or comprises less than 100 ppm of inorganic phosphate, optionally less than 50 ppm of inorganic phosphate, optionally less than 10 ppm of inorganic phosphate, optionally less than 5 ppm of inorganic phosphate, optionally wherein the composition does not comprise inorganic phosphate;
(b) the sense strand of the dsRNA comprises at least one lipophilic modification and the antisense strand of the dsRNA does not comprise a lipophilic modification;
(c) the composition comprises a molar excess of the antisense strand relative to the sense strand;
(d) the composition comprises a molar excess of the antisense strand relative to the sense strand, wherein there is at least a 0.1% molar excess of the antisense strand relative to the sense strand, optionally wherein there is at least a 0.2% molar excess of the antisense strand relative to the sense strand, optionally wherein there is at least a 0.3% molar excess of the antisense strand relative to the sense strand, optionally wherein there is at least a 0.4% molar excess of the antisense strand relative to the sense strand, optionally wherein there is at least a 0.5% molar excess of the antisense strand relative to the sense strand, optionally wherein there is at least a 1% molar excess of the antisense strand relative to the sense strand, optionally wherein there is at least a 2% molar excess of the antisense strand relative to the sense strand, optionally wherein there is at least a 3% molar excess of the antisense strand relative to the sense strand, optionally wherein there is at least a 4% molar excess of the antisense strand relative to the sense strand, optionally wherein there is about a 5% or greater molar excess of the antisense strand relative to the sense strand;
(e) the composition further comprises:
(1) potassium chloride at about 1.0 mM to about 2.5 mM; and
(2) magnesium chloride at about 0.1 mM to about 1.0 mM;
(f) the composition comprises about 60 mg of the dsRNA per mL of the composition;
(g) the composition comprises sodium chloride at about 97.6 mM;
(h) the composition comprises calcium chloride at about 13.0 mM;
(i) the composition comprises potassium chloride at about 1.9 mM;
(i) the composition comprises magnesium chloride at about 0.5 mM;
(k) the composition is a pharmaceutical composition for intrathecal administration of the dsRNA to a subject;
(l) the composition is a pharmaceutical composition for intrathecal administration of the dsRNA to a subject, wherein the subject is a mammal, optionally wherein the subject is human;
(m) the composition further comprises a diluent;
(n) the composition is isotonic to CSF;
(o) the composition has a pH between about 6 and about 10, optionally wherein the pH is between about 6.5 and about 8.0;
(p) the composition has an osmolality between about 200 and 400 mOsm/kg;
(q) the composition does not comprise hydrogen phosphate or dihydrogen phosphate;
(r) the composition does not comprise a buffer; and/or
(s) the composition further comprises a stabilizing agent selected from the group consisting of sucrose, glucose, mannitol, sorbitol, polyethylene glycol (PEG), histidine, arginine, lysine, phospholipids, trehalose and a combination thereof.

34.-66. (canceled)

67. A composition comprising a double-stranded ribonucleic acid (dsRNA) capable of annealing and reducing expression of an amyloid precursor protein (APP) mRNA, wherein the dsRNA comprises a sense strand and an antisense strand, wherein one of the sense strand or the antisense strand of the dsRNA comprises at least one lipophilic modification and the other strand of the dsRNA does not comprise a lipophilic modification; and wherein:
(i) substantially all of the sense strand or the antisense strand of the dsRNA comprising the at least one lipophilic modification in the composition is duplexed with the strand that does not comprise a lipophilic modification, or
(ii) the sense strand or the antisense strand of the dsRNA comprising the at least one lipophilic modification is present at less than 1% molar excess relative to the strand that does not comprise a lipophilic modification, the sense strand and the antisense strand are present at molar equivalence, or the strand that does not comprise a lipophilic modification is present in a molar excess relative to the strand of the dsRNA comprising the at least one lipophilic modification.

68.-69. (canceled)

70. A composition comprising a double-stranded ribonucleic acid (dsRNA) capable of annealing and reducing expression of a superoxide dismutase 1 (SOD1) mRNA, wherein the dsRNA comprises a sense strand and an antisense strand, wherein one of the sense strand or the antisense strand of the dsRNA comprises at least one lipophilic modification and the other strand of the dsRNA does not comprise a lipophilic modification; and wherein:
(i) substantially all of the sense strand or the antisense strand of the dsRNA comprising the at least one lipophilic modification in the composition is duplexed with the strand that does not comprise a lipophilic modification, or
(ii) the sense strand or the antisense strand of the dsRNA comprising the at least one lipophilic modification is present at less than 1% molar excess relative to the strand that does not comprise a lipophilic modification, the sense strand and the antisense strand are present at molar equivalence, or the strand that does not comprise a lipophilic modification is present in a molar excess relative to the strand of the dsRNA comprising the at least one lipophilic modification.

71.-72. (canceled)

73. A composition comprising a double-stranded ribonucleic acid (dsRNA) capable of annealing and reducing expression of a huntingtin (HTT) mRNA, wherein the dsRNA comprises a sense strand and an antisense strand, wherein one of the sense strand or the antisense strand of the dsRNA comprises at least one lipophilic modification and the other strand of the dsRNA does not comprise a lipophilic modification; and wherein:
(i) substantially all of the sense strand or the antisense strand of the dsRNA comprising the at least one lipophilic modification in the composition is duplexed with the strand that does not comprise a lipophilic modification, or
(ii) the sense strand or the antisense strand of the dsRNA comprising the at least one lipophilic modification is present at less than 1% molar excess relative to the strand that does not comprise a lipophilic modification, the sense strand and the antisense strand are present at molar equivalence, or the strand that does not comprise a lipophilic modification is present in a molar excess relative to the strand of the dsRNA comprising the at least one lipophilic modification.

74.-76. (canceled)

77. A composition comprising a double-stranded ribonucleic acid (dsRNA) comprising a sense strand and an antisense strand, wherein one of the sense strand or the antisense strand of the dsRNA comprises at least one lipophilic modification at one or more internal residue(s) of the sense or antisense strand and the other strand of the dsRNA does not comprise a lipophilic modification; and wherein:
(i) substantially all of the sense strand or the antisense strand of the dsRNA comprising the at least one lipophilic modification in the composition is duplexed with the strand that does not comprise a lipophilic modification, or
(ii) the sense strand or the antisense strand of the dsRNA comprising the at least one lipophilic modification is present at less than 1% molar excess relative to the strand that does not comprise a lipophilic modification, the sense strand and the antisense strand are present at molar equivalence, or the strand that does not comprise a lipophilic modification is present in a molar excess relative to the strand of the dsRNA comprising the at least one lipophilic modification.

78. The composition of claim 77, wherein
(a) the composition further comprises a divalent ion source,
(b) the dsRNA comprises at least one lipophilic modification at one or more internal residue(s) of the sense strand, optionally wherein the dsRNA comprises at least one lipophilic modification at any one of positions 4-8 or 13-18 counting from the 5′-end of the strand, optionally wherein the dsRNA comprises at least one lipophilic modification at position 6 counting from the 5′-end of the strand:
(c) the dsRNA comprises at least one lipophilic modification at one or more internal residue(s) of the antisense strand; and/or
(d) the dsRNA comprises at least one lipophilic modification at one or more internal residue(s) of the antisense strand, wherein the at least one lipophilic modification comprises a saturated or unsaturated C₄-C₃₀ hydrocarbon, optionally a C₄-C₃₀ alkyl or alkenyl, optionally a linear C₆-C₁₈ alkyl or alkenyl, optionally a C₁₆ alkyl, optionally wherein the at least one lipophilic modification is attached at the 2′-ribo position of a nucleic acid residue of the dsRNA.

79.-81. (canceled)

82. A composition comprising a double-stranded ribonucleic acid (dsRNA) comprising a sense strand and an antisense strand, wherein one of the sense strand or the antisense strand of the dsRNA comprises at least one lipophilic modification at one or more terminal residue(s) of the sense or antisense strand and the other strand of the dsRNA does not comprise a lipophilic modification; and wherein:
(a) substantially all of the sense strand or the antisense strand of the dsRNA comprising the at least one lipophilic modification in the composition is duplexed with the strand that does not comprise a lipophilic modification, or
(b) the sense strand or the antisense strand of the dsRNA comprising the at least one lipophilic modification is present at less than 1% molar excess relative to the strand that does not comprise a lipophilic modification, the sense strand and the antisense strand are present at molar equivalence, or the strand that does not comprise a lipophilic modification is present in a molar excess relative to the strand of the dsRNA comprising the at least one lipophilic modification.

83. The composition of claim 82, wherein
(a) the composition further comprises a divalent ion source;
(b) the dsRNA comprises at least one lipophilic modification at the 5′-terminal and/or the 3′-terminal residue(s) of the sense strand, optionally wherein the dsRNA comprises at least one lipophilic modification at the 3′-terminal residue of the sense strand;
(c) the dsRNA comprises at least one lipophilic modification at the 5′-terminal residue of the sense strand;
(d) the dsRNA comprises at least one lipophilic modification at the 5′-terminal and/or the 3′-terminal residue(s) of the antisense strand, optionally wherein the dsRNA comprises at least one lipophilic modification at the 3′-terminal residue of the antisense strand;
(e) the dsRNA comprises at least one lipophilic modification at the 5′-terminal residue of the antisense strand; and/or
(f) the dsRNA comprises at least one lipophilic modification at the 5′-terminal and/or the 3′-terminal residue(s) of the sense strand and/or the antisense strand, wherein the at least one lipophilic modification comprises a saturated or unsaturated C₄-C₃₀ hydrocarbon, optionally a C₄-C₃₀ alkyl or alkenyl, optionally a linear C₆-C₁₈ alkyl or alkenyl, optionally a C₁₆ alkyl, optionally wherein the at least one lipophilic modification is attached at the 2′-ribo position of a nucleic acid residue of the dsRNA.

84.-88. (canceled)

89. A solid prepared by lyophilization of the composition of claim 1.

90. A kit comprising:
(a) a diluent comprising a divalent cation source; and
(b) a double-stranded ribonucleic acid (dsRNA) comprising a sense strand and an antisense strand, wherein the dsRNA comprises at least one modified nucleotide that is not a 2′-deoxynucleotide,
wherein the molar ratio of the divalent cation source to the dsRNA is greater than 2 to 1.

91.-93. (canceled)

94. A method of treating a subject having a disorder that would benefit from a reduction in expression of a target gene, the method comprising administering to the subject a therapeutically effective amount of a composition of claim 1, thereby treating said subject.

95.-104. (canceled)

105. A method
(a) of inhibiting the expression of APP in a subject, the method comprising:
administering to said subject a therapeutically effective amount of the composition of claim 67, thereby inhibiting the expression of APP in said subject; or
(b) of treating or preventing an APP-associated disease or disorder in a subject, the method comprising
administering to said subject a therapeutically effective amount of the composition of claim 67, thereby treating or preventing an APP-associated disease or disorder in the subject.

106.-112. (canceled)

113. A method of administering a dsRNA to a subject in need thereof, the method comprising administering the composition of claim 1 intrathecally to the subject, thereby administering the dsRNA to said subject.

114.-116. (canceled)

117. A method of inhibiting the expression of SOD1 in a cell or tissue of a subject, the method comprising administering the composition of claim 70 to the subject in an amount sufficient to reduce SOD1 expression in the cell or tissue of the subject, thereby inhibiting the expression of SOD1 in the cell or tissue of the subject.

118.-121. (canceled)

122. A method of inhibiting the expression of HTT in a cell or tissue of a subject, the method comprising administering the composition of claim 73 to the subject in an amount sufficient to reduce HTT expression in the cell or tissue of the subject, thereby inhibiting the expression of HTT in the cell or tissue of the subject.

123-128. (canceled)

129. A method of
(a)reducing or preventing particle formation in a solution comprising a divalent ion source and a double-stranded ribonucleic acid (dsRNA) having a sense strand and an antisense strand, wherein the dsRNA comprises at least one lipophilic modification of the sense strand or the antisense strand, and either the sense or the antisense strand does not comprise a lipophilic modification, the method comprising maintaining the sense strand or the antisense strand of the dsRNA comprising the lipophilic modification at less than 1% molar excess relative to the strand that does not comprise a lipophilic modification, thereby reducing or preventing particle formation in the solution comprising the divalent ion source and the double-stranded ribonucleic acid (dsRNA); or
(b) preparing a formulation comprising
annealing a sense strand and an antisense strand, wherein one of the sense strand and antisense strand contains a lipophilic modification, to form a duplex solution comprising a double stranded RNA (dsRNA);
lyophilizing the duplex solution to provide a duplex composition; and
dissolving the duplex composition in an injection solution, wherein
the injection solution comprises a divalent cation source (e.g., calcium) and does not comprise a phosphate buffer;
and the duplex composition comprises 0-5% molar excess (e.g., about 1-2% molar excess) of antisense strand over sense strand.

130.-141. (canceled)
